(19) United States
(12) Patent Application Publication
(10) Pub. No.: US 2017/0265474 A1
(43) Pub. Date: Sep. 21, 2017

(54) BICYCLIC COMPOUNDS AS PEST CONTROL AGENTS

(71) Applicants: ALEXANDER ARLT, KOELN (DE); MARTIN FUESSLEIN, DUESSELDORF (DE); SILVIA CEREZO-GALVEZ, LANGENFELD (DE); ARND VOERSTE, KOELN (DE); THOMAS BRETSCHNEIDER, LOHMAR (DE); REINER FISCHER, MONHEIM (DE); PETER JESCHKE, BERGISCH GLADBACH (DE); EIKE KEVIN HEILMANN, Düesseldorf (DE); KERSTIN ILG, KOELN (DE); OLGA MALSAM, ROESRATH (DE); PETER LOESEL, LEVERKUSEN (DE)

(72) Inventors: ALEXANDER ARLT, KOELN (DE); MARTIN FUESSLEIN, DUESSELDORF (DE); SILVIA CEREZO-GALVEZ, LANGENFELD (DE); ARND VOERSTE, KOELN (DE); THOMAS BRETSCHNEIDER, LOHMAR (DE); REINER FISCHER, MONHEIM (DE); PETER JESCHKE, BERGISCH GLADBACH (DE); EIKE KEVIN HEILMANN, Düesseldorf (DE); KERSTIN ILG, KOELN (DE); OLGA MALSAM, ROESRATH (DE); PETER LOESEL, LEVERKUSEN (DE)

(73) Assignee: BAYER CROPSCIENCE AKTIENGESELLSCHAFT, MONHEIM AM RHEIN (DE)

(21) Appl. No.: 15/531,893

(22) PCT Filed: Dec. 1, 2015
(86) PCT No.: PCT/EP2015/078159

§ 371 Date: May 31, 2017

(30) Foreign Application Priority Data
Dec. 2, 2014 (EP) 14195953.6

(51) Int. Cl. A01N 43/90; C07D 263/52; C07D 277/60; C07D 521/00

(52) U.S. Cl. CPC A01N 43/90; C07D 521/00; C07D 263/52; C07D 277/60

(57) ABSTRACT

The present application relates to novel bicyclic compounds, to compositions comprising these compounds, to their use for controlling animal pests and to processes and intermediates for their preparation.

The present application relates to novel bicyclic compounds, to compositions comprising these compounds, to their use for controlling animal pests and to processes and intermediates for their preparation.

Recently, bicyclic compounds having insecticidal properties have been disclosed (WO 2015/038503 A1).

WO 2010/051196 and WO 2009/155017 describe aryl- or heteroaryl-substituted azabenzoxazoles and azabenzthiazoles for pharmaceutical and diagnostic applications.

U.S. Pat. No. 4,038,396 reports the synthesis of various thiazolo[5,4-b]pyridines and oxazolo[5,4-b]pyridines for pharmaceutical applications.

Modern crop protection compositions have to meet many demands, for example in relation to extent, persistence and spectrum of their action and possible use. Questions of toxicity and of combinability with other active compounds or formulation auxiliaries play a role, as does the question of the expense that the synthesis of an active compound requires. In addition, resistances can occur. For all these reasons alone, the search for novel crop protection compositions cannot be considered complete, and there is a constant need for novel compounds having improved properties compared to the known compounds, at least in relation to individual aspects.

It was an object of the present invention to provide compounds which widen the spectrum of the pesticides under various aspects.

This object, and further objects which are not stated explicitly but can be discerned or derived from the connections discussed herein, are achieved by novel compounds of the formula (I)

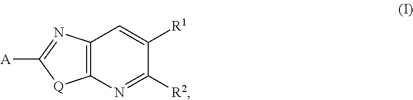

in which
A represents an A radical from the group consisting of (A-b) to (A-f)

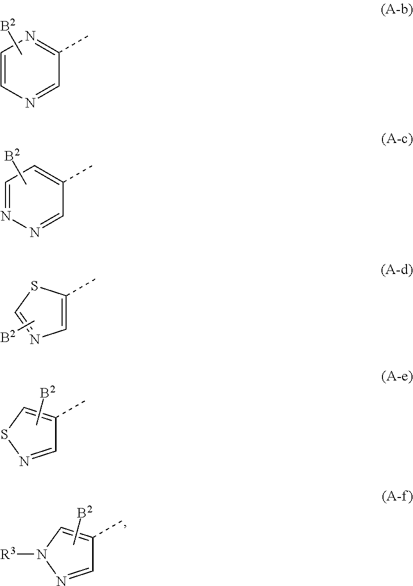

where the broken line denotes the bond to the carbon atom of the bicyclic system of the formula (I) and
B² represents a radical from the group consisting of hydrogen, halogen, cyano, nitro, alkyl, haloalkyl, alkoxy, haloalkoxy and in each case optionally substituted cycloalkyl and cycloalkenyl,
Q represents oxygen or sulfur,
R¹ represents a radical from the group consisting of hydrogen, alkyl, alkoxy and cyano,
R² a) represents a B radical from the group consisting of

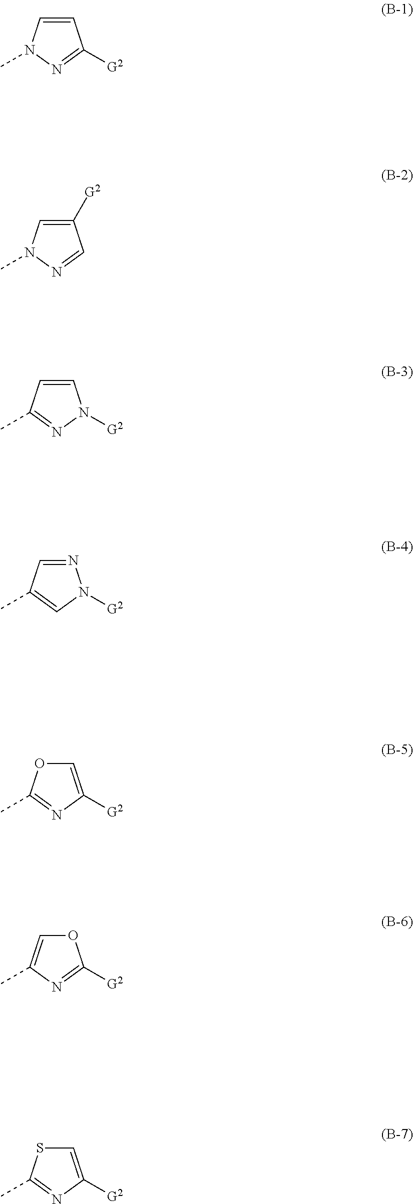

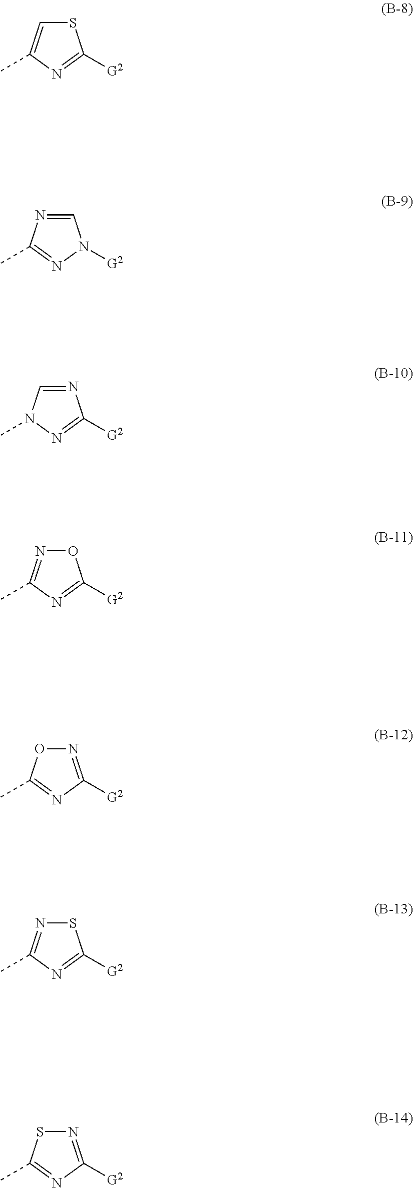

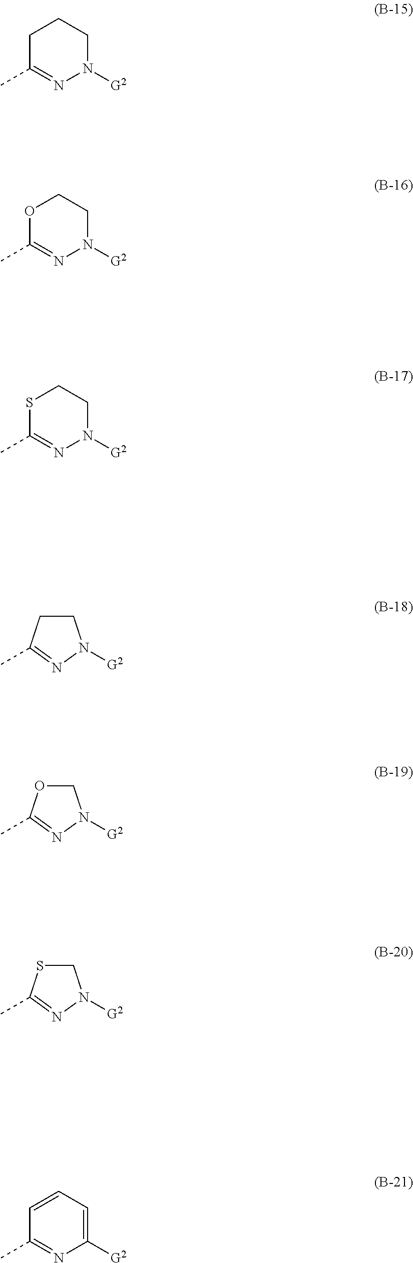

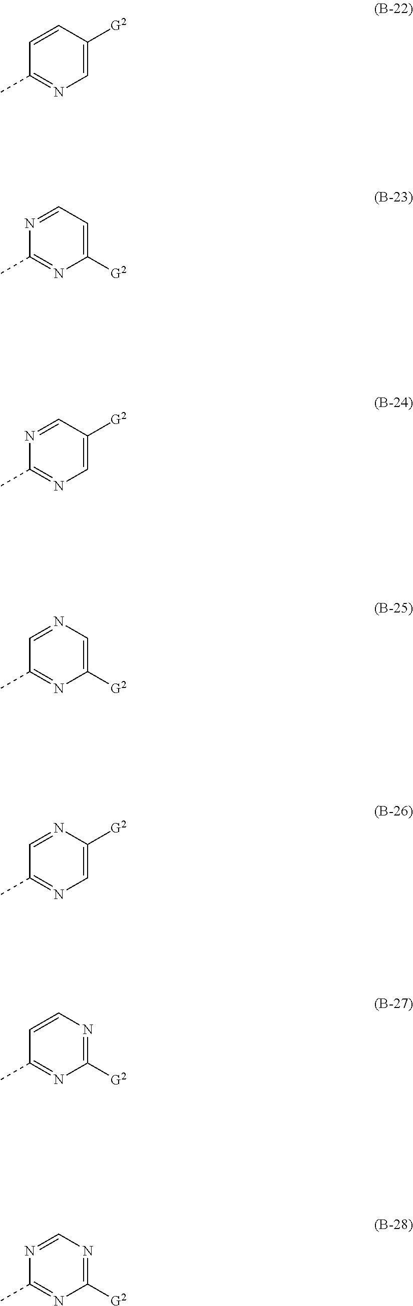

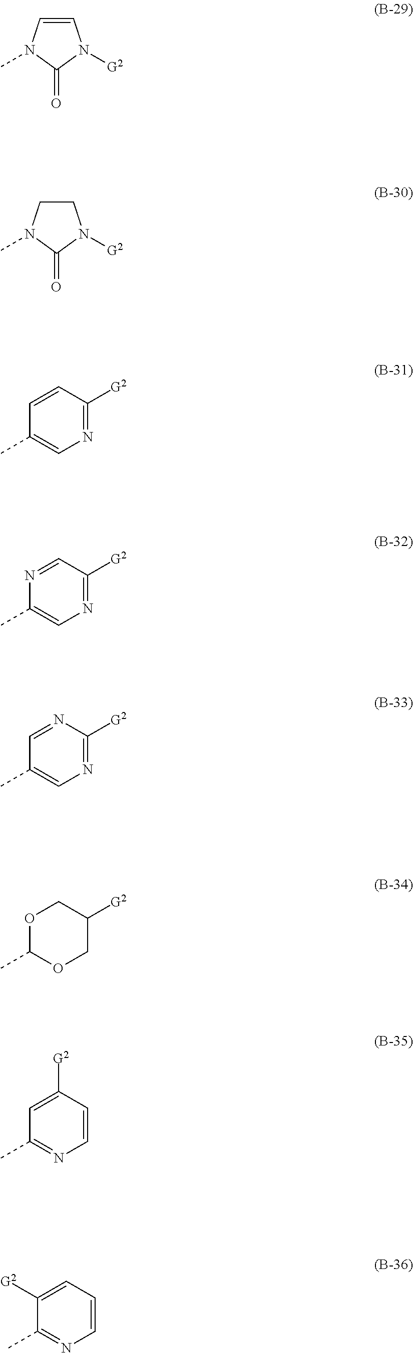

where the broken line denotes the bond to the carbon atom of the bicyclic system of the formula (I), or
R² b) is a D radical from the group consisting of (D-1) to (D-3)

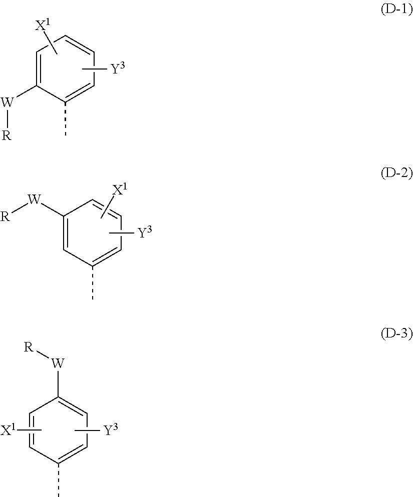

where the broken line denotes the bond to the carbon atom of the bicyclic system of the formula (I), or
R² c) represents a radical of the formula

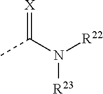

where the broken line denotes the bond to the carbon atom of the bicyclic system of the formula (I), or
R² d) represents a radical of the formula

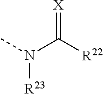

where the broken line denotes the bond to the carbon atom of the bicyclic system of the formula (I), or
R² e) represents an F radical from the group consisting of (F-1) to (F-11)

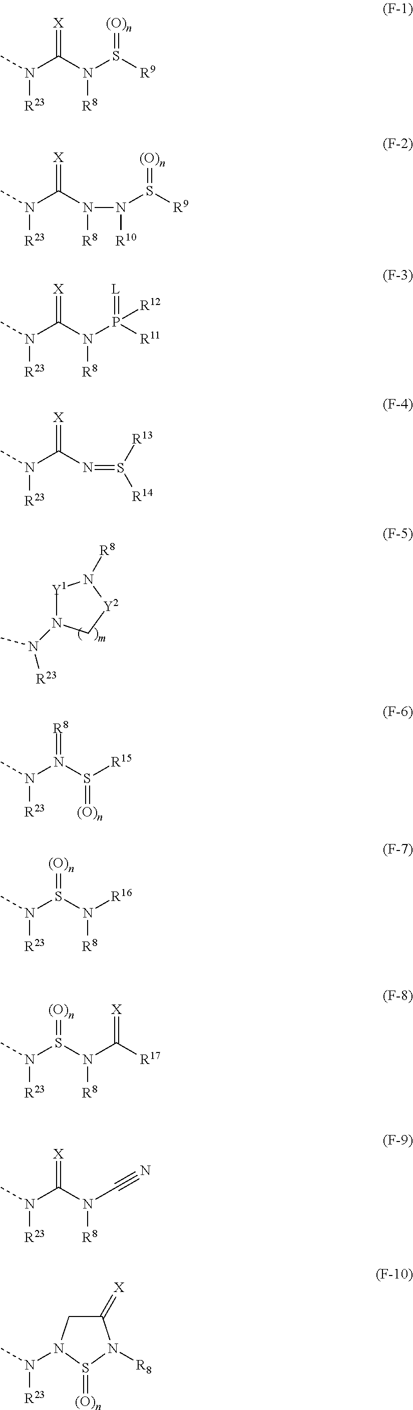

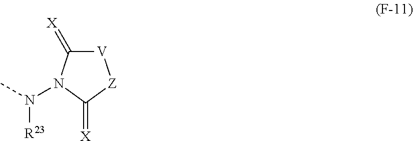

where the broken line denotes the bond to the carbon atom of the bicyclic system of the formula (I), or
R² f) represents a radical from the group consisting of haloalkyl and carboxyl,
R² g) represents a radical of the formula

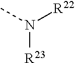

where the broken line denotes the bond to the carbon atom of the bicyclic system of the formula (I),
in which
G² represents hydrogen or a radical from the group consisting of halogen, nitro, amino, cyano, alkylamino, haloalkylamino, dialkylamino, alkyl, haloalkyl, saturated or unsaturated cycloalkyl which is optionally substituted and optionally interrupted by one or more heteroatoms, cycloalkylalkyl, alkoxy, haloalkoxy, alkoxyalkyl, halogenated alkoxyalkyl, alkylthioalkyl, alkylsulfinylalkyl, alkylsulfonylalkyl, bis(alkoxy)alkyl, bis(haloalkoxy)alkyl, alkoxy(alkylsulfanyl)alkyl, alkoxy(alkylsulfinyl)alkyl, alkoxy(alkylsulfonyl)alkyl, bis(alkylsulfanyl)alkyl, bis(haloalkylsulfanyl)alkyl, bis(hydroxyalkylsulfanyl)alkyl, alkoxycarbonyl, alkoxycarbonylalkyl, alpha-hydroxyiminoalkoxycarbonylalkyl, alpha-alkoxyiminoalkoxycarbonylalkyl, C(X²)NR³R⁴, NR⁶R⁷, alkylthio, alkylsulfinyl, alkylsulfonyl, haloalkylthio, haloalkylsulfinyl, haloalkylsulfonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by alkyl, haloalkyl, alkoxy and alkoxyalkyl), phenyl (which for its part may be substituted by halogen, cyano, nitro, alkyl and haloalkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, alkyl, haloalkyl, alkoxy, haloalkoxy, alkoxyalkyl, alkylthio, alkylthioalkyl and cycloalkyl) and the heteroarylalkyl radicals triazolylalkyl, pyridylalkyl, pyrimidylalkyl and oxadiazolylalkyl (which for their part may be substituted by halogen and alkyl), or
G² represents a C radical from the group consisting of (C-1) to (C-9)

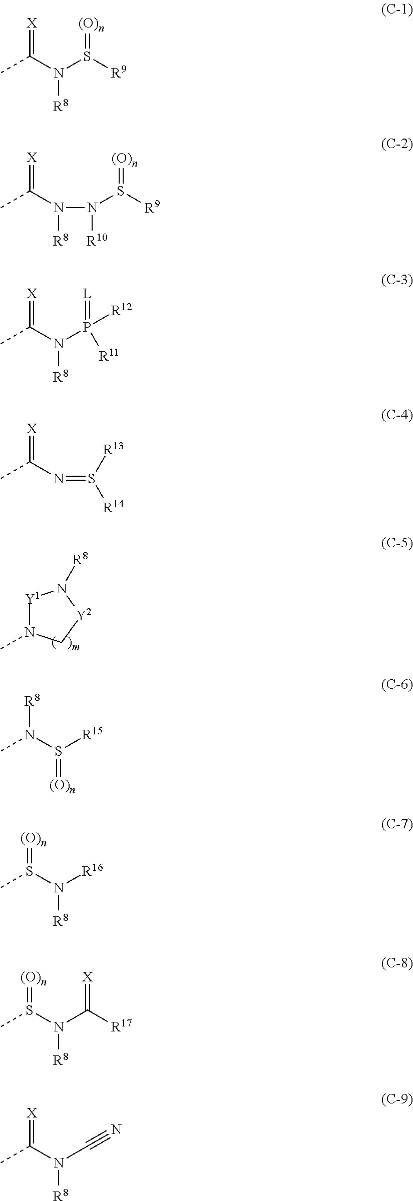

where the broken line denotes the bond to the B radicals,
X represents oxygen or sulfur,
X represents oxygen or sulfur,
X¹ represents a radical from the group consisting of hydrogen, halogen, cyano, nitro, alkyl, haloalkyl, cycloalkyl, alkoxy and haloalkoxy,
X² represents oxygen, sulfur, NR⁵ or NOH,
L represents oxygen or sulfur,
V—Z represents R²⁴CH—CHR²⁵ or R²⁴C═CR²⁵,
n represents 1 or 2,
m represents 1, 2, 3 or 4,
R represents NR¹⁸R¹⁹, or represents an in each case optionally substituted radical from the group consisting of alkyl, alkenyl, alkynyl, alkoxyalkyl, alkyl-S-alkyl, alkyl-S(O)-alkyl, alkyl-S(O)₂-alkyl, R¹⁸—CO-alkyl, NR¹⁸R¹⁹—CO-alkyl, cycloalkyl, cycloalkenyl, cycloalkylalkyl, cycloalkenylalkyl, heterocyclyl, heterocyclylalkyl, phenyl, phenylalkyl, hetaryl and hetarylalkyl,
R³ represents hydrogen or alkyl,
R⁴ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, alkylsulfinylalkyl, alkylsulfonylalkyl, aryl, arylalkyl and hetarylalkyl,
R⁵ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, aryl, arylalkyl and hetarylalkyl, or
R³ and R⁴ together with the nitrogen atom to which they are attached form a ring which may contain one or more further heteroatoms from the group consisting of nitrogen, oxygen and sulfur, or
R³ and R⁵ together with the nitrogen atoms to which they are attached form a ring,
R⁶ represents hydrogen or alkyl,
R⁷ represents a radical from the group consisting of hydrogen, alkyl, haloalkyl, cyanoalkyl, alkynyl, cycloalkyl, cycloalkylalkyl, alkoxy, haloalkoxy, alkoxyalkyl, alkylthioalkyl, alkylsulfinylalkyl, alkylsulfonylalkyl, alkoxycarbonyl, alkoxycarbonylalkyl, alkylthioalkyl, aryl, arylalkyl and hetarylalkyl, or
R⁶ and R⁷ together with the nitrogen atom to which they are attached form a ring which may contain one or more further heteroatoms from the group consisting of nitrogen, oxygen and sulfur,
R⁸ represents a radical from the group consisting of hydrogen, alkyl, haloalkyl, cyanoalkyl, alkoxy, haloalkoxy, alkenyl, alkoxyalkyl, in each case optionally halogen-substituted alkylcarbonyl and alkylsulfonyl, optionally halogen-substituted alkoxycarbonyl, optionally halogen-, alkyl-, alkoxy-, haloalkyl- and cyano-substituted cycloalkylcarbonyl, or a cation, or an optionally alkyl- or arylalkyl-substituted ammonium ion,
R⁹ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R⁹ in the radicals (C-1) and (F-1), together with the N—S(O)n group to which they are attached, may also form a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁰ represents hydrogen or alkyl,
R⁸ and R¹⁰ in the radicals (C-2) and (F-2), together with the nitrogen atoms to which they are attached, may also be a saturated or unsaturated and optionally substituted 4- to 8-membered ring which may contain at least one further heteroatom from the group consisting of sulfur, oxygen (where oxygen atoms must not be directly adjacent to one another) and nitrogen and/or at least one carbonyl group,
R⁹ and R¹⁰ in the radicals (C-2) and (F-2) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹¹ represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹² represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹¹ and R¹² in the radicals (C-3) and (F-3) may also form, together with the phosphorus atom to which they are bonded, a saturated or unsaturated and optionally substituted 5- to 7-membered ring which may contain one or two heteroatoms from the group consisting of oxygen (where oxygen atoms must not be immediately adjacent to one another) and sulfur,
R¹³ represents an in each case optionally substituted radical from the group consisting of alkyl, alkenyl, alkynyl, phenyl and phenylalkyl,
R¹⁴ represents an in each case optionally substituted radical from the group consisting of alkyl, alkenyl, alkynyl, phenyl and phenylalkyl,
R¹⁵ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁵ in the radicals (C-6) and (F-6) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁶ represents a radical from the group consisting of hydrogen, in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁶ in the radicals (C-7) and (F-7) may also form, together with the nitrogen atom to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁷ represents a radical from the group consisting of in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁷ in the radicals (C-8) and (F-8) may also form, together with the N—C(X) group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁸ represents a radical from the group consisting of hydrogen, hydroxy, in each case optionally substituted alkyl, alkoxy, alkoxyalkyl, alkylthioalkyl, alkylsulfinylalkyl, alkylsulfonylalkyl, alkylcarbonyl, alkoxycarbonyl, alkenyl and alkynyl, in each case optionally substituted cycloalkyl, cycloalkylalkyl, cycloalkenyl and cycloalkenylalkyl, in which the rings may contain at least one heteroatom from the group consisting of sulfur, oxygen (where oxygen atoms must not be directly adjacent to one another) and nitrogen, in each case optionally substituted aryl, arylalkyl, heteroaryl and heteroarylalkyl and an optionally substituted amino group,
R¹⁹ represents a radical from the group consisting of hydrogen, represents an alkali metal or alkaline earth metal ion or represents an ammonium ion which is optionally mono- to tetrasubstituted by C₁-C₄-alkyl or represents an in each case optionally halogen- or cyano-substituted radical from the group consisting of alkyl, alkoxy, alkoxyalkyl, alkylthioalkyl, alkylsulfinylalkyl, alkylsulfonylalkyl,
Y¹ and Y² independently of one another represent C═O or S(O)₂,
Y³ represents a radical from the group consisting of hydrogen, halogen, cyano, alkyl, cycloalkyl, haloalkyl, alkoxy, haloalkoxy and NR²⁰R²¹,
W represents a radical from the group consisting of O, S, SO and SO₂,
R²² represents a radical from the group consisting of hydrogen, alkyl, haloalkyl, cyanoalkyl, alkenyl, haloalkenyl, alkynyl, haloalkynyl, alkoxy, haloalkoxy, optionally halogen-substituted alkoxyalkyl, optionally halogen-substituted bis(alkoxy)alkyl, optionally halogen-substituted alkylsulfanylalkyl, optionally halogen-substituted alkylcarbonylalkyl, optionally halogen-substituted alkylsulfinylalkyl, optionally halogen-substituted alkylsulfonylalkyl, dialkylaminosulfanylalkyl, dialkylaminosulfinylalkyl, dialkylaminosulfonylalkyl, optionally halogen-substituted alkoxycarbonyl, optionally halogen-substituted alkoxycarbonylalkyl, optionally halogen-substituted alkynyloxy, optionally halogen-substituted alkynyloxycarbonyl, dialkylaminocarbonyl, N-alkyl-N-cycloalkylaminocarbonyl, dialkylaminocarbonylalkyl, N-alkyl-N-cycloalkylaminocarbonylalkyl, heterocyclylcarbonylalkyl, alkylsulfanyl, haloalkylsulfanyl, alkylsulfinyl, haloalkylsulfinyl, alkylsulfonyl, haloalkylsulfonyl, cycloalkyl which is optionally substituted by halogen, cyano, nitro, alkyl, cycloalkyl, haloalkyl, alkoxy, haloalkoxy, alkoxycarbonyl, haloalkoxycarbonyl or hetaryl (which for its part is optionally substituted by alkyl or halogen), cycloalkylcarbonyl which is optionally substituted by halogen, cyano, nitro, alkyl, haloalkyl, cycloalkyl, alkoxy, haloalkoxy, alkoxycarbonyl, haloalkoxycarbonyl or hetaryl (which for its part is optionally substituted by alkyl or halogen), cycloalkylalkyl which is optionally substituted by halogen, cyano, nitro, alkyl, haloalkyl, cycloalkyl, alkoxy, haloalkoxy, alkoxycarbonyl, haloalkoxycarbonyl or hetaryl (which for its part is optionally substituted by alkyl or halogen), optionally substituted heterocyclyl, heterocyclylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulfinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxyalkyl, alkenyl, alkynyl, cycloalkylalkyl, alkylcarbonyl, alkoxycarbonyl or aminocarbonyl, aryl which is optionally substituted by halogen, cyano, nitro, hydroxy, amino, alkyl, haloalkyl, cycloalkyl (which is optionally substituted), alkoxy or haloalkoxy, arylalkyl which is optionally substituted by halogen, cyano (also in the alkyl moiety), nitro, hydroxy, amino, alkyl, cycloalkyl (which is optionally substituted), haloalkyl, alkoxy or haloalkoxy, hetarylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulfinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxyalkyl, alkenyl, alkynyl, cycloalkylalkyl, alkylcarbonyl, alkoxycarbonyl or aminocarbonyl, or
R²² represents a D radical from the group consisting of (D-1) to (D-3)

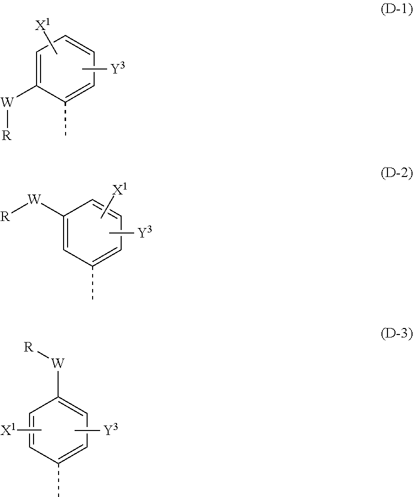

an E radical from the group consisting of (E-1) to (E-11)

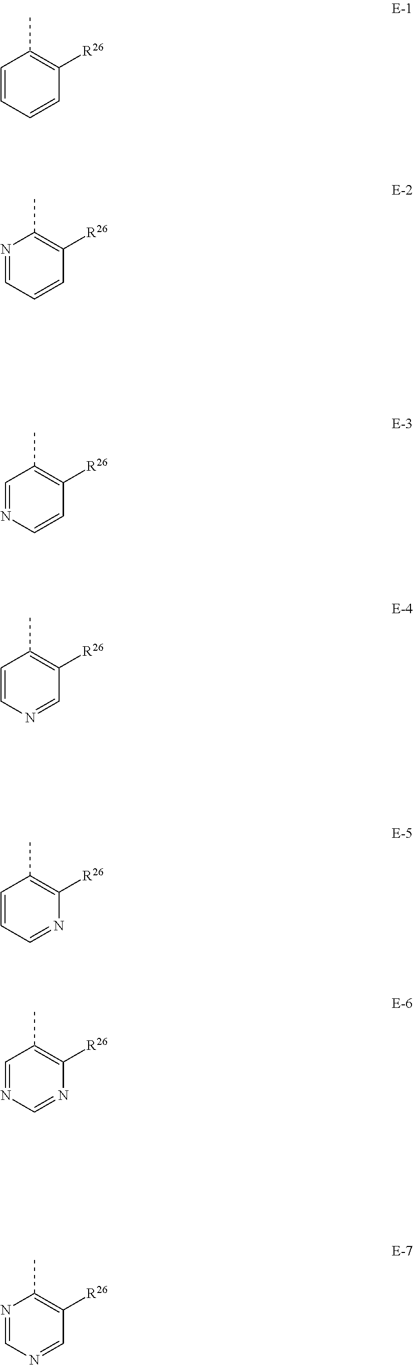

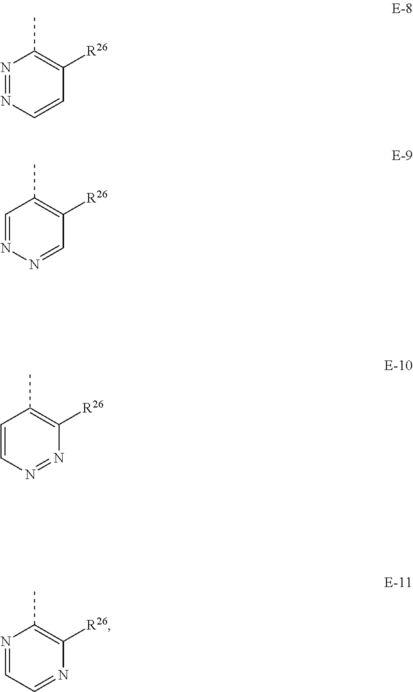

of the group consisting of (E-18) to (E-51)

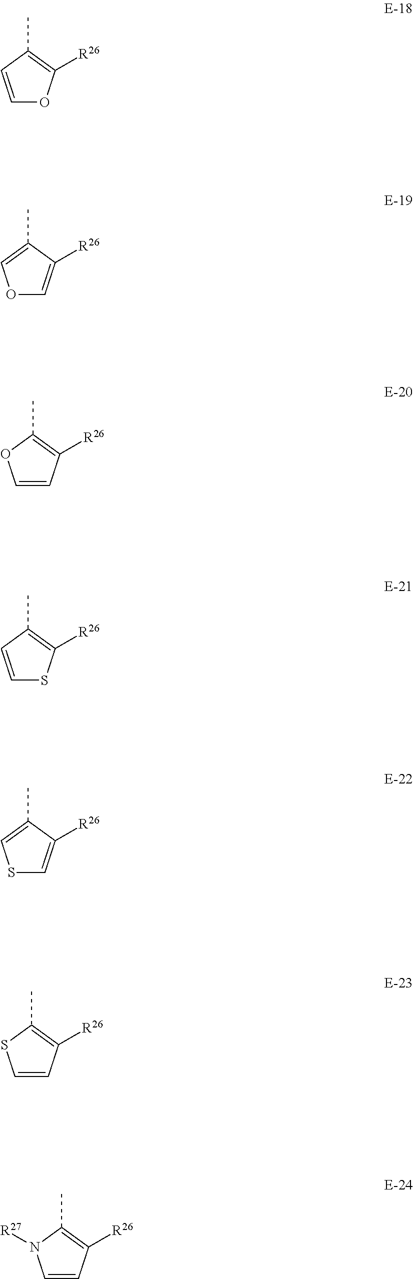

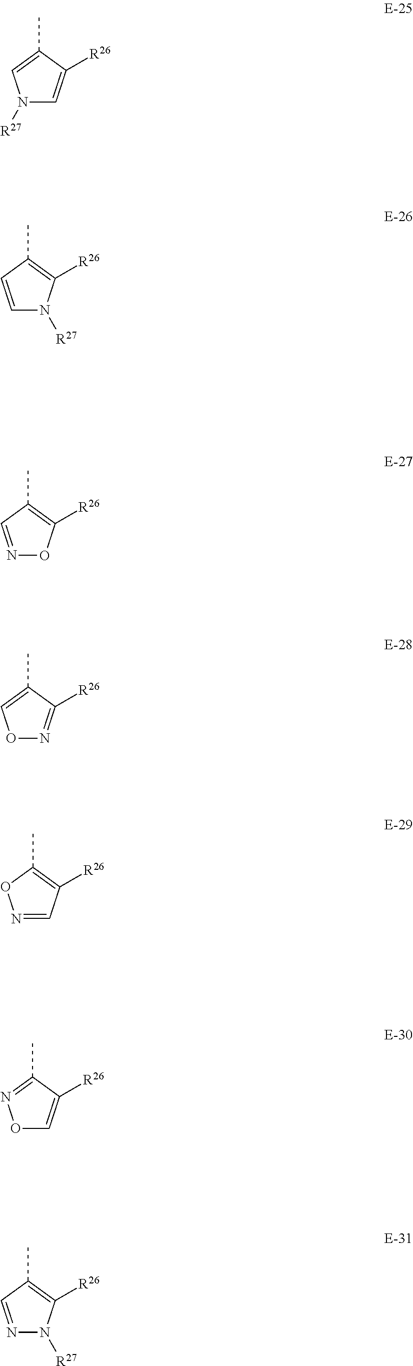

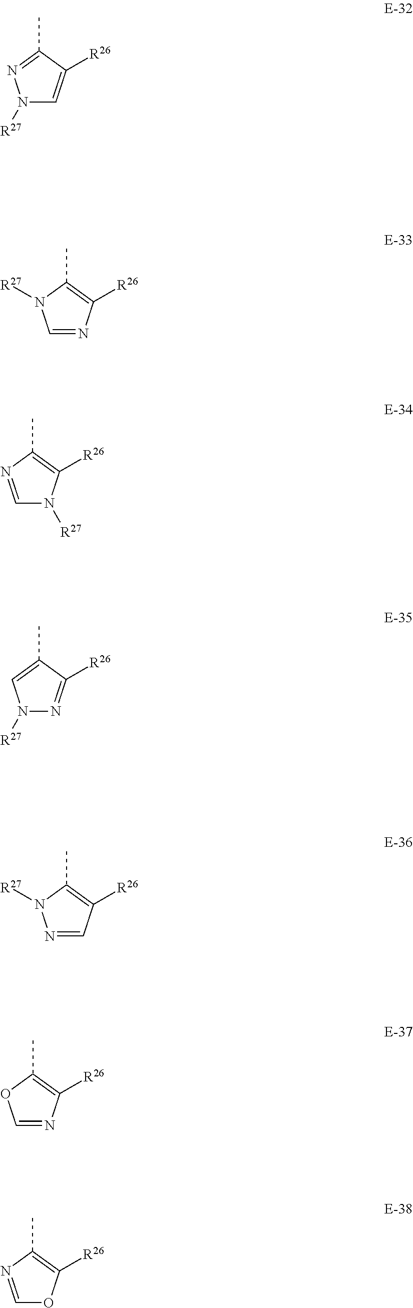

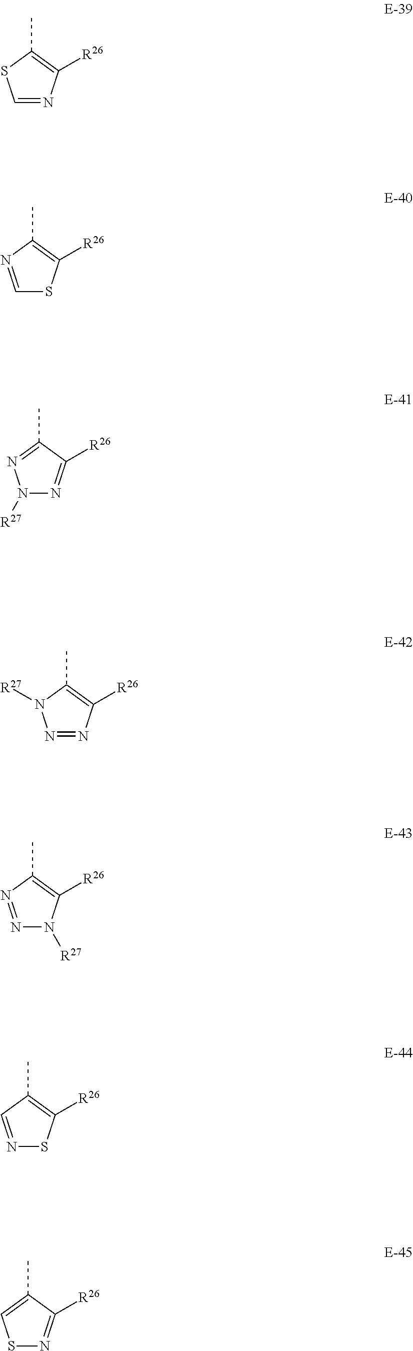

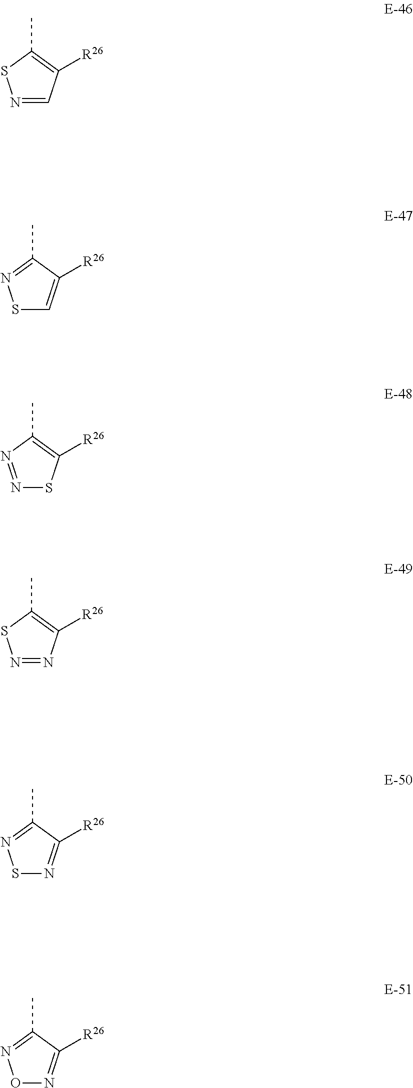

or
in the case R²=d),
R²² also represents an E radical from the group consisting of E-12 to E-17

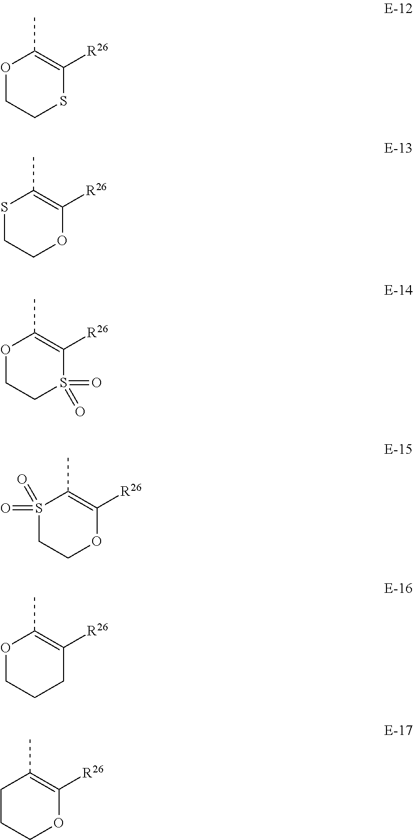

where here and below, the broken line denotes the bond to the appropriate atom in the radicals c), d) and g),
R²⁰ represents a radical from the group consisting of hydrogen, halogen, cyano, nitro, amino, hydroxy and in each case optionally substituted alkyl, alkenyl, alkynyl, cycloalkyl, cycloalkylalkyl, alkoxy, alkenyloxy, alkynyloxy, cycloalkyloxy, alkylcarbonyloxy, alkenylcarbonyloxy, alkynylcarbonyloxy, cycloalkylcarbonyloxy, alkoxycarbonyloxy, alkylsulfonyloxy, alkylamino, alkenylamino, alkynylamino, cycloalkylamino, alkylthio, haloalkylthio, alkenylthio, alkynylthio, cycloalkylthio, alkylsulfinyl, alkylsulfonyl, alkylcarbonyl, alkoxyiminoalkyl, alkoxycarbonyl, aminocarbonyl, alkylaminocarbonyl, dialkylaminocarbonyl, aminothiocarbonyl, alkylaminosulfonyl, alkylsulfonylamino, alkylcarbonylamino, alkenylcarbonylamino, alkynylcarbonylamino, cycloalkylcarbonylamino, alkoxycarbonylamino, alkylthiocarbonylamino, bicycloalkyl, aryl, aryloxy, heteroaryl and heteroaryloxy, where the substituents independently of one another are selected from the group consisting of halogen, cyano, nitro, hydroxy, amino, alkyl and haloalkyl,
R²¹ represents a radical from the group consisting of hydrogen, alkyl, cycloalkyl, haloalkyl, alkenyl, alkynyl, cycloalkylalkyl, cyanoalkyl, alkylcarbonyl, alkenylcarbonyl, haloalkylcarbonyl, haloalkenylcarbonyl, alkoxyalkyl, alkoxycarbonyl, alkylsulfonyl and haloalkylsulfonyl,
R²³ represents a radical from the group consisting of hydrogen, alkyl, alkenyl, alkynyl, cycloalkyl, cycloalkenyl, alkoxy, alkenyloxy, alkynyloxy, cycloalkyloxy, alkylthioalkyl, alkenylthioalkyl, cyanoalkyl, alkoxyalkyl,
or, if R²=c) or g),
R²² and R²³ together with the nitrogen atom to which they are attached form a ring which may contain one or more further heteroatoms from the group consisting of nitrogen, oxygen and sulfur, and
R²⁴ represents hydrogen or an in each case optionally substituted radical from the group consisting of alkyl, alkenyl, alkynyl, phenyl and phenylalkyl and
R²⁵ represents hydrogen or an in each case optionally substituted radical from the group consisting of alkyl, alkenyl, alkynyl, phenyl and phenylalkyl,
R²⁶ represents a radical from the group consisting of hydrogen, alkyl, haloalkyl, alkenyl, alkynyl, cycloalkyl, cycloalkylalkyl, alkoxyalkyl, alkylsulfanyl, haloalkylsulfanyl, alkylsulfinyl, haloalkylsulfinyl, alkylsulfonyl, alkylthioalkyl, alkylsulfinylalkyl, alkylsulfonylalkyl and cyanoalkyl.
R²⁷ represents hydrogen, alkyl, haloalkyl, alkenyl, alkynyl, cycloalkyl, cycloalkylalkyl, alkoxyalkyl, alkylthioalkyl, alkylsulfinylalkyl, alkylsulfonylalkyl or cyanoalkyl and
compounds of the formula (I) in which
A represents the A radical

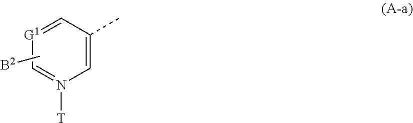

where the broken line denotes the bond to the carbon atom of the bicyclic system of the formula (I) and
G¹ represents N or C—B¹,
B¹ represents a radical from the group consisting of hydrogen, halogen, cyano, nitro, alkyl, haloalkyl, alkoxy, haloalkoxy and in each case optionally substituted cycloalkyl and cycloalkenyl,
B² represents a radical from the group consisting of hydrogen, halogen, cyano, nitro, alkyl, haloalkyl, alkoxy, haloalkoxy and in each case optionally substituted cycloalkyl and cycloalkenyl,
T represents oxygen or an electron pair,
Q represents oxygen or sulfur,
R¹ represents a radical from the group consisting of hydrogen, alkyl, alkoxy and cyano,
R² a) represents a B radical from the group consisting of

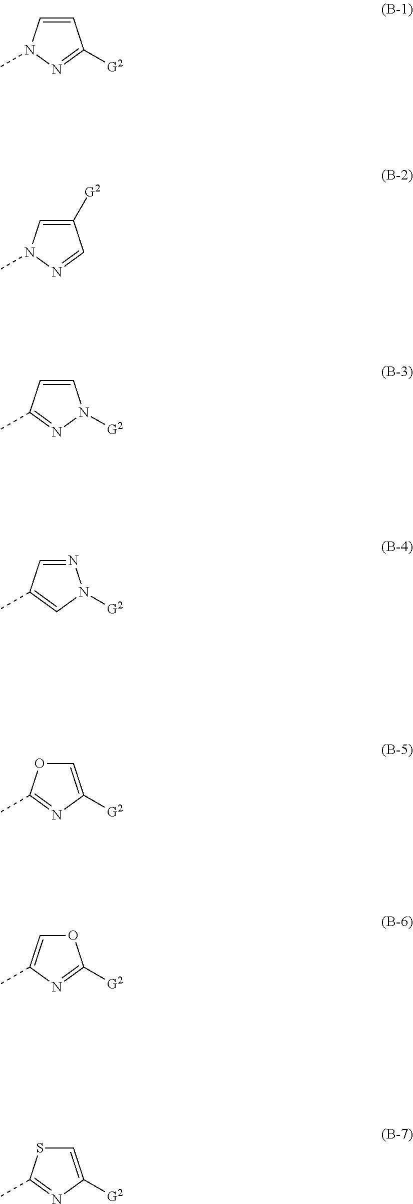

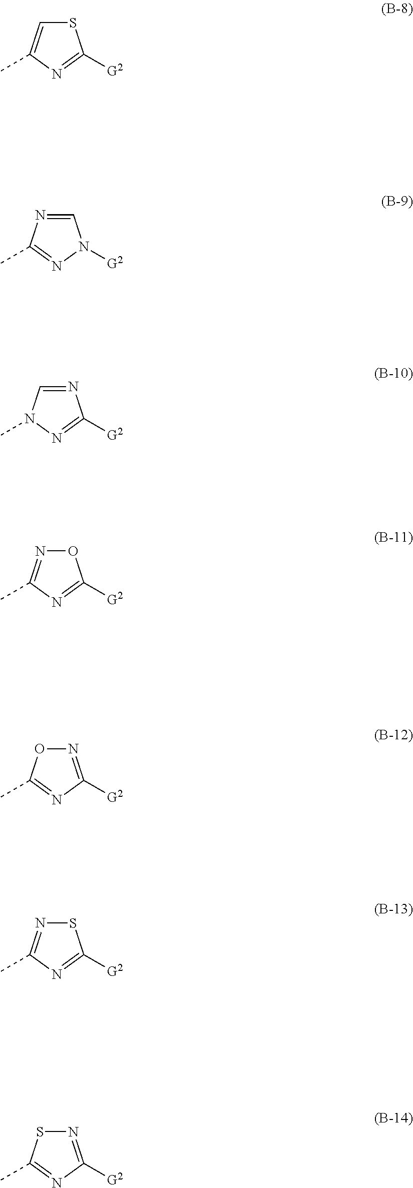

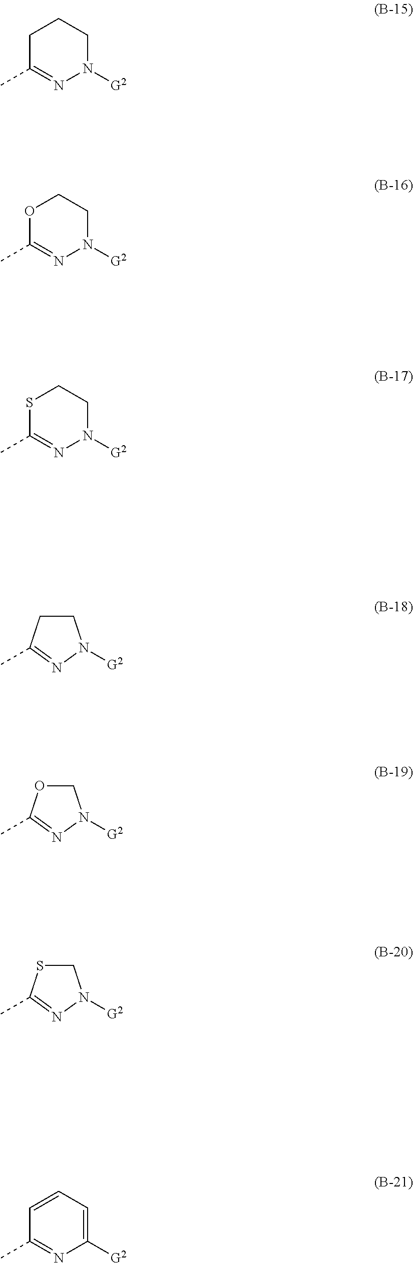

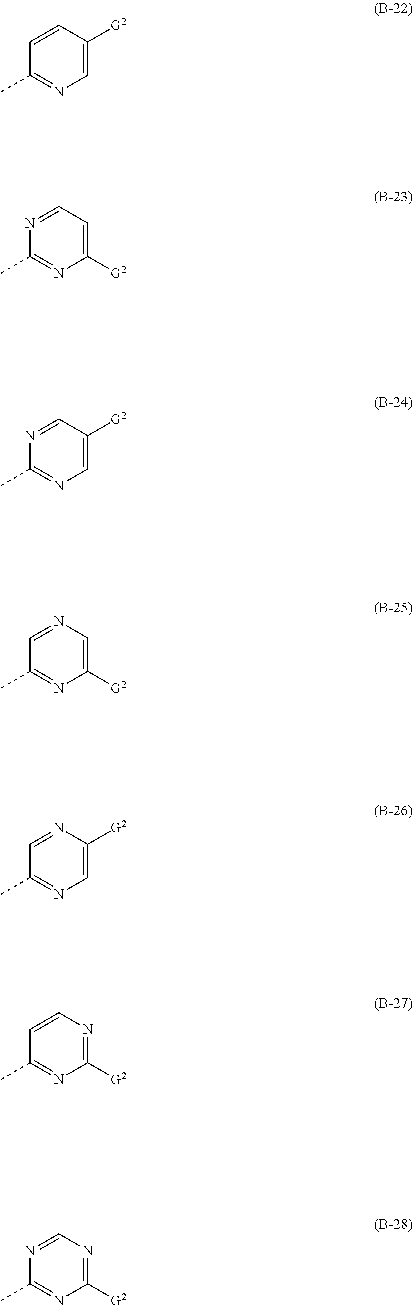

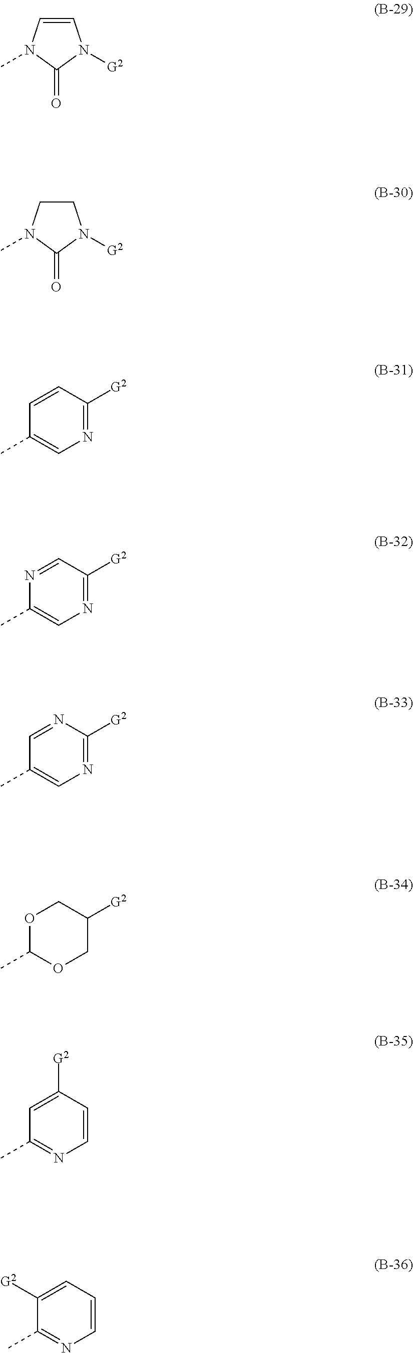

where the broken line denotes the bond to the carbon atom of the bicyclic system of the formula (I), or
R² c) represents a radical of the formula

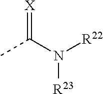

where the broken line denotes the bond to the carbon atom of the bicyclic system of the formula (I), or
R² d) represents a radical of the formula

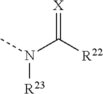

where the broken line denotes the bond to the carbon atom of the bicyclic system of the formula (I), or
R² e) represents an F radical from the group consisting of

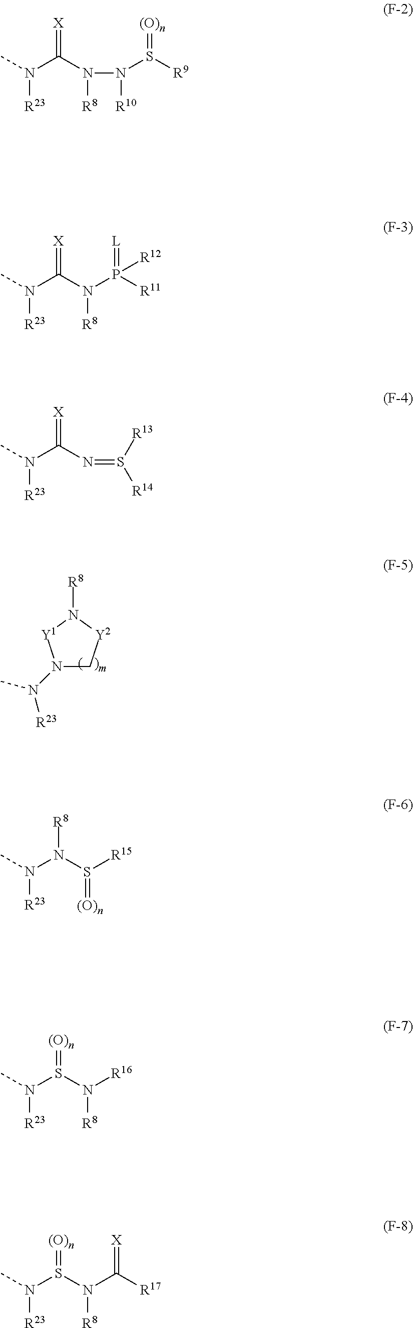

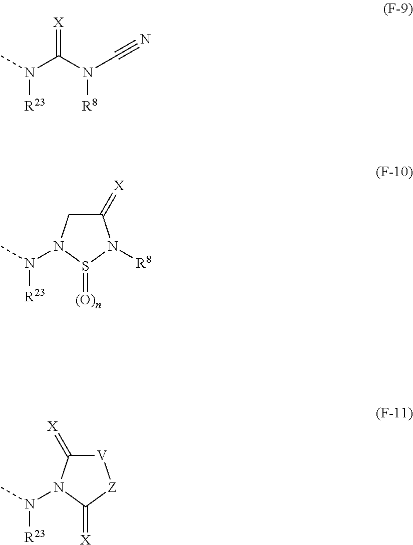

where the broken line denotes the bond to the carbon atom of the bicyclic system of the formula (I), or
R² f) represents a radical from the group consisting of haloalkyl and carboxyl,
R² g) represents a radical of the formula

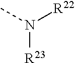

where the broken line denotes the bond to the carbon atom of the bicyclic system of the formula (I), where
G² represents hydrogen or a radical from the group consisting of halogen, nitro, amino, cyano, alkylamino, haloalkylamino, dialkylamino, alkyl, haloalkyl, saturated or unsaturated cycloalkyl which is optionally substituted and optionally interrupted by one or more heteroatoms, cycloalkylalkyl, alkoxy, haloalkoxy, alkoxyalkyl, halogenated alkoxyalkyl, alkylthioalkyl, alkylsulfinylalkyl, alkylsulfonylalkyl, bis(alkoxy)alkyl, bis(haloalkoxy)alkyl, alkoxy(alkylsulfanyl)alkyl, alkoxy(alkylsulfinyl)alkyl, alkoxy(alkylsulfonyl)alkyl, bis(alkylsulfanyl)alkyl, bis(haloalkylsulfanyl)alkyl, bis(hydroxyalkylsulfanyl)alkyl, alkoxycarbonyl, alkoxycarbonylalkyl, alpha-hydroxyiminoalkoxycarbonylalkyl, alpha-alkoxyiminoalkoxycarbonylalkyl, C(X²)NR³R⁴, NR⁶R⁷, alkylthio, alkylsulfinyl, alkylsulfonyl, haloalkylthio, haloalkylsulfinyl, haloalkylsulfonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by alkyl, haloalkyl, alkoxy and alkoxyalkyl), phenyl (which for its part may be substituted by halogen, cyano, nitro, alkyl and haloalkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, alkyl, haloalkyl, alkoxy, haloalkoxy, alkoxyalkyl, alkylthio, alkylthioalkyl and cycloalkyl) and the heteroarylalkyl radicals triazolylalkyl, pyridylalkyl, pyrimidylalkyl and oxadiazolylalkyl (which for their part may be substituted by halogen and alkyl), or
G² represents a C radical from the group consisting of (C-1) to (C-9)

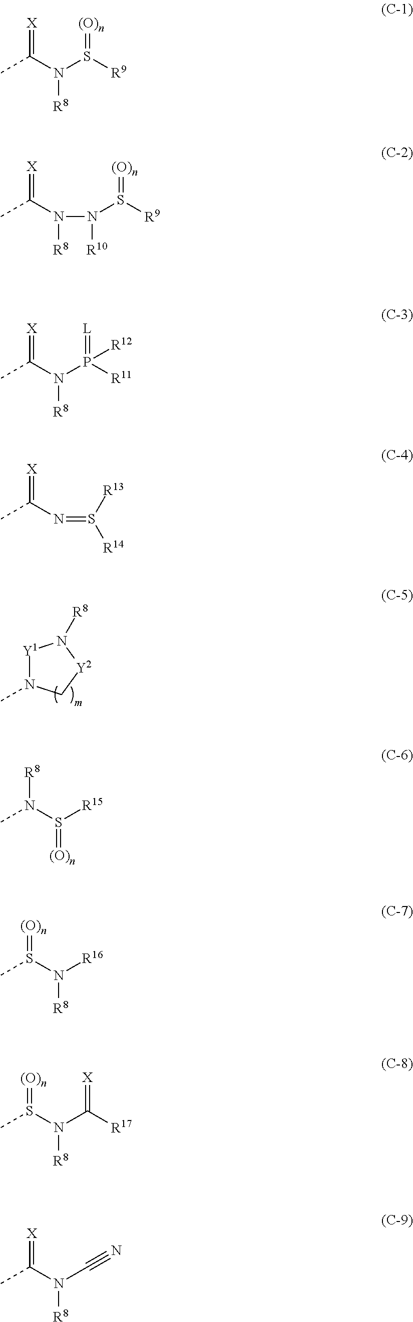

where the broken line represents the bond to the B radicals,
X represents oxygen or sulfur,
X² represents oxygen, sulfur, NR⁵ or NOH,
L represents oxygen or sulfur,
V—Z represents R²⁴CH—CHR²⁵ or R²⁴C═CR²⁵,
n represents 1 or 2,
m represents 1, 2, 3 or 4,
R³ represents hydrogen or alkyl,
R⁴ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, alkylsulfinylalkyl, alkylsulfonylalkyl, aryl, arylalkyl and hetarylalkyl,
R⁵ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, aryl, arylalkyl and hetarylalkyl, or
R³ and R⁴ together with the nitrogen atom to which they are attached form a ring which may contain one or more further heteroatoms from the group consisting of nitrogen, oxygen and sulfur, or
R³ and R⁵ together with the nitrogen atoms to which they are attached form a ring,
R⁶ represents hydrogen or alkyl,
R⁷ represents a radical from the group consisting of hydrogen, alkyl, haloalkyl, cyanoalkyl, alkynyl, cycloalkyl, cycloalkylalkyl, alkoxy, haloalkoxy, alkoxyalkyl, alkylthioalkyl, alkylsulfinylalkyl, alkylsulfonylalkyl, alkoxycarbonyl, alkoxycarbonylalkyl, alkylthioalkyl, aryl, arylalkyl and hetarylalkyl, or
R⁶ and R⁷ together with the nitrogen atom to which they are attached form a ring which may contain one or more further heteroatoms from the group consisting of nitrogen, oxygen and sulfur,
R⁸ represents a radical from the group consisting of hydrogen, alkyl, haloalkyl, cyanoalkyl, alkoxy, haloalkoxy, alkenyl, alkoxyalkyl, in each case optionally halogen-substituted alkylcarbonyl and alkylsulfonyl, optionally halogen-substituted alkoxycarbonyl, optionally halogen-, alkyl-, alkoxy-, haloalkyl- and cyano-substituted cycloalkylcarbonyl, or a cation, or an optionally alkyl- or arylalkyl-substituted ammonium ion,
R⁹ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R⁹ in the radical (C-1), together with the N—S(O)n group to which they are attached, may also form a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁰ represents hydrogen or alkyl,
R⁸ and R¹⁰ in the radicals (C-2) and (F-2), together with the nitrogen atoms to which they are attached, may also be a saturated or unsaturated and optionally substituted 4- to 8-membered ring which may contain at least one further heteroatom from the group consisting of sulfur, oxygen (where oxygen atoms must not be directly adjacent to one another) and nitrogen and/or at least one carbonyl group,
R⁹ and R¹⁰ in the radicals (C-2) and (F-2) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹¹ represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹² represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹¹ and R¹² in the radicals (C-3) and (F-3) may also form, together with the phosphorus atom to which they are bonded, a saturated or unsaturated and optionally substituted 5- to 7-membered ring which may contain one or two heteroatoms from the group consisting of oxygen (where oxygen atoms must not be immediately adjacent to one another) and sulfur,
R¹³ represents an in each case optionally substituted radical from the group consisting of alkyl, alkenyl, alkynyl, phenyl and phenylalkyl,
R¹⁴ represents an in each case optionally substituted radical from the group consisting of alkyl, alkenyl, alkynyl, phenyl and phenylalkyl,
R¹⁵ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁵ in the radicals (C-6) and (F-6) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁶ represents a radical from the group consisting of hydrogen, in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁶ in the radicals (C-7) and (F-7) may also form, together with the nitrogen atom to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁷ represents a radical from the group consisting of in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁷ in the radicals (C-8) and (F-8) may also form, together with the N—C(X) group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
Y¹ and Y² independently of one another represent C═O or S(O)₂,
R²² in the case that R² represents g), represents a radical from the group consisting of hydrogen, alkyl, haloalkyl, cyanoalkyl, alkenyl, haloalkenyl, alkynyl, haloalkynyl, alkoxy, haloalkoxy, optionally halogen-substituted alkoxyalkyl, optionally halogen-substituted bis(alkoxy)alkyl, optionally halogen-substituted alkylsulfanylalkyl, optionally halogen-substituted alkylcarbonylalkyl, optionally halogen-substituted alkylsulfinylalkyl, optionally halogen-substituted alkylsulfonylalkyl, dialkylaminosulfanylalkyl, dialkylaminosulfinylalkyl, dialkylaminosulfonylalkyl, optionally halogen-substituted alkoxycarbonylalkyl, optionally halogen-substituted alkynyloxy, dialkylaminocarbonylalkyl, N-alkyl-N-cycloalkylaminocarbonylalkyl, heterocyclylcarbonylalkyl, alkylsulfanyl, haloalkylsulfanyl, alkylsulfinyl, haloalkylsulfinyl, alkylsulfonyl, haloalkylsulfonyl, cycloalkyl which is optionally substituted by halogen, cyano, nitro, alkyl, cycloalkyl, haloalkyl, alkoxy, haloalkoxy, alkoxycarbonyl, haloalkoxycarbonyl or hetaryl (which for its part is optionally substituted by alkyl or halogen), cycloalkylalkyl which is optionally substituted by halogen, cyano, nitro, alkyl, haloalkyl, cycloalkyl, alkoxy, haloalkoxy, alkoxycarbonyl, haloalkoxycarbonyl or hetaryl (which for its part is optionally substituted by alkyl or halogen), optionally substituted heterocyclyl, heterocyclylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulpinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxy alkyl, alkenyl, alkynyl, cycloalkylalkyl, alkylcarbonyl, alkoxycarbonyl or aminocarbonyl, aryl which is optionally substituted by halogen, cyano, nitro, hydroxy, amino, alkyl, haloalkyl, cycloalkyl (which is optionally substituted), alkoxy or haloalkoxy, arylalkyl which is optionally substituted by halogen, cyano (also in the alkyl moiety), nitro, hydroxy, amino, alkyl, cycloalkyl (which is optionally substituted), haloalkyl, alkoxy or haloalkoxy, hetarylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulfinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxyalkyl, alkenyl, alkynyl, cycloalkylalkyl, alkylcarbonyl, alkoxycarbonyl or aminocarbonyl, or, in the case that R² represents c), d) or g),
R²² represents a D radical from the group consisting of (D-1) to (D-3)

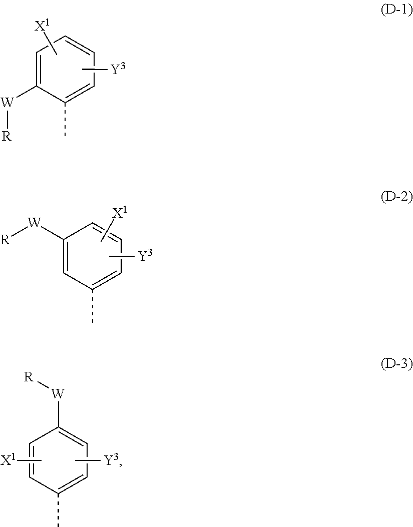

in which
X¹ represents a radical from the group consisting of hydrogen, halogen, cyano, nitro, alkyl, haloalkyl, cycloalkyl, alkoxy and haloalkoxy,
R represents NR¹⁸R¹⁹, or represents an in each case optionally substituted radical from the group consisting of alkyl, alkenyl, alkynyl, alkoxyalkyl, alkyl-S-alkyl, alkyl-S(O)-alkyl, alkyl-S(O)₂-alkyl, R¹⁸—CO-alkyl, NR¹⁸R¹⁹—CO-alkyl, cycloalkyl, cycloalkenyl, cycloalkylalkyl, cycloalkenylalkyl, heterocyclyl, heterocyclylalkyl, phenyl, phenylalkyl, hetaryl and hetarylalkyl,
Y³ represents a radical from the group consisting of hydrogen, halogen, cyano, alkyl, cycloalkyl, haloalkyl, alkoxy, haloalkoxy and NR²⁰R²¹,
W represents a radical from the group consisting of O, S, SO and SO₂,
R²² furthermore represents an E radical from the group consisting of (E-1) to (E-11)

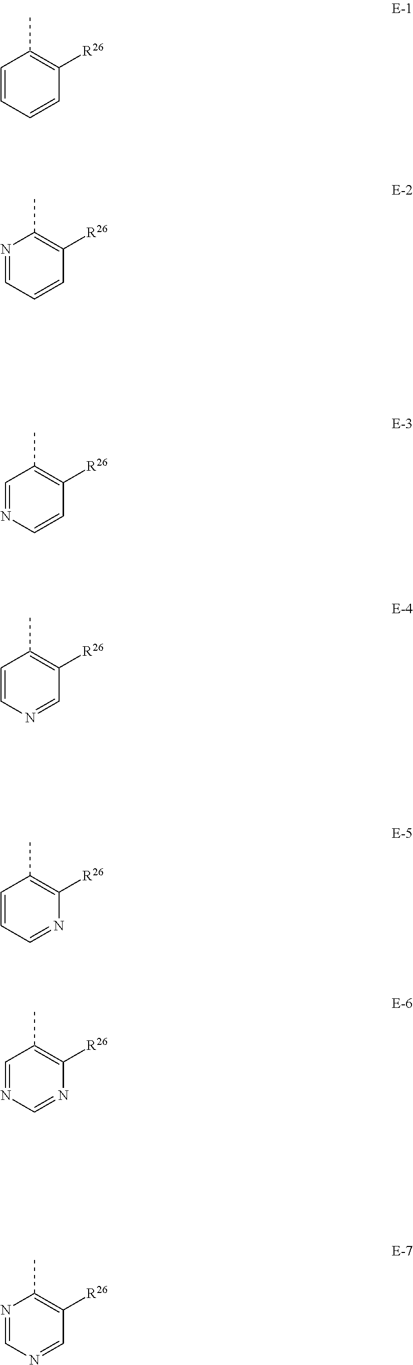

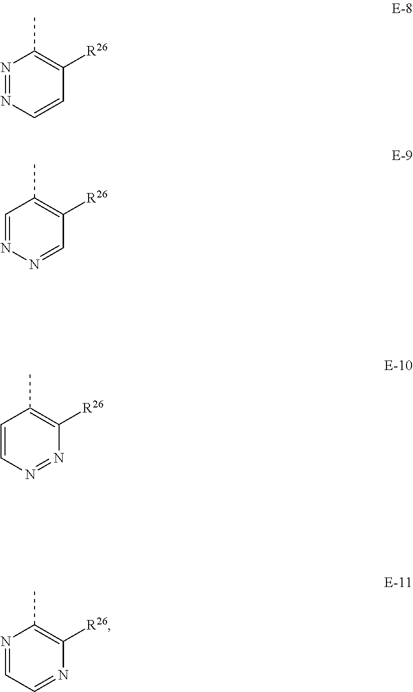

of the group consisting of (E-18) to (E-51)

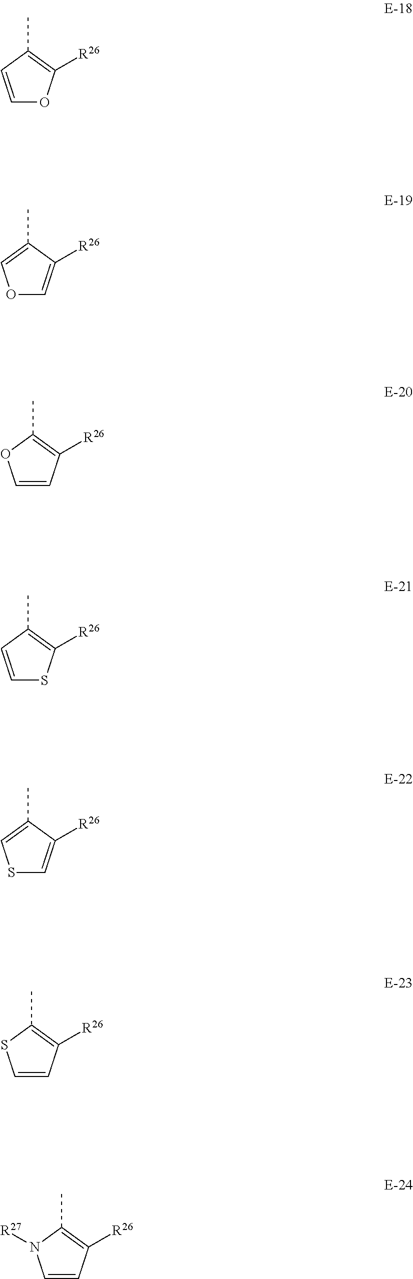

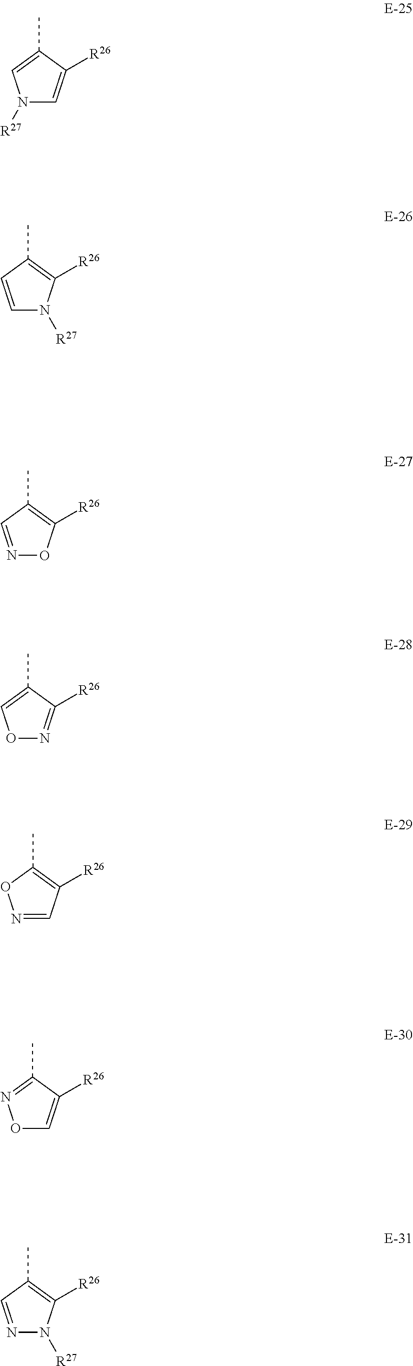

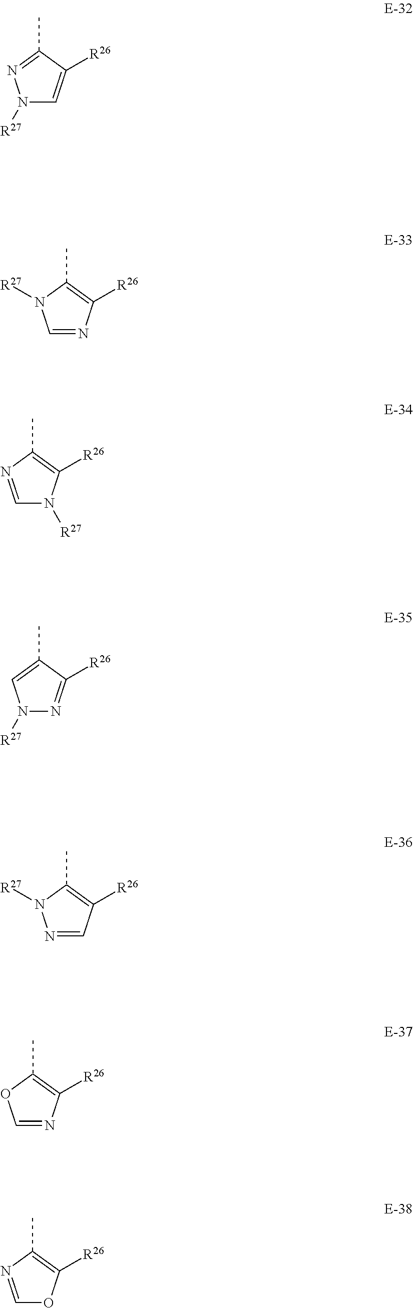

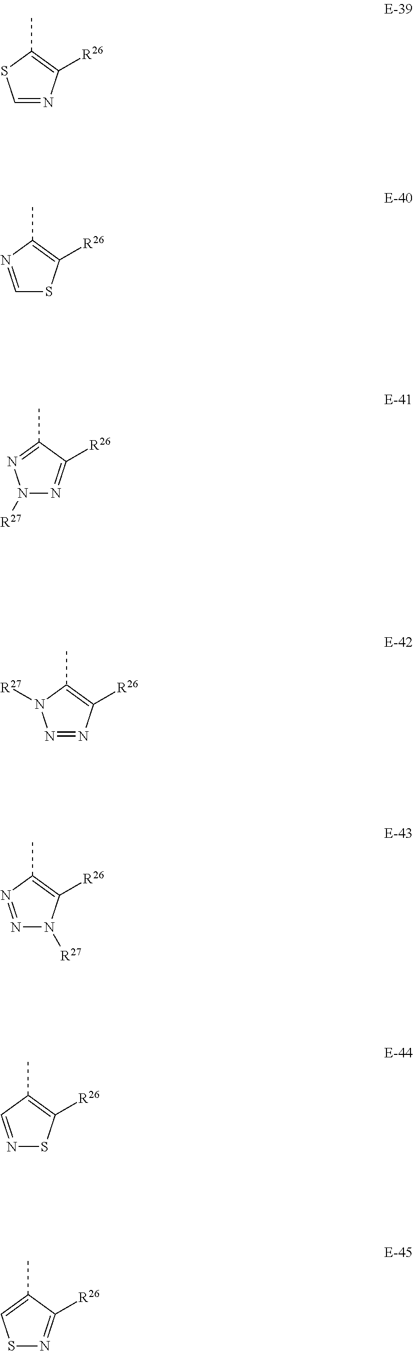

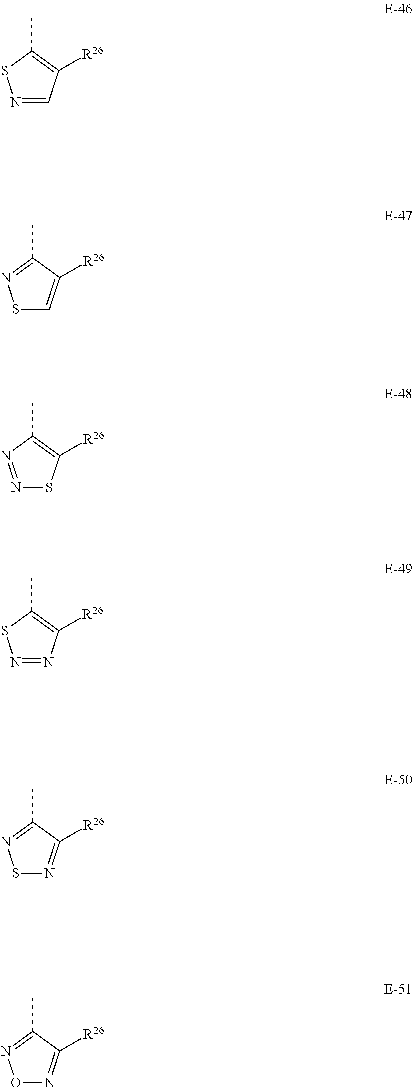

or, in the case that R²=d),
R²² also represents an E radical from the group consisting of E-12 to E-17

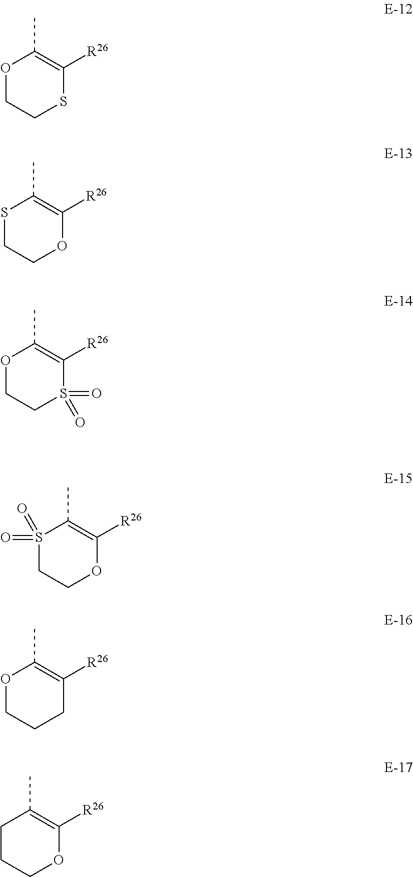

R¹⁸ represents a radical from the group consisting of hydrogen, hydroxy, in each case optionally substituted alkyl, alkoxy, alkoxyalkyl, alkylthioalkyl, alkylsulfinylalkyl, alkylsulfonylalkyl, alkylcarbonyl, alkoxycarbonyl, alkenyl and alkynyl, in each case optionally substituted cycloalkyl, cycloalkylalkyl, cycloalkenyl and cycloalkenylalkyl, in which the rings may contain at least one heteroatom from the group consisting of sulfur, oxygen (where oxygen atoms must not be directly adjacent to one another) and nitrogen, in each case optionally substituted aryl, arylalkyl, heteroaryl and heteroarylalkyl and an optionally substituted amino group,
R¹⁹ represents a radical from the group consisting of hydrogen, represents an alkali metal or alkaline earth metal ion or represents an ammonium ion which is optionally mono- to tetrasubstituted by C₁-C₄-alkyl or represents an in each case optionally halogen- or cyano-substituted radical from the group consisting of alkyl, alkoxy, alkoxyalkyl, alkylthioalkyl, alkylsulfinylalkyl and alkylsulfonylalkyl,
R²⁰ represents a radical from the group consisting of hydrogen, halogen, cyano, nitro, amino, hydroxy and in each case optionally substituted alkyl, alkenyl, alkynyl, cycloalkyl, cycloalkylalkyl, alkoxy, alkenyloxy, alkynyloxy, cycloalkyloxy, alkylcarbonyloxy, alkenylcarbonyloxy, alkynylcarbonyloxy, cycloalkylcarbonyloxy, alkoxycarbonyloxy, alkylsulfonyloxy, alkylamino, alkenylamino, alkynylamino, cycloalkylamino, alkylthio, haloalkylthio, alkenylthio, alkynylthio, cycloalkylthio, alkylsulfinyl, alkylsulfonyl, alkylcarbonyl, alkoxyiminoalkyl, alkoxycarbonyl, aminocarbonyl, alkylaminocarbonyl, dialkylaminocarbonyl, aminothiocarbonyl, alkylaminosulfonyl, alkylsulfonylamino, alkylcarbonylamino, alkenylcarbonylamino, alkynylcarbonylamino, cycloalkylcarbonylamino, alkoxycarbonylamino, alkylthiocarbonylamino, bicycloalkyl, aryl, aryloxy, heteroaryl and heteroaryloxy, where the substituents independently of one another are selected from the group consisting of halogen, cyano, nitro, hydroxy, amino, alkyl and haloalkyl,
R²¹ represents a radical from the group consisting of hydrogen, alkyl, cycloalkyl, haloalkyl, alkenyl, alkynyl, cycloalkylalkyl, cyanoalkyl, alkylcarbonyl, alkenylcarbonyl, haloalkylcarbonyl, haloalkenylcarbonyl, alkoxyalkyl, alkoxycarbonyl, alkylsulfonyl and haloalkylsulfonyl,
R²³ represents a radical from the group consisting of hydrogen, alkyl, alkenyl, alkynyl, cycloalkyl, cycloalkenyl, alkoxy, alkenyloxy, alkynyloxy, cycloalkyloxy, alkylthioalkyl, alkenylthioalkyl, cyanoalkyl, alkoxyalkyl,
or, if R²=g),
R²² and R²³ together with the nitrogen atom to which they are attached form a ring which may contain one or more further heteroatoms from the group consisting of nitrogen, oxygen and sulfur, and
R²⁴ represents hydrogen or an in each case optionally substituted radical from the group consisting of alkyl, alkenyl, alkynyl, phenyl and phenylalkyl and
R²⁵ represents hydrogen or an in each case optionally substituted radical from the group consisting of alkyl, alkenyl, alkynyl, phenyl and phenylalkyl,
R²⁶ represents a radical from the group consisting of hydrogen, alkyl, haloalkyl, alkenyl, alkynyl, cycloalkyl, cycloalkylalkyl, alkoxyalkyl, alkylsulfanyl, haloalkylsulfanyl, alkylsulfinyl, haloalkylsulfinyl, alkylsulfonyl, alkylthioalkyl, alkylsulfinylalkyl, alkylsulfonylalkyl and cyanoalkyl.
R²⁷ represents hydrogen, alkyl, haloalkyl, alkenyl, alkynyl, cycloalkyl, cycloalkylalkyl, alkoxyalkyl, alkylthioalkyl, alkylsulfinylalkyl, alkylsulfonylalkyl or cyanoalkyl.

It has been additionally found that the compounds of the formula (I) and also the compounds listed in Table 1 which are not embraced by formula (I) have good efficacy as pesticides, for example against arthropods and especially insects, and additionally generally have very good compatibility with plants, especially crop plants, and/or have favourable toxicological and/or favourable environmentally relevant properties.

Preferred range (1): Preference is given to compounds of the formula (I) in which
A represents an A radical from the group consisting of (A-b) and (A-f)

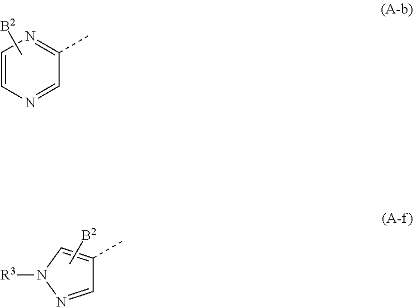

in which the broken line denotes the bond to the carbon atom of the bicyclic system of the formula (I) and
B² represents a radical from the group consisting of hydrogen, halogen, C₁-C₆-alkyl and C₁-C₄-haloalkyl,
Q represents sulfur,
R¹ represents a radical from the group consisting of hydrogen, C₁-C₆-alkyl and C₁-C₆-alkoxy,
R² a) represents a B radical from the group consisting of

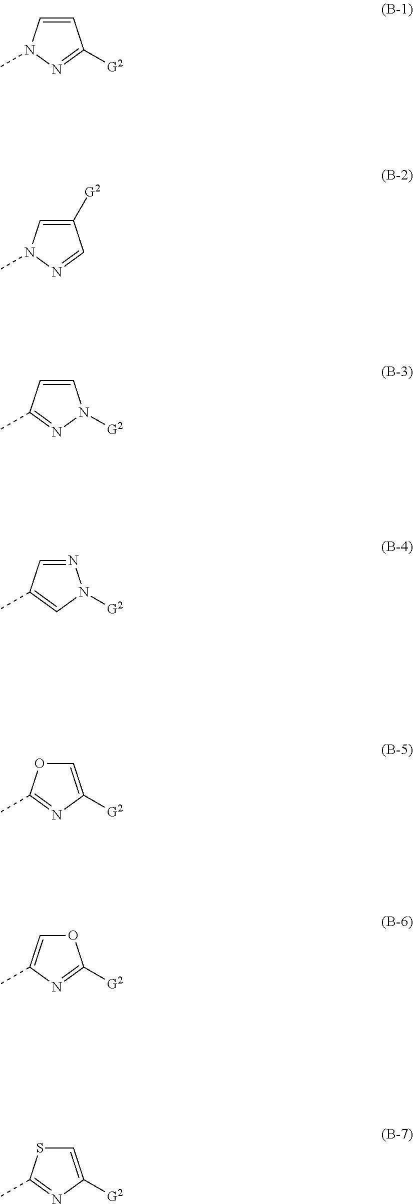

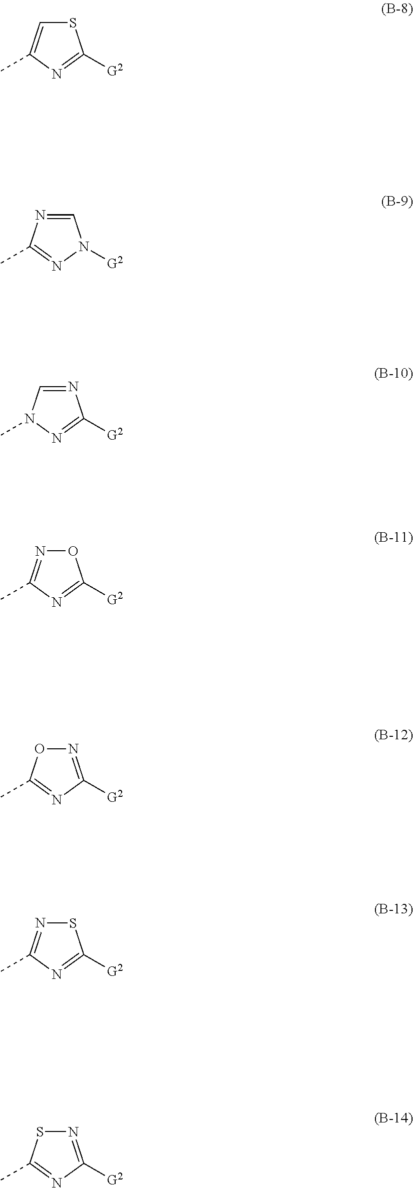

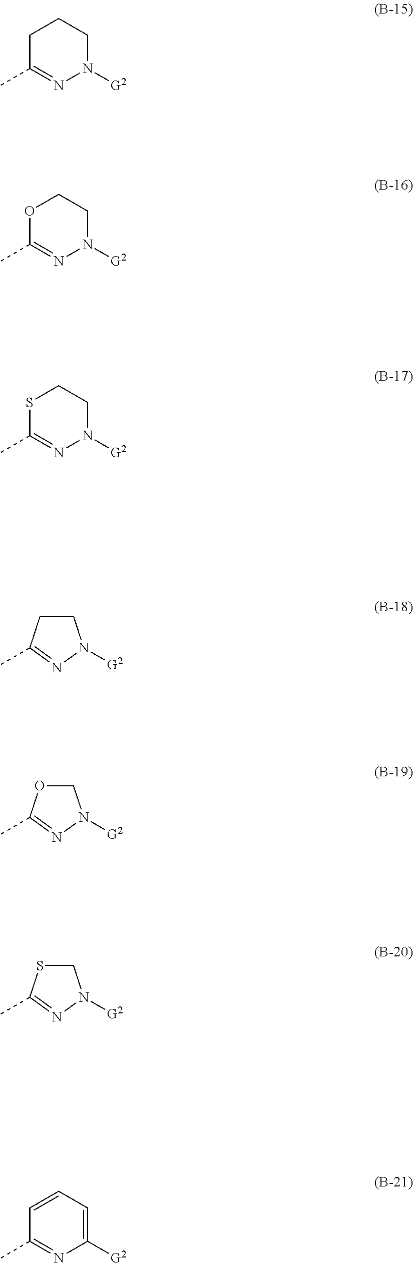

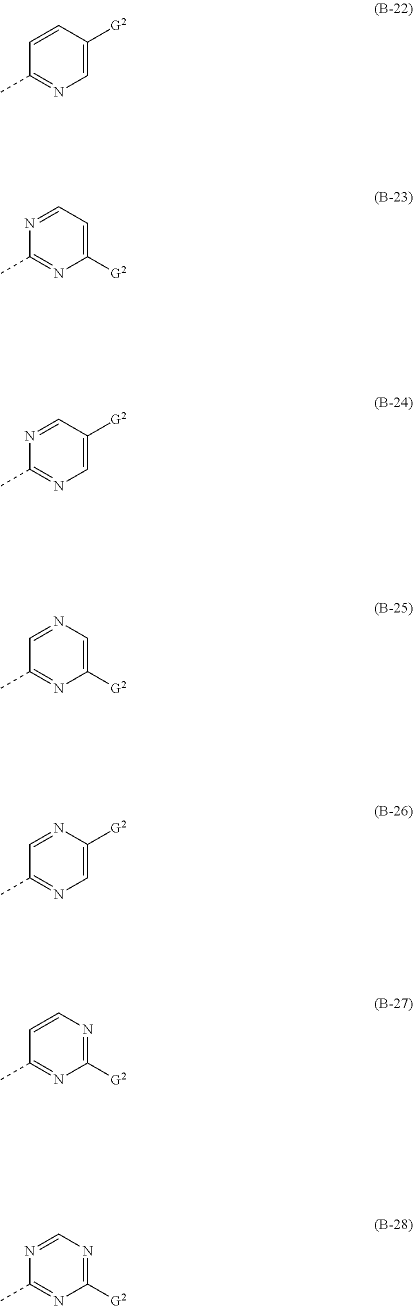

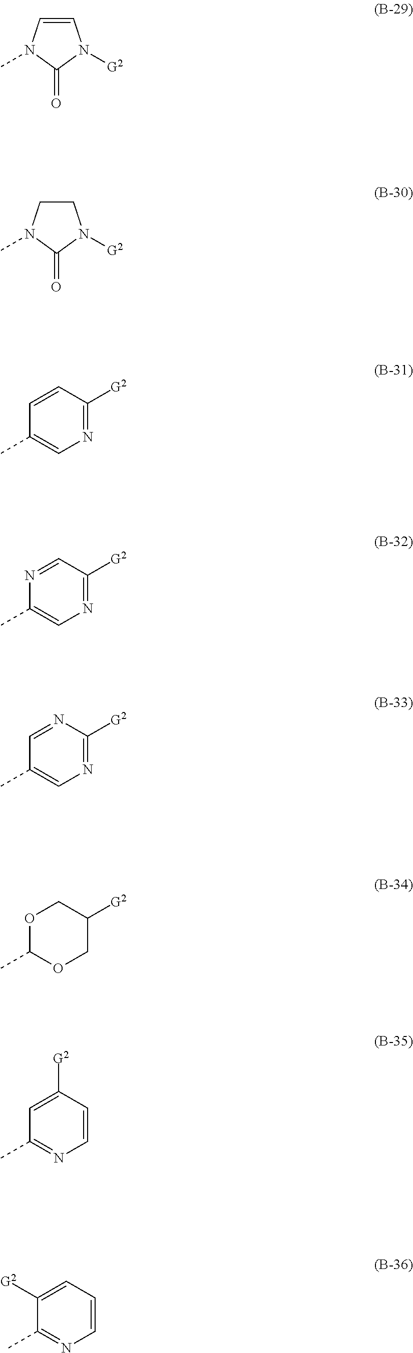

where the broken line denotes the bond to the carbon atom of the bicyclic system of the formula (I), or
R² b) represents a D radical from the group consisting of (D-1) to (D-3)

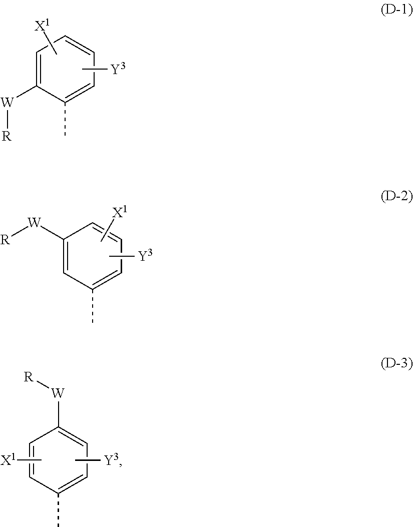

where the broken line denotes the bond to the carbon atom of the bicyclic system of the formula (I), or
R² c) represents a radical of the formula

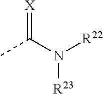

where the broken line denotes the bond to the carbon atom of the bicyclic system of the formula (I), or
R² d) represents a radical of the formula

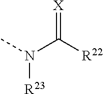

where the broken line denotes the bond to the carbon atom of the bicyclic system of the formula (I), or
R² e) represents an F radical from the group consisting of (F-1), (F-8), (F-10) and (F-11)

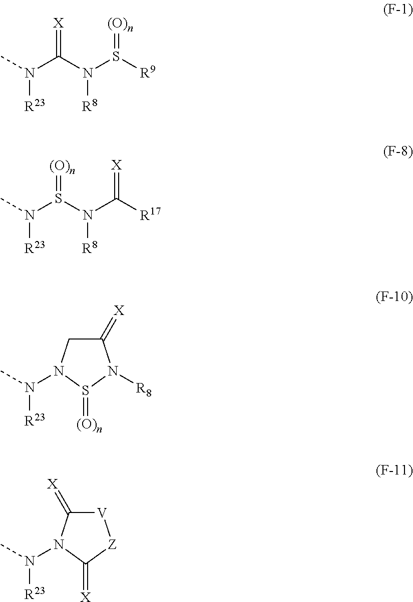

where the broken line denotes the bond to the carbon atom in the formula (I) or
R² f) represents a radical from the group consisting of C₁-C₆-haloalkyl and carboxyl, or
R² g) represents a radical of the formula

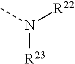

where the broken line represents the bond to the carbon atom of the bicyclic system of the formula (I), where

G² represents hydrogen or represents a radical from the group consisting of halogen, nitro, amino, cyano, C₁-C₄-alkylamino, halo-C₁-C₄-alkylamino, di-(C₁-C₄-alkyl)-amino, C₁-C₄-alkyl, halo-C₁-C₄-alkyl, C₃-C₆-cycloalkyl, C₃-C₆-cycloalkenyl, C₃-C₆-cycloalkyl-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy-(C₁-C₄-alkylsulfanyl)-C₁-C₄-alkyl, C₁-C₄-alkoxy-(C₁-C₄-alkylsulfinyl)-C₁-C₄-alkyl, C₁-C₄-alkoxy-(C₁-C₄-alkylsulfonyl)-C₁-C₄-alkyl, bis(C₁-C₄-alkylsulfanyl)-C₁-C₄-alkyl, bis(halo-C₁-C₄-alkylsulfanyl)-C₁-C₄-alkyl, bis(hydroxy-C₁-C₄-alkylsulfanyl)-C₁-C₄-alkyl, C₁-C₄-alkoxycarbonyl, C₁-C₄-alkoxycarbonyl-C₁-C₄-alkyl, alpha-hydroxyimino-C₁-C₄-alkoxycarbonyl-C₁-C₄-alkyl, alpha-C₁-C₄-alkoxyimino-C₁-C₄-alkoxycarbonyl-C₁-C₄-alkyl, C(X²)NR³R⁴, NR⁶R⁷, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by C₁-C₄-alkyl, halo-C₁-C₄-alkyl, C₁-C₄-alkoxy and C₁-C₄-alkoxy-C₁-C₄-alkyl), phenyl (which for its part may be substituted by halogen, cyano, nitro, C₁-C₄-alkyl and halo-C₁-C₄-alkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, C₁-C₄-alkyl, halo-C₁-C₄-alkyl, C₁-C₄-alkoxy, halo-C₁-C₄-alkoxy, C₁-C₄-alkoxy-C₁-C₄-alkyl, C₁-C₄-alkylthio, C₁-C₄-alkylthio-C₁-C₄-alkyl and C₃-C₆-cycloalkyl) and the heteroaryl-C₁-C₄-alkyl radicals triazolyl-C₁-C₄-alkyl, pyridyl-C₁-C₄-alkyl, pyrimidyl-C₁-C₄-alkyl and oxadiazolyl-C₁-C₄-alkyl (which for their part may be substituted by halogen and C₁-C₄-alkyl), or
G² represents a radical from the group consisting of (C-1) and (C-6) to (C-9)

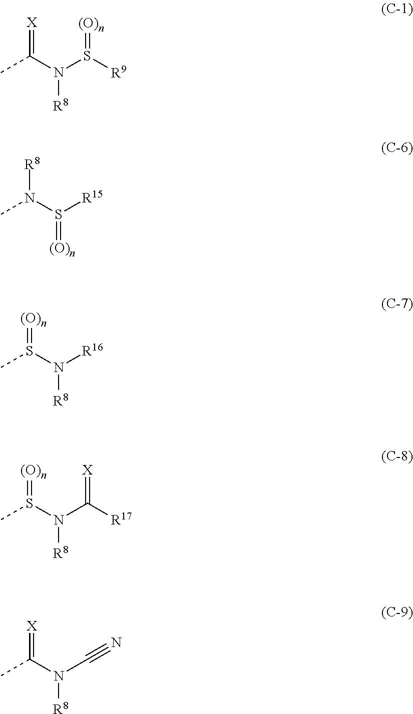

where the broken line represents the bond to the B radicals,
X represents oxygen or sulfur,
X¹ represents a radical from the group consisting of hydrogen, halogen, cyano, nitro, C₁-C₆-alkyl, C₁-C₆-haloalkyl, C₃-C₆-cycloalkyl, C₁-C₆-alkoxy and C₁-C₆-haloalkoxy,
X² represents oxygen, sulfur, NR⁵ or NOH,
V—Z represents R²⁴CH—CHR²⁵ or R²⁴C═CR²⁵,
n represents 1 or 2,
R represents NR¹⁸R¹⁹ or represents C₁-C₆-alkyl, C₃-C₆-alkenyl, C₃-C₆-alkynyl, C₁-C₆-alkoxy-C₁-C₄-alkyl, C₁-C₆-alkyl-S—C₁-C₄-alkyl, C₁-C₆-alkyl-S(O)—C₁-C₄-alkyl, C₁-C₆-alkyl-S(O)₂—C₁-C₄-alkyl, each of which is optionally substituted by halogen, oxygen (leads to C═O) or cyano, represents R¹⁸—CO—C₁-C₄-alkyl, represents NR¹⁸R¹⁹—CO—C₁-C₄-alkyl, represents C₃-C₆-cycloalkyl which is optionally mono- or disubstituted by oxygen (leads to C═O), C₁-C₄-alkyl, C₃-C₈-cycloalkyl, C₁-C₄-alkoxy and C₁-C₄-haloalkyl, represents C₃-C₈-cycloalkenyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkenyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, or represents phenyl, phenyl-C₁-C₄-alkyl, hetaryl and hetaryl-C₁-C₄-alkyl, each of which is optionally mono- to trisubstituted by halogen, cyano, C₁-C₄-alkyl, C₁-C₄-haloalkyl, C₃-C₆-cycloalkyl, C₁-C₄-alkoxy or C₁-C₄-haloalkoxy,
R³ represents hydrogen or C₁-C₆-alkyl,
R⁴ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl,
R⁵ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl, or
R³ and R⁴ together with the nitrogen atom to which they are bonded form a 4- to 7-membered ring which may contain one or two further heteroatoms from the group of nitrogen, oxygen and sulfur (where oxygen and sulfur atoms must not be directly adjacent to one another),
R⁶ represents hydrogen or C₁-C₄-alkyl,
R⁷ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₄-cycloalkyl-C₁-C₄-alkyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl, or
R⁶ and R⁷ together with the nitrogen atom to which they are attached form a 4- to 7-membered ring which may contain one or two further heteroatoms from the group consisting of nitrogen, oxygen and sulfur (where oxygen and sulfur atoms must not be directly adjacent to one another),
R⁸ represents a radical from the group consisting of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulfonyl, optionally halogen-substituted C₁-C₆-alkoxycarbonyl and optionally halogen-, C₁-C₆-alkyl-, C₁-C₆-alkoxy-, C₁-C₆-haloalkyl- and cyano-substituted C₃-C₆-cycloalkylcarbonyl, or represents a cation or an optionally C₁-C₆-alkyl- or aryl-C₁-C₆-alkyl-substituted ammonium ion,
R⁹ represents a radical from the group consisting of in each case optionally halogen-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl- and C₁-C₆-haloalkylsulfonyl-substituted C₁-C₆-alkyl, C₂-C₆-alkenyl and C₂-C₆-alkynyl, in each case optionally halogen-, C₁-C₆-alkyl-, C₁-C₆-haloalkyl-, C₁-C₆-alkoxy- or C₁-C₆-haloalkoxy-substituted C₃-C₆-cycloalkyl, C₃-C₆-cycloalkyl-C₁-C₆-alkyl and C₃-C₆-cycloalkenyl, in which one ring member may be replaced by a heteroatom from the group consisting of sulfur, oxygen (where oxygen atoms must not be directly adjacent to one another) and nitrogen (and here in particular represent

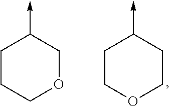

where the arrow in each case marks the bond to the sulfur atom in the radical (C-1) and in the radical (F-1)), in each case optionally halogen-, cyano- (including in the alkyl moiety), nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di(C₁-C₆-alkyl)amino-, C₁-C₆-alkylcarbonylamino-, C₁-C₆-alkoxycarbonylamino-, C₁-C₆-alkoxy-C₁-C₆-alkyl-, C₁-C₆-haloalkoxy-C₁-C₆-alkyl-, C₂-C₆-alkenyl-, C₂-C₆-alkynyl-, C₃-C₆-cycloalkyl-C₁-C₆-alkyl-, C₁-C₆-alkylcarbonyl-, C₁-C₆-alkoxycarbonyl- or aminocarbonyl-substituted aryl, heteroaryl, aryl-C₁-C₆-alkyl, heteroaryl-C₁-C₆-alkyl or represents NR′R″ in which R′ and R″ independently of one another represent a radical from the group consisting of hydrogen, C₁-C₆-alkyl, C₁-C₆-haloalkyl, C₃-C₆-cycloalkyl, C₁-C₆-alkoxy, C₁-C₆-alkylcarbonyl and C₁-C₆-alkoxylcarbonyl, or
R⁸ and R⁹ in the radical (C-1) and in the radical (F-1) may also form, together with the N—S(O)ₙ group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group consisting of sulfur, oxygen (where oxygen atoms must not be directly adjacent to one another) and nitrogen and/or at least one and preferably one carbonyl group, in particular, R⁸ and R⁹ together with the N—S(O)ₙ group to which they are attached may represent a radical from the group consisting of

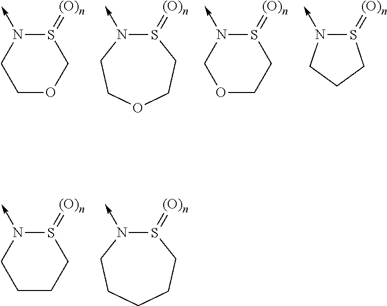

(in which the arrow in each case marks the bond to the C(X) group),
R¹⁵ represents a radical from the group consisting of in each case optionally methyl-, cyano-, carbamoyl-substituted C₁-C₆-alkyl, C₂-C₆-alkenyl and C₂-C₆-alkynyl, in each case optionally methyl-, trifluoromethyl-, halogen-, cyano- or carbamoyl-substituted C₃-C₆-cycloalkyl, C₃-C₆-cycloalkyl-C₁-C₂-alkyl and C₃-C₆-cycloalkenyl, in which the rings may contain at least one heteroatom from the group consisting of sulfur, oxygen (where oxygen atoms must not be directly adjacent to one another) and nitrogen, in each case optionally C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-haloalkylsulfinyl-, C₁-C₄-haloalkylsulfonyl-, C₁-C₄-alkylamino-, di-(C₁-C₄-alkyl)-amino-, halogen-, nitro- or cyano-substituted aryl, heteroaryl, aryl-C₁-C₄-alkyl and heteroaryl-C₁-C₂-alkyl and an optionally C₁-C₄-alkyl-, C₁-C₄-alkylcarbonyl-, C₁-C₄-alkyl-C₁-C₄-alkoxycarbonyl- or C₁-C₄-alkylsulfonyl-substituted amino group,
R⁸ and R¹⁵ in the radical (C-6) may also form, together with the N—S(O)n group to which they are attached, a saturated or unsaturated and optionally substituted 4- to 8-membered ring which may contain one or two further heteroatoms from the group consisting of sulfur, oxygen (where oxygen and sulfur atoms must not be directly adjacent to one another) and nitrogen and/or at least one carbonyl group,
R¹⁶ represents a radical from the group consisting of hydrogen, in each case optionally methyl-, cyano-, carbamoyl- or carboxyl-substituted C₁-C₆-alkyl, C₁-C₆-alkoxy, C₂-C₄-alkenyl and C₂-C₄-alkynyl, in each case optionally halogen-, cyano-, nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di-(C₁-C₆-alkyl)amino-, C₁-C₆-alkylcarbonylamino-, C₁-C₆-alkoxycarbonylamino-, C₂-C₆-alkenyl-, C₂-C₆-alkynyl- or C₁-C₆-alkylcarbonyl-substituted C₃-C₆-cycloalkyl, C₃-C₆-cycloalkyl-C₁-C₄-alkyl and C₃-C₆-cycloalkenyl in which the rings may contain at least one heteroatom from the group consisting of sulfur, oxygen (where oxygen atoms must not be directly adjacent to one another) and nitrogen, in each case optionally halogen-, cyano-, nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di-(C₁-C₆-alkyl)amino-, C₁-C₆-alkylcarbonylamino-, C₁-C₆-alkoxycarbonylamino-, C₂-C₆-alkenyl-, C₂-C₆-alkynyl- or C₁-C₆-alkylcarbonyl-substituted aryl, heteroaryl, aryl-C₁-C₂-alkyl and heteroaryl-C₁-C₂-alkyl and an optionally C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₂-C₆-alkenyl-, C₂-C₆-alkynyl- or C₁-C₆-alkylcarbonyl-substituted amino group,
R¹⁷ represents a radical from the group consisting of in each case optionally halogen-, C₁-C₆-alkoxy-, C₁-C₆-haloalkoxy C₁-C₆-alkylthio-, C₁-C₆-haloalkylthio-, C₁-C₆-alkylsulfinyl-, C₁-C₆-haloalkylsulfinyl-, C₁-C₆-alkylsulfonyl- or C₁-C₆-haloalkylsulfonyl-substituted C₁-C₆-alkyl, C₁-C₆-alkoxy, C₂-C₆-alkenyl and C₂-C₆-alkynyl, in each case optionally halogen-, C₁-C₆-alkyl-, C₁-C₆-haloalkyl-, C₁-C₆-alkoxy- or C₁-C₆-haloalkoxy-substituted C₃-C₆-cycloalkyl, C₃-C₆-cycloalkyl-C₁-C₆-alkyl, C₃-C₆-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl or N-2-oxo-1-methylpiperazinyl, in each case optionally halogen-, cyano- (also in the alkyl moiety), nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di(C₁-C₆-alkyl)amino-, C₁-C₆-alkylcarbonylamino-, C₁-C₆-alkoxycarbonylamino-, C₁-C₆-alkoxy-C₁-C₆-alkyl-, C₁-C₆-haloalkoxy-C₁-C₆-alkyl-, C₂-C₆-alkenyl-, C₂-C₆-alkynyl-, C₃-C₆-cycloalkyl-C₁-C₆-alkyl-, C₁-C₆-alkylcarbonyl-, C₁-C₆-alkoxycarbonyl- or aminocarbonyl-substituted aryl, heteroaryl, aryl-C₁-C₆-alkyl, heteroaryl-C₁-C₆-alkyl or represents NR′R″ in which R′ and R″ independently of one another each represent a radical from the group consisting of hydrogen, C₁-C₆-alkyl, C₁-C₆-haloalkyl, C₃-C₆-cycloalkyl, C₁-C₆-alkoxy, C₁-C₆-alkylcarbonyl and C₁-C₆-alkoxycarbonyl,
R⁸ and R¹⁷ in the radical (C-8) and in the radical (F-8) may also form, together with the N—C(X) group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two further heteroatoms from the group consisting of sulfur, oxygen (where oxygen atoms must not be directly adjacent to one another) and nitrogen and/or one carbonyl group, in particular, R⁸ and R¹⁷ together with the N—C(X) group to which they are attached may represent a radical from the group consisting of

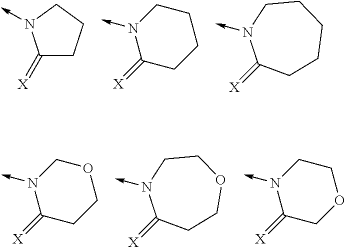

(where the arrow in each case denotes the bond to the sulfur atom in the radical (C-8) and in the radical (F-8)),
R¹⁸ represents a radical from the group consisting of hydrogen, hydroxy, of C₁-C₆-alkyl, C₁-C₆-alkoxy, C₁-C₆-alkoxy-C₁-C₄-alkyl, C₁-C₆-alkyl-S—C₁-C₄-alkyl, C₁-C₆-alkyl-S(O)—C₁-C₄-alkyl, C₁-C₆-alkyl-S(O)₂—C₁-C₄-alkyl, C₁-C₆-alkylcarbonyl, C₁-C₆-alkoxycarbonyl, C₃-C₆-cycloalkyl, C₃-C₆-cycloalkenyl, C₃-C₆-cycloalkyl-C₁-C₃-alkyl, C₃-C₆-cycloalkenyl-C₁-C₃-alkyl, heterocyclyl, heterocyclyl-C₁-C₃-alkyl, each of which is optionally mono- or polysubstituted by halogen or mono- or disubstituted by cyano, and phenyl, phenyl-C₁-C₃-alkyl, hetaryl and hetaryl-C₁-C₃-alkyl, each of which is optionally mono- to tetrasubstituted by C₁-C₄-alkyl, C₁-C₄-haloalkyl, C₁-C₄-alkoxy, C₁-C₄-haloalkoxy, C₃-C₆-cycloalkyl, halogen or cyano,
R¹⁹ represents hydrogen, an alkali or alkaline earth metal ion, or represents an ammonium ion which is optionally mono- to tetrasubstituted by C₁-C₄-alkyl, or represents a radical from the group consisting of C₁-C₄-alkyl, C₁-C₄-alkoxy, C₁-C₄-alkoxy-C₁-C₄-alkyl, C₁-C₄-alkyl-S—C₁-C₄-alkyl, C₁-C₄-alkyl-S(O)—C₁-C₄-alkyl and C₁-C₄-alkyl-S(O)₂—C₁-C₄-alkyl,
Y³ represents a radical from the group consisting of hydrogen, halogen, cyano, C₁-C₆-alkyl, C₁-C₆-haloalkyl, C₃-C₆-cycloalkyl, C₁-C₆-alkoxy, C₁-C₆-haloalkoxy and NR²⁰R²¹,
W represents a radical from the group consisting of O, S, SO and SO₂,
R²² represents a radical from the group consisting of hydrogen, C₁-C₆-alkyl, C₂-C₆-haloalkyl, cyano-C₁-C₆-alkyl, C₂-C₄-alkenyl, C₂-C₄-haloalkenyl, C₂-C₄-alkynyl, C₂-C₄-haloalkynyl, C₁-C₆-alkoxy, C₁-C₆-haloalkoxy, optionally halogen-substituted C₁-C₆-alkoxy-C₁-C₆-alkyl, optionally halogen-substituted bis(C₁-C₆-alkoxy)-C₁-C₆-alkyl, optionally halogen-substituted C₁-C₆-alkylsulfanyl-C₁-C₆-alkyl, optionally halogen-substituted C₁-C₄-alkylcarbonyl-C₁-C₄-alkyl, optionally halogen-substituted C₁-C₆-alkylsulfinyl-C₁-C₆-alkyl, optionally halogen-substituted C₁-C₆-alkylsulfonyl-C₁-C₆-alkyl, di-(C₁-C₆-alkyl)-aminosulfanyl-C₁-C₆-alkyl, di-(C₁-C₆-alkyl)-aminosulfinyl-C₁-C₆-alkyl, di-(C₁-C₆-alkyl)-aminosulfonyl-C₁-C₆-alkyl, optionally halogen-substituted C₁-C₆-alkoxycarbonyl,
optionally halogen-substituted C₁-C₆-alkoxycarbonyl-C₁-C₆-alkyl, optionally halogen-substituted C₂-C₄-alkynyloxy, optionally halogen-substituted C₂-C₄-alkynyloxycarbonyl, di-(C₁-C₆-alkyl)-aminocarbonyl, N—C₁-C₆-alkyl-N—C₃-C₆-cycloalkylaminocarbonyl, di-(C₁-C₆-alkyl)-aminocarbonyl-C₁-C₆-alkyl, N—C₁-C₆-alkyl-N—C₃-C₆-cycloalkylaminocarbonyl-C₁-C₆-alkyl, heterocyclylcarbonyl-C₁-C₆-alkyl, C₁-C₆-alkylsulfanyl, C₁-C₆-haloalkylsulfanyl, C₁-C₆-alkylsulfinyl, C₁-C₆-haloalkylsulfinyl, C₁-C₆-alkylsulfonyl, C₁-C₆-haloalkylsulfonyl, C₃-C₆-cycloalkyl which is optionally substituted by halogen, cyano, nitro, C₁-C₆-alkyl, C₁-C₆-haloalkyl, C₃-C₆-cycloalkyl, C₁-C₆-alkoxy, C₁-C₆-haloalkoxy, C₁-C₆-alkoxycarbonyl, C₁-C₆-haloalkoxycarbonyl or hetaryl (which for its part is optionally substituted by C₁-C₆-alkyl or halogen),
C₃-C₆-cycloalkylcarbonyl which is optionally substituted by halogen, cyano, nitro, C₁-C₆-alkyl, C₁-C₆-haloalkyl, C₃-C₆-cycloalkyl, C₁-C₆-alkoxy, C₁-C₆-haloalkoxy, C₁-C₆-alkoxycarbonyl, C₁-C₆-haloalkoxycarbonyl or hetaryl (which for its part is optionally substituted by C₁-C₆-alkyl or halogen), C₃-C₆-cycloalkyl-C₁-C₆-alkyl which is optionally substituted by halogen, cyano, nitro, C₁-C₆-alkyl, C₁-C₆-haloalkyl, C₃-C₆-cycloalkyl, C₁-C₆-alkoxy, C₁-C₆-haloalkoxy, C₁-C₆-alkoxycarbonyl, C₁-C₆-haloalkoxycarbonyl or hetaryl (which for its part is optionally substituted by C₁-C₆-alkyl or halogen), heterocyclyl-C₁-C₆-alkyl which is optionally substituted by halogen, cyano (also in the C₁-C₆-alkyl moiety of heterocyclyl-C₁-C₆-alkyl), nitro, hydroxy, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy, C₁-C₆-haloalkoxy, C₁-C₆-alkylthio, C₁-C₆-haloalkylthio, C₁-C₆-alkylsulfinyl, C₁-C₆-alkylsulfonyl, C₁-C₆-haloalkylsulfinyl, C₁-C₆-haloalkylsulfonyl, amino, C₁-C₆-alkylamino, di-(C₁-C₆-alkyl)-amino, C₁-C₆-alkylcarbonylamino, C₁-C₆-alkoxycarbonylamino, C₁-C₆-alkoxy-C₁-C₆-alkyl, C₁-C₆-haloalkoxy-C₁-C₆-alkyl, C₂-C₆-alkenyl, C₂-C₆-alkynyl, C₃-C₆-cycloalkyl-C₁-C₆-alkyl, C₁-C₆-alkylcarbonyl, C₁-C₆-alkoxycarbonyl or aminocarbonyl, aryl which is optionally substituted by halogen, cyano, nitro, hydroxy, amino, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy or C₁-C₆-haloalkoxy, aryl-C₁-C₆-alkyl which is optionally substituted by halogen, cyano (also in the C₁-C₆-alkyl moiety of aryl-C₁-C₆-alkyl), nitro, hydroxy, amino, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy or C₁-C₆-haloalkoxy, hetaryl-C₁-C₆-alkyl which is optionally substituted by halogen, cyano (also in the C₁-C₆-alkyl moiety of hetaryl-C₁-C₆-alkyl), nitro, hydroxy, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy, C₁-C₆-haloalkoxy, C₁-C₆-alkylthio, C₁-C₆-haloalkylthio, C₁-C₆-alkylsulfinyl, C₁-C₆-alkylsulfonyl, C₁-C₆-haloalkylsulfinyl, C₁-C₆-haloalkylsulfonyl, amino, C₁-C₆-alkylamino, di-(C₁-C₆-alkyl)-amino, C₁-C₆-alkylcarbonylamino, C₁-C₆-alkoxycarbonylamino, C₁-C₆-alkoxy-C₁-C₆-alkyl, C₁-C₆-haloalkoxy-C₁-C₆-alkyl, C₂-C₆-alkenyl, C₂-C₆-alkynyl, C₁-C₆-alkyl-C₃-C₆-cycloalkyl, C₃-C₆-cycloalkyl-C₁-C₆-alkyl, C₁-C₆-alkylcarbonyl, C₁-C₆-alkoxycarbonyl or aminocarbonyl, or
R²² represents a radical from the group consisting of (D-1) to (D-3)

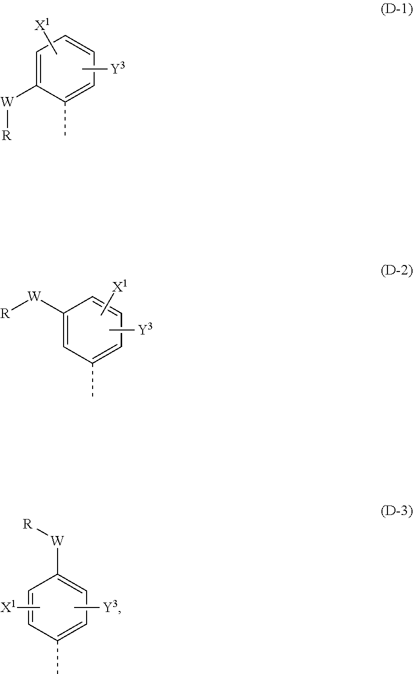

from the group consisting of (E-1) to (E-11)

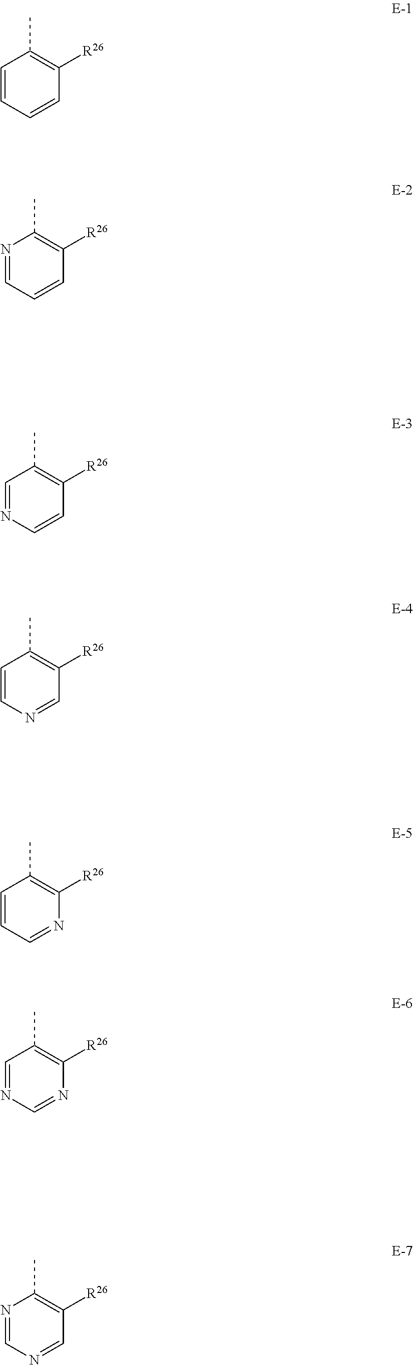

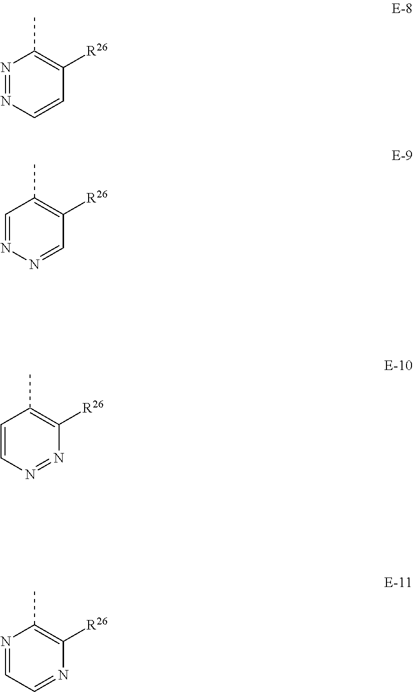

and (E-18) to (E-51)

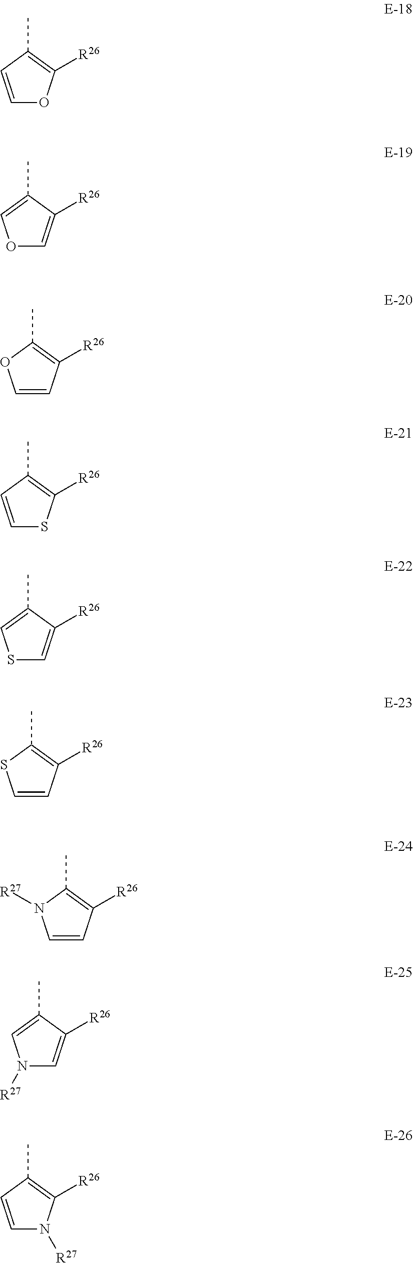

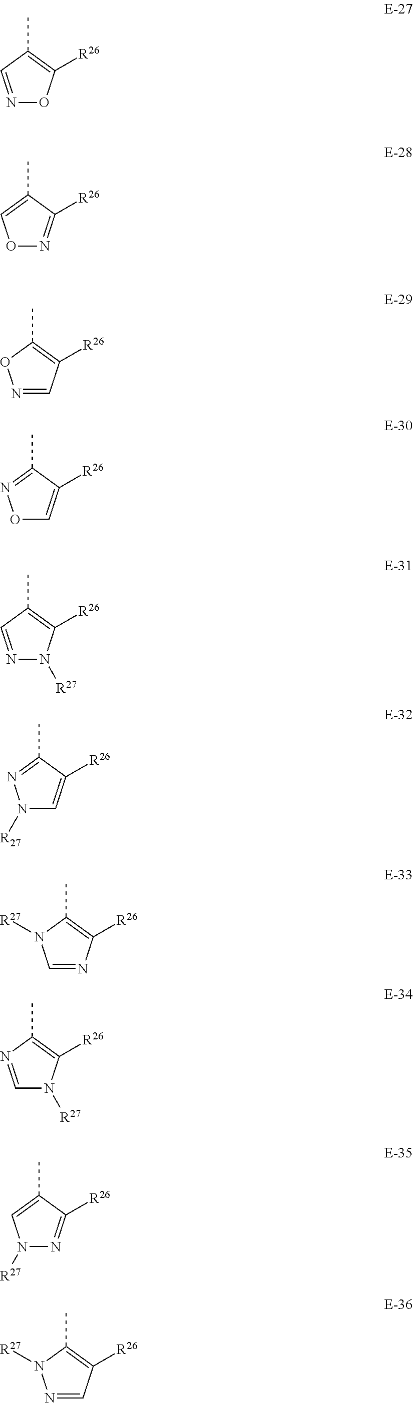

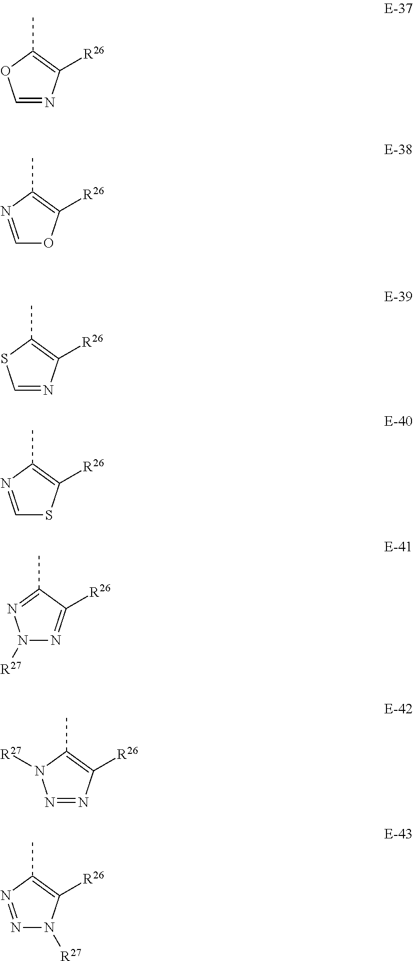

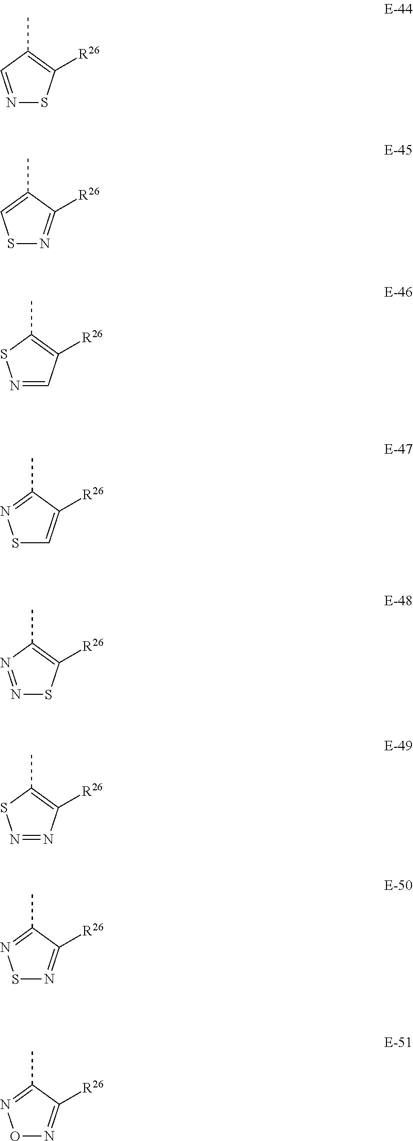

or
in the case R²=d),
R²² also represents an E radical from the group consisting of E-12 to E-17

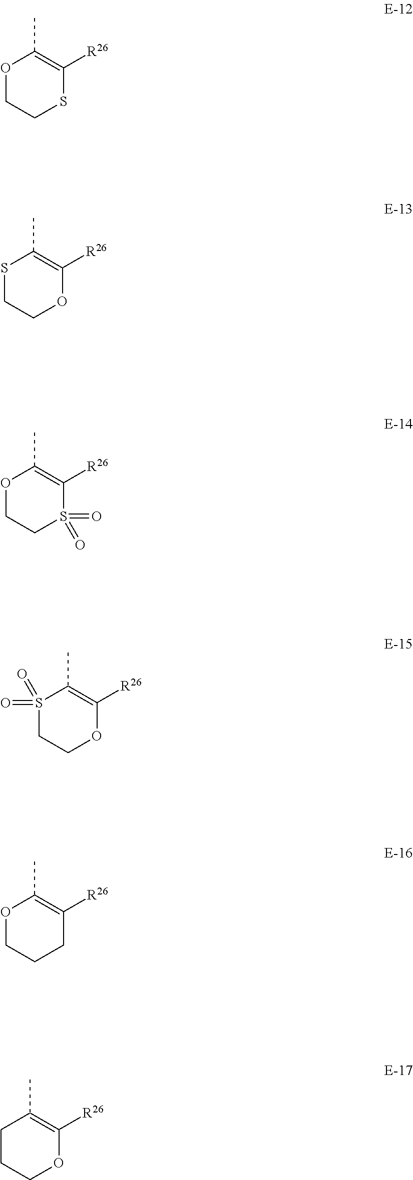

R²⁰ represents a radical from the group consisting of hydrogen, halogen, cyano, nitro, amino, hydroxy, C₁-C₆-alkyl, C₁-C₆-haloalkyl, cyano-C₁-C₆-alkyl, C₂-C₆-alkenyl, C₂-C₆-alkynyl, C₃-C₆-cycloalkyl (which is optionally substituted by halogen, cyano, C₁-C₄-alkyl and C₃-C₆-cycloalkyl), C₃-C₆-cycloalkyl-C₁-C₆-alkyl, C₁-C₆-alkoxy, C₁-C₆-haloalkoxy, C₃-C₆-alkenyloxy, C₃-C₆-alkynyloxy, C₃-C₆-cycloalkyloxy, C₁-C₆-alkylcarbonyloxy, C₂-C₆-alkenylcarbonyloxy, C₂-C₆-alkynylcarbonyloxy, C₃-C₆-cycloalkylcarbonyloxy, C₁-C₆-alkoxycarbonyloxy, C₁-C₆-alkylsulfonyloxy, C₁-C₆-alkylamino, C₃-C₆-alkenylamino, C₃-C₆-alkynylamino, C₃-C₆-cycloalkylamino, C₁-C₆-alkylthio, C₁-C₆-haloalkylthio, C₃-C₆-alkenylthio, C₃-C₆-alkynylthio, C₃-C₆-cycloalkylthio, C₁-C₆-alkylsulfinyl, C₁-C₆-alkylsulfonyl, C₁-C₆-alkylcarbonyl, C₁-C₆-alkoxyimino-C₁-C₆-alkyl, C₁-C₆-alkoxycarbonyl, aminocarbonyl, C₁-C₆-alkylaminocarbonyl, di-(C₁-C₆-alkyl)-aminocarbonyl, aminothiocarbonyl, C₁-C₆-alkylaminosulfonyl, C₁-C₆-alkylsulfonylamino, C₁-C₆-alkylcarbonylamino, C₁-C₆-alkylthiocarbonylamino, of phenyl, phenoxy, pyridinyl and pyridinyloxy, each of which is optionally substituted by a radical from the group consisting of halogen, cyano, nitro, amino, hydroxy, C₁-C₆-alkyl and C₁-C₆-haloalkyl,
R²¹ represents a radical from the group consisting of hydrogen, C₁-C₆-alkyl, C₃-C₆-cycloalkyl, C₁-C₆-haloalkyl, C₃-C₆-alkenyl, C₂-C₆-alkynyl, C₃-C₆-cycloalkyl-C₁-C₆-alkyl, cyano-C₁-C₆-alkyl, C₁-C₆-alkylcarbonyl, C₂-C₆-alkenylcarbonyl, C₁-C₆-haloalkylcarbonyl, C₂-C₆-haloalkenylcarbonyl, C₁-C₆-alkoxy-C₁-C₆-alkyl, C₁-C₆-alkoxycarbonyl, C₁-C₆-alkylsulfonyl and C₁-C₆-haloalkylsulfonyl,
R²³ represents a radical from the group consisting of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄alkyl,
or, if R²=c) or g),
R²² and R²³ together with the nitrogen atom to which they are attached form a saturated ring which may contain one or more further heteroatoms from the group consisting of nitrogen, oxygen and sulfur, and
R²⁴ represents hydrogen or represents an in each case optionally halogen- or cyano-substituted radical from the group consisting of C₁-C₄-alkyl, C₂-C₆-alkenyl, C₂-C₆-alkynyl, phenyl and phenyl-C₁-C₂-alkyl,
R²⁵ represents hydrogen or represents an in each case optionally halogen- or cyano-substituted radical from the group consisting of C₁-C₄-alkyl, C₂-C₆-alkenyl, C₂-C₆-alkynyl, phenyl and phenyl-C₁-C₂-alkyl,
R²⁶ represents a radical from the group consisting of hydrogen, C₁-C₄-alkyl, C₁-C₄-haloalkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₆-alkylsulfanyl, C₁-C₆-haloalkylsulfanyl C₁-C₆-alkylsulfinyl, C₁-C₆-haloalkylsulfinyl, C₁-C₆-alkylsulfonyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl and cyano-C₁-C₄-alkyl and
R²⁷ represents hydrogen, C₁-C₄-alkyl, C₁-C₄-haloalkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl or cyano-C₁-C₄-alkyl and
compounds of the formula (I) in which
A represents the A radical

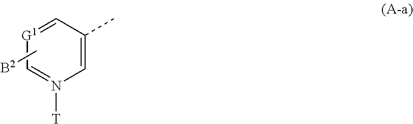

in which the broken line denotes the bond to the carbon atom of the bicyclic system of the formula (I) and
G¹ represents N or C—B¹,
B¹ represents a radical from the group consisting of hydrogen, halogen, C₁-C₆-alkyl and C₁-C₄-haloalkyl,
B² represents a radical from the group consisting of hydrogen, halogen, C₁-C₆-alkyl and C₁-C₄-haloalkyl,
T represents oxygen or an electron pair,
Q represents sulfur,
R¹ represents a radical from the group consisting of hydrogen, C₁-C₆-alkyl and C₁-C₆-alkoxy,
R² a) represents a B radical from the group consisting of

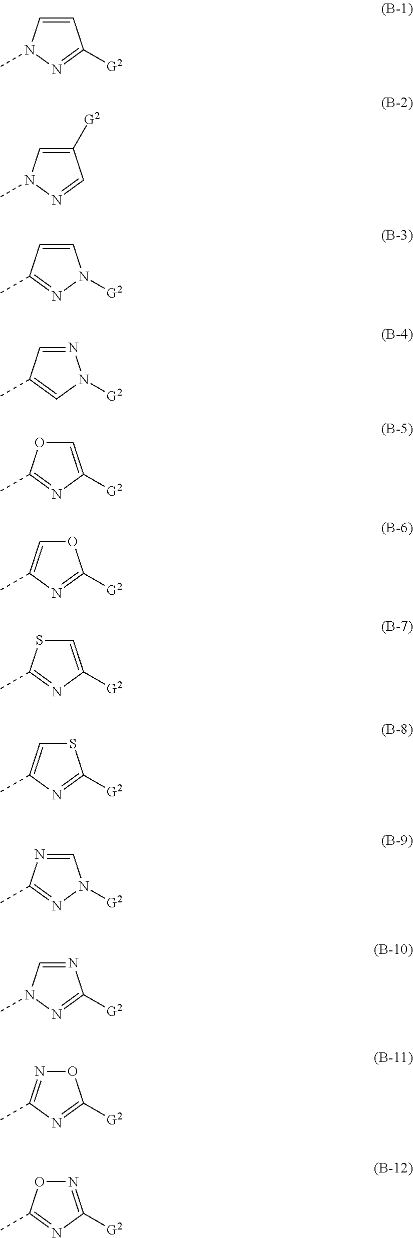

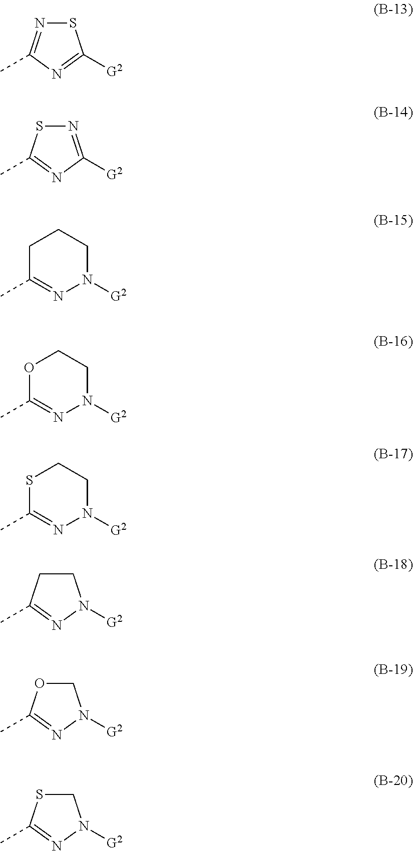

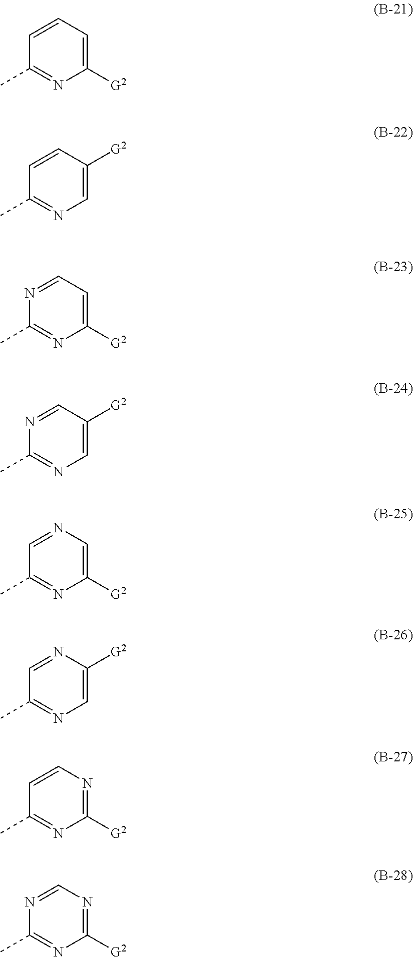

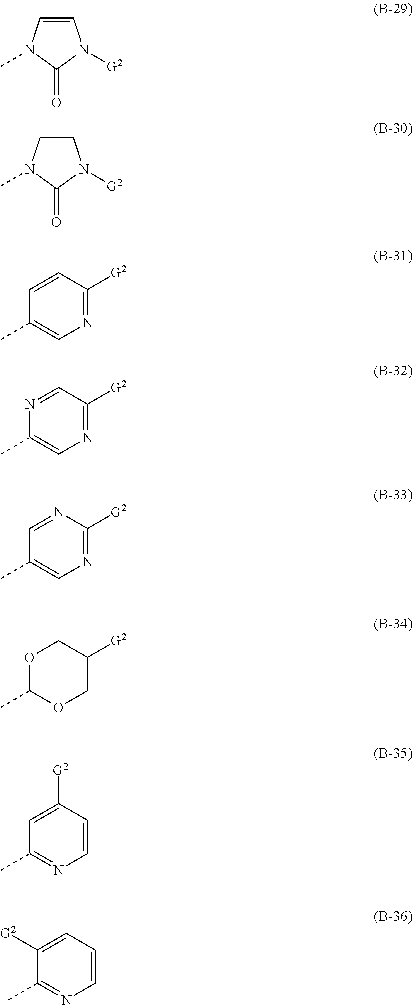

where the broken line denotes the bond to the carbon atom of the bicyclic system of the formula (I), or
R² c) represents a radical of the formula

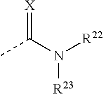

where the broken line denotes the bond to the carbon atom of the bicyclic system of the formula (I), or
R² d) represents a radical of the formula

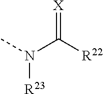

where the broken line denotes the bond to the carbon atom of the bicyclic system of the formula (I), or
R² e) represents an F radical from the group consisting of (F-8), (F-10) and (F-11)

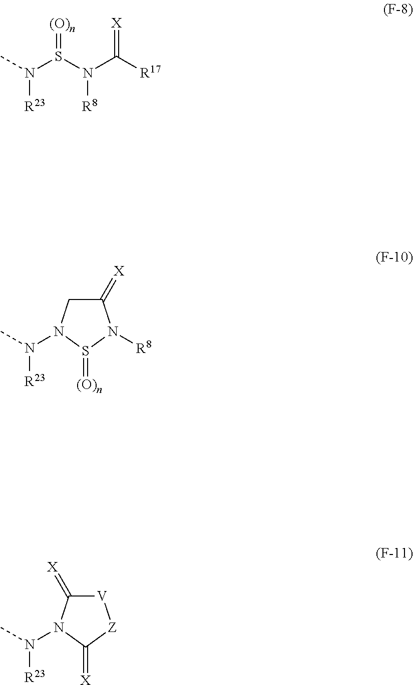

where the broken line denotes the bond to the carbon atom in the formula (I) or
R² f) represents a radical from the group consisting of C₁-C₆-haloalkyl and carboxyl, or
R² g) represents a radical of the formula

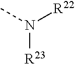

where the broken line represents the bond to the carbon atom of the bicyclic system of the formula (I), where
G² represents hydrogen or represents a radical from the group consisting of halogen, nitro, amino, cyano, C₁-C₄-alkylamino, halo-C₁-C₄-alkylamino, di-(C₁-C₄-alkyl)-amino, C₁-C₄-alkyl, halo-C₁-C₄-alkyl, C₃-C₆-cycloalkyl, C₃-C₆-cycloalkenyl, C₃-C₆-cycloalkyl-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy-(C₁-C₄-alkylsulfanyl)-C₁-C₄-alkyl, C₁-C₄-alkoxy-(C₁-C₄-alkylsulfinyl)-C₁-C₄-alkyl, C₁-C₄-alkoxy-(C₁-C₄-alkylsulfonyl)-C₁-C₄-alkyl, bis(C₁-C₄-alkylsulfanyl)-C₁-C₄-alkyl, bis(halo-C₁-C₄-alkylsulfanyl)-C₁-C₄-alkyl, bis(hydroxy-C₁-C₄-alkylsulfanyl)-C₁-C₄-alkyl, C₁-C₄-alkoxycarbonyl, C₁-C₄-alkoxycarbonyl-C₁-C₄-alkyl, alpha-hydroxyimino-C₁-C₄-alkoxycarbonyl-C₁-C₄-alkyl, alpha-C₁-C₄-alkoxyimino-C₁-C₄-alkoxycarbonyl-C₁-C₄-alkyl, C(X²)NR³R⁴, NR⁶R⁷, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by C₁-C₄-alkyl, halo-C₁-C₄-alkyl, C₁-C₄-alkoxy and C₁-C₄-alkoxy-C₁-C₄-alkyl), phenyl (which for its part may be substituted by halogen, cyano, nitro, C₁-C₄-alkyl and halo-C₁-C₄-alkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, C₁-C₄-alkyl, halo-C₁-C₄-alkyl, C₁-C₄-alkoxy, halo-C₁-C₄-alkoxy, C₁-C₄-alkoxy-C₁-C₄-alkyl, C₁-C₄-alkylthio, C₁-C₄-alkylthio-C₁-C₄-alkyl and C₃-C₆-cycloalkyl) and the heteroaryl-C₁-C₄-alkyl radicals triazolyl-C₁-C₄-alkyl, pyridyl-C₁-C₄-alkyl, pyrimidyl-C₁-C₄-alkyl and oxadiazolyl-C₁-C₄-alkyl (which for their part may be substituted by halogen and C₁-C₄-alkyl), or
G² represents a C radical from the group consisting of (C-1) and (C-6) to (C-9)

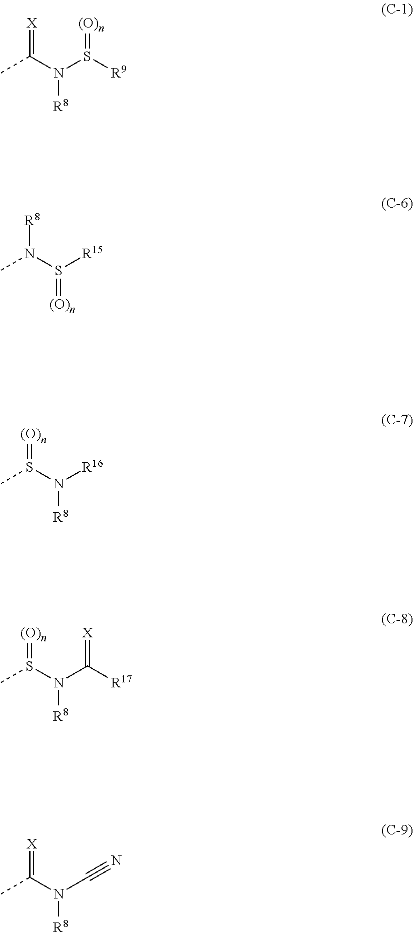

where the broken line denotes the bond to the B radicals,
X represents oxygen or sulfur,
X² represents oxygen, sulfur, NR⁵ or NOH,
V—Z represents R²⁴CH—CHR²⁵ or R²⁴C═CR²⁵,
n represents 1 or 2,
R³ represents hydrogen or C₁-C₆-alkyl,
R⁴ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl,
R⁵ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl, or
R³ and R⁴ together with the nitrogen atom to which they are bonded form a 4- to 7-membered ring which may contain one or two further heteroatoms from the group of nitrogen, oxygen and sulfur (where oxygen and sulfur atoms must not be directly adjacent to one another),
R⁶ represents hydrogen or C₁-C₄-alkyl,
R⁷ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₄-cycloalkyl-C₁-C₄-alkyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl, aryl-C₁-C₄-alkyl and hetaryl-C₁-C₄-alkyl, or
R⁶ and R⁷ together with the nitrogen atom to which they are bonded form a 4- to 7-membered ring which may contain one or two further heteroatoms from the group of nitrogen, oxygen and sulfur (where oxygen and sulfur atoms must not be directly adjacent to one another),
R⁸ is a radical from the group of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulfonyl, optionally halogen-substituted C₁-C₆-alkoxycarbonyl and optionally halogen-, C₁-C₆-alkyl-, C₁-C₆-alkoxy-, C₁-C₆-haloalkyl- and cyano-substituted C₃-C₆-cycloalkylcarbonyl, or is a cation or an optionally C₁-C₆-alkyl- or aryl-C₁-C₆-alkyl-substituted ammonium ion,
R⁹ represents a radical from the group consisting of in each case optionally halogen-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl- and C₁-C₆-haloalkylsulfonyl-substituted C₁-C₆-alkyl, C₂-C₆-alkenyl and C₂-C₆-alkynyl, in each case optionally halogen-, C₁-C₆-alkyl-, C₁-C₆-haloalkyl-, C₁-C₆-alkoxy- or C₁-C₆-haloalkoxy-substituted C₃-C₆-cycloalkyl, C₃-C₆-cycloalkyl-C₁-C₆-alkyl and C₃-C₆-cycloalkenyl, in which one ring member may be replaced by a heteroatom from the group consisting of sulfur, oxygen (where oxygen atoms must not be directly adjacent to one another) and nitrogen (and here in particular represent

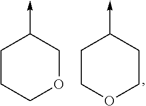

where the arrow in each case marks the bond to the sulfur atom in the radical (C-1) and in the radical (F-1)), in each case optionally halogen-, cyano- (including in the alkyl moiety), nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di(C₁-C₆-alkyl)amino-, C₁-C₆-alkylcarbonylamino-, C₁-C₆-alkoxycarbonylamino-, C₁-C₆-alkoxy-C₁-C₆-alkyl-, C₁-C₆-haloalkoxy-C₁-C₆-alkyl-, C₂-C₆-alkenyl-, C₂-C₆-alkynyl-, C₃-C₆-cycloalkyl-C₁-C₆-alkyl-, C₁-C₆-alkylcarbonyl-, C₁-C₆-alkoxycarbonyl- or aminocarbonyl-substituted aryl, heteroaryl, aryl-C₁-C₆-alkyl, heteroaryl-C₁-C₆-alkyl or represents NR′R″ in which R′ and R″ independently of one another represent a radical from the group consisting of hydrogen, C₁-C₆-alkyl, C₁-C₆-haloalkyl, C₃-C₆-cycloalkyl, C₁-C₆-alkoxy, C₁-C₆-alkylcarbonyl and C₁-C₆-alkoxylcarbonyl, or
R⁸ and R⁹ in the radical (C-1) may also form, together with the N—S(O)ₙ group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group consisting of sulfur, oxygen (where oxygen atoms must not be directly adjacent to one another) and nitrogen and/or at least one and preferably one carbonyl group, in particular, R⁸ and R⁹ together with the N—S(O)ₙ group to which they are attached may represent a radical from the group consisting of

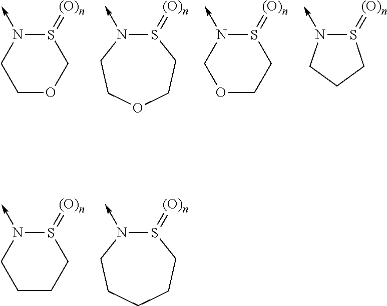

(in which the arrow in each case marks the bond to the C(X) group),
R¹⁵ represents a radical from the group consisting of in each case optionally methyl-, cyano-, carbamoyl-substituted C₁-C₆-alkyl, C₂-C₆-alkenyl and C₂-C₆-alkynyl, in each case optionally methyl-, trifluoromethyl-, halogen-, cyano- or carbamoyl-substituted C₃-C₆-cycloalkyl, C₃-C₆-cycloalkyl-C₁-C₂-alkyl and C₃-C₆-cycloalkenyl, in which the rings may contain at least one heteroatom from the group consisting of sulfur, oxygen (where oxygen atoms must not be directly adjacent to one another) and nitrogen, in each case optionally C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-haloalkylsulfinyl-, C₁-C₄-haloalkylsulfonyl-, C₁-C₄-alkylamino-, di-(C₁-C₄-alkyl)-amino-, halogen-, nitro- or cyano-substituted aryl, heteroaryl, aryl-C₁-C₄-alkyl and heteroaryl-C₁-C₂-alkyl and an optionally C₁-C₄-alkyl-, C₁-C₄-alkylcarbonyl-, C₁-C₄-alkyl-C₁-C₄-alkoxycarbonyl- or C₁-C₄-alkylsulfonyl-substituted amino group,
R⁸ and R¹⁵ in the radical (C-6) may also form, together with the N—S(O)n group to which they are attached, a saturated or unsaturated and optionally substituted 4- to 8-membered ring which may contain one or two further heteroatoms from the group consisting of sulfur, oxygen (where oxygen and sulfur atoms must not be directly adjacent to one another) and nitrogen and/or at least one carbonyl group,
R¹⁶ represents a radical from the group consisting of hydrogen, in each case optionally methyl-, cyano-, carbamoyl- or carboxyl-substituted C₁-C₆-alkyl, C₁-C₆-alkoxy, C₂-C₄-alkenyl and C₂-C₄-alkynyl, in each case optionally halogen-, cyano-, nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di-(C₁-C₆-alkyl)amino-, C₁-C₆-alkylcarbonylamino-, C₁-C₆-alkoxycarbonylamino-, C₂-C₆-alkenyl-, C₂-C₆-alkynyl- or C₁-C₆-alkylcarbonyl-substituted C₃-C₆-cycloalkyl, C₃-C₆-cycloalkyl-C₁-C₄-alkyl and C₃-C₆-cycloalkenyl in which the rings may contain at least one heteroatom from the group consisting of sulfur,
oxygen (where oxygen atoms must not be directly adjacent to one another) and nitrogen, in each case optionally halogen-, cyano-, nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di-(C₁-C₆-alkyl)amino-, C₁-C₆-alkylcarbonylamino-, C₁-C₆-alkoxycarbonylamino-, C₂-C₆-alkenyl-, C₂-C₆-alkynyl- or C₁-C₆-alkylcarbonyl-substituted aryl, heteroaryl, aryl-C₁-C₂-alkyl and heteroaryl-C₁-C₂-alkyl and an optionally C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₂-C₆-alkenyl-, C₂-C₆-alkynyl- or C₁-C₆-alkylcarbonyl-substituted amino group,
R¹⁷ represents a radical from the group consisting of in each case optionally halogen-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl- or C₁-C₆-haloalkylsulfonyl-substituted C₁-C₆-alkyl, C₁-C₆-alkoxy, C₂-C₆-alkenyl and C₂-C₆-alkynyl, in each case optionally halogen-, C₁-C₆-alkyl-, C₁-C₆-haloalkyl-, C₁-C₆-alkoxy- or C₁-C₆-haloalkoxy-substituted C₃-C₆-cycloalkyl, C₃-C₆-cycloalkyl-C₁-C₆-alkyl, C₃-C₆-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl or N-2-oxo-1-methylpiperazinyl, in each case optionally halogen-, cyano- (also in the alkyl moiety), nitro-, C₁-C₆-alkyl-, C₁-C₆-haloalkyl-, C₃-C₆-cycloalkyl-, C₁-C₆-alkoxy-, C₁-C₆-haloalkoxy-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, amino-, C₁-C₆-alkylamino-, di(C₁-C₆-alkyl)amino-, C₁-C₆-alkylcarbonylamino-, C₁-C₆-alkoxycarbonylamino-, C₁-C₆-alkoxy-C₁-C₆-alkyl-, C₁-C₆-haloalkoxy-C₁-C₆-alkyl-, C₂-C₆-alkenyl-, C₂-C₆-alkynyl-, C₃-C₆-cycloalkyl-C₁-C₆-alkyl-, C₁-C₆-alkylcarbonyl-, C₁-C₆-alkoxycarbonyl- or aminocarbonyl-substituted aryl, heteroaryl, aryl-C₁-C₆-alkyl, heteroaryl-C₁-C₆-alkyl or represents NR′R″ in which R′ and R″ independently of one another each represent a radical from the group consisting of hydrogen, C₁-C₆-alkyl, C₁-C₆-haloalkyl, C₃-C₆-cycloalkyl, C₁-C₆-alkoxy, C₁-C₆-alkylcarbonyl and C₁-C₆-alkoxylcarbonyl,
R⁸ and R¹⁷ in the radical (C-8) and in the radical (F-8) may also form, together with the N—C(X) group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two further heteroatoms from the group consisting of sulfur, oxygen (where oxygen atoms must not be directly adjacent to one another) and nitrogen and/or one carbonyl group, in particular, R⁸ and R¹⁷ together with the N—C(X) group to which they are attached may represent a radical from the group consisting of

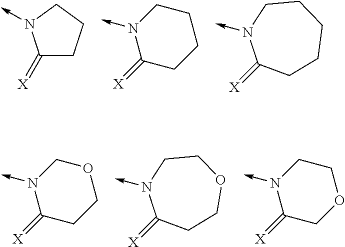

(where the arrow in each case denotes the bond to the sulfur atom in the radical (C-8) and in the radical (F-8)),
and, in the case that R² represents g),
R²² represents a radical from the group consisting of hydrogen, C₁-C₆-alkyl, C₂-C₆-haloalkyl, cyano-C₁-C₆-alkyl, C₂-C₄-alkenyl, C₂-C₄-haloalkenyl, C₂-C₄-alkynyl, C₂-C₄-haloalkynyl, C₁-C₆-alkoxy, C₁-C₆-haloalkoxy, optionally halogen-substituted C₁-C₆-alkoxy-C₁-C₆-alkyl, optionally halogen-substituted bis(C₁-C₆-alkoxy)-C₁-C₆-alkyl, optionally halogen-substituted C₁-C₆-alkylsulfanyl-C₁-C₆-alkyl, optionally halogen-substituted C₁-C₄-alkylcarbonyl-C₁-C₄-alkyl, optionally halogen-substituted C₁-C₆-alkylsulfinyl-C₁-C₆-alkyl, optionally halogen-substituted C₁-C₆-alkylsulfonyl-C₁-C₆-alkyl, di-(C₁-C₆-alkyl)-aminosulfanyl-C₁-C₆-alkyl, di-(C₁-C₆-alkyl)-aminosulfinyl-C₁-C₆-alkyl, di-(C₁-C₆-alkyl)-aminosulfonyl-C₁-C₆-alkyl, optionally halogen-substituted C₁-C₆-alkoxycarbonyl-C₁-C₆-alkyl, optionally halogen-substituted C₂-C₄-alkynyloxy, di-(C₁-C₆-alkyl)-aminocarbonyl-C₁-C₆-alkyl, N—C₁-C₆-alkyl-N—C₃-C₆-cycloalkylaminocarbonyl-C₁-C₆-alkyl, heterocyclylcarbonyl-C₁-C₆-alkyl, C₁-C₆-alkylsulfanyl, C₁-C₆-haloalkylsulfanyl, C₁-C₆-alkylsulfinyl, C₁-C₆-haloalkylsulfinyl, C₁-C₆-alkylsulfonyl, C₁-C₆-haloalkylsulfonyl, C₃-C₆-cycloalkyl which is optionally substituted by halogen, cyano, nitro, C₁-C₆-alkyl, C₁-C₆-haloalkyl, C₃-C₆-cycloalkyl, C₁-C₆-alkoxy, C₁-C₆-haloalkoxy, C₁-C₆-alkoxycarbonyl, C₁-C₆-haloalkoxycarbonyl or hetaryl (which for its part is optionally substituted by C₁-C₆-alkyl or halogen), C₃-C₆-cycloalkyl-C₁-C₆-alkyl which is optionally substituted by halogen, cyano, nitro, C₁-C₆-alkyl, C₁-C₆-haloalkyl, C₃-C₆-cycloalkyl, C₁-C₆-alkoxy, C₁-C₆-haloalkoxy, C₁-C₆-alkoxycarbonyl, C₁-C₆-haloalkoxycarbonyl or hetaryl (which for its part is optionally substituted by C₁-C₆-alkyl or halogen), heterocyclyl-C₁-C₆-alkyl which is optionally substituted by halogen, cyano (also in the C₁-C₆-alkyl moiety of heterocyclyl-C₁-C₆-alkyl), nitro, hydroxy, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy, C₁-C₆-haloalkoxy, C₁-C₆-alkylthio, C₁-C₆-haloalkylthio, C₁-C₆-alkylsulfinyl, C₁-C₆-alkylsulfonyl, C₁-C₆-haloalkylsulfinyl, C₁-C₆-haloalkylsulfonyl, amino, C₁-C₆-alkylamino, di-(C₁-C₆-alkyl)-amino, C₁-C₆-alkylcarbonylamino, C₁-C₆-alkoxycarbonylamino, C₁-C₆-alkoxy-C₁-C₆-alkyl, C₁-C₆-haloalkoxy-C₁-C₆-alkyl, C₂-C₆-alkenyl, C₂-C₆-alkynyl, C₃-C₆-cycloalkyl-C₁-C₆-alkyl, C₁-C₆-alkylcarbonyl, C₁-C₆-alkoxycarbonyl or aminocarbonyl, aryl which is optionally substituted by halogen, cyano, nitro, hydroxy, amino, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy or C₁-C₆-haloalkoxy, aryl-C₁-C₆-alkyl which is optionally substituted by halogen, cyano (also in the C₁-C₆-alkyl moiety of aryl-C₁-C₆-alkyl), nitro, hydroxy, amino, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy or C₁-C₆-haloalkoxy, hetaryl-C₁-C₆-alkyl which is optionally substituted by halogen, cyano (also in the C₁-C₆-alkyl moiety of hetaryl-C₁-C₆-alkyl), nitro, hydroxy, C₁-C₆-alkyl, C₁-C₆-haloalkyl, C₃-C₆-cycloalkyl (which is optionally substituted by halogen, cyano, C₁-C₆-alkyl and C₃-C₆-cycloalkyl), C₁-C₆-alkoxy, C₁-C₆-haloalkoxy, C₁-C₆-alkylthio, C₁-C₆-haloalkylthio, C₁-C₆-alkylsulfinyl, C₁-C₆-alkylsulfonyl, C₁-C₆-haloalkylsulfinyl, C₁-C₆-haloalkylsulfonyl, amino, C₁-C₆-alkylamino, di-C₁-C₆-alkyl)-amino, C₁-C₆-alkylcarbonylamino, C₁-C₆-alkoxycarbonylamino, C₁-C₆-alkoxy-C₁-C₆-alkyl, C₁-C₆-haloalkoxy-C₁-C₆-alkyl, C₂-C₆-alkenyl, C₂-C₆-alkynyl, C₁-C₆-alkyl-C₃-C₆-cycloalkyl, C₃-C₆-cycloalkyl-C₁-C₆-alkyl, C₁-C₆-alkylcarbonyl, C₁-C₆-alkoxycarbonyl or aminocarbonyl, and, in the case that R² represents c), d) or g),
R²² represents a radical from the group consisting of (D-1) to (D-3)

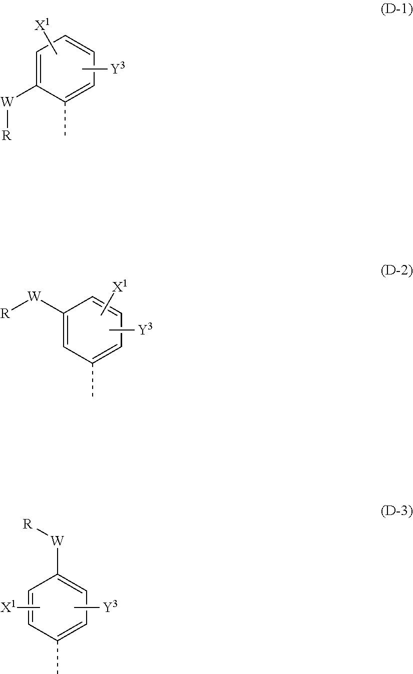

in which
X¹ represents a radical from the group consisting of hydrogen, halogen, cyano, nitro, C₁-C₆-alkyl, C₁-C₆-haloalkyl, C₃-C₆-cycloalkyl, C₁-C₆-alkoxy and C₁-C₆-haloalkoxy,
R represents NR¹⁸R¹⁹ or represents C₁-C₆-alkyl, C₃-C₆-alkenyl, C₃-C₆-alkynyl, C₁-C₆-alkoxy-C₁-C₄-alkyl, C₁-C₆-alkyl-S—C₁-C₄-alkyl, C₁-C₆-alkyl-S(O)—C₁-C₄-alkyl, C₁-C₆-alkyl-S(O)₂—C₁-C₄-alkyl, each of which is optionally mono- or disubstituted by halogen, oxygen (leads to C═O) or cyano, represents R¹⁸—CO—C₁-C₄-alkyl, represents NR¹⁸R¹⁹—CO—C₁-C₄-alkyl, represents C₃-C₆-cycloalkyl which is optionally mono- or disubstituted by oxygen (leads to C═O), C₁-C₄-alkyl, C₃-C₈-cycloalkyl, C₁-C₄-alkoxy and C₁-C₄-haloalkyl, represents C₃-C₈-cycloalkenyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkenyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, or represents phenyl, phenyl-C₁-C₄-alkyl, hetaryl and hetaryl-C₁-C₄-alkyl, each of which is optionally mono- to trisubstituted by halogen, cyano, C₁-C₄-alkyl, C₁-C₄-haloalkyl, C₃-C₆-cycloalkyl, C₁-C₄-alkoxy or C₁-C₄-haloalkoxy,
Y³ represents a radical from the group consisting of hydrogen, halogen, cyano, C₁-C₆-alkyl, C₁-C₆-haloalkyl, C₃-C₆-cycloalkyl, C₁-C₆-alkoxy, C₁-C₆-haloalkoxy and NR²⁰R²¹,
W represents a radical from the group consisting of O, S, SO and SO₂,
R²² furthermore represents a radical from the group consisting of (E-1) to (E-11)

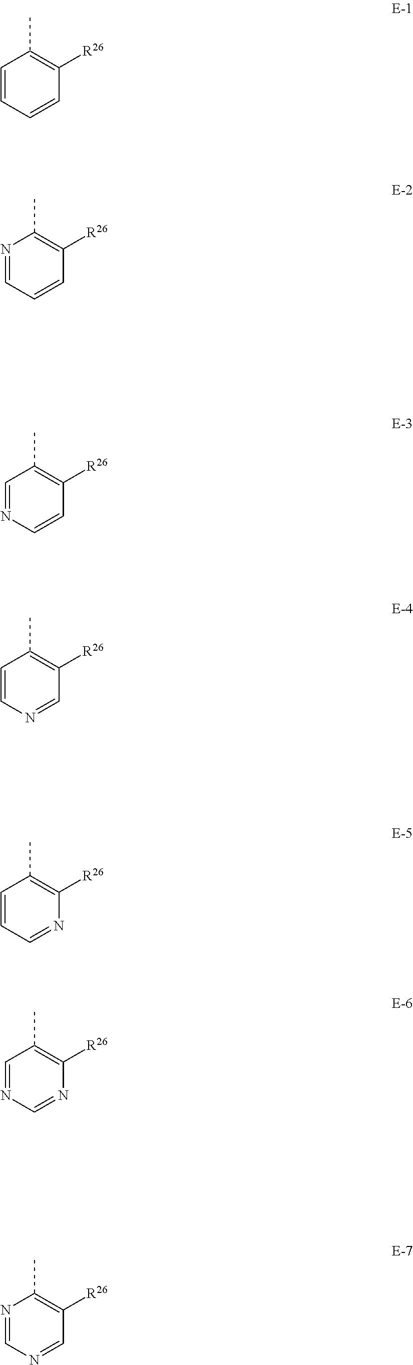

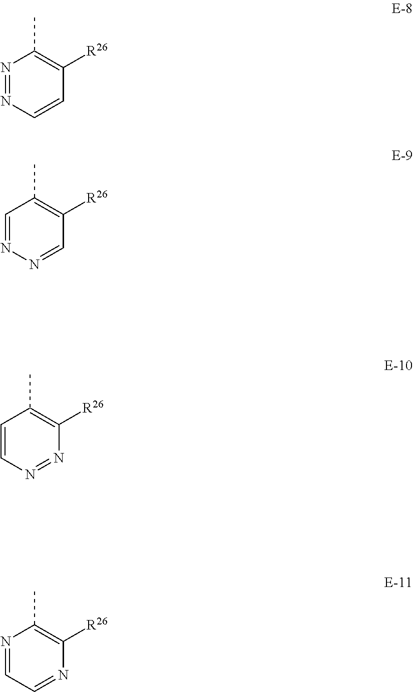

and (E-18) to (E-51)

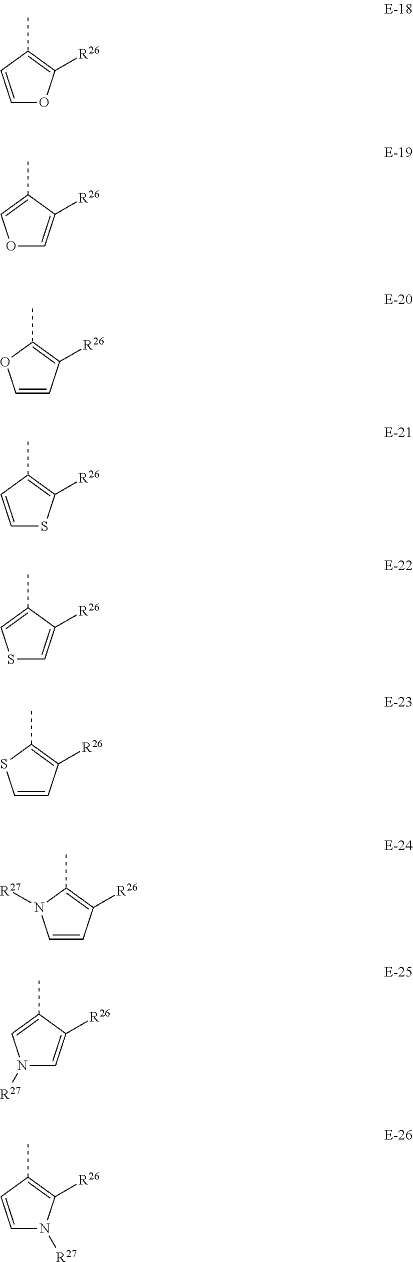

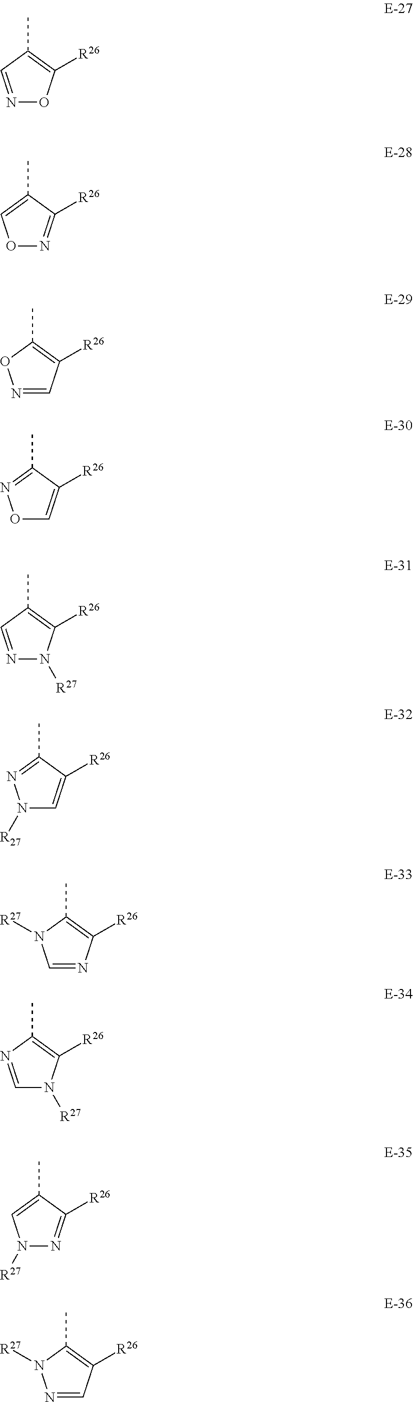

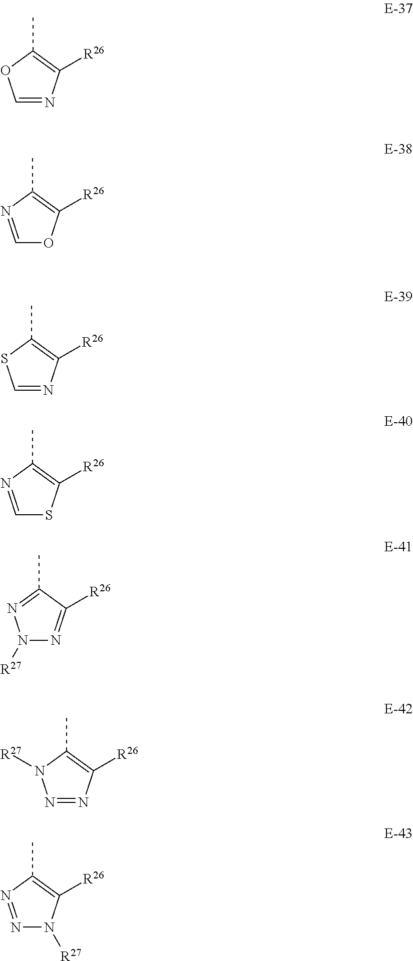

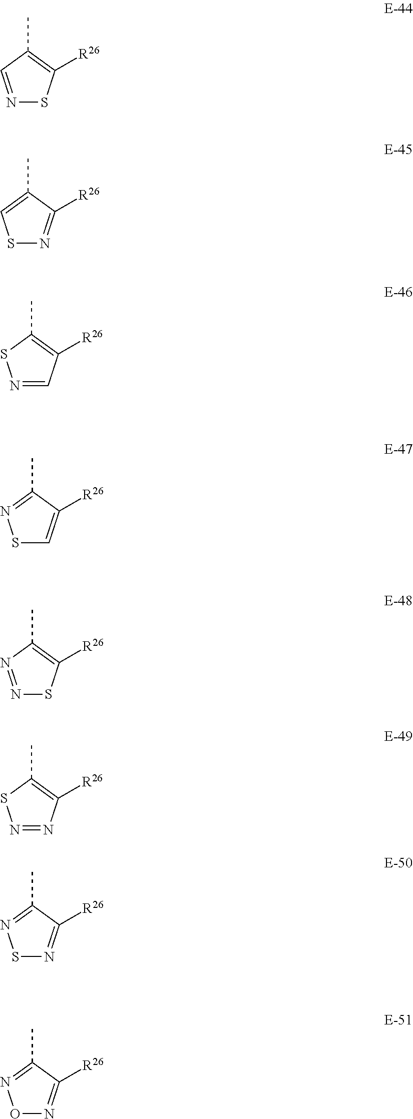

or
in the case R²=d),
R²² also represents an E radical from the group consisting of E-12 to E-17

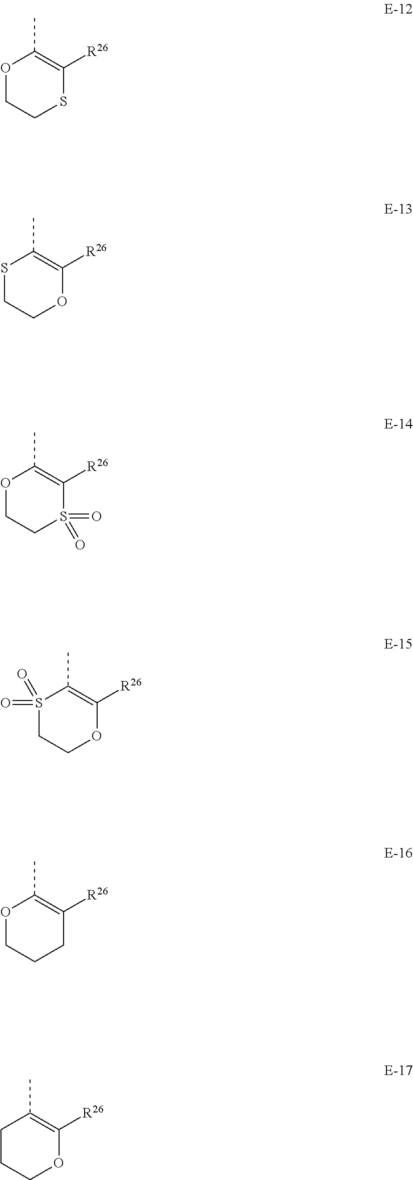

R¹⁸ represents a radical from the group consisting of hydrogen, hydroxy, of C₁-C₆-alkyl, C₁-C₆-alkoxy, C₁-C₆-alkoxy-C₁-C₄-alkyl, C₁-C₆-alkyl-S—C₁-C₄-alkyl, C₁-C₆-alkyl-S(O)—C₁-C₄-alkyl, C₁-C₆-alkyl-S(O)₂—C₁-C₄-alkyl, C₁-C₆-alkylcarbonyl, C₁-C₆-alkoxycarbonyl, C₃-C₆-cycloalkyl, C₃-C₆-cycloalkenyl, C₃-C₆-cycloalkyl-C₁-C₃-alkyl, C₃-C₆-cycloalkenyl-C₁-C₃-alkyl, heterocyclyl, heterocyclyl-C₁-C₃-alkyl, each of which is optionally mono- or polysubstituted by halogen or mono- or disubstituted by cyano, and phenyl, phenyl-C₁-C₃-alkyl, hetaryl and hetaryl-C₁-C₃-alkyl, each of which is optionally mono- to tetrasubstituted by C₁-C₄-alkyl, C₁-C₄-haloalkyl, C₁-C₄-alkoxy, C₁-C₄-haloalkoxy, C₃-C₆-cycloalkyl, halogen or cyano,
R¹⁹ represents hydrogen, an alkali or alkaline earth metal ion, or represents an ammonium ion which is optionally mono- to tetrasubstituted by C₁-C₄-alkyl, or represents a radical from the group consisting of C₁-C₄-alkyl, C₁-C₄-alkoxy, C₁-C₄-alkoxy-C₁-C₄-alkyl, C₁-C₄-alkyl-S—C₁-C₄-alkyl, C₁-C₄-alkyl-S(O)—C₁-C₄-alkyl and C₁-C₄-alkyl-S(O)₂—C₁-C₄-alkyl, each of which is optionally mono- or polysubstituted by halogen or mono- or disubstituted by cyano,
R²⁰ represents a radical from the group consisting of hydrogen, halogen, cyano, nitro, amino, hydroxy, C₁-C₆-alkyl, C₁-C₆-haloalkyl, cyano-C₁-C₆-alkyl, C₂-C₆-alkenyl, C₂-C₆-alkynyl, C₃-C₆-cycloalkyl (which is optionally substituted by halogen, cyano, C₁-C₄-alkyl and C₃-C₆-cycloalkyl), C₃-C₆-cycloalkyl-C₁-C₆-alkyl, C₁-C₆-alkoxy, C₁-C₆-haloalkoxy, C₃-C₆-alkenyloxy, C₃-C₆-alkynyloxy, C₃-C₆-cycloalkyloxy, C₁-C₆-alkylcarbonyloxy, C₂-C₆-alkenylcarbonyloxy, C₂-C₆-alkynylcarbonyloxy, C₃-C₆-cycloalkylcarbonyloxy, C₁-C₆-alkoxycarbonyloxy, C₁-C₆-alkylsulfonyloxy, C₁-C₆-alkylamino, C₃-C₆-alkenylamino, C₃-C₆-alkynylamino, C₃-C₆-cycloalkylamino, C₁-C₆-alkylthio, C₁-C₆-haloalkylthio, C₃-C₆-alkenylthio, C₃-C₆-alkynylthio, C₃-C₆-cycloalkylthio, C₁-C₆-alkylsulfinyl, C₁-C₆-alkylsulfonyl, C₁-C₆-alkylcarbonyl, C₁-C₆-alkoxyimino-C₁-C₆-alkyl, C₁-C₆-alkoxycarbonyl, aminocarbonyl, C₁-C₆-alkylaminocarbonyl, di-(C₁-C₆-alkyl)-aminocarbonyl, aminothiocarbonyl, C₁-C₆-alkylaminosulfonyl, C₁-C₆-alkylsulfonylamino, C₁-C₆-alkylcarbonylamino, C₁-C₆-alkylthiocarbonylamino, of phenyl, phenoxy, pyridinyl and pyridinyloxy, each of which is optionally substituted by a radical from the group consisting of halogen, cyano, nitro, amino, hydroxy, C₁-C₆-alkyl and C₁-C₆-haloalkyl,
R²¹ represents a radical from the group consisting of hydrogen, C₁-C₆-alkyl, C₃-C₆-cycloalkyl, C₁-C₆-haloalkyl, C₃-C₆-alkenyl, C₂-C₆-alkynyl, C₃-C₆-cycloalkyl-C₁-C₆-alkyl, cyano-C₁-C₆-alkyl, C₁-C₆-alkylcarbonyl, C₂-C₆-alkenylcarbonyl, C₁-C₆-haloalkylcarbonyl, C₂-C₆-haloalkenylcarbonyl, C₁-C₆-alkoxy-C₁-C₆-alkyl, C₁-C₆-alkoxycarbonyl, C₁-C₆-alkylsulfonyl and C₁-C₆-haloalkylsulfonyl,
R²³ represents a radical from the group consisting of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄alkyl,
or, if R²=g),
R²² and R²³ together with the nitrogen atom to which they are attached form a saturated ring which may contain one or more further heteroatoms from the group consisting of nitrogen, oxygen and sulfur, and
R²⁴ represents hydrogen or represents an in each case optionally halogen- or cyano-substituted radical from the group consisting of C₁-C₄-alkyl, C₂-C₆-alkenyl, C₂-C₆-alkynyl, phenyl and phenyl-C₁-C₂-alkyl,
R²⁵ represents hydrogen or represents an in each case optionally halogen- or cyano-substituted radical from the group consisting of C₁-C₄-alkyl, C₂-C₆-alkenyl, C₂-C₆-alkynyl, phenyl and phenyl-C₁-C₂-alkyl,
R²⁶ represents a radical from the group consisting of hydrogen, C₁-C₄-alkyl, C₁-C₄-haloalkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₆-alkylsulfanyl, C₁-C₆-haloalkylsulfanyl C₁-C₆-alkylsulfinyl, C₁-C₆-haloalkylsulfinyl, C₁-C₆-alkylsulfonyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl and cyano-C₁-C₄-alkyl and
R²⁷ represents hydrogen, C₁-C₄-alkyl, C₁-C₄-haloalkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl or cyano-C₁-C₄-alkyl.

Preferred range (2): Particular preference is given to compounds of the formula (I) in which
A represents an A radical from the group consisting of (A-b) and (A-f)

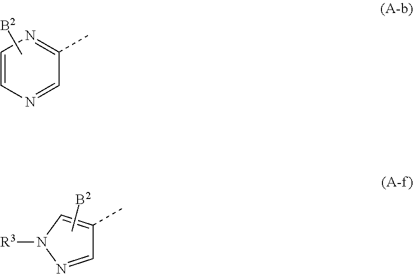

where the broken line denotes the bond to the carbon atom of the bicyclic system of the formula (I),
B² represents a radical from the group consisting of hydrogen, halogen, C₁-C₆-alkyl and C₁-C₄-haloalkyl,
Q represents sulfur,
R¹ represents a radical from the group consisting of hydrogen, C₁-C₄-alkyl and C₁-C₄-alkoxy,
R² a) represents a B radical from the group consisting of

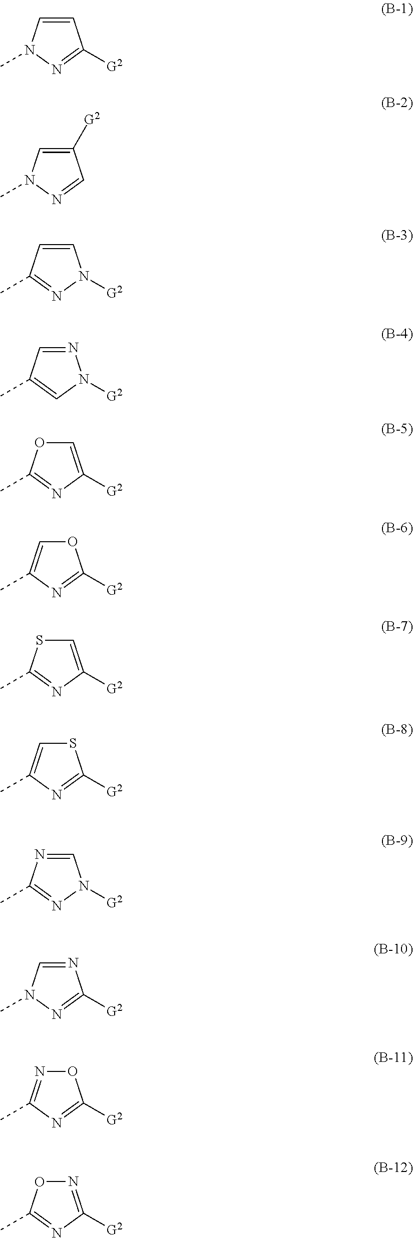

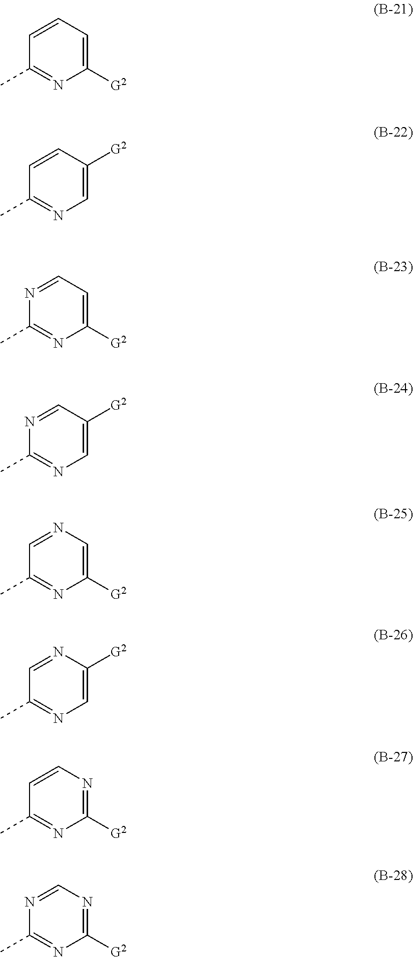

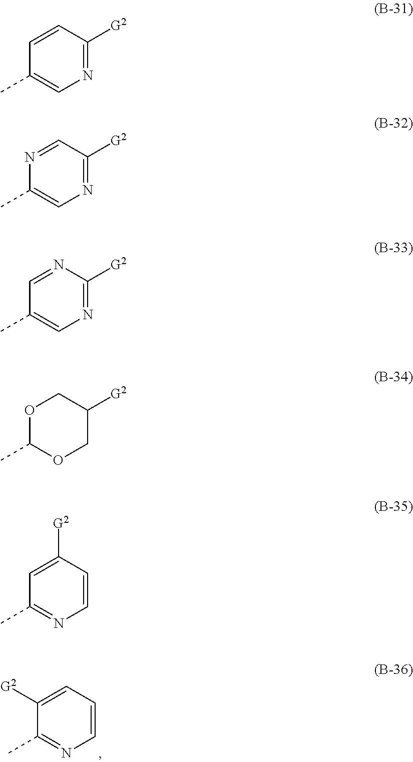

where the broken line denotes the bond to the carbon atom of the bicyclic system of the formula (I), or
R² b) represents a D radical from the group consisting of (D-1) to (D-3)

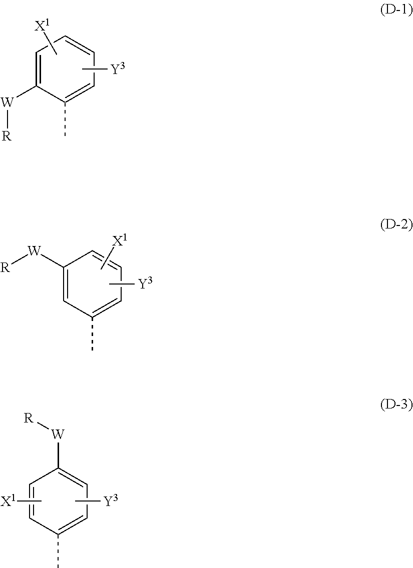

where the broken line denotes the bond to the carbon atom of the bicyclic system of the formula (I), or
R² c) represents a radical of the formula

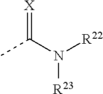

where the broken line denotes the bond to the carbon atom of the bicyclic system of the formula (I), or
R² d) represents a radical of the formula

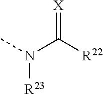

where the broken line denotes the bond to the carbon atom of the bicyclic system of the formula (I), or
R² e) represents an F radical from the group consisting of (F-1), (F-8) and (F-10)

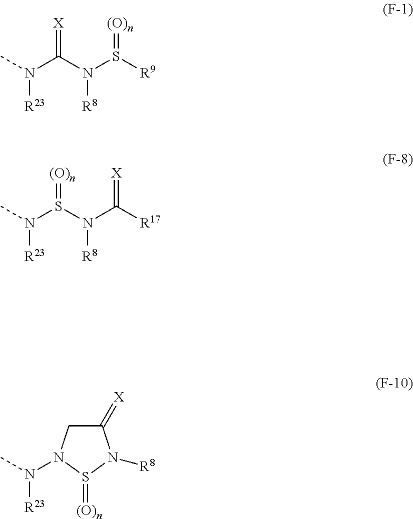

where the broken line denotes the bond to the carbon atom in the formula (I), or
R² f) represents a radical from the group consisting of C₁-C₆-haloalkyl and carboxyl,
R² g) represents a radical of the formula

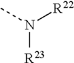

where the broken line denotes the bond to the carbon atom of the bicyclic system of the formula (I), in which
G² represents hydrogen or a radical from the group consisting of halogen, nitro, amino, cyano, C₁-C₄-alkylamino, halo-C₁-C₄-alkylamino, C₁-C₄-dialkylamino, C₁-C₄-alkyl, halo-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy(C₁-C₄-alkylsulfanyl)-C₁-C₄-alkyl, C₁-C₄-alkoxy(C₁-C₄-alkylsulfinyl)-C₁-C₄-alkyl, C₁-C₄-alkoxy(C₁-C₄-alkylsulfonyl)-C₁-C₄-alkyl, bis(C₁-C₄-alkylsulfanyl)-C₁-C₄-alkyl, bis(halo-C₁-C₄-alkylsulfanyl)-C₁-C₄-alkyl, bis(hydroxy-C₁-C₄-alkylsulfanyl)-C₁-C₄-alkyl, C₁-C₄-alkoxycarbonyl, C₁-C₄-alkoxycarbonyl-C₁-C₄-alkyl, C(X²)NR³R⁴, NR⁶R⁷, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl, the heterocyclyl radicals dioxanyl, dioxolanyl, dioxepanyl, dioxocanyl,
oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by C₁-C₄-alkyl, halo-C₁-C₄-alkyl, C₁-C₄-alkoxy and C₁-C₄-alkoxy-C₁-C₄-alkyl), phenyl (which for its part may be substituted by halogen, cyano, nitro, C₁-C₄-alkyl and halo-C₁-C₄-alkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, C₁-C₄-alkyl, halo-C₁-C₄-alkyl, C₁-C₄-alkoxy, halo-C₁-C₄-alkoxy, C₁-C₄-alkoxy-C₁-C₄-alkyl, C₁-C₄-alkylthio, C₁-C₄-alkylthio-C₁-C₄-alkyl and C₃-C₆-cycloalkyl) and the heteroaryl-C₁-C₄-alkyl radicals triazolyl-C₁-C₄-alkyl, pyridyl-C₁-C₄-alkyl, pyrimidyl-C₁-C₄-alkyl and oxadiazolyl-C₁-C₄-alkyl (which for their part may be substituted by halogen and C₁-C₄-alkyl), or
G² represents a C radical from the group consisting of (C-1), (C-6) and (C-9)

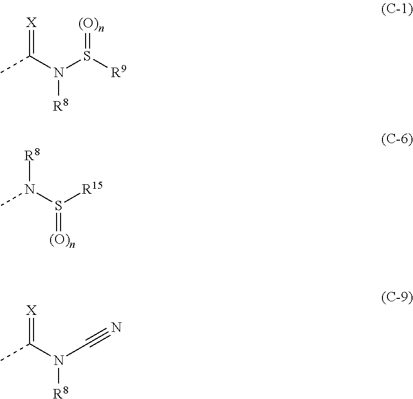

where the broken line represents the bond to the B radicals,
X represents oxygen,
X¹ represents a radical from the group consisting of hydrogen, fluorine, chlorine, bromine, iodine, cyano, nitro, C₁-C₄-alkyl, C₁-C₄-haloalkyl, C₃-C₆-cycloalkyl, C₁-C₄-alkoxy and C₁-C₄-haloalkoxy,
X² represents oxygen, sulfur, NR⁵ or NOH,
n represents 2,
R represents NR¹⁸R¹⁹ or represents C₁-C₆-alkyl, C₃-C₆-alkenyl, C₃-C₆-alkynyl, C₁-C₄-alkoxy-C₁-C₃-alkyl, C₁-C₄-alkyl-S—C₁-C₃-alkyl, C₁-C₄-alkyl-S(O)—C₁-C₃-alkyl, C₁-C₄-alkyl-S(O)₂—C₁-C₃-alkyl, each of which is optionally mono- to hepta substituted by halogen, mono- or disubstituted by oxygen (leads to C═O) or mono- or disubstituted by cyano, represents R¹⁸—CO—C₁-C₂-alkyl, represents NR¹⁸R¹⁹—CO—C₁-C₂-alkyl, represents C₃-C₆-cycloalkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₈-cycloalkenyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkenyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, or represents phenyl, phenyl-C₁-C₄-alkyl, hetaryl and hetaryl-C₁-C₄-alkyl, each of which is optionally mono- to trisubstituted by halogen, cyano, C₁-C₄-alkyl, C₁-C₄-haloalkyl, C₃-C₆-cycloalkyl, C₁-C₄-alkoxy or C₁-C₄-haloalkoxy,
R³ represents C₁-C₄-alkyl,
R⁴ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁵ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁶ represents hydrogen or C₁-C₄-alkyl,
R⁷ represents a radical from the group consisting of hydrogen, C₁-C₄-alkyl, halo-C₁-C₄-alkyl, cyano-C₁-C₄-alkyl, C₂-C₄-alkynyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl-C₁-C₄-alkyl or hetaryl-C₁-C₄-alkyl, or
R⁶ and R⁷ together with the nitrogen atom to which they are attached form a 4- to 7-membered ring which may contain one or two further heteroatoms from the group consisting of nitrogen, oxygen and sulfur (where oxygen and sulfur atoms must not be directly adjacent to one another),
R⁸ represents a radical from the group consisting of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulfonyl, optionally halogen-substituted C₁-C₆-alkoxycarbonyl and optionally halogen-, C₁-C₆-alkyl-, C₁-C₆-alkoxy-, C₁-C₆-haloalkyl- and cyano-substituted C₃-C₆-cycloalkylcarbonyl, or represents a cation or an optionally C₁-C₆-alkyl- or aryl-C₁-C₆-alkyl-substituted ammonium ion,
R⁹ represents a radical from the group consisting of in each case optionally halogen-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, in which one or two ring members may in each case be replaced by a heteroatom from the group consisting of sulfur, oxygen (where oxygen atoms must not be directly adjacent to one another) and nitrogen (and here in particular represent

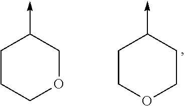

where the arrow in each case marks the bond to the sulfur atom in the radical (C-1) and in the radical (F-1)), in each case optionally halogen-, cyano- (including in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-,
C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl and heteroaryl-C₁-C₄-alkyl, or represents NR′R″ in which R′ and R″ independently of one another represent a radical from the group consisting of hydrogen and C₁-C₄-alkyl, or
R⁸ and R⁹ in the radical (C-1) and in the radical (F-1) may also form, together with the N—S(O)n group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group consisting of sulfur, oxygen (where oxygen atoms must not be directly adjacent to one another) and nitrogen and/or at least one and preferably exactly one carbonyl group, in particular, R⁸ and R⁹ together with the N—S(O)n group to which they are attached may represent a radical from the group consisting of

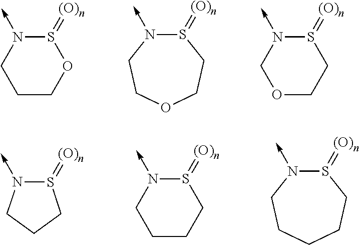

(in which the arrow in each case marks the bond to the C(X) group),
R¹⁵ represents a radical from the group consisting of in each case optionally methyl-substituted C₁-C₆-alkyl, C₂-C₆-alkenyl and C₂-C₆-alkynyl, in each case optionally methyl-, halogen-, cyano- or carbamoyl-substituted C₃-C₆-cycloalkyl, C₃-C₆-cycloalkyl-C₁-C₂-alkyl and C₃-C₆-cycloalkenyl,
R⁸ and R¹⁵ in the radical (C-6) may also form, together with the N—S(O)n group to which they are attached, a saturated or unsaturated and optionally substituted 4- to 8-membered ring which may contain one or two further heteroatoms from the group consisting of sulfur, oxygen (where oxygen and sulfur atoms must not be directly adjacent to one another) and nitrogen and/or at least one carbonyl group,
R¹⁷ represents a radical from the group consisting of in each case optionally halogen-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl or N-2-oxo-1-methylpiperazinyl, in each case optionally halogen-, cyano- (also in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulfinyl-,
C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl, heteroaryl-C₁-C₄-alkyl or represents NR′R″ in which R′ and R″ independently of one another represent a radical from the group consisting of hydrogen and C₁-C₄-alkyl,
R¹⁸ represents a radical from the group consisting of hydrogen, hydroxy, of C₁-C₄-alkyl, C₁-C₄-alkoxy, C₁-C₄-alkoxy-C₁-C₄-alkyl, C₁-C₄-alkyl-S—C₁-C₃-alkyl, C₁-C₄-alkyl-S(O)—C₁-C₃-alkyl, C₁-C₄-alkyl-S(O)₂—C₁-C₃-alkyl, C₁-C₄-alkylcarbonyl, C₁-C₄-alkoxycarbonyl, C₃-C₆-cycloalkyl, C₃-C₆-cycloalkyl-C₁-C₃-alkyl, heterocyclyl, heterocyclyl-C₁-C₃-alkyl, each of which is optionally mono- or polysubstituted by halogen or mono- or disubstituted by cyano, and of phenyl, benzyl, pyridyl, pyrimidyl, thiazolyl, oxazolyl, pyrazolyl, thienyl, furanyl, pyridinylmethyl and thiazolylmethyl, each of which is optionally mono- to trisubstituted by C₁-C₄-alkyl, C₁-C₃-haloalkyl, C₁-C₃-alkoxy, C₁-C₃-haloalkoxy, cyclopropyl, fluorine, chlorine, bromine or cyano,
R¹⁹ represents hydrogen, represents an alkali metal or alkaline earth metal ion, or represents an optionally mono- to tetra-C₁-C₄-alkyl-substituted ammonium ion or an in each case optionally mono- or poly-halogen-substituted or mono- or di-cyano-substituted radical from the group consisting of C₁-C₄-alkyl, C₁-C₄-alkoxy, C₁-C₄-alkoxy-C₁-C₂-alkyl, C₁-C₄-alkyl-S—C₁-C₂-alkyl, C₁-C₄-alkyl-S(O)—C₁-C₂-alkyl and C₁-C₄-alkyl-S(O)₂—C₁-C₂-alkyl,
Y³ represents a radical from the group consisting of hydrogen, fluorine, chlorine, bromine, iodine, cyano, C₁-C₄-alkyl, C₁-C₄-haloalkyl, C₃-C₆-cycloalkyl, C₁-C₄-alkoxy and C₁-C₄-haloalkoxy,
W represents a radical from the group consisting of S, SO and SO₂,
R²² radical from the group consisting of hydrogen, C₁-C₄-alkyl, C₂-C₄-haloalkyl, cyano-C₁-C₄-alkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, optionally halogen-substituted C₁-C₂-alkoxy-C₁-C₄-alkyl, optionally halogen-substituted bis(C₁-C₂-alkoxy)-C₁-C₄-alkyl, optionally halogen-substituted C₁-C₄-alkylsulfanyl-C₁-C₄-alkyl, optionally halogen-substituted C₁-C₄-alkylcarbonyl-C₁-C₄-alkyl, optionally halogen-substituted C₁-C₄-alkylsulfinyl-C₁-C₄-alkyl, optionally halogen-substituted C₁-C₄-alkylsulfonyl-C₁-C₄-alkyl, di-(C₁-C₄-alkyl)-aminosulfanyl-C₁-C₄-alkyl, di-(C₁-C₄-alkyl)-aminosulfinyl-C₁-C₄-alkyl, di-(C₁-C₄-alkyl)-aminosulfonyl-C₁-C₄-alkyl, optionally halogen-substituted C₁-C₄-alkoxycarbonyl, optionally halogen-substituted C₁-C₄-alkoxycarbonyl-C₁-C₄-alkyl, optionally halogen-substituted C₂-C₄-alkynyloxy, optionally halogen-substituted C₂-C₄-alkynyloxycarbonyl, di-(C₁-C₄-alkyl)-aminocarbonyl, N—C₁-C₄-alkyl-N—C₃-C₆-cycloalkylaminocarbonyl, di-(C₁-C₄-alkyl)-aminocarbonyl-C₁-C₄-alkyl, N—C₁-C₄-alkyl-N—C₃-C₆-cycloalkylaminocarbonyl-C₁-C₄-alkyl, heterocyclylcarbonyl-C₁-C₄-alkyl, C₁-C₄-alkylsulfanyl, C₁-C₄-haloalkylsulfanyl, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl which is optionally substituted by halogen, cyano, nitro, C₁-C₄-alkyl, C₁-C₄-haloalkyl, C₃-C₆-cycloalkyl, C₁-C₄-alkoxy, C₁-C₄-haloalkoxy, C₁-C₄-alkoxycarbonyl, C₁-C₄-haloalkoxycarbonyl oder pyridyl (which for its part is optionally substituted by C₁-C₄-alkyl or halogen), C₃-C₆-cycloalkylcarbonyl which is optionally substituted by halogen, cyano, nitro, C₁-C₄-alkyl, C₁-C₄-haloalkyl, C₃-C₆-cycloalkyl, C₁-C₄-alkoxy, C₁-C₄-haloalkoxy, C₁-C₄-alkoxycarbonyl, C₁-C₄-haloalkoxycarbonyl or pyridyl (which for its part is optionally substituted by C₁-C₄-alkyl or halogen), C₃-C₆-cycloalkyl-C₁-C₄-alkyl which is optionally substituted by halogen, cyano, nitro, C₁-C₄-alkyl, C₁-C₄-haloalkyl, C₃-C₆-cycloalkyl, C₁-C₄-alkoxy, C₁-C₄-haloalkoxy, C₁-C₄-alkoxycarbonyl, C₁-C₄-haloalkoxycarbonyl, pyridyl, pyrimidyl, pyrazanyl, pyridazinyl, thiazolyl, isothiazolyl, thiadiazolyl, oxazolyl, isoxazolyl, oxadiazolyl, pyrazolyl, triazinyl or triazolyl (where the hetaryl radicals mentioned are for their part optionally substituted by C₁-C₄-alkyl or halogen), heterocyclyl-C₁-C₄-alkyl which is optionally substituted by halogen, cyano (also in the C₁-C₄-alkyl moiety of heterocyclyl-C₁-C₄-alkyl), nitro, hydroxy, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy, C₁-C₄-haloalkoxy, C₁-C₄-alkylthio, C₁-C₄-haloalkylthio, C₁-C₄-alkylsulfinyl, C₁-C₄-alkylsulfonyl, C₁-C₄-haloalkylsulfinyl, C₁-C₄-haloalkylsulfonyl, amino, C₁-C₄-alkylamino, di-(C₁-C₄-alkyl)-amino, C₁-C₄-alkylcarbonylamino, C₁-C₄-alkoxycarbonylamino, C₁-C₄-alkoxy-C₁-C₄-alkyl, C₁-C₄-haloalkoxy-C₁-C₄-alkyl, C₂-C₄-alkenyl, C₂-C₄-alkynyl, C₃-C₆-cycloalkyl-C₁-C₄-alkyl, C₁-C₄-alkylcarbonyl, C₁-C₄-alkoxycarbonyl or aminocarbonyl, aryl which is optionally substituted by halogen, cyano, nitro, hydroxy, amino, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy or C₁-C₄-haloalkoxy, aryl-C₁-C₄-alkyl which is optionally substituted by halogen, cyano (also in the C₁-C₄-alkyl moiety of aryl-C₁-C₄-alkyl), nitro, hydroxy, amino, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy or C₁-C₄-haloalkoxy, hetaryl-C₁-C₄-alkyl which is optionally substituted by halogen, cyano (also in the C₁-C₄-alkyl moiety of hetaryl-C₁-C₄-alkyl), nitro, hydroxy, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy, C₁-C₄-haloalkoxy, C₁-C₄-alkylthio, C₁-C₄-haloalkylthio, C₁-C₄-alkylsulfinyl, C₁-C₄-alkylsulfonyl, C₁-C₄-haloalkylsulfinyl, C₁-C₄-haloalkylsulfonyl, amino, C₁-C₄-alkylamino, di-(C₁-C₄-alkyl)-amino, C₁-C₄-alkylcarbonylamino, C₁-C₄-alkoxycarbonylamino, C₁-C₄-alkoxy-C₁-C₄-alkyl, C₁-C₄-haloalkoxy-C₁-C₄-alkyl, C₂-C₄-alkenyl, C₂-C₄-alkynyl, C₃-C₆-cycloalkyl-C₁-C₄-alkyl, C₁-C₄-alkylcarbonyl, C₁-C₄-alkoxycarbonyl or aminocarbonyl, or
R²² represents a D radical from the group consisting of (D-1) to (D-3)

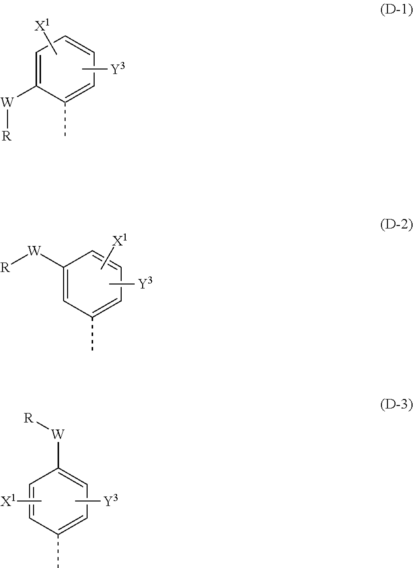

or represents one of the E radicals below

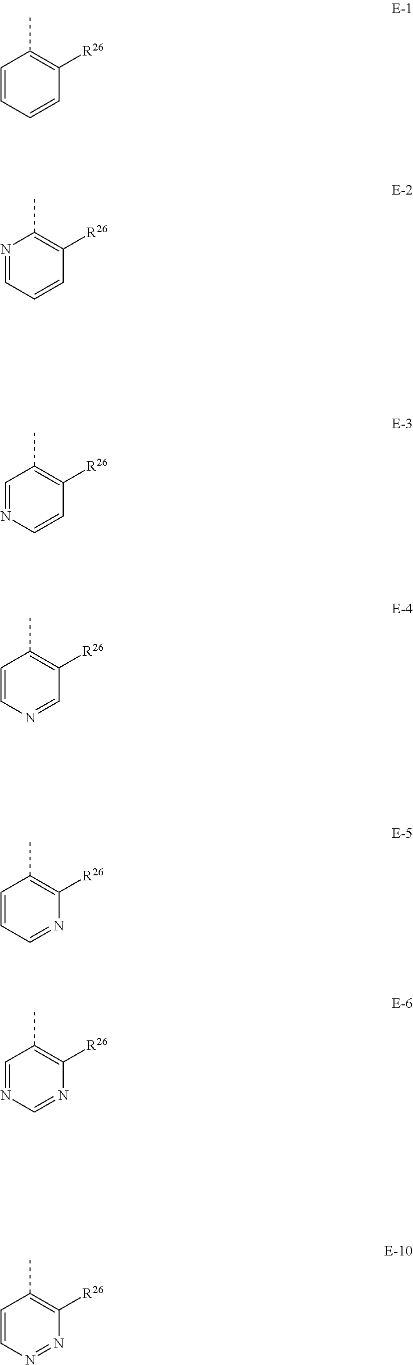

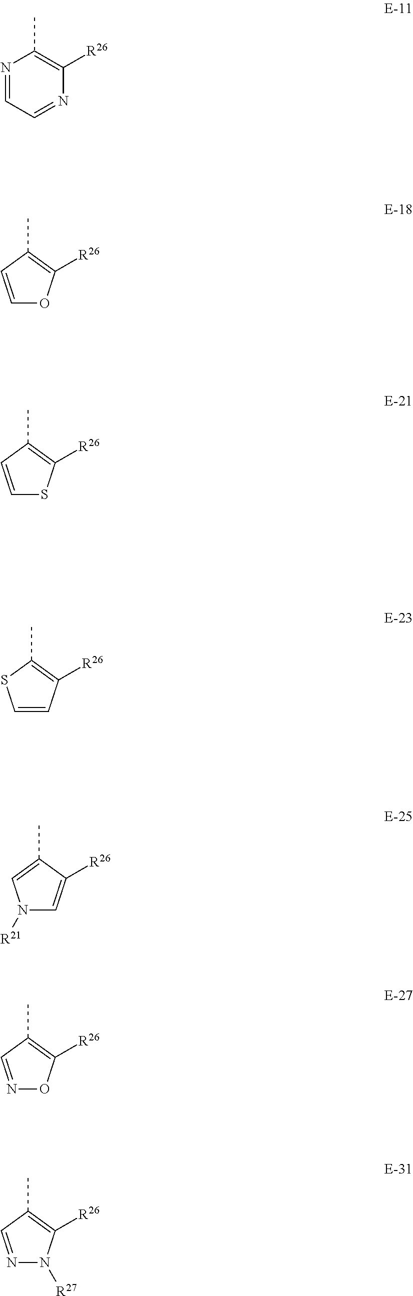

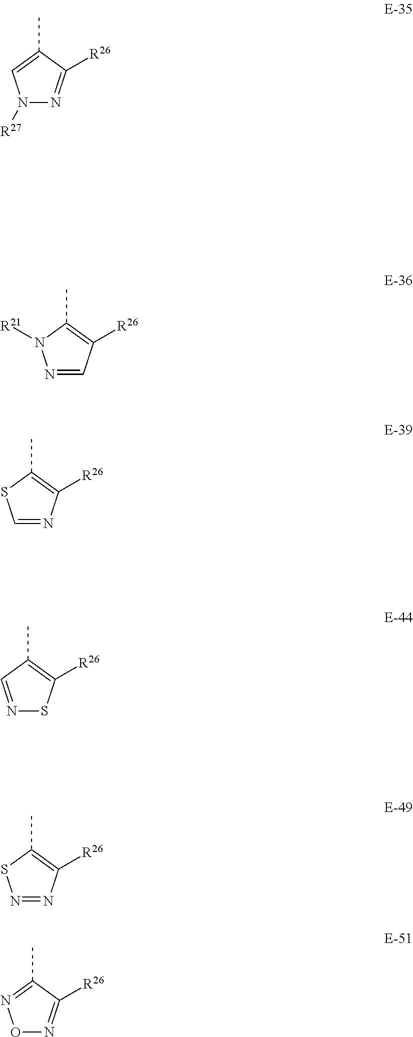

or, in the case that R²=d),
R²² also represents the radical (E-13)

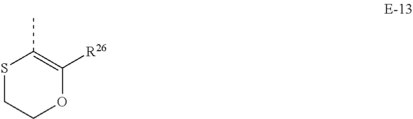

R²³ represents a radical from the group consisting of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl and C₁-C₄-alkoxy-C₁-C₄alkyl,
or, if R²=c) or g),
R²² and R²³ together with the nitrogen atom to which they are attached form a saturated four- to six-membered ring which may contain a further heteroatom from the group consisting of nitrogen, oxygen and sulfur,
R²⁶ represents a radical from the group consisting of hydrogen, C₁-C₄-alkyl, C₁-C₄-haloalkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylsulfanyl, C₁-C₄-haloalkylsulfanyl C₁-C₄-alkylsulfinyl, C₁-C₄-haloalkylsulfinyl, C₁-C₄-alkylsulfonyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl and cyano-C₁-C₄-alkyl and
R²⁷ represents hydrogen, C₁-C₄-alkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl or cyano-C₁-C₄-alkyl and
compounds of the formula (I) in which
A represents the A radical

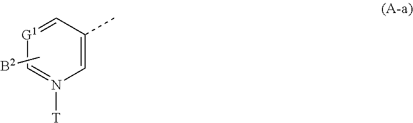

where the broken line denotes the bond to the carbon atom of the bicyclic system of the formula (I),
G¹ represents N or C—B¹,
B¹ represents a radical from the group consisting of hydrogen, halogen, C₁-C₆-alkyl and C₁-C₄-haloalkyl,
B² represents a radical from the group consisting of hydrogen, halogen, C₁-C₆-alkyl and C₁-C₄-haloalkyl,
T represents oxygen or an electron pair,
Q represents sulfur,
R¹ represents a radical from the group consisting of hydrogen, C₁-C₄-alkyl and C₁-C₄-alkoxy,
R² a) represents a B radical from the group consisting of

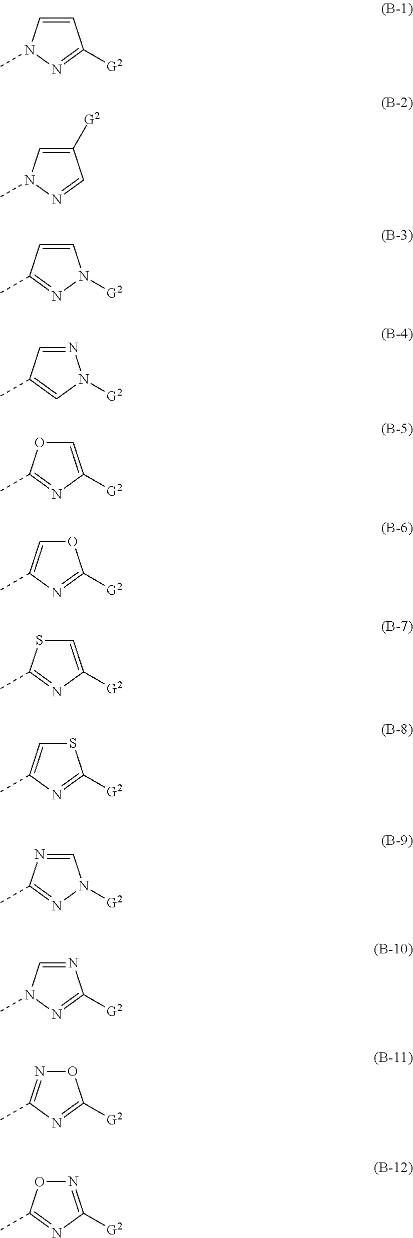

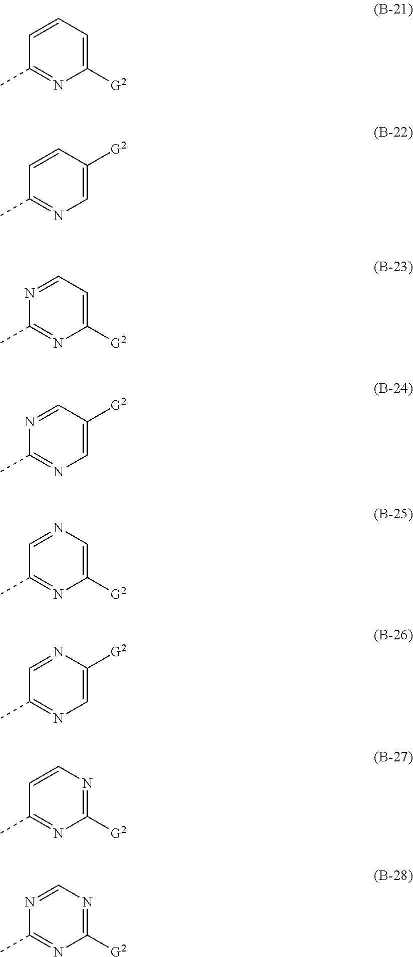

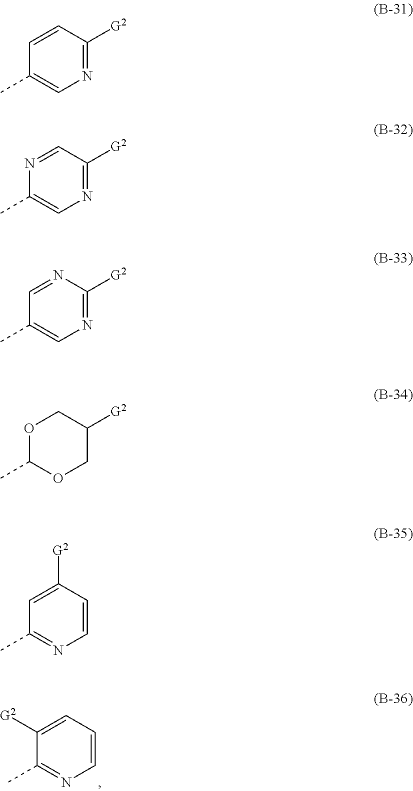

where the broken line denotes the bond to the carbon atom of the bicyclic system of the formula (I), or
R² c) represents a radical of the formula

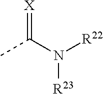

where the broken line denotes the bond to the carbon atom of the bicyclic system of the formula (I), or
R² d) represents a radical of the formula

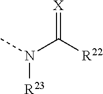

where the broken line denotes the bond to the carbon atom of the bicyclic system of the formula (I), or
R² e) represents an F radical from the group consisting of (F-8) and (F-10)

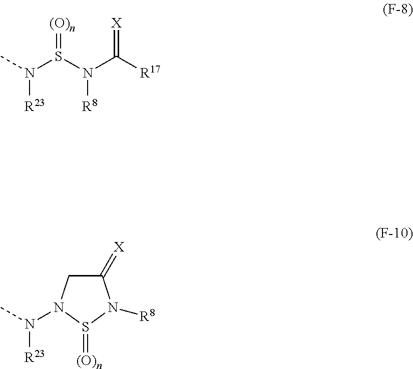

where the broken line denotes the bond to the carbon atom in the formula (I), or R² f) represents a radical from the group consisting of C₁-C₆-haloalkyl and carboxyl,
R² g) represents a radical of the formula

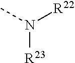

where the broken line denotes the bond to the carbon atom of the bicyclic system of the formula (I), where
G² represents hydrogen or a radical from the group consisting of halogen, nitro, amino, cyano, C₁-C₄-alkylamino, halo-C₁-C₄-alkylamino, C₁-C₄-dialkylamino, C₁-C₄-alkyl, halo-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy(C₁-C₄-alkylsulfanyl)-C₁-C₄-alkyl, C₁-C₄-alkoxy(C₁-C₄-alkylsulfinyl)-C₁-C₄-alkyl, C₁-C₄-alkoxy(C₁-C₄-alkylsulfonyl)-C₁-C₄-alkyl, bis(C₁-C₄-alkylsulfanyl)-C₁-C₄-alkyl, bis(halo-C₁-C₄-alkylsulfanyl)-C₁-C₄-alkyl, bis(hydroxy-C₁-C₄-alkylsulfanyl)-C₁-C₄-alkyl, C₁-C₄-alkoxycarbonyl, C₁-C₄-alkoxycarbonyl-C₁-C₄-alkyl, C(X²)NR³R⁴, NR⁶R⁷, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by C₁-C₄-alkyl, halo-C₁-C₄-alkyl, C₁-C₄-alkoxy and C₁-C₄-alkoxy-C₁-C₄-alkyl), phenyl (which for its part may be substituted by halogen, cyano, nitro, C₁-C₄-alkyl and halo-C₁-C₄-alkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, C₁-C₄-alkyl, halo-C₁-C₄-alkyl, C₁-C₄-alkoxy, halo-C₁-C₄-alkoxy, C₁-C₄-alkoxy-C₁-C₄-alkyl, C₁-C₄-alkylthio, C₁-C₄-alkylthio-C₁-C₄-alkyl and C₃-C₆-cycloalkyl) and the heteroaryl-C₁-C₄-alkyl radicals triazolyl-C₁-C₄-alkyl, pyridyl-C₁-C₄-alkyl, pyrimidyl-C₁-C₄-alkyl and oxadiazolyl-C₁-C₄-alkyl (which for their part may be substituted by halogen and C₁-C₄-alkyl), or
G² represents a C radical from the group consisting of (C-1), (C-6) and (C-9)

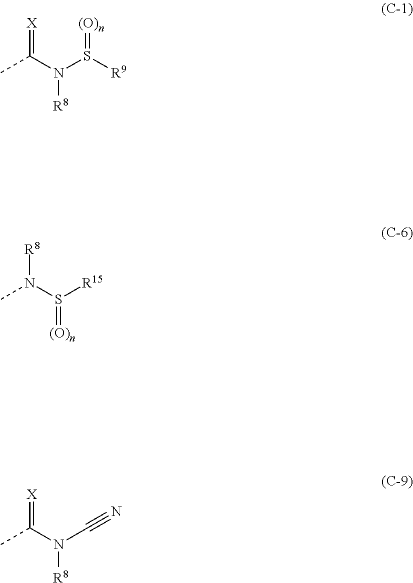

where the broken line denotes the bond to the B radicals,
X represents oxygen,
X² represents oxygen, sulfur, NR⁵ or NOH,
n represents 2,
R³ represents C₁-C₄-alkyl,
R⁴ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁵ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁶ represents hydrogen or C₁-C₄-alkyl,
R⁷ represents a radical from the group consisting of hydrogen, C₁-C₄-alkyl, halo-C₁-C₄-alkyl, cyano-C₁-C₄-alkyl, C₂-C₄-alkynyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl-C₁-C₄-alkyl or hetaryl-C₁-C₄-alkyl, or
R⁶ and IC together with the nitrogen atom to which they are attached form a 4- to 7-membered ring which may contain one or two further heteroatoms from the group consisting of nitrogen, oxygen and sulfur (where oxygen and sulfur atoms must not be directly adjacent to one another),
R⁸ represents a radical from the group consisting of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulfonyl, optionally halogen-substituted C₁-C₆-alkoxycarbonyl and optionally halogen-, C₁-C₆-alkyl-, C₁-C₆-alkoxy-, C₁-C₆-haloalkyl- and cyano-substituted C₃-C₆-cycloalkylcarbonyl, or represents a cation or an optionally C₁-C₆-alkyl- or aryl-C₁-C₆-alkyl-substituted ammonium ion,
R⁹ represents a radical from the group consisting of in each case optionally halogen-, C₁-C₄-alkoxy-, C₁-C₄-haloalkoxy C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, in which one or two ring members may in each case be replaced by a heteroatom from the group consisting of sulfur, oxygen (where oxygen atoms must not be directly adjacent to one another) and nitrogen (and here in particular represent

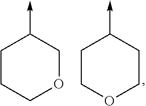

where the arrow in each case marks the bond to the sulfur atom in the (C-1) radical), in each case optionally halogen-, cyano- (including in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl and heteroaryl-C₁-C₄-alkyl, or is NR′R″ in which R′ and R″ are independently a radical from the group of hydrogen and C₁-C₄-alkyl, or
R⁸ and R⁹ in the radical (C-1) may also form, together with the N—S(O)n group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group consisting of sulfur, oxygen (where oxygen atoms must not be directly adjacent to one another) and nitrogen and/or at least one and preferably exactly one carbonyl group, in particular, R⁸ and R⁹ together with the N—S(O)n group to which they are attached may represent a radical from the group consisting of

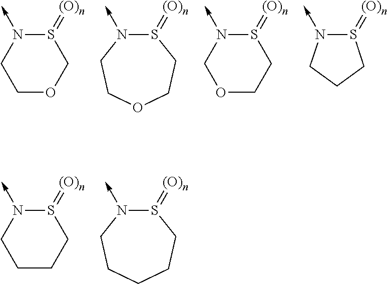

(in which the arrow in each case marks the bond to the C(X) group),
R¹⁵ represents a radical from the group consisting of in each case optionally methyl-substituted C₁-C₆-alkyl, C₂-C₆-alkenyl and C₂-C₆-alkynyl, in each case optionally methyl-, halogen-, cyano- or carbamoyl-substituted C₃-C₆-cycloalkyl, C₃-C₆-cycloalkyl-C₁-C₂-alkyl and C₃-C₆-cycloalkenyl,
R⁸ and R¹⁵ in the radical (C-6) may also form, together with the N—S(O)n group to which they are attached, a saturated or unsaturated and optionally substituted 4- to 8-membered ring which may contain one or two further heteroatoms from the group consisting of sulfur, oxygen (where oxygen and sulfur atoms must not be directly adjacent to one another) and nitrogen and/or at least one carbonyl group,
R¹⁷ represents a radical from the group consisting of in each case optionally halogen-, C₁-C₄-alkoxy-, C₁-C₄-haloalkoxy C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl or N-2-oxo-1-methylpiperazinyl, in each case optionally halogen-, cyano- (also in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl, heteroaryl-C₁-C₄-alkyl or represents NR′R″ in which R′ and R″ independently of one another represent a radical from the group consisting of hydrogen and C₁-C₄-alkyl,
and, in the case that R² represents g),
R²² represents a radical from the group consisting of hydrogen, C₁-C₄-alkyl, C₂-C₄-haloalkyl, cyano-C₁-C₄-alkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, optionally halogen-substituted C₁-C₂-alkoxy-C₁-C₄-alkyl, optionally halogen-substituted bis(C₁-C₂-alkoxy)-C₁-C₄-alkyl, optionally halogen-substituted C₁-C₄-alkylsulfanyl-C₁-C₄-alkyl, optionally halogen-substituted C₁-C₄-alkylcarbonyl-C₁-C₄-alkyl, optionally halogen-substituted C₁-C₄-alkylsulfinyl-C₁-C₄-alkyl, optionally halogen-substituted C₁-C₄-alkylsulfonyl-C₁-C₄-alkyl, di-(C₁-C₄-alkyl)-aminosulfanyl-C₁-C₄-alkyl, di-(C₁-C₄-alkyl)-aminosulfinyl-C₁-C₄-alkyl, di-(C₁-C₄-alkyl)-aminosulfonyl-C₁-C₄-alkyl, optionally halogen-substituted C₁-C₄-alkoxycarbonyl-C₁-C₄-alkyl, optionally halogen-substituted C₁-C₄-alkynyloxy, optionally halogen-substituted C₁-C₄-alkynyloxycarbonyl, di-(C₁-C₄-alkyl)-aminocarbonyl, N—C₁-C₄-alkyl-N—C₃-C₆-cycloaklaminocarbonyl, di-(C₁-C₄-alkyl)-aminocarbonyl-C₁-C₄-alkyl, N—C₁-C₄-alkyl-N—C₃-C₆-cycloaklaminocarbonyl-C₁-C₄-alkyl, heterocyclylcarbonyl-C₁-C₄-alkyl, C₁-C₄-alkylsulfanyl, C₁-C₄-haloalkylsulfanyl, C₁-C₄-alkylsulfinyl, C₁-C₄-haloalkylsulfinyl, C₁-C₄-alkylsulfonyl, C₁-C₄-haloalkylsulfonyl, C₃-C₆-cycloalkyl which is optionally substituted by halogen, cyano, nitro, C₁-C₄-alkyl, C₁-C₄-haloalkyl, C₃-C₆-cycloalkyl, C₁-C₄-alkoxy, C₁-C₄-haloalkoxy, C₁-C₄-alkoxycarbonyl, C₁-C₄-haloalkoxycarbonyl or pyridyl (which for its part is optionally substituted by C₁-C₄-alkyl or halogen), C₃-C₆-cycloalkylcarbonyl which is optionally substituted by halogen, cyano, nitro, C₁-C₄-alkyl, C₁-C₄-haloalkyl, C₃-C₆-cycloalkyl, C₁-C₄-alkoxy, C₁-C₄-haloalkoxy, C₁-C₄-alkoxycarbonyl, C₁-C₄-haloalkoxycarbonyl or pyridyl (which for its part is optionally substituted by C₁-C₄-alkyl or halogen), C₃-C₆-cycloalkyl-C₁-C₄-alkyl which is optionally substituted by halogen, cyano, nitro, C₁-C₄-alkyl, C₁-C₄-haloalkyl, C₃-C₆-cycloalkyl, C₁-C₄-alkoxy, C₁-C₄-haloalkoxy, C₁-C₄-alkoxycarbonyl, C₁-C₄-haloalkoxycarbonyl, pyridyl, pyrimidyl, pyrazanyl, pyridazinyl, thiazolyl, isothiazolyl, thiadiazolyl, oxazolyl, isoxazolyl, oxadiazolyl, pyrazolyl, triazinyl or triazolyl (where the hetaryl radicals mentioned are for their part optionally substituted by C₁-C₄-alkyl or halogen), heterocyclyl-C₁-C₄-alkyl which is optionally substituted by halogen, cyano (also in the C₁-C₄-alkyl moiety of heterocyclyl-C₁-C₄-alkyl), nitro, hydroxy, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy, C₁-C₄-haloalkoxy, C₁-C₄-alkylthio, C₁-C₄-haloalkylthio, C₁-C₄-alkylsulfinyl, C₁-C₄-alkylsulfonyl, C₁-C₄-haloalkylsulfinyl, C₁-C₄-haloalkylsulfonyl, amino, C₁-C₄-alkylamino, di-(C₁-C₄-alkyl)-amino, C₁-C₄-alkylcarbonylamino, C₁-C₄-alkoxycarbonylamino, C₁-C₄-alkoxy-C₁-C₄-alkyl, C₁-C₄-haloalkoxy-C₁-C₄-alkyl, C₂-C₄-alkenyl, C₂-C₄-alkynyl, C₃-C₆-cycloalkyl-C₁-C₄-alkyl, C₁-C₄-alkylcarbonyl, C₁-C₄-alkoxycarbonyl or aminocarbonyl, aryl which is optionally substituted by halogen, cyano, nitro, hydroxy, amino, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy or C₁-C₄-haloalkoxy, aryl-C₁-C₄-alkyl which is optionally substituted by halogen, cyano (also in the C₁-C₄-alkyl moiety of aryl-C₁-C₄-alkyl), nitro, hydroxy, amino, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy or C₁-C₄-haloalkoxy, hetaryl-C₁-C₄-alkyl which is optionally substituted by halogen, cyano (also in the C₁-C₄-alkyl moiety of hetaryl-C₁-C₄-alkyl), nitro, hydroxy, C₁-C₄-alkyl, C₁-C₄-haloalkyl, C₃-C₆-cycloalkyl (which is optionally substituted by halogen, cyano, C₁-C₄-alkyl and C₃-C₆-cycloalkyl), C₁-C₄-alkoxy, C₁-C₄-haloalkoxy, C₁-C₄-alkylthio, C₁-C₄-haloalkylthio, C₁-C₄-alkylsulfinyl,
C₁-C₄-alkylsulfonyl, C₁-C₄-haloalkylsulfinyl, C₁-C₄-haloalkylsulfonyl, amino, C₁-C₄-alkylamino, di-(C₁-C₄-alkyl)-amino, C₁-C₄-alkylcarbonylamino, C₁-C₄-alkoxycarbonylamino, C₁-C₄-alkoxy-C₁-C₄-alkyl, C₁-C₄-haloalkoxy-C₁-C₄-alkyl, C₂-C₄-alkenyl, C₂-C₄-alkynyl, C₃-C₆-cycloalkyl-C₁-C₄-alkyl, C₁-C₄-alkylcarbonyl, C₁-C₄-alkoxycarbonyl or aminocarbonyl, and, in the case that R² represents g),
R²² represents a D radical from the group consisting of (D-1) to (D-3)

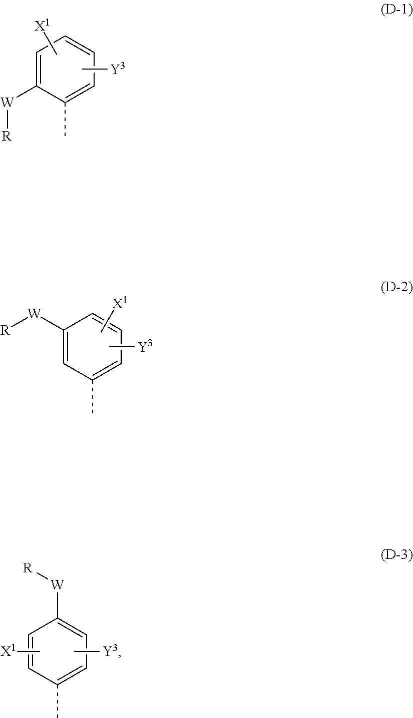

in which
X¹ represents a radical from the group consisting of hydrogen, fluorine, chlorine, bromine, iodine, cyano, nitro, C₁-C₄-alkyl, C₁-C₄-haloalkyl, C₃-C₆-cycloalkyl, C₁-C₄-alkoxy and C₁-C₄-haloalkoxy,
R represents NR¹⁸R¹⁹ or represents C₁-C₆-alkyl, C₃-C₆-alkenyl, C₃-C₆-alkynyl, C₁-C₄-alkoxy-C₁-C₃-alkyl, C₁-C₄-alkyl-S—C₁-C₃-alkyl, C₁-C₄-alkyl-S(O)—C₁-C₃-alkyl, C₁-C₄-alkyl-S(O)₂—C₁-C₃-alkyl, each of which is optionally mono- to hepta substituted by halogen, mono- or disubstituted by oxygen (leads to C═O) or mono- or disubstituted by cyano, represents R¹⁸—CO—C₁-C₂-alkyl, represents NR¹⁸R¹⁹—CO—C₁-C₂-alkyl, represents C₃-C₆-cycloalkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₈-cycloalkenyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents C₃-C₆-cycloalkenyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl which is optionally mono- or disubstituted by oxygen (leads to C═O), C₁-C₄-alkyl, C₃-C₆-cycloalkyl, C₁-C₄-alkoxy and C₁-C₄-haloalkyl, represents heterocyclyl-C₁-C₄-alkyl which is optionally mono- or disubstituted by oxygen (leads to C═O), C₁-C₄-alkyl, C₃-C₆-cycloalkyl, C₁-C₄-alkoxy and C₁-C₄-haloalkyl, or represents phenyl, phenyl-C₁-C₄-alkyl, hetaryl and hetaryl-C₁-C₄-alkyl, each of which is optionally mono- to trisubstituted by halogen, cyano, C₁-C₄-alkyl, C₁-C₄-haloalkyl, C₃-C₆-cycloalkyl, C₁-C₄-alkoxy or C₁-C₄-haloalkoxy,
Y³ represents a radical from the group consisting of hydrogen, fluorine, chlorine, bromine, iodine, cyano, C₁-C₄-alkyl, C₁-C₄-haloalkyl, C₃-C₆-cycloalkyl, C₁-C₄-alkoxy and C₁-C₄-haloalkoxy,
W represents a radical from the group consisting of S, SO and SO₂,
R²² furthermore represents one of the E radicals below

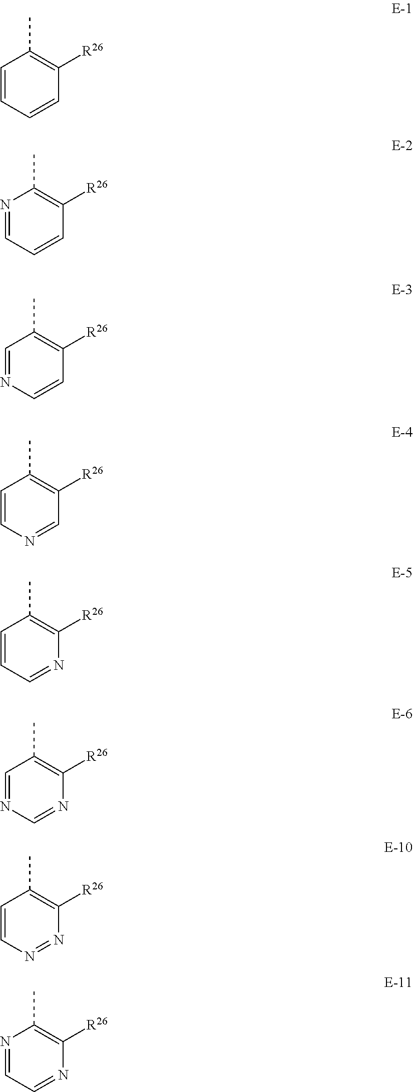

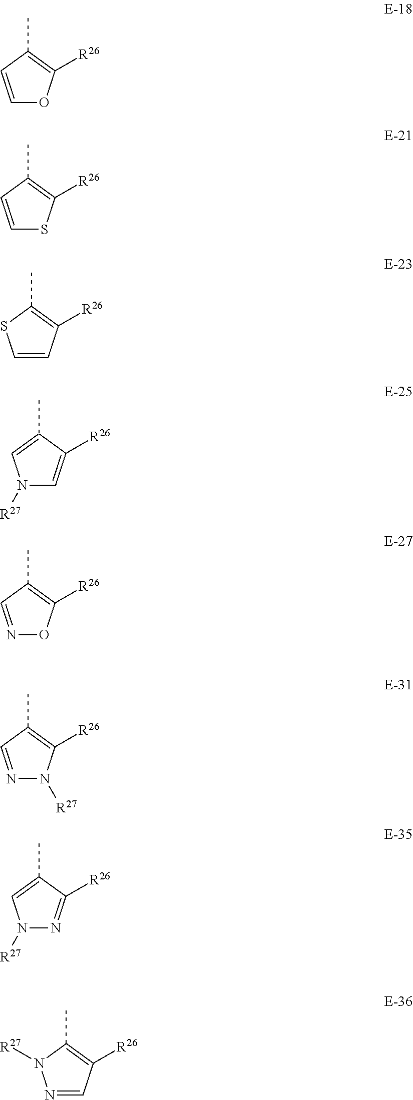

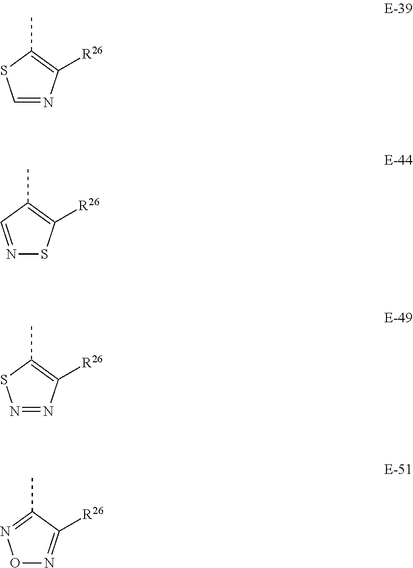

or, in the case that R²=d),
R²² also represents the radical (E-13)

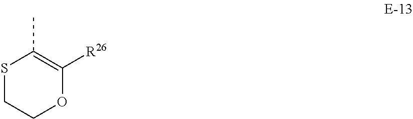

R¹⁸ represents a radical from the group consisting of hydrogen, hydroxy, of C₁-C₄-alkyl, C₁-C₄-alkoxy, C₁-C₄-alkoxy-C₁-C₄-alkyl, C₁-C₄-alkyl-S—C₁-C₃-alkyl, C₁-C₄-alkyl-S(O)—C₁-C₃-alkyl, C₁-C₄-alkyl-S(O)₂—C₁-C₃-alkyl, C₁-C₄-alkylcarbonyl, C₁-C₄-alkoxycarbonyl, C₃-C₆-cycloalkyl, C₃-C₆-cycloalkyl-C₁-C₃-alkyl, heterocyclyl, heterocyclyl-C₁-C₃-alkyl, each of which is optionally mono- or polysubstituted by halogen or mono- or disubstituted by cyano, and of phenyl, benzyl, pyridyl, pyrimidyl, thiazolyl, oxazolyl, pyrazolyl, thienyl, furanyl, pyridinylmethyl and thiazolylmethyl, each of which is optionally mono- to trisubstituted by C₁-C₄-alkyl, C₁-C₃-haloalkyl, C₁-C₃-alkoxy, C₁-C₃-haloalkoxy, cyclopropyl, fluorine, chlorine, bromine or cyano,
R¹⁹ represents hydrogen, represents an alkali metal or alkaline earth metal ion, or represents an optionally mono- to tetra-C₁-C₄-alkyl-substituted ammonium ion or an in each case optionally mono- or poly-halogen-substituted or mono- or di-cyano-substituted radical from the group consisting of C₁-C₄-alkyl, C₁-C₄-alkoxy, C₁-C₄-alkoxy-C₁-C₂-alkyl, C₁-C₄-alkyl-S—C₁-C₂-alkyl, C₁-C₄-alkyl-S(O)—C₁-C₂-alkyl and C₁-C₄-alkyl-S(O)₂—C₁-C₂-alkyl,
R²³ represents a radical from the group consisting of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl and C₁-C₄-alkoxy-C₁-C₄alkyl,
or, if R²=g),
R²² and R²³ together with the nitrogen atom to which they are attached form a saturated four- to six-membered ring which may contain a further heteroatom from the group consisting of nitrogen, oxygen and sulfur,
R²⁶ represents a radical from the group consisting of hydrogen, C₁-C₄-alkyl, C₁-C₄-haloalkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylsulfanyl, C₁-C₄-haloalkylsulfanyl C₁-C₄-alkylsulfinyl, C₁-C₄-haloalkylsulfinyl, C₁-C₄-alkylsulfonyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl and cyano-C₁-C₄-alkyl and
R²⁷ represents hydrogen, C₁-C₄-alkyl, C₂-C₄-alkenyl, C₂-C₄-alkynyl, C₃-C₆-cycloalkyl, C₃-C₆-cycloalkyl-C₁-C₄-alkyl, C₁-C₄-alkoxy-C₁-C₂-alkyl, C₁-C₄-alkylthio-C₁-C₂-alkyl, C₁-C₄-alkylsulfinyl-C₁-C₂-alkyl, C₁-C₄-alkylsulfonyl-C₁-C₂-alkyl or cyano-C₁-C₄-alkyl.

Preferred range (3): Very particular preference is given to compounds of the formula (I) in which
A represents an A radical from the group consisting of (A-b) and (A-f)

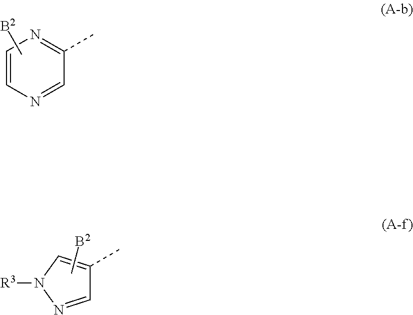

where the broken line denotes the bond to the carbon atom of the bicyclic system of the formula (I),
B² represents hydrogen,
Q represents sulfur,
R¹ represents hydrogen,
R² represents a B radical from the group consisting of

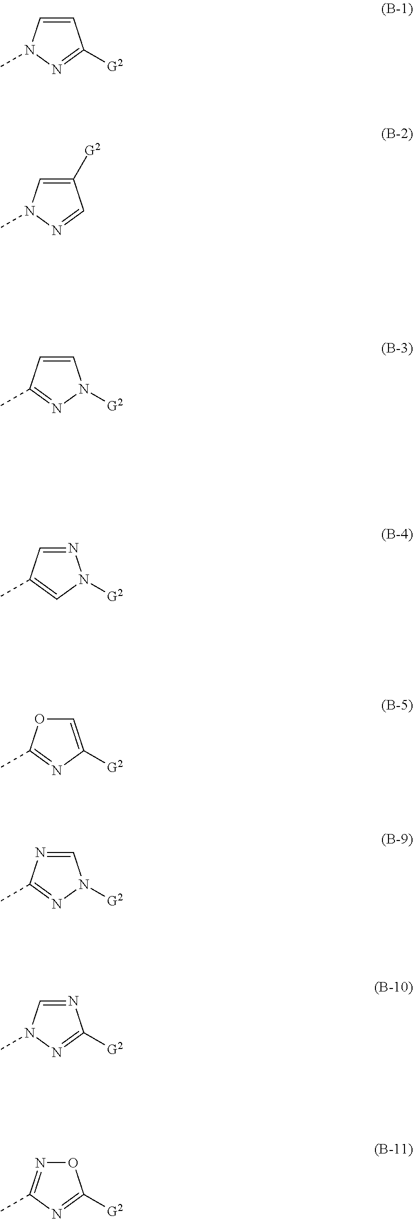

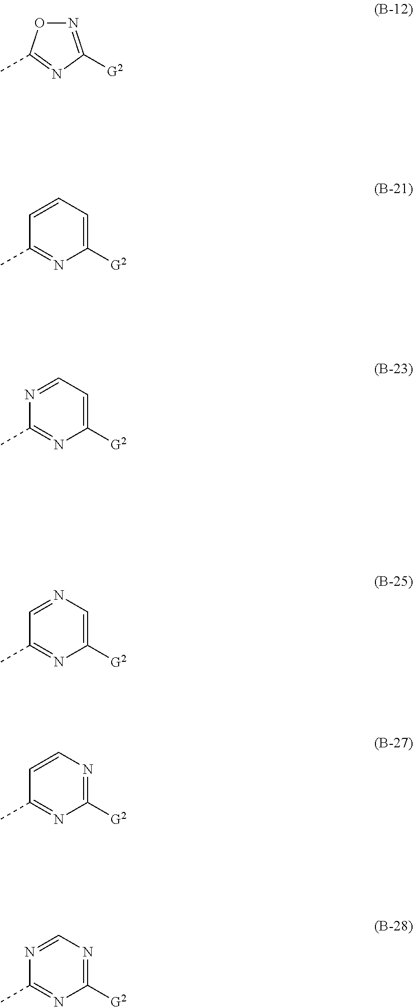

where the broken line denotes the bond to the carbon atom of the bicyclic system of the formula (I), or
R² b) represents a D radical from the group consisting of (D-1) to (D-3)

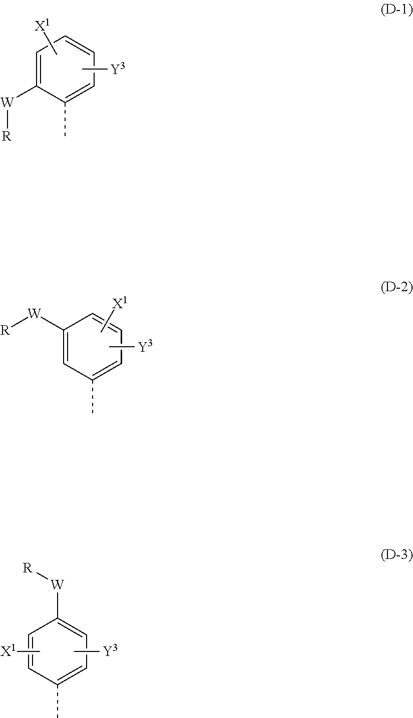

where the broken line denotes the bond to the carbon atom of the bicyclic system of the formula (I), or
R² c) represents a radical of the formula

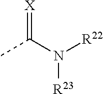

where the broken line denotes the bond to the carbon atom of the bicyclic system of the formula (I), or
R² d) represents a radical of the formula

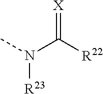

where the broken line denotes the bond to the carbon atom of the bicyclic system of the formula (I), or
R² e) represents an F radical from the group consisting of (F-1), (F-8) and (F-10)

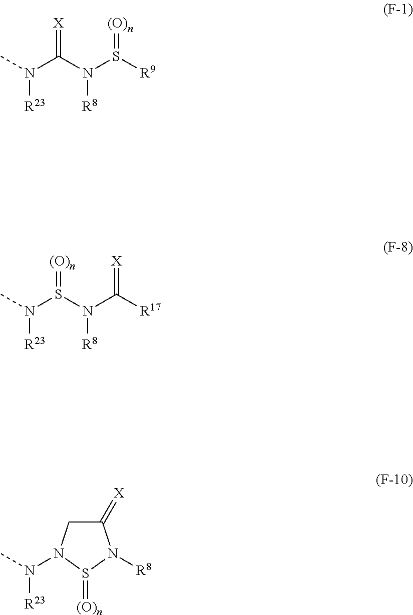

where the broken line denotes the bond to the carbon atom of the bicyclic system of the formula (I), or
R² f) represents a radical from the group consisting of C₁-C₆-haloalkyl and carboxyl, or
R² g) represents a radical of the formula

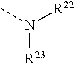

where the broken line denotes the bond to the carbon atom of the bicyclic system of the formula (I), where
G² represents hydrogen or a radical from the group consisting of halogen, nitro, amino, cyano, C₁-C₄-alkylamino, halo-C₁-C₄-alkylamino, C₁-C₄-dialkylamino, C₁-C₄-alkyl, halo-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy(C₁-C₄-alkylsulfanyl)-C₁-C₄-alkyl, C₁-C₄-alkoxy(C₁-C₄-alkylsulfinyl)-C₁-C₄-alkyl, C₁-C₄-alkoxy(C₁-C₄-alkylsulfonyl)-C₁-C₄-alkyl, bis(C₁-C₄-alkylsulfanyl)-C₁-C₄-alkyl, bis(halo-C₁-C₄-alkylsulfanyl)-C₁-C₄-alkyl, bis(hydroxy-C₁-C₄-alkylsulfanyl)-C₁-C₄-alkyl, C₁-C₄-alkoxycarbonyl, C₁-C₄-alkoxycarbonyl-C₁-C₄-alkyl, C(X²)NR³R⁴, NR⁶R⁷, C₁-C₄-alkylthio, C₁-C₄-alkylsulfinyl, C₁-C₄-alkylsulfonyl, C₁-C₄-haloalkylthio, C₁-C₄-haloalkylsulfinyl and C₁-C₄-haloalkylsulfonyl, or
G² represents a C radical (C-1) or (C-9)

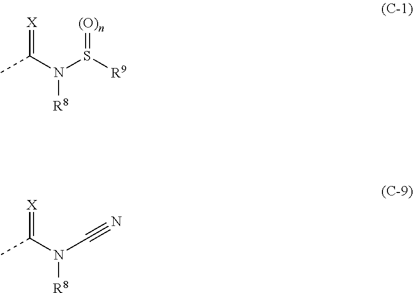

where the broken line denotes the bond to the B radicals,
X represents oxygen,
X¹ represents a radical from the group consisting of hydrogen, fluorine, chlorine, bromine, cyano, methyl, ethyl, trifluoromethyl, methoxy, ethoxy, difluoromethoxy and trifluoromethoxy,
X² represents oxygen, sulfur, NR⁵ or NOH,
n represents 2,
R represents NR¹⁸R¹⁹ or represents a radical from the group consisting of C₁-C₄-alkyl, C₃-C₄-alkenyl, C₃-C₄-alkynyl, C₁-C₂-alkoxy-C₁-C₂-alkyl and C₁-C₂-alkyl-S—C₁-C₂-alkyl, C₁-C₂-alkyl-S(O)—C₁-C₂-alkyl, C₁-C₂-alkyl-S(O)₂—C₁-C₂-alkyl, each of which is optionally mono-, di-, tri-, tetra- or pentasubstituted by fluorine, chlorine or mono- or disubstituted by cyano, represents R¹⁸—CO—C₁-C₂-alkyl, represents NR¹⁸R¹⁹—CO—C₁-C₂-alkyl, represents C₃-C₆-cycloalkyl which is optionally mono- or disubstituted by C₁-C₂-alkyl, C₁-C₂-alkoxy or C₁-C₂-haloalkyl or by an oxygen atom (leads to C═O), represents C₃-C₆-cycloalkenyl which is optionally mono- or disubstituted by C₁-C₂-alkyl, C₁-C₂-alkoxy or C₁-C₂-haloalkyl or by an oxygen atom (leads to C═O), represents C₃-C₆-cycloalkyl-C₁-C₂-alkyl which is optionally mono- to disubstituted by C₁-C₂-alkyl, C₁-C₂-alkoxy or C₁-C₂-haloalkyl,
represents C₃-C₆-cycloalkenyl-C₁-C₂-alkyl which is optionally mono- or disubstituted by C₁-C₂-alkyl, C₁-C₂-alkoxy or C₁-C₂-haloalkyl, represents heterocyclyl which is optionally mono- or disubstituted by C₁-C₂-alkyl, C₁-C₂-alkoxy or C₁-C₂-haloalkyl, represents heterocyclyl-C₁-C₂-alkyl which is optionally mono- or disubstituted by C₁-C₂-alkyl, C₁-C₂-alkoxy or C₁-C₂-haloalkyl or represents phenyl, benzyl, pyridyl, pyrimidyl, thiazolyl, oxazolyl, pyrazolyl, thienyl, furanyl, pyridinylmethyl or thiazolylmethyl, each of which is optionally mono- or disubstituted by fluorine, chlorine, bromine, cyano, methyl, ethyl, difluoromethyl, trifluoromethyl, methoxy, ethoxy, difluoromethoxy or trifluoromethoxy,
R³ represents C₁-C₄-alkyl,
R⁴ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁵ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁶ represents hydrogen or C₁-C₄-alkyl,
R⁷ represents a radical from the group consisting of hydrogen, C₁-C₄-alkyl, halo-C₁-C₄-alkyl, cyano-C₁-C₄-alkyl, C₂-C₄-alkynyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl-C₁-C₄-alkyl and hetaryl-C₁-C₄-alkyl or
R⁶ and R⁷ together with the nitrogen atom to which they are attached form a 4- to 7-membered ring which may contain one or two further heteroatoms from the group consisting of nitrogen, oxygen and sulfur (where oxygen and sulfur atoms must not be directly adjacent to one another),
R⁸ represents a radical from the group consisting of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulfonyl, optionally halogen-substituted C₁-C₆-alkoxycarbonyl, optionally halogen-, C₁-C₆-alkyl-, C₁-C₆-alkoxy-, C₁-C₆-haloalkyl- and cyano-substituted C₃-C₆-cycloalkylcarbonyl, or represents a cation or an optionally C₁-C₆-alkyl- or aryl-C₁-C₆-alkyl-substituted ammonium ion,
R⁹ represents a radical from the group consisting of in each case optionally halogen-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, in which one or two ring members may in each case be replaced by a heteroatom from the group consisting of sulfur, oxygen (where oxygen atoms must not be directly adjacent to one another) and nitrogen (and here in particular represent

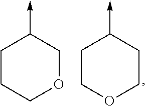

where the arrow in each case marks the bond to the sulfur atom in the radical (C-1) and in the radical (F-1)), in each case optionally halogen-, cyano- (also in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl, heteroaryl-C₁-C₄-alkyl or represents NR′R″ in which R′ and R″ independently of one another represent a radical from the group consisting of hydrogen and C₁-C₄-alkyl,
R⁸ and R⁹ in the radical (C-1) and in the radical (F-1) may also form, together with the N—S(O)n group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group consisting of sulfur, oxygen (where oxygen atoms must not be directly adjacent to one another) and nitrogen and/or at least one and preferably one carbonyl group, in particular, R⁸ and R⁹ together with the N—S(O)n group to which they are attached may represent a radical from the group consisting of

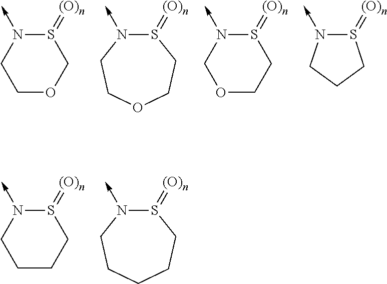

(in which the arrow in each case marks the bond to the C(X) group),
R¹⁷ represents a radical from the group consisting of in each case optionally halogen-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl and N-2-oxo-1-methylpiperazinyl, in each case optionally halogen-, cyano- (also in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulfinyl-,
C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl, heteroaryl-C₁-C₄-alkyl or represents NR′R″ in which R′ and R″ independently of one another represent a radical from the group consisting of hydrogen and C₁-C₄-alkyl,
R¹⁸ represents a radical from the group consisting of hydrogen, hydroxy, represents C₁-C₄-alkyl, C₁-C₄-alkoxy, C₁-C₄-alkoxy-C₁-C₄-alkyl, C₁-C₄-alkyl-S—C₁-C₂-alkyl, C₁-C₄-alkyl-S(O)—C₁-C₂-alkyl, C₁-C₄-alkyl-S(O)₂—C₁-C₂-alkyl, C₁-C₄-alkylcarbonyl, C₁-C₄-alkoxycarbonyl, C₃-C₆-cycloalkyl, C₃-C₆-cycloalkyl-C₁-C₃-alkyl, heterocyclyl and heterocyclyl-C₁-C₃-alkyl, each of which is optionally mono-, di-, tri-, tetra- or pentasubstituted by fluorine, chlorine or mono- or disubstituted by cyano, and of phenyl, benzyl, pyridyl, pyrimidyl, thiazolyl, oxazolyl, pyrazolyl, thienyl, furanyl, pyridinylmethyl and thiazolylmethyl, each of which is optionally mono- to trisubstituted by C₁-C₄-alkyl, C₁-C₃-haloalkyl, C₁-C₃-alkoxy, C₁-C₃-haloalkoxy, cyclopropyl, fluorine, chlorine, bromine or cyano,
R¹⁹ represents hydrogen, an alkali or alkaline earth metal ion, represents an ammonium ion which is optionally mono- to tetrasubstituted by C₁-C₄-alkyl or represents a radical from the group consisting of C₁-C₄-alkyl, C₁-C₄-alkoxy, C₁-C₄-alkoxy-C₁-C₂-alkyl and C₁-C₄-alkyl-S—C₁-C₂-alkyl, C₁-C₄-alkyl-S(O)—C₁-C₂-alkyl and C₁-C₄-alkyl-S(O)₂—C₁-C₂-alkyl, each of which is optionally mono-, di-, tri-, tetra- or pentasubstituted by fluorine, chlorine or mono- or disubstituted by cyano,
W represents a radical from the group consisting of S, SO and SO₂,
Y³ represents a radical from the group consisting of hydrogen, fluorine, chlorine, bromine, cyano, methyl, ethyl, trifluoromethyl, methoxy, ethoxy, difluoromethoxy and trifluoromethoxy,
R²² represents a radical from the group consisting of methyl, ethyl, isopropyl, n-propyl, n-butyl, isobutyl, sec-butyl, tert-butyl, 2,2,2-trifluoroethyl, 2,2-difluoroethyl, 2,2-difluoro-n-propyl, methylsulfanylmethyl, methylsulfanylethyl, methylsulfanyl-n-propyl, trifluoromethylsulfonylmethyl, ethylsulfonylmethyl, 2,2,2-trifluoroethylsulfonylmethyl, 2,2-difluoroethylsulfonylmethyl, isopropylsulfanylmethyl, methylsulfinylmethyl, trifluoromethylsulfinylmethyl, ethylsulfinylmethyl, 2,2,2-trifluoroethylsulfinylmethyl, 2,2-difluoroethylsulfinylmethyl, isopropylsulfinylmethyl, methylsulfonylmethyl, trifluoromethylsulfonylmethyl, ethylsulfonylmethyl, 2,2,2-trifluoroethylsulfonylmethyl, 2,2-difluoroethylsulfonylmethyl, isopropylsulfonylmethyl, methoxycarbonyl, ethoxycarbonyl, methoxycarbonylmethyl, ethoxycarbonylmethyl, methoxycarbonylethyl, ethoxycarbonylethyl, dimethylaminocarbonyl, diethylaminocarbonyl, N-ethyl-N-methylaminocarbonyl, N-isopropyl-N-methylaminocarbonyl, dimethylaminocarbonylmethyl, diethylaminocarbonylmethyl, N-ethyl-N-methylaminocarbonylmethyl, N-isopropyl-N-methylaminocarbonylmethyl, dimethylaminocarbonylethyl, diethylaminocarbonylethyl, N-ethyl-N-methylaminocarbonylethyl, N-isopropyl-N-methylaminocarbonylethyl, N-cyclopropyl-N-methylaminocarbonylmethyl, N-cyclopropyl-N-methylaminocarbonylethyl, methylsulfanyl, trifluoromethylsulfanyl, ethylsulfanyl, 2,2,2-trifluoroethylsulfanyl, 2,2-difluoroethylsulfanyl, isopropylsulfanyl, methylsulfinyl, trifluoromethylsulfinyl, ethylsulfinyl, 2,2,2-trifluoroethylsulfinyl, 2,2-difluoroethylsulfinyl, isopropylsulfinyl, methylsulfonyl, trifluoromethylsulfonyl, ethylsulfonyl, 2,2,2-trifluoroethylsulfonyl, 2,2-difluoroethylsulfonyl, isopropylsulfonyl, cyclopropyl, 1-cyanocyclopropyl, 1-chlorocyclopropyl, 1-fluorocyclopropyl, 2-cyanocyclopropyl, 2-chlorocyclopropyl, 2-fluorocyclopropyl, 2,2,3,3-tetrafluorocyclopropyl, 2-cyclopropylcyclopropyl, cyclobutyl, cyclopentyl, cyclohexyl, 4-trifluoromethylcyclohexyl, cyclopropylmethyl, cyclopropylethyl, cyclobutylmethyl, N-cyclopropyl-N-methylaminocarbonyl, morpholin-4-ylcarbonylmethyl, piperazin-1-ylcarbonylmethyl, 4-methylpiperazin-1-ylcarbonylmethyl, heterocyclylmethyl and heterocyclylethyl, each of which is optionally mono-, di- or trisubstituted by identical or different substituents from the group consisting of fluorine, chlorine, bromine, cyano, nitro, hydroxyl, amino, methyl, ethyl, isopropyl, tert-butyl, trifluoromethyl, difluoromethyl, methoxy, trifluoromethoxy and difluoromethoxy, in each case cyclopropyl-substituted heterocyclylmethyl and heterocyclylethyl, where the cyclopropyl radical is optionally mono- or disubstituted by methyl, fluorine, chlorine or cyano or monosubstituted by cyclopropyl, aryl, which is optionally mono-, di- or trisubstituted by identical or different substituents from the group consisting of fluorine, chlorine, bromine, cyano, nitro, hydroxyl, amino, methyl, ethyl, isopropyl, tert-butyl, trifluoromethyl, difluoromethyl, methoxy, trifluoromethoxy and difluoromethoxy, cyclopropyl-substituted aryl, where the cyclopropyl radical is optionally mono- or disubstituted by methyl, fluorine, chlorine or cyano or monosubstituted by cyclopropyl, arylmethyl and arylethyl, each optionally mono-, di- or trisubstituted by identical or different substituents from the group consisting of fluorine, chlorine, bromine, cyano, nitro, hydroxyl, amino, methyl, ethyl, isopropyl, tert-butyl, trifluoromethyl, difluoromethyl, methoxy, trifluoromethoxy and difluoromethoxy, cyclopropyl-substituted arylmethyl and arylethyl, where the cyclopropyl radical is optionally mono- or disubstituted by methyl, fluorine, chlorine or cyano or monosubstituted by cyclopropyl, hetarylmethyl and hetarylethyl, each optionally mono- or disubstituted by identical or different substituents from the group consisting of fluorine, chlorine, bromine, cyano, nitro, hydroxy, amino, methyl, ethyl, isopropyl, tert-butyl, trifluoromethyl, difluoromethyl, methoxy, trifluoromethoxy, difluoromethoxy, cyclopropyl-substituted hetarylmethyl and hetarylethyl, where the cyclopropyl radical is optionally mono- or disubstituted by methyl, fluorine, chlorine, cyano or monosubstituted by cyclopropyl, or
R²² represents a D radical from the group consisting of (D-1) to (D-3)

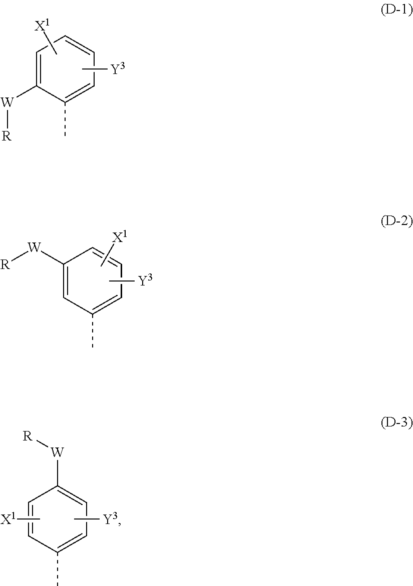

represents one of the E radicals below

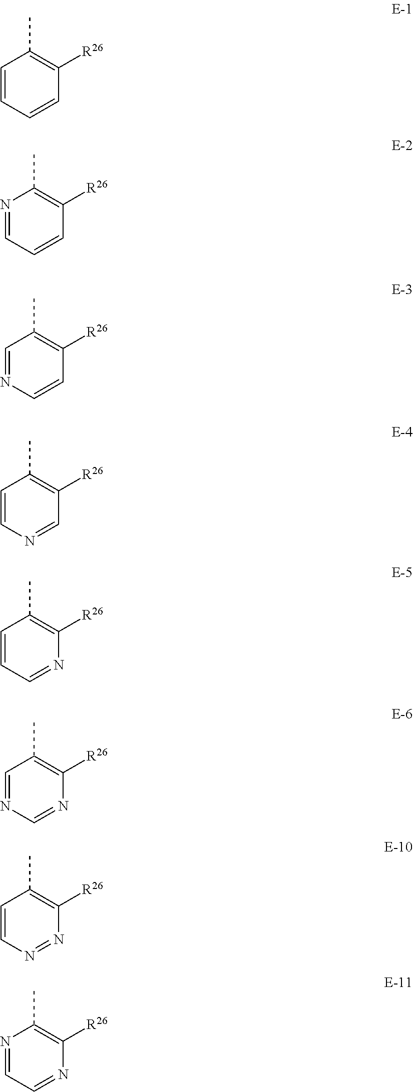

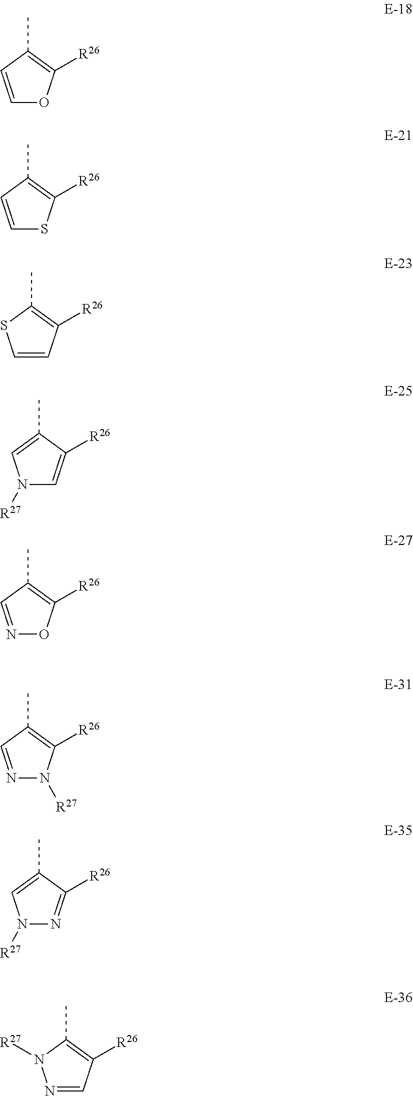

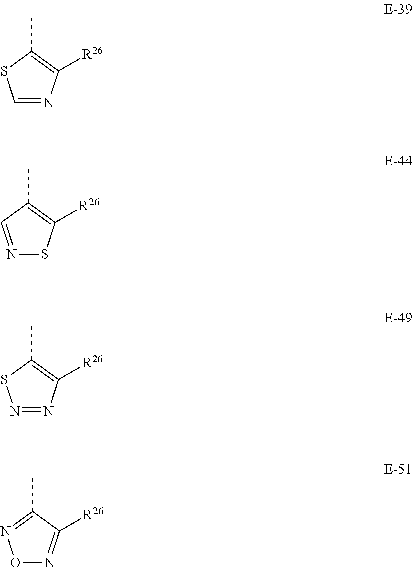

or in the case that R²=d)
R²² also represents the radical (E-13)

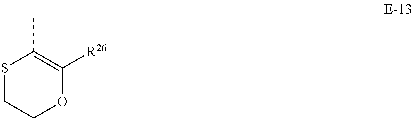

R²³ represents a radical from the group consisting of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄alkyl,
or, if R²=c) or g),
R²² and R²³ together with the nitrogen atom to which they are attached also represent pyrrolidinyl, piperidinyl, morpholinyl, thiomorpholinyl, thiomorpholinyl 1-oxide, thiomorpholinyl 1,1-dioxide, piperazinyl, 1-methylpiperazinyl or 2-oxo-1-methylpiperazinyl,
R²⁶ represents a radical from the group consisting of hydrogen, methyl, 2,2,2-trifluoroethyl, 2,2-difluoroethyl, propenyl, propargyl, cyclopropyl, cyclopropylmethyl, methoxymethyl, methylsulfanyl, trifluoromethylsulfanyl, ethylsulfanyl, trifluoroethylsulfanyl, methylsulfinyl, trifluoromethylsulfinyl, ethylsulfinyl, trifluoroethylsulfinyl, methylthioethyl, methylsulfinylethyl, methylsulfonylethyl and cyanomethyl and
R²⁷ represents hydrogen, methyl, 2,2,2-trifluoroethyl, 2,2-difluoroethyl, propenyl, propargyl, cyclopropyl, cyclopropylmethyl, methoxymethyl, methylthioethyl, methylsulfinylethyl, methylsulfonylethyl or cyanomethyl and
compounds of the formula (I) in which
A represents the A radical

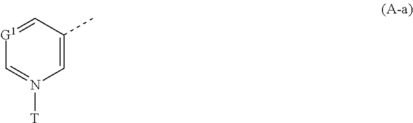

where the broken line denotes the bond to the carbon atom of the bicyclic system of the formula (I),
G¹ represents N or C—B¹,
B¹ represents a radical from the group consisting of hydrogen and fluorine,
T represents oxygen or an electron pair,
Q represents sulfur,
R¹ represents hydrogen,
R² represents a B radical from the group consisting of

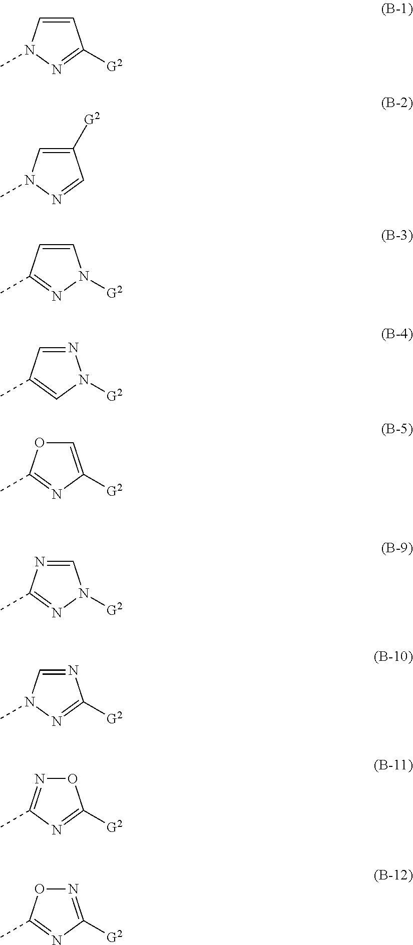

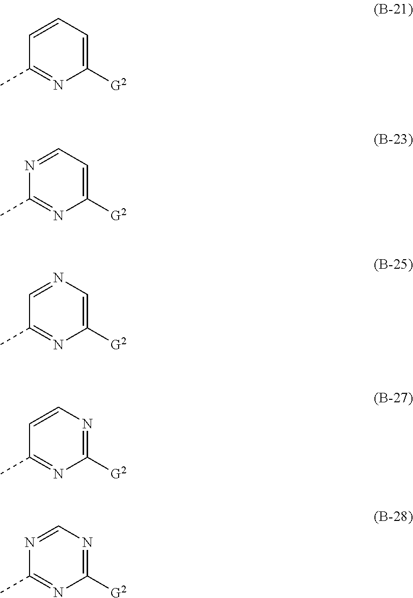

where the broken line denotes the bond to the carbon atom of the bicyclic system of the formula (I), or
R² c) represents a radical of the formula

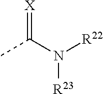

where the broken line denotes the bond to the carbon atom of the bicyclic system of the formula (I), or
R² d) represents a radical of the formula

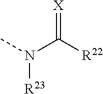

where the broken line denotes the bond to the carbon atom of the bicyclic system of the formula (I), or
R² e) represents an F radical from the group consisting of (F-8) and (F-10)

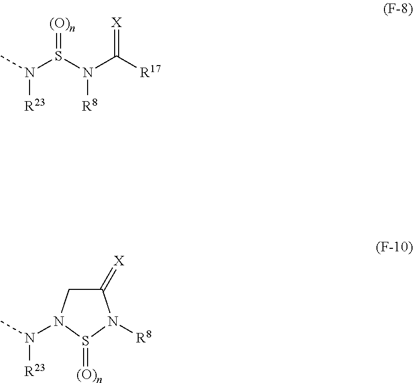

where the broken line denotes the bond to the carbon atom of the bicyclic system of the formula (I), or
R² f) represents a radical from the group consisting of C₁-C₆-haloalkyl and carboxyl, or
R² g) represents a radical of the formula

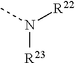

where the broken line denotes the bond to the carbon atom of the bicyclic system of the formula (I), where
G² represents hydrogen or a radical from the group consisting of halogen, nitro, amino, cyano, C₁-C₄-alkylamino, halo-C₁-C₄-alkylamino, C₁-C₄-dialkylamino, C₁-C₄-alkyl, halo-C₁-C₄-alkyl, C₁-C₄-alkoxy, halo-C₁-C₄-alkoxy, C₁-C₄-alkoxy-C₁-C₄-alkyl, halogenated C₁-C₄-alkoxy-C₁-C₄-alkyl, bis(C₁-C₄-alkoxy)-C₁-C₄-alkyl, bis(halo-C₁-C₄-alkoxy)-C₁-C₄-alkyl, C₁-C₄-alkoxy(C₁-C₄-alkylsulfanyl)-C₁-C₄-alkyl, C₁-C₄-alkoxy(C₁-C₄-alkylsulfinyl)-C₁-C₄-alkyl, C₁-C₄-alkoxy(C₁-C₄-alkylsulfonyl)-C₁-C₄-alkyl, bis(C₁-C₄-alkylsulfanyl)-C₁-C₄-alkyl, bis(halo-C₁-C₄-alkylsulfanyl)-C₁-C₄-alkyl, bis(hydroxy-C₁-C₄-alkylsulfanyl)-C₁-C₄-alkyl, C₁-C₄-alkoxycarbonyl, C₁-C₄-alkoxycarbonyl-C₁-C₄-alkyl, C(X²)NR³R⁴, NR⁶R⁷, C₁-C₄-alkylthio, C₁-C₄-alkylsulfinyl, C₁-C₄-alkylsulfonyl, C₁-C₄-haloalkylthio, C₁-C₄-haloalkylsulfinyl and C₁-C₄-haloalkylsulfonyl, or
G² represents a radical (C-1) or (C-9)

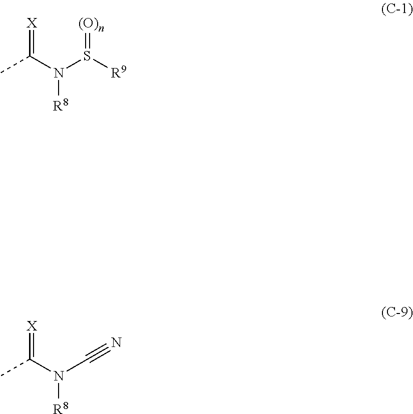

where the broken line represents the bond to the B radicals,
X represents oxygen,
X² represents oxygen, sulfur, NR⁵ or NOH,
n represents 2,
R³ represents C₁-C₄-alkyl,
R⁴ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁵ represents a radical from the group consisting of hydrogen, C₁-C₄-alkyl, halo-C₁-C₄-alkyl, cyano-C₁-C₄-alkyl, C₂-C₄-alkynyl, C₃-C₆-cycloalkyl, C₃-C₆-cycloalkyl-C₁-C₄-alkyl, C₁-C₄-alkoxy-C₁-C₄-alkyl, C₁-C₄-alkoxycarbonyl, C₁-C₄-alkoxycarbonyl-C₁-C₄-alkyl and C₁-C₄-alkylthio-C₁-C₄-alkyl,
R⁶ represents hydrogen or C₁-C₄-alkyl,
R⁷ represents a radical from the group consisting of hydrogen, C₁-C₄-alkyl, halo-C₁-C₄-alkyl, cyano-C₁-C₄-alkyl, C₂-C₄-alkynyl, C₁-C₄-alkoxy, halo-C₁-C₄-alkoxy, C₁-C₄-alkoxy-C₁-C₄-alkyl, C₁-C₄-alkoxycarbonyl, C₁-C₄-alkoxycarbonyl-C₁-C₄-alkyl, C₁-C₄-alkylthio-C₁-C₄-alkyl, aryl-C₁-C₄-alkyl and hetaryl-C₁-C₄-alkyl or
R⁶ and R⁷ together with the nitrogen atom to which they are attached form a 4- to 7-membered ring which may contain one or two further heteroatoms from the group consisting of nitrogen, oxygen and sulfur (where oxygen and sulfur atoms must not be directly adjacent to one another),
R⁸ represents a radical from the group consisting of hydrogen, C₁-C₆-alkyl, C₁-C₆-haloalkyl, cyano-C₁-C₆-alkyl, C₁-C₆-alkoxy, C₁-C₆-haloalkoxy, C₂-C₆-alkenyl, C₁-C₆-alkoxy-C₁-C₆-alkyl, in each case optionally halogen-substituted C₁-C₆-alkylcarbonyl and C₁-C₆-alkylsulfonyl, optionally halogen-substituted C₁-C₆-alkoxycarbonyl, optionally halogen-, C₁-C₆-alkyl-, C₁-C₆-alkoxy-, C₁-C₆-haloalkyl- and cyano-substituted C₃-C₆-cycloalkylcarbonyl, or represents a cation or an optionally C₁-C₆-alkyl- or aryl-C₁-C₆-alkyl-substituted ammonium ion,
R⁹ represents a radical from the group consisting of in each case optionally halogen-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, in which one or two ring members may in each case be replaced by a heteroatom from the group consisting of sulfur, oxygen (where oxygen atoms must not be directly adjacent to one another) and nitrogen (and here in particular represent

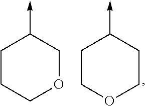

where the arrow in each case marks the bond to the sulfur atom in the (C-1) radical), in each case optionally halogen-, cyano- (including in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl and heteroaryl-C₁-C₄-alkyl, or is NR′R″ in which R′ and R″ are independently a radical from the group of hydrogen and C₁-C₄-alkyl,
R⁸ and R⁹ in the radical (C-1) may also form, together with the N—S(O)n group to which they are attached, a saturated or unsaturated and optionally halogen-, C₁-C₆-alkyl-, C₁-C₆-haloalkyl-, C₁-C₆-alkoxy-, C₁-C₆-haloalkoxy-substituted 5- to 7-membered ring which may contain one or two heteroatoms from the group consisting of sulfur, oxygen (where oxygen atoms must not be directly adjacent to one another) and nitrogen and/or at least one and preferably one carbonyl group, in particular, R⁸ and R⁹ together with the N—S(O)n group to which they are attached may represent a radical from the group consisting of

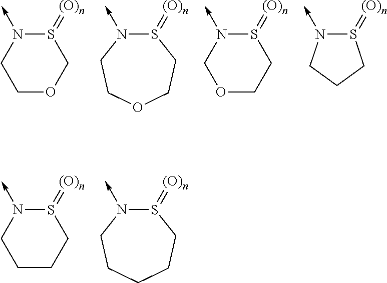

(in which the arrow in each case marks the bond to the C(X) group),
R¹⁷ represents a radical from the group consisting of in each case optionally halogen-, C₁-C₄-alkoxy-, C₁-C₄-haloalkoxy C₁-C₄-alkylthio-, C₁-C₄-haloalkylthio-, C₁-C₄-alkylsulfinyl-, C₁-C₄-haloalkylsulfinyl-, C₁-C₄-alkylsulfonyl- and C₁-C₄-haloalkylsulfonyl-substituted C₁-C₄-alkyl, C₂-C₄-alkenyl and C₂-C₄-alkynyl, in each case optionally halogen-, C₁-C₄-alkyl-, C₁-C₄-haloalkyl-, C₁-C₄-alkoxy-, C₁-C₄-haloalkoxy-substituted C₃-C₆-cycloalkyl, C₃-C₆-cycloalkyl-C₁-C₄-alkyl and C₃-C₄-cycloalkenyl, N-pyrrolidinyl, N-piperidinyl, N-morpholinyl, N-thiomorpholinyl, N-thiomorpholinyl 1-oxide, N-thiomorpholinyl 1,1-dioxide, N-piperazinyl, N-1-methylpiperazinyl and N-2-oxo-1-methylpiperazinyl, in each case optionally halogen-, cyano- (also in the alkyl moiety), nitro-, C₁-C₄-alkyl-, C₁-C₄-haloalkyl-, C₃-C₆-cycloalkyl-, C₁-C₄-alkoxy-, C₁-C₄-haloalkoxy-, C₁-C₄-alkylthio-, C₁-C₄-haloalkylthio-, C₁-C₄-alkylsulfinyl-, C₁-C₄-haloalkylsulfinyl-, C₁-C₄-alkylsulfonyl-, C₁-C₄-haloalkylsulfonyl-, amino-, C₁-C₄-alkylamino-, di(C₁-C₄-alkyl)amino-, C₁-C₄-alkylcarbonylamino-, C₁-C₄-alkoxycarbonylamino-, C₁-C₄-alkoxy-C₁-C₄-alkyl-, C₁-C₄-haloalkoxy-C₁-C₄-alkyl-, C₂-C₄-alkenyl-, C₂-C₄-alkynyl-, C₃-C₆-cycloalkyl-C₁-C₄-alkyl-, C₁-C₄-alkylcarbonyl-, C₁-C₄-alkoxycarbonyl- or aminocarbonyl-substituted aryl, heteroaryl, aryl-C₁-C₄-alkyl, heteroaryl-C₁-C₄-alkyl or represents NR′R″ in which R′ and R″ independently of one another represent a radical from the group consisting of hydrogen and C₁-C₄-alkyl,
and, in the case that R² represents g),
R²² represents a radical from the group consisting of methyl, ethyl, isopropyl, n-propyl, n-butyl, isobutyl, sec-butyl, tert-butyl, 2,2,2-trifluoroethyl, 2,2-difluoroethyl, 2,2-difluoro-n-propyl, methylsulfanylmethyl, methylsulfanylethyl, methylsulfanyl-n-propyl, trifluoromethylsulfonylmethyl, ethylsulfonylmethyl, 2,2,2-trifluoroethylsulfonylmethyl, 2,2-difluoroethylsulfonylmethyl, isopropylsulfanylmethyl, methylsulfinylmethyl, trifluoromethylsulfinylmethyl, ethylsulfinylmethyl, 2,2,2-trifluoroethylsulfinylmethyl, 2,2-difluoroethylsulfinylmethyl, isopropylsulfinylmethyl, methylsulfonylmethyl, trifluoromethylsulfonylmethyl, ethylsulfonylmethyl, 2,2,2-trifluoroethylsulfonylmethyl, 2,2-difluoroethylsulfonylmethyl, isopropylsulfonylmethyl, methoxycarbonylmethyl, ethoxycarbonylmethyl, methoxycarbonylethyl, ethoxycarbonylethyl, dimethylaminocarbonylmethyl, diethylaminocarbonylmethyl, N-ethyl-N-methylaminocarbonylmethyl, N-isopropyl-N-methylaminocarbonylmethyl, dimethylaminocarbonylethyl, diethylaminocarbonylethyl, N-ethyl-N-methylaminocarbonylethyl, N-isopropyl-N-methylaminocarbonylethyl, N-cyclopropyl-N-methylaminocarbonylmethyl, N-cyclopropyl-N-methylaminocarbonylethyl, methylsulfanyl, trifluoromethylsulfanyl, ethylsulfanyl, 2,2,2-trifluoroethylsulfanyl, 2,2-difluoroethylsulfanyl, isopropylsulfanyl, methylsulfinyl, trifluoromethylsulfinyl, ethylsulfinyl, 2,2,2-trifluoroethylsulfinyl, 2,2-difluoroethylsulfinyl, isopropylsulfinyl, methylsulfonyl, trifluoromethylsulfonyl, ethylsulfonyl, 2,2,2-trifluoroethylsulfonyl, 2,2-difluoroethylsulfonyl, isopropylsulfonyl, cyclopropyl, 1-cyanocyclopropyl, 1-chlorocyclopropyl, 1-fluorocyclopropyl, 2-cyanocyclopropyl, 2-chlorocyclopropyl, 2-fluorocyclopropyl, 2,2,3,3-tetrafluorocyclopropyl, 2-cyclopropylcyclopropyl, cyclobutyl, cyclopentyl, cyclohexyl, 4-trifluoromethylcyclohexyl, cyclopropylmethyl, cyclopropylethyl, cyclobutylmethyl, morpholin-4-ylcarbonylmethyl, piperazin-1-ylcarbonylmethyl, 4-methylpiperazin-1-ylcarbonylmethyl, heterocyclylmethyl and heterocyclylethyl, each of which is optionally mono-, di- or trisubstituted by identical or different substituents from the group consisting of fluorine, chlorine, bromine, cyano, nitro, hydroxyl, amino, methyl, ethyl, isopropyl, tert-butyl, trifluoromethyl, difluoromethyl, methoxy, trifluoromethoxy and difluoromethoxy, in each case cyclopropyl-substituted heterocyclylmethyl and heterocyclylethyl, where the cyclopropyl radical is optionally mono- or disubstituted by methyl, fluorine, chlorine or cyano or monosubstituted by cyclopropyl, aryl, which is optionally mono-, di- or trisubstituted by identical or different substituents from the group consisting of fluorine, chlorine, bromine, cyano, nitro, hydroxyl, amino, methyl, ethyl, isopropyl, tert-butyl, trifluoromethyl, difluoromethyl, methoxy, trifluoromethoxy and difluoromethoxy, cyclopropyl-substituted aryl, where the cyclopropyl radical is optionally mono- or disubstituted by methyl, fluorine, chlorine or cyano or monosubstituted by cyclopropyl, arylmethyl and arylethyl, each optionally mono-, di- or trisubstituted by identical or different substituents from the group consisting of fluorine, chlorine, bromine, cyano, nitro, hydroxyl, amino, methyl, ethyl, isopropyl, tert-butyl, trifluoromethyl, difluoromethyl, methoxy, trifluoromethoxy and difluoromethoxy, cyclopropyl-substituted arylmethyl and arylethyl, where the cyclopropyl radical is optionally mono- or disubstituted by methyl, fluorine, chlorine or cyano or monosubstituted by cyclopropyl, hetarylmethyl and hetarylethyl, each optionally mono- or disubstituted by identical or different substituents from the group consisting of fluorine, chlorine, bromine, cyano, nitro, hydroxy, amino, methyl, ethyl, isopropyl, tert-butyl, trifluoromethyl, difluoromethyl, methoxy, trifluoromethoxy and difluoromethoxy, cyclopropyl-substituted hetarylmethyl and hetarylethyl, where the cyclopropyl radical is optionally mono- or disubstituted by methyl, fluorine, chlorine or cyano or monosubstituted by cyclopropyl, and, in the case that R2 represents c), d) or f),
R²² represents a D radical from the group consisting of (D-1) to (D-3)

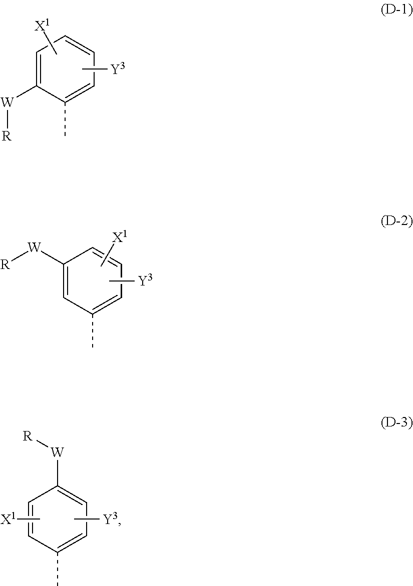

in which
X¹ represents a radical from the group consisting of hydrogen, fluorine, chlorine, bromine, cyano, methyl, ethyl, trifluoromethyl, methoxy, ethoxy, difluoromethoxy and trifluoromethoxy,
R represents NR¹⁸R¹⁹ or represents a radical from the group consisting of C₁-C₄-alkyl, C₃-C₄-alkenyl, C₃-C₄-alkynyl, C₁-C₂-alkoxy-C₁-C₂-alkyl and C₁-C₂-alkyl-S—C₁-C₂-alkyl, C₁-C₂-alkyl-S(O)—C₁-C₂-alkyl, C₁-C₂-alkyl-S(O)₂—C₁-C₂-alkyl, each of which is optionally mono-, di-, tri-, tetra- or pentasubstituted by fluorine, chlorine or mono- or disubstituted by cyano, represents R¹⁸—CO—C₁-C₂-alkyl, represents NR¹⁸R¹⁹—CO—C₁-C₂-alkyl, represents C₃-C₆-cycloalkyl which is optionally mono- or disubstituted by C₁-C₂-alkyl, C₁-C₂-alkoxy or C₁-C₂-haloalkyl or by an oxygen atom (leads to C═O), represents C₃-C₆-cycloalkenyl which is optionally mono- or disubstituted by C₁-C₂-alkyl, C₁-C₂-alkoxy or C₁-C₂-haloalkyl or by an oxygen atom (leads to C═O), represents C₃-C₆-cycloalkyl-C₁-C₂-alkyl which is optionally mono- or disubstituted by C₁-C₂-alkyl, C₁-C₂-alkoxy or C₁-C₂-haloalkyl, represents C₃-C₆-cycloalkenyl-C₁-C₂-alkyl which is optionally mono- or disubstituted by C₁-C₂-alkyl, C₁-C₂-alkoxy or C₁-C₂-haloalkyl, represents heterocyclyl which is optionally mono- or disubstituted by C₁-C₂-alkyl, C₁-C₂-alkoxy or C₁-C₂-haloalkyl, represents heterocyclyl-C₁-C₂-alkyl which is optionally mono- or disubstituted by C₁-C₂-alkyl, C₁-C₂-alkoxy or C₁-C₂-haloalkyl or represents phenyl, benzyl, pyridyl, pyrimidyl, thiazolyl, oxazolyl, pyrazolyl, thienyl, furanyl, pyridinylmethyl or thiazolylmethyl, each of which is optionally mono- or disubstituted by fluorine, chlorine, bromine, cyano, methyl, ethyl, difluoromethyl, trifluoromethyl, methoxy, ethoxy, difluoromethoxy or trifluoromethoxy,
W represents a radical from the group consisting of S, SO and SO₂,
Y³ represents a radical from the group consisting of hydrogen, fluorine, chlorine, bromine, cyano, methyl, ethyl, trifluoromethyl, methoxy, ethoxy, difluoromethoxy and trifluoromethoxy,
R²² furthermore represents one of the E radicals below

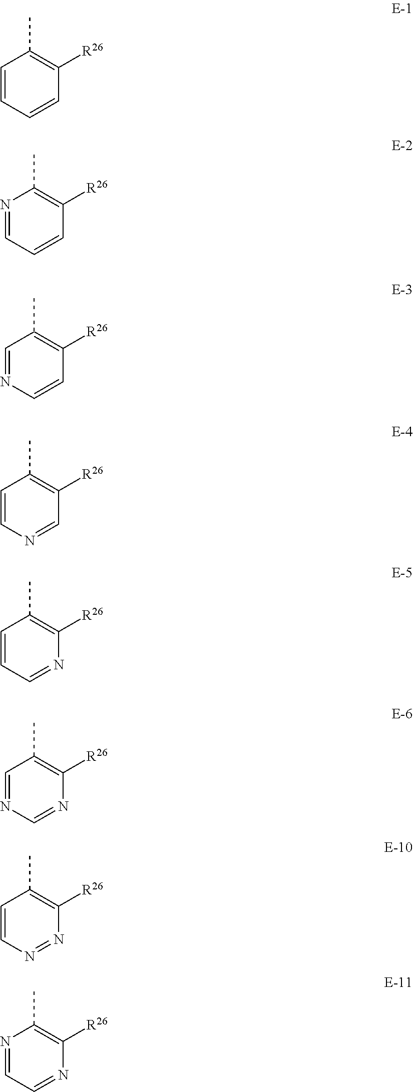

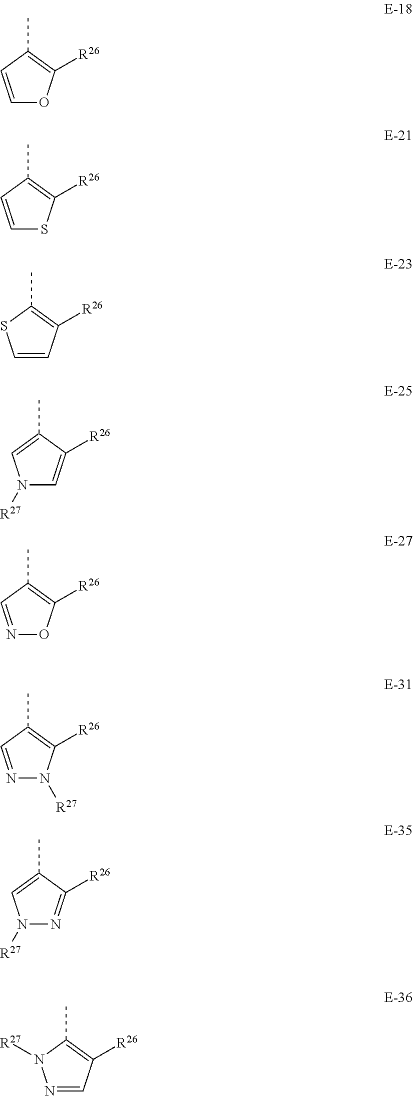

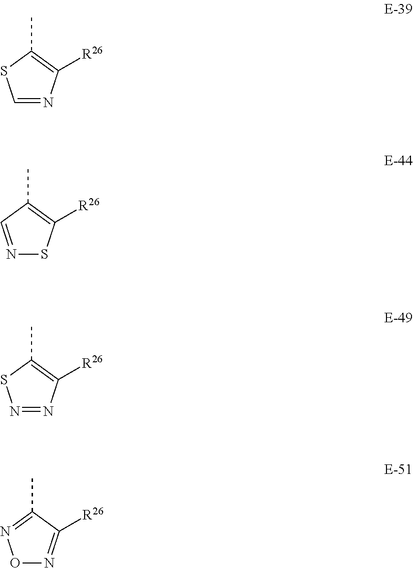

or, in the case that R²=d),
R²² also represents the radical (E-13)

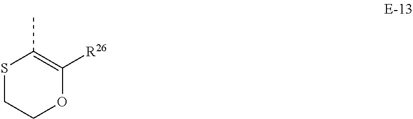

R¹⁸ represents a radical from the group consisting of hydrogen, hydroxy, represents C₁-C₄-alkyl, C₁-C₄-alkoxy, C₁-C₄-alkoxy-C₁-C₄-alkyl, C₁-C₄-alkyl-S—C₁-C₂-alkyl, C₁-C₄-alkyl-S(O)—C₁-C₂-alkyl, C₁-C₄-alkyl-S(O)₂—C₁-C₂-alkyl, C₁-C₄-alkylcarbonyl, C₁-C₄-alkoxycarbonyl, C₃-C₆-cycloalkyl, C₃-C₆-cycloalkyl-C₁-C₃-alkyl, heterocyclyl and heterocyclyl-C₁-C₃-alkyl, each of which is optionally mono-, di-, tri-, tetra- or pentasubstituted by fluorine, chlorine or mono- or disubstituted by cyano, and of phenyl, benzyl, pyridyl, pyrimidyl, thiazolyl, oxazolyl, pyrazolyl, thienyl, furanyl, pyridinylmethyl and thiazolylmethyl, each of which is optionally mono- to trisubstituted by C₁-C₄-alkyl, C₁-C₃-haloalkyl, C₁-C₃-alkoxy, C₁-C₃-haloalkoxy, cyclopropyl, fluorine, chlorine, bromine or cyano,
R¹⁹ represents hydrogen, an alkali or alkaline earth metal ion, represents an ammonium ion which is optionally mono- to tetrasubstituted by C₁-C₄-alkyl or represents a radical from the group consisting of C₁-C₄-alkyl, C₁-C₄-alkoxy, C₁-C₄-alkoxy-C₁-C₂-alkyl and C₁-C₄-alkyl-S—C₁-C₂-alkyl, C₁-C₄-alkyl-S(O)—C₁-C₂-alkyl and C₁-C₄-alkyl-S(O)₂—C₁-C₂-alkyl, each of which is optionally mono-, di-, tri-, tetra- or pentasubstituted by fluorine, chlorine or mono- or disubstituted by cyano,
R²³ represents a radical from the group consisting of hydrogen, C₁-C₆-alkyl, C₂-C₄-alkenyl, C₂-C₄-alkynyl, C₃-C₆-cycloalkyl, C₃-C₆-cycloalkenyl, C₁-C₆-alkoxy, C₂-C₆-alkenyloxy, C₂-C₆-alkynyloxy, C₃-C₆-cycloalkyloxy, C₁-C₄-alkylthio-C₁-C₄-alkyl, C₂-C₄-alkenylthio-C₁-C₄-alkyl, cyano-C₁-C₄-alkyl, C₁-C₄-alkoxy-C₁-C₄alkyl,
or, if R²=g),
R²² and R²³ together with the nitrogen atom to which they are attached represent pyrrolidinyl, piperidinyl, morpholinyl, thiomorpholinyl, thiomorpholinyl 1-oxide, thiomorpholinyl 1,1-dioxide, piperazinyl, 1-methylpiperazinyl or 2-oxo-1-methylpiperazinyl,
R²⁶ represents a radical from the group consisting of hydrogen, methyl, 2,2,2-trifluoroethyl, 2,2-difluoroethyl, propenyl, propargyl, cyclopropyl, cyclopropylmethyl, methoxymethyl, methylsulfanyl, trifluoromethylsulfanyl, ethylsulfanyl, trifluoroethylsulfanyl, methylsulfinyl, trifluoromethylsulfinyl, ethylsulfinyl, trifluoroethylsulfinyl, methylthioethyl, methylsulfinylethyl, methylsulfonylethyl and cyanomethyl and
R²⁷ represents a radical from the group consisting of hydrogen, methyl, 2,2,2-trifluoroethyl, 2,2-difluoroethyl, propenyl, propargyl, cyclopropyl, cyclopropylmethyl, methoxymethyl, methylthioethyl, methylsulfinylethyl, methylsulfonylethyl and cyanomethyl.

Preferred range (4): A particular group of compounds of the formula (I) is that of those in which
A represents the A radical (A-a)

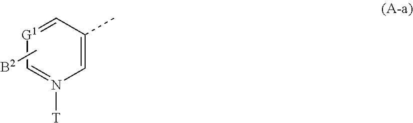

where the broken line denotes the bond to the carbon atom of the bicyclic system of the formula (I),
G¹ represents C—B¹,
B¹ represents hydrogen,
B² represents hydrogen,
T represents an electron pair,
R¹ represents hydrogen,
R² a) represents one of the radicals below

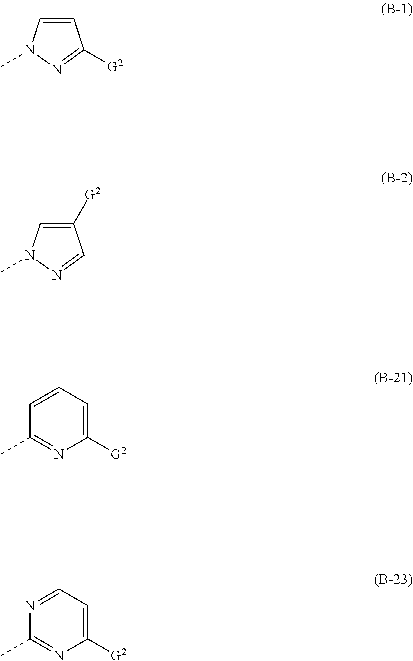

where the broken line denotes the bond to the carbon atom of the bicyclic system of the formula (I), or
R² c) represents a radical of the formula

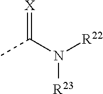

where the broken line denotes the bond to the carbon atom of the bicyclic system of the formula (I), or
R² d) represents a radical of the formula

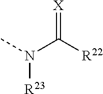

where the broken line denotes the bond to the carbon atom of the bicyclic system of the formula (I), or
R² f) represents C₁-C₆-haloalkyl or
R² g) represents a radical of the formula

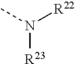

where the broken line in each case denotes the bond to the carbon atom of the bicyclic system of the formula (I), in which
G² represents a radical from the group consisting of hydrogen, C₁-C₄-alkyl and halo-C₁-C₄-alkyl,
R²² in the case that R² represents g), represents a radical from the group consisting of methyl, ethyl, isopropyl, n-propyl, n-butyl, isobutyl, sec-butyl, tert-butyl, 2,2,2-trifluoroethyl, 2,2-difluoroethyl, 2,2-difluoro-n-propyl, methylsulfanylmethyl, methylsulfanylethyl, methylsulfanyl-n-propyl, methylsulfonyl, ethylsulfonyl, methylsulfonylmethyl, ethylsulfonylmethyl, isopropylsulfonylmethyl, cyclopropyl and, in the case that R² represents c), d) or g),
R²² represents (D-2)

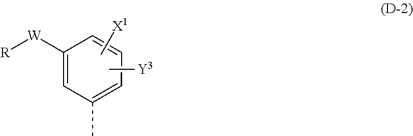

in which
X¹ represents a radical from the group consisting of hydrogen, fluorine, chlorine and bromine,
R represents C₁-C₄-alkyl, optionally mono-, di-, tri-, tetra- or pentasubstituted by fluorine, chlorine,
W represents a radical from the group consisting of S, SO and SO₂,
Y³ represents hydrogen or methyl and
R²³ represents hydrogen or C₁-C₆-alkyl or, in the case that R² represents g),
R²² and R²³ together with the nitrogen atom to which they are attached represent pyrrolidinyl or morpholinyl.

When sulfur and/or nitrogen occur in rings in the above definitions, for example in expressions such as “in which the rings may contain at least one heteroatom from the group consisting of sulfur, oxygen (where oxygen and sulfur atoms must not be directly adjacent to one another) and nitrogen” or “in which one or two ring members may each be replaced by a heteroatom from the group consisting of sulfur, oxygen (where oxygen atoms must not be directly adjacent to one another) and nitrogen”, unless stated otherwise, the sulfur may also be present in the form of SO or SO₂; the nitrogen, if it is not in the form of —N═, as well as NH, may also be present in the form of N-alkyl (in particular N—C₁-C₆-alkyl).

In the preferred definitions whose combination forms the range of preference (1), unless stated otherwise,

cation represents an alkali metal ion selected from the group consisting of lithium, sodium, potassium, rubidium, caesium, preferably from the group consisting of lithium, sodium, potassium, or an

alkaline earth metal ion selected from the group consisting of beryllium, magnesium, calcium, strontium, barium, preferably from the group consisting of magnesium and calcium,

halogen is selected from the group of fluorine, chlorine, bromine and iodine, preferably in turn from the group of fluorine, chlorine and bromine,

aryl (including as part of a larger unit, for example arylalkyl) is selected from the group of phenyl, naphthyl, anthryl, phenanthrenyl, and preferably in turn represents phenyl,

hetaryl (synonymous with heteroaryl, including as part of a larger unit, for example hetarylalkyl) is selected from the group consisting of furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzoisofuryl, benzothienyl, benzoisothienyl, indolyl, isoindolyl, indazolyl, benzothiazolyl, benzoisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl,

heterocyclyl represents a saturated 4-, 5- or 6-membered ring containing 1 or 2 nitrogen atoms and/or one oxygen atom and/or one sulfur atom, for example azetidinyl, azolidinyl, azinanyl, oxetanyl, oxolanyl, oxanyl, dioxanyl, thiethanyl, thiolanyl, thianyl, tetrahydrofuryl, piperazinyl, morpholinyl.

In the particularly preferred definitions whose combination forms the range of preference (2), unless stated otherwise,

cation represents an alkali metal ion selected from the group consisting of lithium, sodium, potassium, rubidium, caesium, preferably from the group consisting of lithium, sodium, potassium, or an

alkaline earth metal ion selected from the group consisting of beryllium, magnesium, calcium, strontium, barium, preferably from the group consisting of magnesium and calcium,

halogen is selected from the group of fluorine, chlorine, bromine and iodine, preferably in turn from the group of fluorine, chlorine and bromine,

aryl (including as part of a larger unit, for example arylalkyl) is selected from the group of phenyl, naphthyl, anthryl, phenanthrenyl, and preferably in turn represents phenyl,

hetaryl (synonymous with heteroaryl, also as part of a larger unit such as, for example, hetarylalkyl) is selected from the group consisting of pyrazolyl, imidazolyl, 1,2,3-triazolyl, 1,2,4-triazolyl, oxazolyl, isoxazolyl, thiazolyl, isothiazolyl, pyridyl, pyrimidinyl, pyridazinyl, pyrazinyl, 1,2,3-triazinyl, 1,2,4-triazinyl, 1,3,5-triazinyl,

heterocyclyl is selected from the group consisting of azetidinyl, azolidinyl, azinanyl, oxetanyl, oxolanyl, oxanyl, dioxanyl, thiethanyl, thiolanyl, thianyl, tetrahydrofuryl, piperazinyl, morpholinyl.

In the very particularly preferred definitions and the especially preferred definitions whose combination forms the range of preference (3), unless stated otherwise,

cation represents an alkali metal ion from the group consisting of lithium, sodium, potassium, rubidium, caesium, preferably from the group consisting of lithium, sodium, potassium, or an

alkaline earth metal ion from the group consisting of beryllium, magnesium, calcium, strontium, barium, preferably from the group consisting of magnesium and calcium,

heterocyclyl represents oxetanyl, thiethanyl, tetrahydrofuryl and morpholinyl.

Aryl represents phenyl,

hetaryl (synonymous with heteroaryl, including as part of a larger unit such as, for example, hetarylalkyl) represents a radical from the group consisting of pyridyl, pyrimidyl, pyrazinyl, pyridazinyl, thiazolyl and pyrazolyl.

In the definitions which form the range of preference (4),

halogen represents fluorine, chlorine, bromine and iodine, preferably in turn fluorine, chlorine and bromine.

Halogen-substituted radicals, for example haloalkyl, are mono- or polyhalogenated, up to the maximum number of possible substituents. In the case of polyhalogenation, the halogen atoms may be identical or different. In this case, halogen is fluorine, chlorine, bromine or iodine, especially fluorine, chlorine or bromine.

Saturated or unsaturated hydrocarbon radicals, such as alkyl or alkenyl, may each be straight-chain or branched if possible, including in combination with heteroatoms, as, for example, in alkoxy.

Unless stated otherwise, optionally substituted radicals may be mono- or polysubstituted, where the substituents in the case of poly substitutions may be the same or different.

If T in the radical A of the formula (A-a)

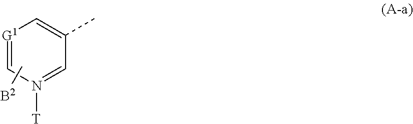

represents an electron pair, the radical is present as a pyridine derivative of the formula

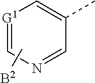

If T in the radical A of the formula (A-a)

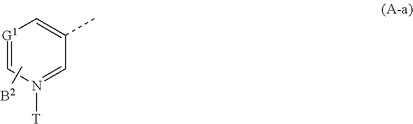

represents oxygen, the radical is present as a pyridine N-oxide derivative of the formula

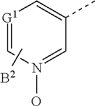

Here, the formal charges (+ at nitrogen and − at oxygen) were omitted from the illustration.

The radical definitions or elucidations given in general terms or listed within areas of preference apply correspondingly to end products and to starting materials and intermediates. These radical definitions can be combined with one another as desired, i.e. including combinations between the respective ranges of preference.

According to the invention, preference is given to compounds of the formula (I) which contain a combination of the meanings listed above as being preferred (range of preference (1)).

According to the invention, particular preference is given to compounds of the formula (I) which contain a combination of the meanings listed above as being particularly preferred (range of preference (2)).

According to the invention, very particular preference is given to compounds of the formula (I) which contain a combination of the meanings listed above as being very particularly preferred (range of preference (3)).

According to the invention, special preference is given to compounds of the formula (I) which contain a combination of the meanings listed above as being special (range of preference (4)).

A further preferred embodiment of the invention relates to compounds of the formula (I) in which A represents the radical of the formula (A-a)

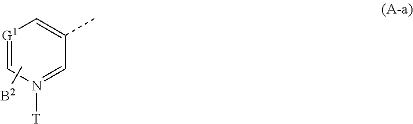

A further preferred embodiment of the invention relates to compounds of the formula (I) in which A represents pyridin-3-yl.

A further preferred embodiment of the invention relates to compounds of the formula (I) in which A represents 5-fluoropyridin-3-yl.

A further preferred embodiment of the invention relates to compounds of the formula (I) in which A represents pyrimidin-5-yl.

A further preferred embodiment of the invention relates to compounds of the formula (I) in which A represents pyridazin-4-yl.

A further preferred embodiment of the invention relates to compounds of the formula (I) in which R² represents the radicals mentioned under a).

A further preferred embodiment of the invention relates to compounds of the formula (I) in which R² represents the radicals mentioned under b).

A further preferred embodiment of the invention relates to compounds of the formula (I) in which R² represents the radicals mentioned under c).

A further preferred embodiment of the invention relates to compounds of the formula (I) in which R² represents the radicals mentioned under d).

A further preferred embodiment of the invention relates to compounds of the formula (I) in which R² represents the radicals mentioned under e).

A further preferred embodiment of the invention relates to compounds of the formula (I) in which R² represents the radicals mentioned under f).

A further preferred embodiment of the invention relates to compounds of the formula (I) in which R² represents the radicals mentioned under g).

A further preferred embodiment of the invention relates to compounds of the formula (I) in which R² represents the radical (D-2)

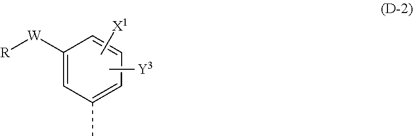

The radical definitions or elucidations given above in general terms or within preferred ranges apply correspondingly to the end products (including the compounds of the formulae (I-A) to (I-N) shown later), and to the starting materials and intermediates. These radical definitions can be combined with one another as desired, i.e. including combinations between the respective ranges of preference.

In a preferred embodiment, the invention relates to compounds of the formula (I-A)

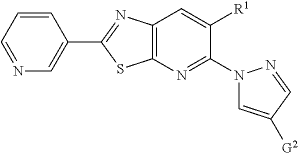

In a further preferred embodiment, the invention relates to compounds of the formula (I-B)

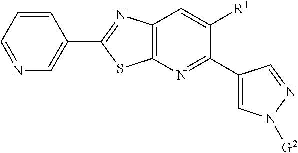

In a further preferred embodiment, the invention relates to compounds of the formula (I-C)

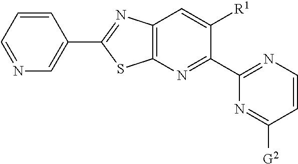

In a further preferred embodiment, the invention relates to compounds of the formula (I-D)

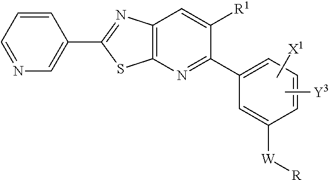

In a further preferred embodiment, the invention relates to compounds of the formula (I-E)

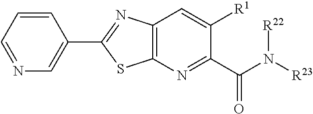

In a further preferred embodiment, the invention relates to compounds of the formula (I-F)

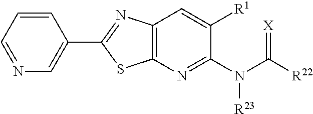

In a further preferred embodiment, the invention relates to compounds of the formula (I-G)

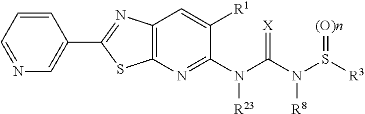

In a further preferred embodiment, the invention relates to compounds of the formula (I-H)

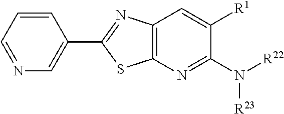

In a further preferred embodiment, the invention relates to compounds of the formula (I-I)

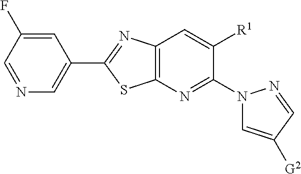

In a further preferred embodiment, the invention relates to compounds of the formula (I-J)

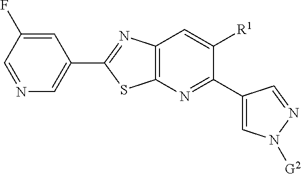

In a further preferred embodiment, the invention relates to compounds of the formula (I-K)

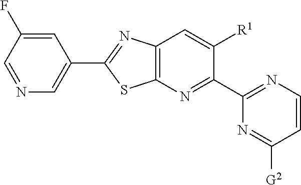

In a further preferred embodiment, the invention relates to compounds of the formula (I-L)

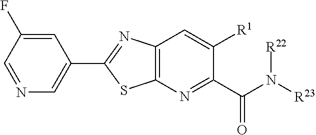

In a further preferred embodiment, the invention relates to compounds of the formula (I-M)

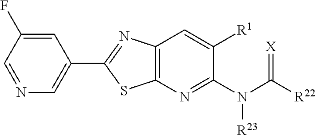

In a further preferred embodiment, the invention relates to compounds of the formula (I-N)

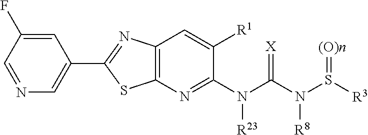

In a further preferred embodiment, the invention relates to compounds of the formula (I-O)

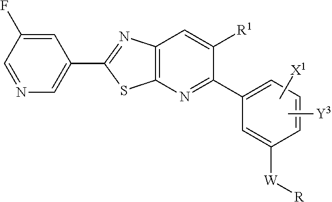

In a further preferred embodiment, the invention relates to compounds of the formula (I-P)

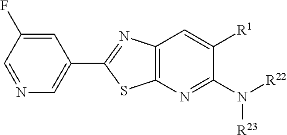

In the formulae (I-A) to (I-P), the variables have the definitions given further up.

The inventive compounds of the formula (I) and their acid addition salts and metal salt complexes have good efficacy, especially for control of animal pests including arthropods and especially insects.

Suitable salts of the compounds of the formula (I) which may be mentioned are customary nontoxic salts, i.e. salts with appropriate bases and salts with added acids. Preference is given to salts with inorganic bases, such as alkali metal salts, for example sodium, potassium or caesium salts, alkaline earth metal salts, for example calcium or magnesium salts, ammonium salts, salts with organic bases and with inorganic amines, for example triethylammonium, dicyclohexylammonium, N,N′-dibenzylethylenediammonium, pyridinium, picolinium or ethanolammonium salts, salts with inorganic acids, for example hydrochlorides, hydrobromides,

dihydrosulfates, trihydrosulfates, or phosphates, salts with organic carboxylic acids or organic sulfonic acids, for example formates, acetates, trifluoroacetates, maleates, tartrates, methanesulfonates, benzenesulfonates or para-toluenesulfonates, salts with basic amino acids, for example arginates, aspartates or glutamates, and the like.

The compounds of the formula (I) may possibly also, depending on the nature of the substituents, be in the form of stereoisomers, i.e. in the form of geometric and/or optical isomers or isomer mixtures of varying composition. This invention provides both the pure stereoisomers and any desired mixtures of these isomers, even though it is generally only compounds of the formula (I) that are discussed here.

The invention therefore relates both to the pure enantiomers and diastereomers and to mixtures thereof for controlling animal pests, including arthropods and particularly insects.

However, according to the invention preference is given to using the optically active, stereoisomeric forms of the compounds of the formula (I) and their salts.

It has additionally been found that the novel compounds of the formula (I) and also those compounds listed in Table 1 that are not embraced by formula (I) can be prepared by the processes described below.

Compounds of the formula (I) in which the heterocycle A represents an optionally B²-radical-substituted pyrimidin-5-yl (A-a; G¹=N), pyridin-3-yl (A-a; G¹=C—B¹), pyrazin-2-yl (A-b), pyridazin-3-yl (A-c), thiazol-5-yl (A-d), isothiazol-4-yl (A-e) and pyrazol-4-yl (A-f) can, for example, be prepared according to Reaction Scheme I in two or three steps.

Reaction Scheme I—Method A

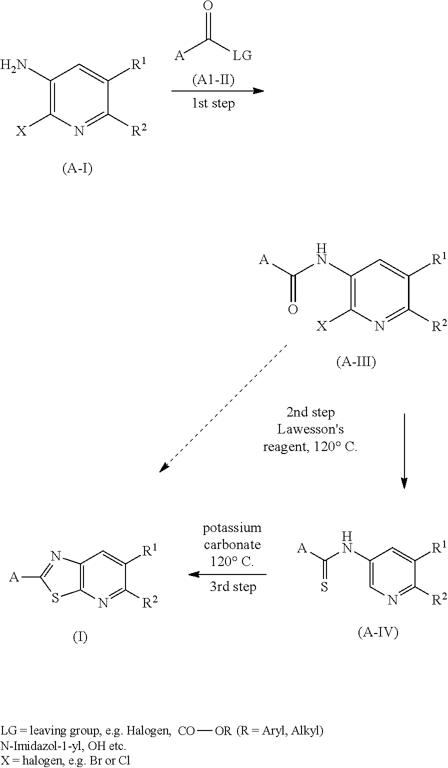

In Reaction Scheme I, A, R¹ and R² have the meanings mentioned above and optionally further meanings relevant for the preparation processes, which meanings are evident from the text or context.

For example, the substituted aminopyridines of the formula (A-I) can be reacted with the appropriate activated carboxylic acids (for example in the form of the carbonyl chloride or the hydrochloride thereof) of the formula (A-II) in the presence of basic reaction auxiliaries in a first reaction step to give compounds of the formula (A-III). These are then thionated by appropriate sulfur donors, for example Lawesson's reagent, to give compounds of the formula (A-IV). If this reaction is carried out at elevated temperature, the thioamides of the formula (A-IV) formed may cyclize directly to compounds of the formula (I). Otherwise, compounds of the formula (A-IV) may be cyclized in a third reaction step in the presence of a suitable base, for example potassium carbonate, to compounds of the formula (I).

Method A—Step 1:

Some of the compounds of the formula (A-I) are known and commercially available or can be obtained by preparation processes known in principle (for example for R¹═H, R²═Cl; 2,6-dichloropyridin-3-amine, cf. WO 2007/015877 and WO 2014/02234).

Some of the compounds of the formula (A-II) are known and commercially available or can be obtained by preparation processes known in principle (for example for A=pyridin-3-yl, LG=Cl; nicotinyl chloride (Journal of the American Chemical Society (1953), 75, 4364 or for A=5-fluoropyridin-3-yl, LG=Cl; 5-fluoronicotinyl chloride (U.S. Pat. No. 2,516,830).

A number of reaction conditions have been described for the amidation step, for example G. Benz in Comprehensive Organic Synthesis, 1^{st} Ed., Pergamon Press, Oxford, 1991, Vol. 6, pp. 381-417; P. D. Bailey et al. in Comprehensive Organic Functional Group Transformation, 1st Ed., Elsevier Science Ltd., Oxford, 1995, Vol. 5, pp. 257-308 and R. C. Larock in Comprehensive Organic Transformations, 2nd Ed., Wiley-VCH, New York, Weinheim, 1999, pp. 1929-1994. Some of these reactions proceed via intermediate carbonyl chlorides, which can be used in isolated form or having been generated in situ from A-II (LG=OH).

The amidation reactions are optionally effected in the presence of a condensing agent, optionally in the presence of an acid acceptor and optionally in the presence of a solvent.

Useful condensing agents are all the condensing agents typically usable for such amidation reactions. Examples include acid halide formers such as phosgene, phosphorus trichloride, oxalyl chloride or thionyl chloride; carbodiimides such as N,N′-dicyclohexylcarbodiimide (DCC) and 1-(3-dimethylaminopropyl)-3-ethylcarbodiimide (EDCI), or other customary condensing agents such as phosphorus pentoxide, polyphosphoric acid, N,N′-carbonyldiimidazole, 2-chloropyridine 1-methoiodide (Mukaiyama's reagent), 2-ethoxy-N-ethoxycarbonyl-1,2-dihydroquinoline (EEDQ), triphenylphosphine/carbon tetrachloride, bromotripyrrolidinophosphonium hexafluorophosphate (BROP), O-(1H-benzotriazol-1-yloxy)tris(dimethylamino)phosphonium hexafluorophosphate (BOP), N,N,N′,N′-bis(tetramethylene)chlorouronium tetrafluoroborate, O-(1H-benzotriazol-1-yl)-N,N,N′,N′-tetramethyluronium hexafluorophosphate (HBTU),

O-(1H-benzotriazol-1-yl)-N,N,N′,N′-bis(tetramethylene)uronium hexafluorophosphate, O-(1H-benzotriazol-1-yl)-N,N,N′,N′-tetramethyluronium tetrafluoroborate (TBTU), O-(1H-benzotriazol-1-yl)-N,N,N′,N′-bis(tetramethylene)uronium tetrafluoroborate, O-(7-azabenzotriazol-1-yl)-N,N,N′,N′-tetramethyluronium hexafluorophosphate (HATU), 1-hydroxybenzotriazole (HOBt) and 4-(4,6-dimethoxy-1,3,5-triazin-2-yl)-4-methylmorpholinium salt (DMT.MM), usually available as chloride. These reagents can be used separately or, if appropriate, in combination.

Useful acid acceptors are all customary inorganic or organic bases, for example triethylamine, diisopropylethylamine, N-methylmorpholine or N,N-dimethylaminopyridine. Process A (step 1) according to the invention is optionally carried out in the presence of a suitable reaction auxiliary, for example N,N-dimethylformamide or N,N-dimethylaminopyridine.

Solvents or diluents include all inert organic solvents, for example aliphatic or aromatic hydrocarbons (such as petroleum ether, toluene), halogenated hydrocarbons (such as chlorotoluene, dichloromethane, chloroform, 1,2-dichloroethane), ethers (such as diethyl ether, dioxane, tetrahydrofuran, 1,2-dimethoxyethane), esters (such as ethyl or methyl acetate), nitrohydrocarbons (such as nitromethane, nitroethane, nitrobenzene), nitriles (such as acetonitrile, benzonitrile), amides (such as N,N-dimethylformamide, N,N-dimethylacetamide, N-methylformanilide, N-methylpyrrolidone, hexamethylphosphoramide), and also dimethyl sulfoxide or water or mixtures of the solvents mentioned.

For synthesis of the compounds of the formula (A-III), it is alternatively possible to use mixed anhydrides (LG=COOR) (cf. G. W. Anderson et al. J. Am. Chem. Soc. 1967, 89, 5012-5017). In this process, which proceeds via compounds of the formula (A-II, LG=CO—OR, R=alkyl, aryl), it is possible to use chloroformic esters, for example isobutyl chloroformate (LG=COOR with R=isobutyl) and isopropyl chloroformate (LG=COOR with R=isopropyl). Likewise, it is possible for this purpose to use diethylacetyl chloride, trimethylacetyl chloride and similar compounds.

Method A—Step 2:

The amide function of the carboxamides of the (A-III) type can be converted to a thioamide function by means of suitable thionating reagents, for example Lawesson's reagent or phosphorus(V) sulfide, while heating in a suitable solvent, for example toluene or anisole, which gives rise to compounds of the (A-IV) type [cf., for example, EP 2 560 008 for N-(2,6-dichloro-3-pyridypbenzenecarbothioamide]. In this type of reaction, there may already be partial or, at a longer reaction time, even complete cyclization to compounds of the formula (I) (cf., for example, WO 02/36580 for 2-chloro-N-(2-chloropyridin-3-yl)-5-nitrobenzamide or the synthesis, given below in an exemplary manner, of 5-chloro-2-(3-pyridypthiazolo[5,4-b]pyridine).

Method A—Step 3:

Finally, the compounds of the formula (A-IV) can be converted according to methods known from the literature (cf., for example, EP 2 560 008 for 5-chloro-2-phenylthiazolo[5,4-b]pyridine) by heating in a suitable solvent, for example toluene or DMF, in the presence of a base, for example sodium hydride or potassium carbonate, into the compounds of the formula (I).

If, in the process according to the invention for preparing the compounds of the formula (I), the compound of the formula (A-I) used is 2,6-dichloropyridine-3-amine (R¹═H, R²═Cl, X═Cl) and the compound of the formula (A-II) used is 3-(chlorocarbonyl)pyridinium chloride (A=3-pyridin-3-yl), this at first forms N-(2,6-dichloropyridin-2-yl)nicotinamide (corresponds to A-IV where A=3-pyridin-3-yl, R¹═H, R²═Cl, X═Cl). Subsequent thionation proceeds with cyclization and leads directly to 5-chloro-2-(3-pyridypthiazolo[5,4-b]pyridine, (corresponds to (I) where A=pyridin-3-yl, R¹═H, R²═Cl), cf. also the synthesis of 5-chloro-2-(3-pyridypthiazolo[5,4-b]pyridine, shown below by way of example.

The compound 5-chloro-2-(3-pyridyl)thiazolo[5,4-b]pyridine is novel and also forms part of the subject-matter of the invention.

Compounds of the formula (I) in which R² represents halogen, for example chlorine or bromine, can be obtained according to Reaction Scheme I from halogenated 2-chloroaniline derivatives (A-I). From these, it is possible to generate further compounds of the formula (I) by literature methods (B to F). By way of example, methods B to F and the resulting compound types are shown in Reaction Scheme II.

Reaction Scheme II—Methods B-F

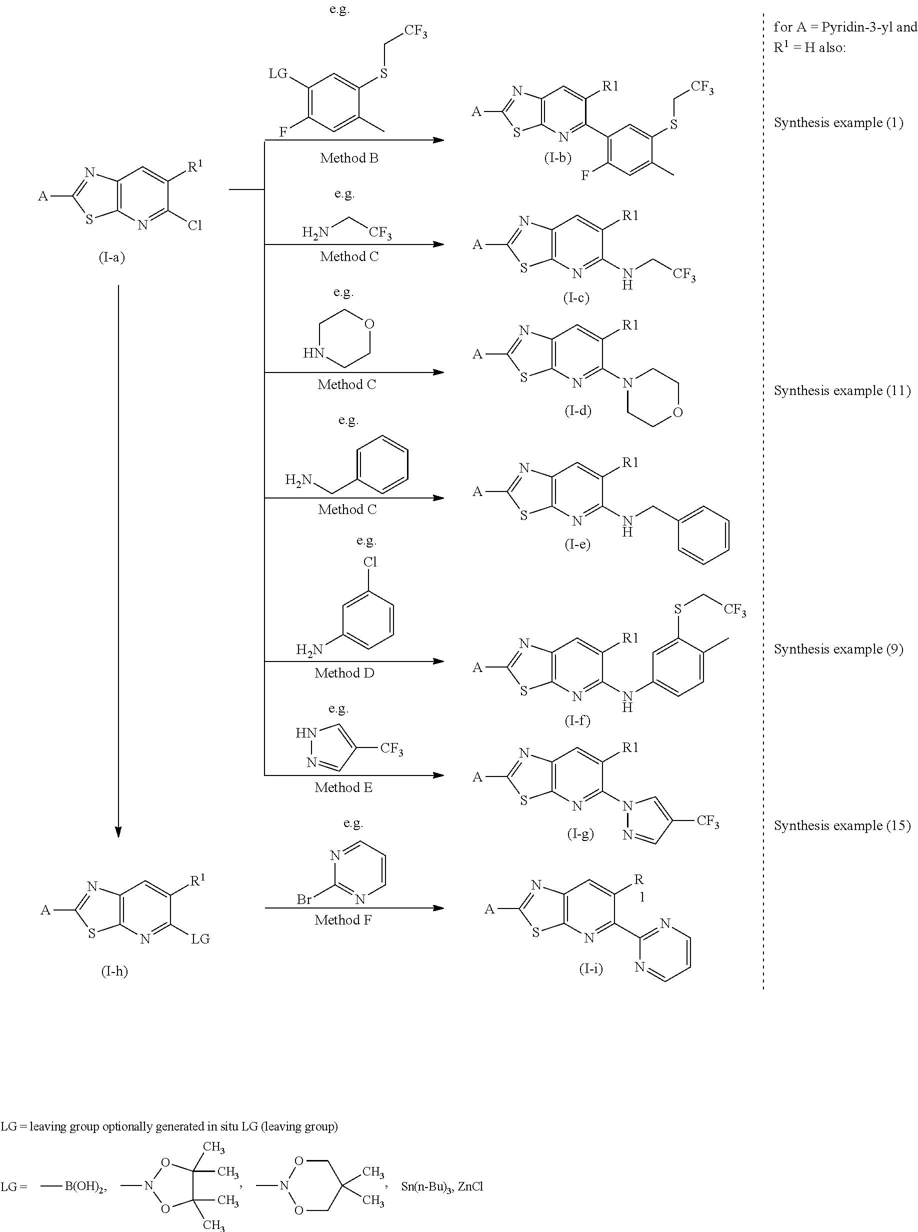

Method B/F:

According to WO 2010/071819 or WO 2010/008847, chlorinated or brominated thiazolopyridines can be arylated with substituted arylboronic acids or arylboronic acid pinacol esters, optionally generated in situ, in the presence of suitable coupling catalysts such as, for example, tetrakis(triphenylphosphine)palladium(0) or [1,1′-bis(diphenylphosphino)ferrocene]dichloropalladium(II) in the presence of a base such as, for example, sodium carbonate or sodium bicarbonate, in an inert solvent or diluent such as 1,2-dimethoxyethane or 1,4-dioxane in combination with water (Method B),

which allows compounds of the formula (I-b) [cf. also Synthesis Example (1)] to be obtained. It is also possible to react optionally substituted heteroaromatic boronic acids or the pinacol esters thereof analogously with chlorinated bicyclic heteroaromatic systems by Method B (cf., for example, WO 2010/071819 for 6-(6-fluoro-3-pyridyl)-2-[4- (1-piperidyl)-1-piperidyl]thiazolo[4,5-c]pyridine).

Moreover, chlorinated thiazolopyridines can be converted by known methods (cf. Metal-Catalyzed Cross-Coupling Reactions (Eds.: A. de Meijere, F. Diederich), 2nd ed., Wiley-VCH, Weinheim, 2004 and Handbook of Organopalladium Chemistry for Organic Synthesis (Ed.: Ei-ichi Negishi), John Wiley & Sons, New York, 2002) with optionally substituted aromatic and heteroaromatic stannanes (LG=SnR₃) in the presence of suitable catalysts from the group of the transition metal salts into compounds of the formula (I-b).

Chlorinated thiazolopyridines can be arylated with 2-(trialkylstannyl)pyrimidines (analogously to WO 2013/159064) in the presence of suitable coupling catalysts such as, for example, tetrakis(triphenylphosphine)palladium(0) in combination with copper(I) iodide in an inert solvent or diluent such as 1,4-dioxane. (cf. also Synthesis Example (7)).

Chlorinated thiazolopyridines can also be converted by known methods (cf. Metal-Catalyzed Cross-Coupling Reactions (Eds.: A. de Meijere, F. Diederich), 2nd ed., Wiley-VCH, Weinheim, 2004 and Handbook of Organopalladium Chemistry for Organic Synthesis (Ed.: Ei-ichi Negishi), John Wiley & Sons, New York, 2002) with optionally substituted aromatic and heteroaromatic arylzinc halides (LG=ZnX; X=Halogen) in the presence of suitable catalysts from the group of the transition metal salts into compounds of the formula (I-b). Chlorinated thiazolopyridines (analogously to Journal of Organic Chemistry (2010), 75, 8830-8832) can furthermore by arylated with 2-pyridylzinc halides in the presence of suitable catalyst systemes,

for example tris(dibenzylideneacetone)dipalladium in combination with a ligand such as 2-dicyclohexylphosphino-2′,4′,6′-triisopropylbiphenyl in an inert solvent or diluent such as tetrahydrofuran. (cf. also Synthesis Example (8)).

Some of the structures (B-3) to (B-9), (B-11) to (B-13) and (B-21) to (B-33) having a suitable leaving group (LG=B(OH)₂) or (hetero)arylboronic ester (LG=B(OR)₂) are known, and/or they can be prepared by known methods: e.g. 1-(methyl-1H-pyrazol-4-yl)boronic acid [(B-3), LG=B(OH)₂, G²=hydrogen, WO 2009/155527], 2-phenyl-4-(4,4,5,5-tetramethyl-1,3,2-dioxaborolan-2-yl)oxazole [(B-6), LG=B(OCMe₂)₂, G²=phenyl, WO 2010/094755]; thiazol-2-ylboronic acid [(B-7), LG=B(OH)₂, G²=hydrogen, U.S. Pat. No. 6,310,095 B1]; 5-phenyl-1,2,4-thiadiazol-3-ylboronic acid [(B-13), LG=B(OH)₂, G²=phenyl, DE 19710614 A1], pyridin-3-ylboronic acid [(B-21) vs (B-22), LG=B(OH)₂, G²=hydrogen, WO 2013/186089]; 1,3,5-triazin-2-ylboronic acid [(B-28), LG=B(OH)₂, G²=hydrogen, Korean Kongkae Taeho Kongbo (2011), KR 2011/079401].

Some compounds having a suitable leaving group (LG=SnR₃) are known, and/or they can be prepared by known methods: for example 5-methyl-2-(tributylstannyl)oxazole [(B-5), LG=SnBu₃, G²=methyl, WO 2014/030128 A1]; 5-ethyl-2-(tributylstannyl)pyrimidine [(B-23), LG=SnBu₃, G²=ethyl, WO 2003/039469 A2]; 2-chloro-6-(tributylstannyl)pyrazine [(B-26), LG=SnBu₃, G²=Cl, WO 2012/129338 A1].

Some compounds having a suitable leaving group (LG=ZnX, X=halogen) are known, and/or they can be prepared by known methods, if appropriate also in situ (cf. Handbook of Functionalized Organometallics (Ed.: P. Knochel), Wiley-VCH, Weinheim, 2005).

Alternatively, the compounds of the formula (I-a) can initially be converted by methods known from the literature into compounds of the formula (I-h), which are subsequently reacted further with halogen-activated heterocycles according to Reaction Scheme II (Method F) in an inert organic solvent or diluent analogously to compounds of the formula (I-i) (cf. WO 2009/154775 or WO 2010/116282).

Some of the halogen-activated structures according to (B-3) to (B-9), (B-11) to (B-13) and (B-21) to (B-33) are known, and/or they can be prepared by generally known methods: e.g. 3-bromo-4,5-dihydro-1-phenyl-1H-pyrazole [(B-18), LG=Br, G²=phenyl, J. Elguero et al., Bull. Soc. Chim. France 1996, 5, 1683-1686].

Suitable coupling catalysts include palladium catalysts such as [1,1′-bis(diphenylphosphino)ferrocene]dichloropalladium (II) or tetrakis(triphenylphosphine)palladium.

Suitable basic reaction auxiliaries used for carrying out the processes according to Reaction Scheme III are preferably carbonates of sodium or potassium.

Preferably, the diluents used are nitriles such as acetonitrile, benzonitrile, in particular acetonitrile, or ethers such as diethyl ether, dioxane, tetrahydrofuran, 1,2-dimethoxyethane, in particular 1,2-dimethoxyethane in combination with water.

Method C:

Chlorinated heteroaromatic bicyclic systems can be converted by heating in the presence of optionally substituted aliphatic primary or secondary amines or arylmethyl- or hetarylmethylamines in an inert organic solvent or diluent such as dimethylformamide into compounds of (types (1-c), (1-d) and (1-e)). This reaction optionally proceeds in the presence of a suitable base such as potassium carbonate.

(cf., for example, EP 2 560 008 for the synthesis of N-benzyl-2-phenylthiazolo[5,4-b]pyridine-5-amine, and Synthesis Example (6)).

Optionally substituted cyclic secondary amines react with chlorinated heteroaromatic bicyclic systems, preferably in the presence of suitable coupling catalysts such as, for example, Pd(OAc)₂ and suitable ligands such as 2,2′-bis(diphenylphosphino)-1,1′-binaphthyl (BINAP), with addition of a base such as sodium tert-butoxide and in the presence of an inert organic solvent or diluent, to give compounds of the formula (I-d). (cf., for example, WO 2007/148093 for the synthesis of 4-pyridin-2-ylmorpholine and Synthesis Example (11)). Useful solvents or diluents include all inert organic solvents, for example aliphatic or aromatic hydrocarbons. Preference is given to using aromatic hydrocarbons, for example toluene.

Method D:

With optionally substituted anilines, chlorinated heteroaromatic bicyclic systems react in an inert organic solvent or diluent to give the corresponding arylamino compounds of the formula (I-f), preferably with catalysis by suitable coupling catalysts such as Pd(OAc)₂ or tris-(dibenzylideneacetone)dipalladium(0), in the presence of a suitable ligand such as 2,2′-bis-(diphenylphosphino)-1,1′-binaphthyl (BINAP) or 2-dicyclohexylphosphino-2′,4′,6′-triisopropylbiphenyl and a base such as sodium tert-butoxide or potassium tert-butoxide (cf., for example, Bioorganic & Medicinal Chemistry 2012, 20, 5600-5615 for the preparation of tert-butyl [5-({2-[(cyclopropylcarbonyl)amine][1,3]-thiazolo[5,4-b]pyridin-5-yl}amino)-2-fluorophenyl]carbamate and Synthesis Example (9)).

Useful solvents or diluents include all inert organic solvents, for example aliphatic or aromatic hydrocarbons. Preference is given to using aromatic hydrocarbons such as toluene or alcohols such as tert-butanol.

By alkylation with suitable electrophiles, compounds of the formula (I) in which R² represents —NHR²² (cf. formula (1-f)) can be converted into compounds of the formula (I) in which R² represents —NR²³R²². This reaction is optionally carried out in the presence of a base such as, for example, sodium hydride, and in an organic solvent or diluent. Here, preference is given to using amides such as, for example, dimethylformamide Preferred electrophiles are alkyl halides of the formula R²³—X, where in this case R²³ represents in particular alkyl, cycloalkyl, alkylthioalkyl, cyanoalkyl and alkoxyalkyl and X represents halogen such as iodine, bromine or chlorine (see Synthesis Example (14)).

Method E:

Similar to the literature method E shown in Reaction Scheme II, optionally substituted heterocycles such as imidazoles, pyrazoles and triazoles can be introduced into chlorinated bicyclic systems, preferably in the presence of suitable catalysts such as copper(I) iodide in the presence of basic ligands, for example trans-N,N′-dimethylcyclohexane-1,2-diamine, and a base such as potassium carbonate, in an inert organic solvent or diluent (see Journal of Organic Chemistry (2010), 69, 5578, cf. also Synthesis Example (13)).

Useful solvents or diluents include all inert organic solvents, for example aliphatic or aromatic hydrocarbons. Preference is given to using aromatic hydrocarbons, for example toluene, but also more polar solvents such as DMF.

Compounds of the formula (I-a) can be obtained according to Reaction Scheme I. From these, it is possible by literature methods (G) to generate compounds of the formula (I), as shown in Reaction Scheme III.

Reaction Scheme III—Method G

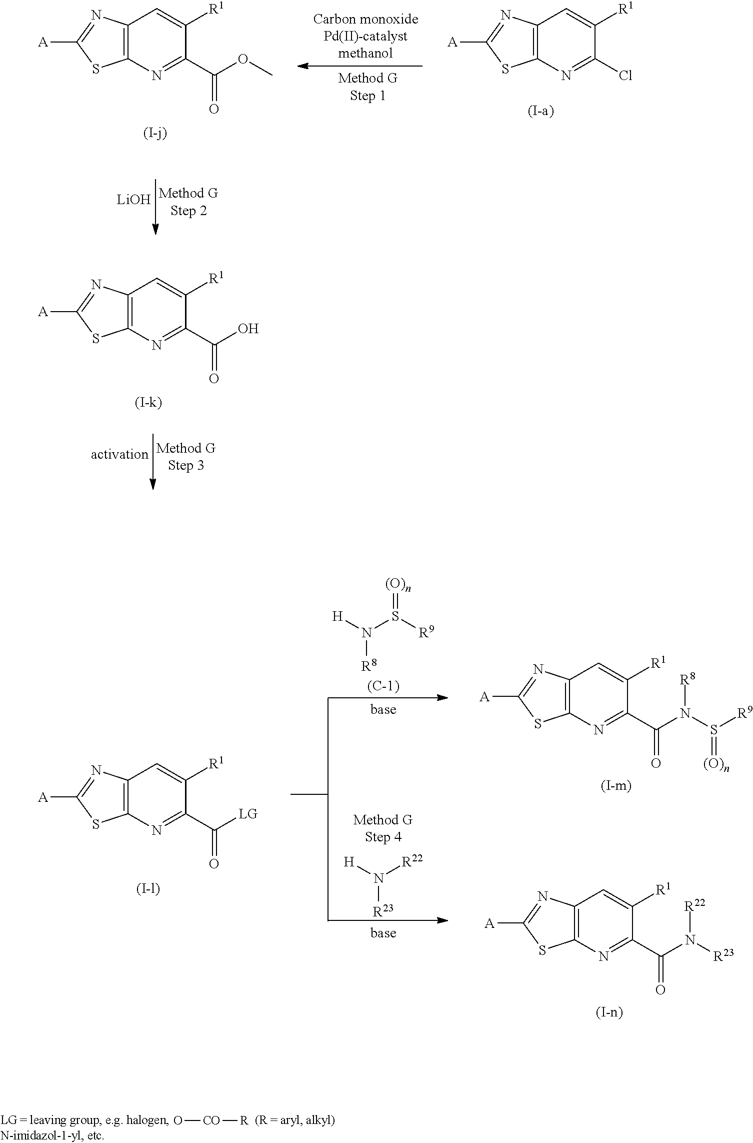

Method G—Step 1:

Compounds of the formula (I-a) can be converted by literature methods with carbon monoxide and an alcohol, for example methanol, with catalysis by suitable metal compounds in combination with suitable ligands such as, for example, palladium(II) acetate in combination with 1,3-bis(diphenylphosphino)propane and in the presence of bases such as triethylamine or potassium carbonate, in suitable solvents, for example the alcohol used itself, THF and/or DMF, into the corresponding carboxylic esters of the formula (I j) [cf. WO 2007/016392 for methyl 2-(isopropylamino)thiazolo[5,4-b]pyridine-5-carboxylate].

Method G—Step 2:

Esters of the formula (I j) can be converted into the compounds of the formula (I-k) having a free acid function by literature methods using suitable bases, for example aqueous lithium hydroxide or sodium hydroxide solution, in suitable solvents or diluents, for example dioxane or THF.

Method G—Step 3:

Compounds of the formula (I) in which R² represents a radical from the group consisting of (C-1) to (C-9) or represents C(X)NR²²R²³ can be prepared from compounds of the formula (I-k) following suitable activation (i.e. LG represents a nucleofugic leaving group optionally generated in situ) by known methods [cf. Method A, step 1].

For example, the compounds of the formula (I) in which R² represents a radical (C-1) or represents —C(X)NR²²R²³ can be obtained according to Reaction Scheme III.

Suitable condensing agents for activating the carboxylic acids of the formula (I-k) are all condensing agents customarily used for such amidation reactions.

Method G—Step 4:

The subsequent reactions of the activated compounds of the formula (I-1) with the respective components according to Reaction Scheme III are optionally conducted in the presence of a suitable reaction auxiliary and in the presence of a suitable solvent or diluent.

Suitable reaction auxiliaries for carrying out the processes according to Reaction Scheme IV are basic reaction auxiliaries.

Suitable for use as basic reaction auxiliaries are all suitable acid binders, for example amines, in particular tertiary amines, and alkali metal and alkaline earth metal compounds.

For preparing the compounds of the formulae (I-m) and (I-n), use is preferably made of tertiary amines such as N-propyldiisopropylamine or N-ethyldiisopropylamine (DIEA; Hünig's base).

Suitable solvents or diluents are all inert organic solvents.

Preference is given to using amides as solvents, for example N,N-dimethylformamide.

Compounds of the formula (I) in which R² is —NR²³—C(X)—R²² can be obtained, for example, from compounds of the formula (I) in which R² is —NHR²³ by means of N-acylation reaction using activated compounds of the formula LG-C(X)—R²² in which LG is a nucleofugic leaving group which is optionally generated in situ.

These compounds of the formula (I) in which R² represents —NHR²³ can be prepared from compounds of the formula (I-k) according to Reaction Scheme IV.

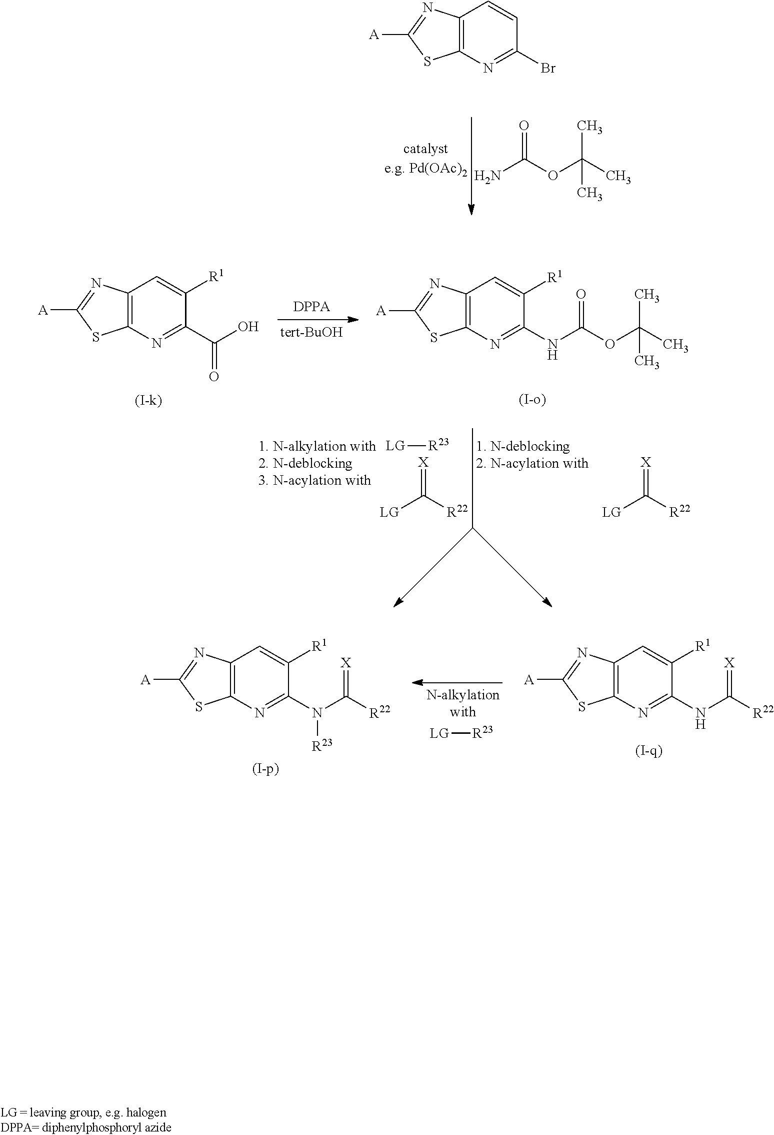

Reaction Scheme IV—Method H

For example, compounds of the formula (I-o) can be obtained by Curtius degradation as described, for example, in Houben-Weyl, Methoden der Organischen Chemie [Methods of Organic Chemistry], Volume XI/1 (Georg Thieme Verlag Stuttgart), p. 865.

In this case, the compounds of the formula (I-k) can, for example, react directly with diphenylphosphoryl azide (DPPA) in the presence of tert-butanol to give compounds of the formula (I-o).

Furthermore, compounds of the formula (I, where R²=halogen) can be converted by exchange of the halogen, for example bromine or chlorine, for tert-butyl carbamate under catalysis by suitable metal compounds, for example palladium(II) acetate, if appropriate in combination with suitable ligands such as 4,5-bis(diphenylphosphino)-9,9-dimethylxanthene, and in the presence of bases such as caesium carbonate, in suitable solvents such as 1,4-dioxane, into the corresponding carbamates of type (I-o) (cf. Journal of Medicinal Chemistry (2011), 54, 1511-1528).

From the compounds of the formula (I-o), it is possible to obtain the compounds of the formula (I-p) by N-alkylation in a first reaction step, N-deblocking (i.e. detachment of the Boc group) in a second reaction step, and subsequent N-acylation in a third reaction step.

The compounds of the formula (I-q) can be prepared by N-deblocking (i.e. cleavage of the Boc group) in a first reaction step and subsequent N-acylation in a second reaction step.

The compounds of the formula (I-p) can also be obtained from the compounds of the formula (I-q) by N-alkylation.

Furthermore, the compounds of the formula (I-p) can be obtained by N-acylation of compounds of the formula (I-c).

In general, for the removal of the protecting group, it is possible to use acidic or basic reaction auxiliaries according to the literature procedure. When protecting groups of the carbamate type are used, preference is given to using acidic reaction auxiliaries. When the tert-butyl carbamate protective group (Boc group) is used, for example, mixtures of mineral acids such as hydrochloric acid, hydrobromic acid, nitric acid, sulfuric acid, phosphoric acid or of organic acids such as benzoic acid, formic acid, acetic acid, trifluoroacetic acid, methanesulfonic acid, benzenesulfonic acid or toluenesulfonic acid, in a suitable diluent such as water and/or an organic solvent such as tetrahydrofuran, dioxane, dichloromethane, chloroform, ethyl acetate, ethanol or methanol are used. Preference is given to mixtures of hydrochloric acid or acetic acid with water and/or an organic solvent such as ethyl acetate.

Method I—General Processes for the Oxidation of Thioethers to Sulfoxides and Sulfones

Compounds of the formula (I) in which W represents SO (sulfoxides) or SO₂ (sulfones) can be prepared from compounds of the formula (I) in which W represents S (thioethers) by oxidation by processes known from the literature, for example by means of an oxidizing agent in a suitable solvent or diluent. Suitable oxidizing agents are, for example, diluted nitric acid, hydrogen peroxide, Oxone® and peroxycarboxylic acids, for example meta-chloroperbenzoic acid. Suitable solvents or diluents are inert organic solvents, typically acetonitrile and halogenated solvents such as dichloromethane, chloroform or dichloroethane, and water and alcohols such as methanol for the reaction with Oxone®.

Also possible is the introduction, according to process B or D, of suitable anilines R′—NH₂ or boronic acids R¹—B(OH)₂ in which W represents SO or SO₂. These can be oxidized at the stage of the corresponding precursors in which W represents S, by processes known from the literature as described, for example, in WO 2013/092350.

A variety of methods are suitable for producing enantiomerically enriched sulfoxides, as described by G. E. O'Mahony et al., in ARKIVOC (Gainesville, Fla., United States), 2011, 1, 1-110: metal-catalysed asymmetric oxidations of thioethers, for example with titanium or vanadium as the most frequently employed catalyst sources, in the form of Ti(OⁱPr₄) or VO(acac)₂, together with a chiral ligand and an oxidizing agent such as tert-butyl hydroperoxide (TBHP), 2-phenylpropan-2-yl hydroperoxide (CHP) or hydrogen peroxide; non-metal-catalysed asymmetric oxidations employing chiral oxidizing agents or chiral catalysts; electrochemical or biological asymmetric oxidations and also kinetic resolution of sulfoxides and nucleophilic shift (according to Andersen's method).

The enantiomers can also be obtained from the racemate, for example by preparative separation by means of a chiral HPLC.

If compounds of the formula (I) are referred to hereinbelow, this also includes those compounds in Table 1 which are not embraced by formula (I).

Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. Accordingly, the invention encompasses both pure stereoisomers and any mixtures of these isomers.

Methods and Uses

The invention also relates to methods for controlling animal pests, in which compounds of the formula (I) are allowed to act on animal pests and/or their habitat. The control of the animal pests is preferably conducted in agriculture and forestry, and in material protection. This preferably excludes methods for surgical or therapeutic treatment of the human or animal body and diagnostic methods carried out on the human or animal body.

The invention further relates to the use of the compounds of the formula (I) as pesticides, especially crop protection agents.

In the context of the present application, the term “pesticide” also always encompasses the term “crop protection agent”.

The compounds of the formula (I), given good plant tolerance, favourable homeotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, nematodes and molluscs, which are encountered in agriculture, in horticulture, in animal husbandry, in aquatic cultures, in forests, in gardens and leisure facilities, in the protection of stored products and of materials, and in the hygiene sector. They can preferably be used as pesticides. They are active against normally sensitive and resistant species and also against all or specific stages of development. The abovementioned pests include:

pests from the phylum of the Arthropoda, in particular from the class of the Arachnida, for example Acarus spp., for example Acarus siro, Aceria kuko, Aceria sheldoni, Aculops spp., Aculus spp., for example Aculus fockeui, Aculus schlechtendali, Amblyomma spp., Amphitetranychus viennensis, Argas spp., Boophilus spp., Brevipalpus spp., for example Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., for example Eotetranychus hicoriae, Epitrimerus pyri, Eutetranychus spp., for example Eutetranychus banksi, Eriophyes spp., for example Eriophyes pyri, Glycyphagus domesticus, Halotydeus destructor, Hemitarsonemus spp., for example Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., for example Oligonychus coniferarum, Oligonychus ilicis, Oligonychus indicus,

Oligonychus mangiferus, Oligonychus pratensis, Oligonychus punicae, Oligonychus yothersi, Ornithodorus spp., Ornithonyssus spp., Panonychus spp., for example Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., for example Tarsonemus confusus, Tarsonemus pallidus, Tetranychus spp., for example Tetranychus canadensis, Tetranychus cinnabarinus, Tetranychus turkestani, Tetranychus urticae, Trombicula alfreddugesi, Vaejovis spp., Vasates lycopersici;

from the class of the Chilopoda, for example Geophilus spp., Scutigera spp.;

from the order or the class of the Collembola, for example Onychiurus armatus; Sminthurus viridis;

from the class of the Diplopoda, for example Blaniulus guttulatus;

from the class of the Insecta, for example from the order of the Blattodea, for example Blatta orientalis, Blattella asahinai, Blattella germanica, Leucophaea maderae, Panchlora spp., Parcoblatta spp., Periplaneta spp., for example Periplaneta americana, Periplaneta australasiae, Supella longipalpa;

from the order of the Coleoptera, for example Acalymma vittatum, Acanthoscelides obtectus, Adoretus spp., Agelastica alni, Agriotes spp., for example Agriotes linneatus, Agriotes mancus, Alphitobius diaperinus, Amphimallon solstitialis, Anobium punctatum, Anoplophora spp., Anthonomus spp., for example Anthonomus grandis, Anthrenus spp., Apion spp., Apogonia spp., Atomaria spp., for example Atomaria linearis, Attagenus spp., Baris caerulescens, Bruchidius obtectus, Bruchus spp., for example Bruchus pisorum, Bruchus rufimanus, Cassida spp., Cerotoma trifurcata, Ceutorrhynchus spp., for example Ceutorrhynchus assimilis, Ceutorrhynchus quadridens, Ceutorrhynchus rapae, Chaetocnema spp., for example Chaetocnema confinis, Chaetocnema denticulata, Chaetocnema ectypa, Cleonus mendicus, Conoderus spp., Cosmopolites spp., for example Cosmopolites sordidus, Costelytra zealandica, Ctenicera spp., Curculio spp., for example Curculio caryae, Curculio caryatrypes, Curculio obtusus, Curculio sayi, Cryptolestes ferrugineus, Cryptolestes pusillus,

Cryptorhynchus lapathi, Cryptorhynchus mangiferae, Cylindrocopturus spp., Cylindrocopturus adspersus, Cylindrocopturus furnissi, Dermestes spp., Diabrotica spp., for example Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epilachna spp., for example Epilachna borealis, Epilachna varivestis, Epitrix spp., for example Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., for example Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., for example Leucoptera coffeella,

Lissorhoptrus oryzophilus, Lixus spp., Luperomorpha xanthodera, Luperodes spp., Lyctus spp., Megascelis spp., Melanotus spp., for example Melanotus longulus oregonensis, Meligethes aeneus, Melolontha spp., for example Melolontha melolontha, Migdolus spp., Monochamus spp., Naupactus xanthographus, Necrobia spp., Niptus hololeucus, Oryctes rhinoceros, Oryzaephilus surinamensis, Oryzaphagus oryzae, Otiorhynchus spp., for example Otiorhynchus cribricollis, Otiorhynchus ligustici, Otiorhynchus ovatus, Otiorhynchus rugosostriarus, Otiorhynchus sulcatus, Oxycetonia jucunda, Phaedon cochleariae, Phyllophaga spp., Phyllophaga helleri, Phyllotreta spp., for example Phyllotreta armoraciae, Phyllotreta pusilla, Phyllotreta ramosa, Phyllotreta striolata, Popillia japonica, Premnotrypes spp., Prostephanus truncatus, Psylliodes spp., for example Psylliodes affinis, Psylliodes chrysocephala, Psylliodes punctulata, Ptinus spp., Rhizobius ventralis, Rhizopertha dominica, Sitophilus spp., for example Sitophilus granarius, Sitophilus linearis,

Sitophilus oryzae, Sitophilus zeamais, Sphenophorus spp., Stegobium paniceum, Sternechus spp., for example Sternechus paludatus, Symphyletes spp., Tanymecus spp., for example Tanymecus dilaticollis, Tanymecus indicus, Tanymecus palliatus, Tenebrio molitor, Tenebrioides mauretanicus, Tribolium spp., for example Tribolium audax, Tribolium castaneum, Tribolium confusum, Trogoderma spp., Tychius spp., Xylotrechus spp., Zabrus spp., for example Zabrus tenebrioides;

from the order of the Diptera, for example Aedes spp., for example Aedes aegypti, Aedes albopictus, Aedes sticticus, Aedes vexans, Agromyza spp., for example Agromyza frontella, Agromyza parvicornis, Anastrepha spp., Anopheles spp., for example Anopheles quadrimaculatus, Anopheles gambiae, Asphondylia spp., Bactrocera spp., for example Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., for example Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., for example Culex pipiens, Culex quinquefasciatus, Culicoides spp., Culiseta spp., Cuterebra spp., Dacus oleae,

Dasineura spp., for example Dasineura brassicae, Delia spp., for example Delia antiqua, Delia coarctata, Delia florilega, Delia platura, Delia radicum, Dermatobia hominis, Drosophila spp., for example Drosphila melanogaster, Drosophila suzukii, Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., for example Liriomyza brassicae, Liriomyza huidobrensis, Liriomyza sativae, Lucilia spp., for example Lucilia cuprina, Lutzomyia spp., Mansonia spp., Musca spp., for example Musca domestica, Musca domestica vicina, Oestrus spp., Oscinella frit, Paratanytarsus spp., Paralauterborniella subcincta,

Pegomya spp., for example Pegomya betae, Pegomya hyoscyami, Pegomya rubivora, Phlebotomus spp., Phorbia spp., Phormia spp., Piophila casei, Prodiplosis spp., Psila rosae, Rhagoletis spp., for example Rhagoletis cingulata, Rhagoletis completa, Rhagoletis fausta, Rhagoletis indifferens, Rhagoletis mendax, Rhagoletis pomonella, Sarcophaga spp., Simulium spp., for example Simulium meridionale, Stomoxys spp., Tabanus spp., Tetanops spp., Tipula spp., for example Tipula paludosa, Tipula simplex;

from the order of the Hemiptera, for example Acizzia acaciaebaileyanae, Acizzia dodonaeae, Acizzia uncatoides, Acrida turrita, Acyrthosipon spp., for example Acyrthosiphon pisum, Acrogonia spp., Aeneolamia spp., Agonoscena spp., Aleyrodes proletella, Aleurolobus barodensis, Aleurothrixus floccosus, Allocaridara malayensis, Amrasca spp., for example Amrasca bigutulla, Amrasca devastans, Anuraphis cardui, Aonidiella spp., for example Aonidiella aurantii, Aonidiella citrina, Aonidiella inornata, Aphanostigma piri, Aphis spp., for example Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., for example Aspidiotus nerii, Atanus spp., Aulacorthum solani, Bemisia tabaci, Blastopsylla occidentalis, Boreioglycaspis melaleucae,

Brachycaudus helichrysi, Brachycolus spp., Brevicoryne brassicae, Cacopsylla spp., for example Cacopsylla pyricola, Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., for example Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes citri, Diaphorina citri, Diaspis spp., Drosicha spp., Dysaphis spp., for example Dysaphis apiifolia, Dysaphis plantaginea, Dysaphis tulipae, Dysmicoccus spp., Empoasca spp., for example Empoasca abrupta, Empoasca fabae, Empoasca maligna, Empoasca solana, Empoasca stevensi, Eriosoma spp., for example Eriosoma americanum, Eriosoma lanigerum,

Eriosoma pyricola, Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Hyalopterus pruni, Icerya spp., for example Icerya purchasi, Idiocerus spp., Idioscopus spp., Laodelphax striatellus, Lecanium spp., for example Lecanium corni (=Parthenolecanium corni), Lepidosaphes spp., for example Lepidosaphes ulmi, Lipaphis erysimi, Lycorma delicatula, Macrosiphum spp., for example Macrosiphum euphorbiae, Macrosiphum Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., for example Myzus ascalonicus, Myzus cerasi, Myzus ligustri, Myzus ornatus, Myzus persicae, Myzus nicotianae, Nasonovia ribisnigri, Nephotettix spp., for example Nephotettix cincticeps Nephotettix nigropictus, Nilaparvata lugens, Oncometopia spp., Orthezia praelonga,

Oxya chinensis, Pachypsylla spp., Parabemisia myricae, Paratrioza spp., for example Paratrioza cockerelli, Parlatoria spp., Pemphigus spp., for example Pemphigus bursarius, Pemphigus populivenae, Peregrinus maidis, Phenacoccus spp., for example Phenacoccus madeirensis, Phloeomyzus passerinii, Phorodon humuli, Phylloxera spp., for example Phylloxera devastatrix, Phylloxera notabilis, Pinnaspis aspidistrae, Planococcus spp., for example Planococcus citri, Prosopidopsylla flava, Protopulvinaria pyriformis, Pseudaulacaspis pentagona, Pseudococcus spp., for example Pseudococcus calceolariae, Pseudococcus comstocki, Pseudococcus longispinus, Pseudococcus maritimus, Pseudococcus viburni, Psyllopsis spp., Psylla spp., for example Psylla buxi, Psylla mali, Psylla pyri, Pteromalus spp., Pyrilla spp., Quadraspidiotus spp., for example Quadraspidiotus juglansregiae, Quadraspidiotus ostreaeformis, Quadraspidiotus perniciosus, Quesada gigas, Rastrococcus spp., Rhopalosiphum spp., for example Rhopalosiphum maidis, Rhopalosiphum oxyacanthae, Rhopalosiphum padi,

Rhopalosiphum rufiabdominale, Saissetia spp., for example Saissetia coffeae, Saissetia miranda, Saissetia neglecta, Saissetia oleae, Scaphoideus titanus, Schizaphis graminum, Selenaspidus articulatus, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., for example Toxoptera aurantii, Toxoptera citricidus, Trialeurodes vaporariorum, Trioza spp., for example Trioza diospyri, Typhlocyba spp., Unaspis spp., Viteus vitifolii, Zygina spp.;

from the suborder of the Heteroptera, for example Anasa tristis, Antestiopsis spp., Boisea spp., Blissus spp., Calocoris spp., Campylomma livida, Cavelerius spp., Cimex spp., for example Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., for example Euschistus heros, Euschistus servus, Euschistus tristigmus, Euschistus variolarius, Eurygaster spp., Halyomorpha halys, Heliopeltis spp., Horcias nobilellus, Leptocorisa spp., Leptocorisa varicornis, Leptoglossus occidentalis,

Leptoglossus phyllopus, Lygocoris spp., for example Lygocoris pabulinus, Lygus spp., for example Lygus elisus, Lygus hesperus, Lygus lineolaris, Macropes excavatus, Monalonion atratum, Nezara spp., for example Nezara viridula, Oebalus spp., Piesma quadrata, Piezodorus spp., for example Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;

from the order of the Hymenoptera, for example Acromyrmex spp., Athalia spp., for example Athalia rosae, Atta spp., Diprion spp., for example Diprion similis, Hoplocampa spp., for example Hoplocampa cookei, Hoplocampa testudinea, Lasius spp., Linepithema humile, Monomorium pharaonis, Sirex spp., Solenopsis invicta, Tapinoma spp., Urocerus spp., Vespa spp., for example Vespa crabro, Xeris spp.;

from the order of the Isopoda, for example Armadillidium vulgare, Oniscus asellus, Porcellio scaber;

from the order of the Isoptera, for example Coptotermes spp., for example Coptotermes formosanus, Cornitermes cumulans, Cryptotermes spp., Incisitermes spp., Microtermes obesi, Odontotermes spp., Reticulitermes spp., for example Reticulitermes flavipes, Reticulitermes hesperus;

from the order of the Lepidoptera, for example Achroia grisella, Acronicta major, Adoxophyes spp., for example Adoxophyes orana, Aedia leucomelas, Agrotis spp., for example Agrotis segetum, Agrotis ipsilon, Alabama spp., for example Alabama argillacea, Amyelois transitella, Anarsia spp., Anticarsia spp., for example Anticarsia gemmatalis, Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., for example Chilo plejadellus, Chilo suppressalis, Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., for example Cydia nigricana, Cydia pomonella, Dalaca noctuides, Diaphania spp., Diatraea saccharalis, Earias spp., Ecdytolopha aurantium,

Elasmopalpus lignosellus, Eldana saccharina, Ephestia spp., for example Ephestia elutella, Ephestia kuehniella, Epinotia spp., Epiphyas postvittana, Etiella spp., Eulia spp., Eupoecilia ambiguella, Euproctis spp., for example Euproctis chrysorrhoea, Euxoa spp., Feltia spp., Galleria mellonella, Gracillaria spp., Grapholitha spp., for example Grapholita molesta, Grapholita prunivora, Hedylepta spp., Helicoverpa spp., for example Helicoverpa armigera, Helicoverpa zea, Heliothis spp., for example Heliothis virescens Hofmannophila pseudospretella, Homoeosoma spp., Homona spp., Hyponomeuta padella, Kakivoria flavofasciata, Laphygma spp., Leucinodes orbonalis, Leucoptera spp., for example Leucoptera coffeella, Lithocolletis spp., for example Lithocolletis blancardella, Lithophane antennata, Lobesia spp., for example Lobesia botrana, Loxagrotis albicosta, Lymantria spp., for example Lymantria dispar, Lyonetia spp., for example Lyonetia clerkella, Malacosoma neustria, Maruca testulalis, Mamestra brassicae, Melanitis leda, Mocis spp., Monopis obviella, Mythimna separata,

Nemapogon cloacellus, Nymphula spp., Oiketicus spp., Oria spp., Orthaga spp., Ostrinia spp., for example Ostrinia nubilalis, Oulema melanopus, Oulema oryzae, Panolis flammea, Parnara spp., Pectinophora spp., for example Pectinophora gossypiella, Perileucoptera spp., Phthorimaea spp., for example Phthorimaea operculella, Phyllocnistis citrella, Phyllonorycter spp., for example Phyllonorycter blancardella, Phyllonorycter crataegella, Pieris spp., for example Pieris rapae, Platynota stultana, Plodia interpunctella, Plusia spp., Plutella xylostella (=Plutella maculipennis), Prays spp., Prodenia spp., Protoparce spp., Pseudaletia spp., for example Pseudaletia unipuncta,

Pseudoplusia includens, Pyrausta nubilalis, Rachiplusia nu, Schoenobius spp., for example Schoenobius bipunctifer, Scirpophaga spp., for example Scirpophaga innotata, Scotia segetum, Sesamia spp., for example Sesamia inferens, Sparganothis spp., Spodoptera spp., for example Spodoptera eradiana, Spodoptera exigua, Spodoptera frugiperda, Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., for example Trichoplusia ni, Tryporyza incertulas, Tuta absoluta, Virachola spp.;

from the order of the Orthoptera or Saltatoria, for example Acheta domesticus, Dichroplus spp., Gryllotalpa spp., for example Gryllotalpa gryllotalpa, Hieroglyphus spp., Locusta spp., for example Locusta migratoria, Melanoplus spp., for example Melanoplus devastator, Paratlanticus ussuriensis, Schistocerca gregaria;

from the order of the Phthiraptera, for example Damalinia spp., Haematopinus spp., Linognathus spp., Pediculus spp., Phylloxera vastatrix, Phthirus pubis, Trichodectes spp.;

from the order of the Psocoptera, for example Lepinotus spp., Liposcelis spp.;

from the order of the Siphonaptera, for example Ceratophyllus spp., Ctenocephalides spp., for example Ctenocephalides canis, Ctenocephalides felis, Pulex irritans, Tunga penetrans, Xenopsylla cheopis;

from the order of the Thysanoptera, for example Anaphothrips obscurus, Baliothrips biformis, Drepanothrips reuteri, Enneothrips flavens, Frankliniella spp., for example Frankliniella fusca, Frankliniella occidentalis, Frankliniella schultzei, Frankliniella tritici, Frankliniella vaccinii, Frankliniella williamsi, Heliothrips spp., Hercinothrips femoralis, Rhipiphorothrips cruentatus, Scirtothrips spp., Taeniothrips cardamomi, Thrips spp., for example Thrips palmi, Thrips tabaci;

from the order of the Zygentoma (=Thy sanura), for example Ctenolepisma spp., Lepisma saccharina, Lepismodes inquilinus, Thermobia domestica;

from the class of the Symphyla, for example Scutigerella spp., for example Scutigerella immaculata;

pests from the phylum of the Mollusca, in particular from the class of the Bivalvia, for example Dreissena spp.;

and also from the class of the Gastropoda, for example Anion spp., for example Anion ater rufus, Biomphalaria spp., Bulinus spp., Deroceras spp., for example Deroceras laeve, Galba spp., Lymnaea spp., Oncomelania spp., Pomacea spp., Succinea spp.;

animal and human parasites from the phyla of the Platyhelminths and Nematoda, for example Aelurostrongylus spp., Amidostomum spp., Ancylostoma spp, Angiostrongylus spp., Anisakis spp., Anoplocephala spp., Ascaris spp., Ascaridia spp., Baylisascaris spp., Brugia spp., Bunostomum spp., Capillaria spp., Chabertia spp., Clonorchis spp., Cooperia spp., Crenosoma spp., Cyathostoma spp., Dicrocoelium spp., Dictyocaulus spp., Diphyllobothrium spp., Dipylidium spp., Dirofilaria spp., Dracunculus spp., Echinococcus spp., Echinostoma spp., Enterobius spp., Eucoleus spp., Fasciola spp., Fascioloides spp., Fasciolopsis spp., Filaroides spp., Gongylonema spp., Gyrodactylus spp., Habronema spp., Haemonchus spp., Heligmosomoides spp., Heterakis spp., Hymenolepis spp., Hyostrongylus spp., Litomosoides spp., Loa spp., Metastrongylus spp., Metorchis spp., Mesocestoides spp., Moniezia spp., Muellerius spp.,

Necator spp., Nematodirus spp., Nippostrongylus spp., Oesophagostomum spp., Ollulanus spp., Onchocerca spp, Opisthorchis spp., Oslerus spp., Ostertagia spp., Oxyuris spp., Paracapillaria spp., Parafilaria spp., Paragonimus spp., Paramphistomum spp., Paranoplocephala spp., Parascaris spp., Passalurus spp., Protostrongylus spp., Schistosoma spp., Setaria spp., Spirocerca spp., Stephanofilaria spp., Stephanurus spp., Strongyloides spp., Strongylus spp., Syngamus spp., Taenia spp., Teladorsagia spp., Thelazia spp., Toxascaris spp., Toxocara spp., Trichinella spp., Trichobilharzia spp., Trichostrongylus spp., Trichuris spp., Uncinaria spp., Wuchereria spp.;

plant pests from the phylum of the Nematoda, i.e. phytoparasitic nematodes, in particular Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax (=Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp.,

Heterodera spp., for example Heterodera avenae, Heterodera glycines, Heterodera schachtii, Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paraphelenchus spp., Paratrichodorus spp., for example Paratrichodorus minor, Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.

In addition, it is possible to control, from the sub-kingdom of the Protozoa, the order of the Coccidia, for example Eimeria spp.

The compounds of the formula (I) can optionally, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). They can, as the case may be, also be used as intermediates or precursors for the synthesis of other active compounds.

Formulations

The present invention further relates to formulations and use forms prepared therefrom as pesticides, for example drench, drip and spray liquors, comprising at least one compound of the formula (I). Optionally, the use forms comprise further pesticides and/or adjuvants which improve action, such as penetrants,

e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulfate or diammonium hydrogenphosphate and/or retention promoters, for example dioctyl sulfosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers 173, prepared by the FAO/WHO Joint Meeting on Pesticide Specifications, 2004, ISBN: 9251048576. The formulations, in addition to one or more compounds of the formula (I), optionally comprise further agrochemically active compounds.

Preference is given to formulations or use forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are prepared in a known way, for example by mixing the compounds of the formula (I) with auxiliaries such as, for example, extenders, solvents and/or solid carriers and/or other auxiliaries such as, for example, surfactants. The formulations are produced either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the simple and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and water.

In principle, it is possible to use all suitable solvents. Examples of suitable solvents are aromatic hydrocarbons, such as xylene, toluene or alkylnaphthalenes, chlorinated aromatic or aliphatic hydrocarbons, such as chlorobenzene, chloroethylene or methylene chloride, aliphatic hydrocarbons, such as cyclohexane, paraffins, mineral oil fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulfoxide, and also water.

In principle, it is possible to use all suitable carriers. Useful carriers especially include: for example ammonium salts and ground natural minerals such as kaolins, clays, talc, chalk, quartz, attapulgite, montmorillonite or diatomaceous earth, and ground synthetic minerals such as finely divided silica, alumina and natural or synthetic silicates, resins, waxes and/or solid fertilizers. It is likewise possible to use mixtures of such carriers. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, maize cobs and tobacco stalks.

It is also possible to use liquefied gaseous extenders or solvents. Especially suitable are those extenders or carriers which are gaseous at standard temperature and under atmospheric pressure, for example aerosol propellants such as halogenated hydrocarbons, and also butane, propane, nitrogen and carbon dioxide.

Examples of emulsifiers and/or foam formers, dispersants or wetting agents having ionic or nonionic properties or mixtures of these surface-active substances are salts of polyacrylic acid, salts of lignosulfonic acid, salts of phenolsulfonic acid or naphthalenesulfonic acid, polycondensates of ethylene oxide with fatty alcohols or with fatty acids or with fatty amines, with substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolysates, lignosulfite waste liquors and methylcellulose.

The presence of a surfactant is advantageous if one of the compounds of the formula (I) and/or one of the inert carriers is insoluble in water and if the application takes place in water.

Further auxiliaries which may be present in the formulations and the use forms derived therefrom are dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components which may be present are stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further auxiliaries may be mineral and vegetable oils.

It is possible if appropriate for still further auxiliaries to be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce dynamic surface tension, for example dioctyl sulfosuccinate, or increase viscoelasticity, for example hydroxypropylguar polymers.

Useful penetrants in the present context are all those substances which are typically used to improve the penetration of agrochemically active compounds into plants. Penetrants are defined in this context by their ability to penetrate from the (generally aqueous) application liquor and/or from the spray coating into the cuticle of the plant and hence increase the mobility of the active compounds in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxy late (12),

fatty acid esters, for example rapeseed oil methyl ester or soya oil methyl ester, fatty amine alkoxylates, for example tallowamine ethoxylate (15), or ammonium and/or phosphonium salts, for example ammonium sulfate or diammonium hydrogenphosphate.

The formulations preferably comprise between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (in particular pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. Application is accomplished in a customary manner appropriate for the use forms.

Mixtures

The compounds of the formula (I) can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance. In addition, active compound combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compounds of the formula (I) may be present in a mixture with other active compounds or semiochemicals such as attractants and/or bird repellents and/or plant activators and/or growth regulators and/or fertilizers. Likewise, the compounds of the formula (I) can be used in mixtures with agents to improve plant properties, for example growth, yield and quality of the harvested material.

In a particular embodiment according to the invention, the compounds of the formula (I) are present in formulations or in the use forms prepared from these formulations in a mixture with further compounds, preferably those as described below.

If one of the compounds mentioned below can occur in different tautomeric forms, these forms are also included even if not explicitly mentioned in each case.

Insecticides/Acaricides/Nematicides

The active compounds specified here with their common names are known and are described for example in “The Pesticide Manual”, 16th ed., British Crop Protection Council 2012, or can be searched for on the Internet (e.g. http://www.alanwood.net/pesticides).

(1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate,

dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl)salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion.

(2) GABA-gated chloride channel antagonists, for example cyclodiene-organochlorines, e.g. chlordane and endosulfan or phenylpyrazoles (fiproles), e.g. ethiprole and fipronil.

(3) Sodium channel modulators/voltage-gated sodium channel blockers such as, for example, pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin or DDT or methoxychlor.

(4) Nicotinergic acetylcholine receptor (nAChR) agonists, for example neonicotinoids, e.g. acetamiprid, clothianidin, dinotefuran, imidacloprid, nitenpyram, thiacloprid and thiamethoxam or nicotine or sulfoxaflor or flupyradifurone.

(5) Allosteric activators of the nicotinergic acetylcholine receptor (nAChR), for example spinosyns, e.g. spinetoram and spinosad.

(6) Chloride channel activators, for example avermectins/milbemycins, e.g. abamectin, emamectin benzoate, lepimectin and milbemectin.

(7) Juvenile hormone imitators, for example, juvenile hormone analogues, e.g. hydroprene, kinoprene and methoprene or fenoxycarb or pyriproxyfen.

(8) Active compounds having unknown or nonspecific mechanisms of action, for example alkyl halides, e.g. methyl bromide and other alkyl halides; or chloropicrine or sulfuryl fluoride or borax or tartar emetic.

(9) Selective antifeedants, e.g. pymetrozine or flonicamid.

(10) Mite growth inhibitors, e.g. clofentezine, hexythiazox and diflovidazin or etoxazole.

(11) Microbial disruptors of the insect gut membrane, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.

(12) Oxidative phosphorylation inhibitors, ATP disruptors, for example diafenthiuron or organotin compounds, e.g. azocyclotin, cyhexatin and fenbutatin oxide or propargite or tetradifon.

(13) Oxidative phosphorylation decouplers that interrupt the H proton gradient, for example chlorfenapyr, DNOC and sulfluramid.

(14) Nicotinergic acetylcholine receptor antagonists, for example bensultap, cartap hydrochloride, thiocyclam, and thiosultap-sodium.

(15) Inhibitors of chitin biosynthesis, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1, for example buprofezin.

(17) Moulting inhibitors (especially for Diptera, i.e. dipterans), for example cyromazine.

(18) Ecdysone receptor agonists, for example chromafenozide, halofenozide, methoxyfenozide and tebufenozide.

(19) Octopaminergic agonists, for example amitraz.

(20) Complex-III electron transport inhibitors, for example hydramethylnon or acequinocyl or fluacrypyrim.

(21) Complex-I electron transport inhibitors, for example METI acaricides, e.g. fenazaquin, fenpyroximate, pyrimidifen, pyridaben, tebufenpyrad and tolfenpyrad or rotenone (Derris).

(22) Voltage-gated sodium channel blockers, for example indoxacarb or metaflumizone.

(23) Inhibitors of acetyl-CoA carboxylase, for example tetronic and tetramic acid derivatives, e.g. spirodiclofen, spiromesifen and spirotetramat.

(24) Complex-IV electron transport inhibitors, for example phosphines, e.g. aluminium phosphide, calcium phosphide, phosphine and zinc phosphide or cyanide.

(25) Complex-II electron transport inhibitors, for example cyenopyrafen and cyflumetofen.

(28) Ryanodine receptor effectors, for example diamides, e.g. chlorantraniliprole, cyantraniliprole and flubendiamide.

Further active compounds having an unknown or unclear mechanism of action, for example afidopyropen, afoxolaner, azadirachtin, benclothiaz, benzoximate, bifenazate, bromopropylate, chinomethionat, cryolite, cyclaniliprole, cycloxaprid, cyhalodiamide, dicloromezotiaz, dicofol, diflovidazin, flometoquin, fluensulfone, flufenerim, flufenoxystrobin, flufiprole, fluhexafon, fluopyram, fluralaner, fufenozide, guadipyr, heptafluthrin, imidaclothiz, iprodione, meperfluthrin, paichongding, pyflubumide, pyridalyl, pyrifluquinazon, pyriminostrobin, tetramethylfluthrin, tetraniliprole, tetrachlorantraniliprole, tioxazafen, triflumezopyrim and iodomethane; and additionally preparations based on Bacillus firmus (1-1582, BioNeem, Votivo), and the following known active compounds: 1-{2-fluoro-4-methyl-5-[(2,2,2-trifluoroethyl)sulfinyl]phenyl}-3-(trifluoromethyl)-1H-1,2,4-triazole-5-amine (known from WO2006/043635), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-ylethyl carbonate (known from WO2009/049851), 4-(but-2-yn-1-yloxy)-6-(3,5-dimethylpiperidin-1-yl)-5-fluoropyrimidine (known from WO2004/099160), 4-(but-2-yn-1-yloxy)-6-(3-chlorophenyl)pyrimidine (known from WO2003/076415), PF1364 (CAS Reg. No. 1204776-60-2), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO2005/085216), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 8-chloro-N-[(2-chloro-5-methoxyphenyl)sulfonyl]-6-(trifluoromethyl)imidazo[1,2-a]pyridine-2-carboxamide (known from WO2010/129500),

4-[5-(3,5-dichlorophenyl)-5-(trifluoromethyl)-4,5-dihydro-1,2-oxazol-3-yl]-2-methyl-N-(1-oxidothietan-3-yl)benzamide (known from WO2009/080250), N-[(2E)-1-[(6-chloropyridin-3-yl)methyl]pyridin-2(1H)-ylidene]-2,2,2-trifluoroacetamide (known from WO2012/029672), 1-[(2-chloro-1,3-thiazol-5-yl)methyl]-4-oxo-3-phenyl-4H-pyrido[1,2-a]pyrimidin-1-ium-2-olate (known from WO2009/099929), 1-[(6-chloropyridin-3-yl)methyl]-4-oxo-3-phenyl-4H-pyrido[1,2-a]pyrimidin-1-ium-2-olate (known from WO2009/099929), 4-(3-{2,6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008/134969), 3-[benzoyl(methyl)amino]-N-[2-bromo-4-[1,2,2,2-tetrafluoro-1-(trifluoromethyl)ethyl]-6-(trifluoromethyl)phenyl]-2-fluorobenzamide (known from WO 2010018714), butyl [2-(2,4-dichlorophenyl)-3-oxo-4-oxaspiro[4.5]dec-1-en-1-yl]carbonate (known from CN 102060818), 4-[5-(3,5-dichlorophenyl)-5-(trifluoromethyl)-4H-isoxazol-3-yl]-N-[(Z)-methoxyiminomethyl]-2-methylbenzamide (known from WO2007/026965), (3E)-3-[1-[(6-chloro-3-pyridyl)methyl]-2-pyridylidene]-1,1,1-trifluoropropan-2-one (known from WO2013/144213), N-(methylsulfonyl)-6-[2-(pyridin-3-yl)-1,3-thiazol-5-yl]pyridine-2-carboxamide (known from WO2012/000896), N-[3-(benzylcarbamoyl)-4-chlorophenyl]-1-methyl-3-(pentafluoroethyl)-4-(trifluoromethyl)-1H-pyrazole-5-carboxamide (known from WO2010/051926),

Fungicides

The active compounds specified herein by their common name are known and described, for example, in the “Pesticide Manual” or on the Internet (for example: http://www.alanwood.net/pesticides).

All the fungicidal mixing components listed in classes (1) to (15) may optionally form salts with corresponding bases or acids if suitable functional groups are present. In addition, the fungicidal mixing components listed in classes (1) to (15) also include tautomeric forms if tautomerism is possible.

1) inhibitors of the ergosterol biosynthesis, for example (1.01) aldimorph, (1.02) azaconazole, (1.03) bitertanol, (1.04) bromuconazole, (1.05) cyproconazole, (1.06) diclobutrazole, (1.07) difenoconazole, (1.08) diniconazole, (1.09) diniconazole-M, (1.10) dodemorph, (1.11) dodemorph acetate, (1.12) epoxiconazole, (1.13) etaconazole, (1.14) fenarimol, (1.15) fenbuconazole, (1.16) fenhexamide, (1.17) fenpropidin, (1.18) fenpropimorph, (1.19) fluquinconazole, (1.20) flurprimidol, (1.21) flusilazole, (1.22) flutriafol, (1.23) furconazole, (1.24) furconazole-cis, (1.25) hexaconazole, (1.26) imazalil, (1.27) imazalil sulfate, (1.28) imibenconazole, (1.29) ipconazole, (1.30) metconazole, (1.31) myclobutanil, (1.32) naftifine, (1.33) nuarimol, (1.34) oxpoconazole, (1.35) paclobutrazole, (1.36) pefurazoate, (1.37) penconazole, (1.38) piperalin, (1.39) prochloraz, (1.40) propiconazole, (1.41) prothioconazole, (1.42) pyributicarb, (1.43) pyrifenox, (1.44) quinconazole, (1.45) simeconazole, (1.46) spiroxamine, (1.47) tebuconazole,

(1.48) terbinafin, (1.49) tetraconazole, (1.50) triadimefon, (1.51) triadimenol, (1.52) tridemorph, (1.53) triflumizole, (1.54) triforin, (1.55) triticonazole, (1.56) uniconazole, (1.57) uniconazole-p, (1.58) viniconazole, (1.59) voriconazole, (1.60) 1-(4-chlorophenyl)-2-(1H-1,2,4-triazol-1-yl)cycloheptanol, (1.61) 1-(2,2-dimethyl-2,3-dihydro-1H-inden-1-yl)-1H-imidazole-5-carboxylic acid methyl ester, (1.62) N′-{5-(difluoromethyl)-2-methyl-4-[3-(trimethylsilyl)propoxy]phenyl}-N-ethyl-N-methylimidoformamide, (1.63) N-ethyl-N-methyl-N′-{2-methyl-5-(trifluoromethyl)-4-[3-(trimethylsilyl)propoxy]phenyl}imidoformamide, (1.64) O-[1-(4-methoxyphenoxy)-3,3-dimethylbutan-2-yl]-1H-imidazole-1-carbothioate, (1.65) pyrisoxazole, (1.66) 2-{[3-(2-chlorophenyl)-2-(2,4-difluorophenyl)oxiran-2-yl]methyl}-2,4-dihydro-3H-1,2,4-triazole-3-thione, (1.67) 1-{[3-(2-chlorophenyl)-2-(2,4-difluorophenyl)oxiran-2-yl]methyl}-1-H-1,2,4-triazol-5-ylthiocyanate,

(1.68) 5-(allylsulfanyl)-1-{[3-(2-chlorophenyl)-2-(2,4-difluorophenyl)oxiran-2-yl]methyl}-1H-1,2,4-triazole, (1.69) 2-[1-(2,4-dichlorophenyl)-5-hydroxy-2,6,6-trimethylheptan-4-yl]-2,4-dihydro-3H-H-1,2,4-triazole-3-thione, (1.70) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.71) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.72) 1-{[rel(2R,3S)-3-(2-chlorophenyl)-2-(2,4-difluorophenyl)oxiran-2-yl]methyl}-1H-1,2,4-triazol-5-ylthiocyanate, (1.73) 1-{[rel(2R,3R)-3-(2-chlorophenyl)-2-(2,4-difluorophenyl)oxiran-2-yl]methyl}-1H-,2,4-triazol-5-ylthiocyanate, (1.74) 5-(allylsulfanyl)-1-{[rel(2R,3S)-3-(2-chlorophenyl)-2-(2,4-difluorophenyl)oxiran-2-yl]methyl}-1H-1,2,4-triazole, (1.75) 5-(allylsulfanyl)-1-{[rel(2R,3R)-3-(2-chlorophenyl)-2-(2,4-difluorophenyl)oxiran-2-yl]methyl}-1H-1,2,4-triazole,

(1.76) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.77) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.78) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.79) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.80) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.81) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.82) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.83) 2-[(2S,4R,5S)-1-(2,4-dichlorophenyl)-5-hydroxy-2,6,6-trimethylheptan-4-yl]-2,4-dihydro-3H-1,2,4-triazole-3-thione,

(1.84) 2-[4-(4-chlorophenoxy)-2-(trifluoromethyl)phenyl]-1-(1H-1,2,4-triazol-1-yl)propan-2-ol, (1.85) 2-[4-(4-chlorophenoxy)-2-(trifluoromethyl)phenyl]-1-(1H-1,2,4-triazol-1-yl)butan-2-ol, (1.86) 2-[4-(4-chlorophenoxy)-2-(trifluoromethyl)phenyl]-1-(1H-1,2,4-triazol-1-yl)pentan-2-ol, (1.87) 2-[2-chloro-4-(4-chlorophenoxy)phenyl]-1-(1H-,2,4-triazol-1-yl)butan-2-ol, (1.88) 2-[2-chloro-4-(2,4-dichlorophenoxy)phenyl]-1-(1H-1,2,4-triazol-1-yl)propan-2-ol, (1.89) (2R)-2-(1-chlorocyclopropyl)-4-[(1R)-2,2-dichlorocyclopropyl]-1-(1H-1,2,4-triazol-1-yl)butan-2-ol, (1.90) (2R)-2-(1-chlorocyclopropyl)-4-[(1S)-2,2-dichlorocyclopropyl]-1-(1H-1,2,4-triazol-1-yl)butan-2-ol, (1.91) (2S)-2-(1-chlorocyclopropyl)-4-[(1 S)-2,2-dichlorocyclopropyl]-1-(1H-1,2,4-triazol-1-yl)butan-2-ol, (1.92) (2S)-2-(1-chlorocyclopropyl)-4-[(1R)-2,2-dichlorocyclopropyl]-1-(1H-1,2,4-triazol-1-yl)butan-2-ol, (1.93) (1S,2R,5R)-5-(4-chlorobenzyl)-2-(chloromethyl)-2-methyl-1-(1H-1,2,4-triazol-1-ylmethyl)cyclopentanol, (1.94) (1R,2S,5S)-5-(4-chlorobenzyl)-2-(chloromethyl)-2-methyl-1-(1H-1,2,4-triazol-1-ylmethyl)cyclopentanol, (1.95) 5-(4-chlorobenzyl)-2-(chloromethyl)-2-methyl-1-(1H-1,2,4-triazol-1-ylmethyl)cyclopentanol.

2) Inhibitors of the respiratory chain on complex I or II, for example (2.01) bixafen, (2.02) boscalid, (2.03) carboxin, (2.04) diflumetorim, (2.05) fenfuram, (2.06) fluopyram, (2.07) flutolanil, (2.08) fluxapyroxad, (2.09) furametpyr, (2.10) furmecyclox, (2.11) isopyrazam (mixture of syn-epimeric racemate 1RS,4SR,9RS and anti-epimeric racemate 1RS,4SR,9SR), (2.12) isopyrazam (anti-epimeric racemate 1RS,4SR,9SR), (2.13) isopyrazam (anti-epimeric enantiomer 1R,4S,9S), (2.14) isopyrazam (anti-epimeric enantiomer 1S,4R,9R), (2.15) isopyrazam (syn-epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil, (2.19) oxycarboxin, (2.20) penflufen, (2.21) penthiopyrad,

(2.22) sedaxane, (2.23) thifluzamide, (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methyl-1H-pyrazole-4-carboxamide, (2.27) N-[1-(2,4-dichlorophenyl)-1-methoxypropan-2-yl]-3-(difluoromethyl)-1-methyl-1H-pyrazole-4-carboxamide, (2.28) 5,8-difluoro-N-[2-(2-fluoro-4-{[4-(trifluoromethyl)pyridin-2-yl]oxy}phenyl)ethyl]quinazoline-4-amine, (2.29) benzovindiflupyr, (2.30) N-[(1S,4R)-9-(dichloromethylene)-1,2,3,4-tetrahydro-1,4-methanonaphthalin-5-yl]-3-(difluoromethyl)-1-methyl-1H-pyrazole-4-carboxamide, (2.31) N-[(1R,4S)-9-(dichloromethylene)-1,2,3,4-tetrahydro-1,4-methanonaphthalin-5-yl]-3-(difluoromethyl)-1-methyl-1H-pyrazole-4-carboxamide,

(2.32) 3-(difluoromethyl)-1-methyl-N-(1,1,3-trimethyl-2,3-dihydro-1H-inden-4-yl)-1H-pyrazole-4-carboxamide, (2.33) 1,3,5-trimethyl-N-(1,1,3-trimethyl-2,3-dihydro-1H-inden-4-yl)-1H-pyrazole-4-carboxamide, (2.34) 1-methyl-3-(trifluoromethyl)-N-(1,1,3-trimethyl-2,3-dihydro-1H-inden-4-yl)-1H-pyrazole-4-carboxamide, (2.35) 1-methyl-3-(trifluoromethyl)-N-[(3R)-1,1,3-trimethyl-2,3-dihydro-1H-inden-4-yl]-1H-pyrazole-4-carboxamide, (2.36) 1-methyl-3-(trifluoromethyl)-N-[(3S)-1,1,3-trimethyl-2,3-dihydro-1H-inden-4-yl]-1H-pyrazole-4-carboxamide, (2.37) 3-(difluoromethyl)-1-methyl-N-[(3S)-1,1,3-trimethyl-2,3-dihydro-1H-inden-4-yl]-1H-pyrazole-4-carboxamide, (2.38) 3-(difluoromethyl)-1-methyl-N-[(3R)-1,1,3-trimethyl-2,3-dihydro-1H-inden-4-yl]-1H-pyrazole-4-carboxamide, (2.39) 1,3,5-trimethyl-N-[(3R)-, 1,3-trimethyl-2,3-dihydro-1H-inden-4-yl]-1H-pyrazole-4-carboxamide, (2.40) 1,3,5-trimethyl-N-[(3S)-1,1,3-trimethyl-2,3-dihydro-1H-inden-4-yl]-1H-pyrazole-4-carboxamide, (2.41) benodanil,

(2.42) 2-chloro-N-(1,1,3-trimethyl-2,3-dihydro-1H-inden-4-yl)pyridine-3-carboxamide, (2.43) isofetamid, (2.44) 1-methyl-3-(trifluoromethyl)-N-[2′-(trifluoromethyl)biphenyl-2-yl]-1H-pyrazole-4-carboxamide, (2.45) N-(4′-chlorobiphenyl-2-yl)-3-(difluoromethyl)-1-methyl-1H-pyrazole-4-carboxamide, (2.46) N-(2′,4′-dichlorobiphenyl-2-yl)-3-(difluoromethyl)-1-methyl-1H-pyrazole-4-carboxamide, (2.47) 3-(difluoromethyl)-1-methyl-N-[4′-(trifluoromethyl)biphenyl-2-yl]-1H-pyrazole-4-carboxamide, (2.48) N-(2′,5′-difluorobiphenyl-2-yl)-1-methyl-3-(trifluoromethyl)-1H-pyrazole-4-carboxamide, (2.49) 3-(difluoromethyl)-1-methyl-N-[4′-(prop-1-yn-1-yl)biphenyl-2-yl]-1H-pyrazole-4-carboxamide, (2.50) 5-fluoro-1,3-dimethyl-N-[4′-(prop-1-yn-1-yl)biphenyl-2-yl]-1H-pyrazole-4-carboxamide, (2.51) 2-chloro-N-[4′-(prop-1-yn-1-yl)biphenyl-2-yl]nicotinamide, (2.52) 3-(difluoromethyl)-N-[4′-(3,3-dimethylbut-1-yn-1-yl)biphenyl-2-yl]-1-methyl-1H-pyrazole-4-carboxamide,

(2.53) N-[4′-(3,3-dimethylbut-1-yn-1-yl)biphenyl-2-yl]-5-fluoro-1,3-dimethyl-1H-pyrazole-4-carboxamide, (2.54) 3-(difluoromethyl)-N-(4′-ethynylbiphenyl-2-yl)-1-methyl-1H-pyrazole-4-carboxamide, (2.55) N-(4′-ethynylbiphenyl-2-yl)-5-fluoro-1,3-dimethyl-1H-pyrazole-4-carboxamide, (2.56) 2-chloro-N-(4′-ethynylbiphenyl-2-yl)nicotinamide, (2.57) 2-chloro-N-[4′-(3,3-dimethylbut-1-yn-1-yl)biphenyl-2-yl]nicotinamide, (2.58) 4-(difluoromethyl)-2-methyl-N-[4′-(trifluoromethyl)biphenyl-2-yl]-1,3-thiazole-5-carboxamide, (2.59) 5-fluoro-N-[4′-(3-hydroxy-3-methylbut-1-yn-1-yl)biphenyl-2-yl]-1,3-dimethyl-1H-pyrazole-4-carboxamide, (2.60) 2-chloro-N-[4′-(3-hydroxy-3-methylbut-1-yn-1-yl)biphenyl-2-yl]nicotinamide, (2.61) 3-(difluoromethyl)-N-[4′-(3-methoxy-3-methylbut-1-yn-1-yl)biphenyl-2-yl]-1-methyl-1H-pyrazole-4-carboxamide, (2.62) 5-fluoro-N-[4′-(3-methoxy-3-methylbut-1-yn-1-yl)biphenyl-2-yl]-1,3-dimethyl-1H-pyrazole-4-carboxamide,

(2.63) 2-chloro-N-[4′-(3-methoxy-3-methylbut-1-yn-1-yl)biphenyl-2-yl]nicotinamide, (2.64) 1,3-dimethyl-N-(1,1,3-trimethyl-2,3-dihydro-1H-inden-4-yl)-1H-pyrazole-4-carboxamide, (2.65) 1,3-dimethyl-N-[(3R)-,1,1,3-trimethyl-2,3-dihydro-1H-inden-4-yl]-1H-pyrazole-4-carboxamide, (2.66) 1,3-dimethyl-N-[(3S)-1,1,3-trimethyl-2,3-dihydro-1H-inden-4-yl]-1H-pyrazole-4-carboxamide, (2.67) 3-(difluoromethyl)-N-methoxy-1-methyl-N-[1-(2,4,6-trichlorophenyl)propan-2-yl]-1H-pyrarazole-4-carboxamide, (2.68) 3-(difluoromethyl)-N-(7-fluoro-1,1,3-trimethyl-2,3-dihydro-1H-inden-4-yl)-1-methyl-1H-pyrazole-4-carboxamide, (2.69) 3-(difluoromethyl)-N-[(3R)-7-fluoro-1,1,3-trimethyl-2,3-dihydro-1H-inden-4-yl]-1-methyl-1H-pyrazole-4-carboxamide, (2.70) 3-(difluoromethyl)-N-[(3S)-7-fluoro-1,1,3-trimethyl-2,3-dihydro-1H-inden-4-yl]-1-methyl-1H-pyrazole-4-carboxamide.

3) Inhibitors of the respiratory chain on complex III, for example (3.01) ametoctradin, (3.02) amisulbrom, (3.03) azoxystrobin, (3.04) cyazofamid, (3.05) coumethoxystrobin, (3.06) coumoxystrobin, (3.07) dimoxystrobin, (3.08) enoxastrobin, (3.09) famoxadon, (3.10) fenamidon, (3.11) flufenoxystrobin, (3.12) fluoxastrobin, (3.13) kresoxim-methyl, (3.14) metominostrobin, (3.15) orysastrobin, (3.16) picoxystrobin, (3.17) pyraclostrobin, (3.18) pyrametostrobin, (3.19) pyraoxystrobin, (3.20) pyribencarb, (3.21) triclopyricarb, (3.22) trifloxystrobin, (3.23) (2E)-2-(2-{[6-(3-chloro-2-methylphenoxy)-5-fluoropyrimidin-4-yl]oxy}phenyl)-2-(methoxyimino)-N-methylacetamide, (3.24) (2E)-2-(methoxyimino)-N-methyl-2-(2-{[({(1E)-1-[3-(trifluoromethyl)phenyl]ethyliden}amino)oxy]methyl}phenyl)acetamide, (3.25) (2E)-2-(methoxyimino)-N-methyl-2-{2-[(E)-({1-[3-(trifluoromethyl)phenyl]ethoxy}imino)methyl]phenyl}acetamide,

(3.26) (2E)-2-{2-[({[(1E)-1-(3-{[(E)-1-fluoro-2-phenylvinyl]oxy}phenyl)ethyliden]amino}oxy)methyl]phenyl}-2-(methoxyimino)-N-methylacetamide, (3.27) fenaminostrobin, (3.28) 5-methoxy-2-methyl-4-(2-{[({(1E)-1-[3-(trifluoromethyl)phenyl]ethyliden}amino)oxy]methyl}phenyl)-2,4-dihydro-3H-1,2,4-triazol-3-one, (3.29) (2E)-2-{2-[({cyclopropyl[(4-methoxyphenyl)imino]methyl}sulfanyl)methyl]phenyl}-3-methoxyacrylic acid methyl ester, (3.30) N-(3-ethyl-3,5,5-trimethylcyclohexyl)-3-formamido-2-hydroxybenzamide, (3.31) 2-{2-[(2,5-dimethylphenoxy)methyl]phenyl}-2-methoxy-N-methylacetamide, (3.32) 2-{2-[(2,5-dimethylphenoxy)methyl]phenyl}-2-methoxy-N-methylacetamide, (3.33) (2E,3Z)-5-{[1-(4-chlorophenyl)-1H-pyrazol-3-yl]oxy}-2-(methoxyimino)-N,3-dimethylpent-3-enamide.

4) Inhibitors of mitosis and cell division, for example (4.01) benomyl, (4.02) carbendazim, (4.03) chlorfenazole, (4.04) diethofencarb, (4.05) ethaboxam, (4.06) fluopicolide, (4.07) fuberidazole, (4.08) pencycuron, (4.09) thiabendazole, (4.10) thiophanate-methyl, (4.11) thiophanate, (4.12) zoxamide, (4.13) 5-chloro-7-(4-methylpiperidin-1-yl)-6-(2,4,6-trifluorophenyl)[1,2,4]triazolo[1,5-a]pyrimidine, (4.14) 3-chloro-5-(6-chloropyridin-3-yl)-6-methyl-4-(2,4,6-trifluorophenyl)pyridazine.

5) Compounds capable of having multisite action, for example (5.01) Bordeaux mixture, (5.02) captafol, (5.03) captan, (5.04) chlorothalonil, (5.05) copper hydroxide, (5.06) copper naphthenate, (5.07) copper oxide, (5.08) copper oxychloride, (5.09) copper(2+) sulfate, (5.10) dichlofluanid, (5.11) dithianon, (5.12) dodine, (5.13) dodine free base, (5.14) ferbam, (5.15) fluorofolpet, (5.16) folpet, (5.17) guazatine, (5.18) guazatine acetate, (5.19) iminoctadine, (5.20) iminoctadine albesilate, (5.21) iminoctadine triacetate, (5.22) mancopper, (5.23) mancozeb, (5.24) maneb, (5.25) metiram, (5.26) metiram zinc, (5.27) oxine-copper, (5.28) propamidine, (5.29) propineb, (5.30) sulfur and sulfur preparations including calcium polysulfide, (5.31) thiram, (5.32) tolylfluanid, (5.33) zineb, (5.34) ziram, (5.35) anilazine.

6) Compounds capable of inducing host defence, for example (6.01) acibenzolar-S-methyl, (6.02) isotianil, (6.03) probenazole, (6.04) tiadinil, (6.05) laminarin.

7) Inhibitors of the amino acid and/or protein biosynthesis, for example (7.01) andoprim, (7.02) blasticidin-S, (7.03) cyprodinil, (7.04) kasugamycin, (7.05) kasugamycin hydrochloride hydrate, (7.06) mepanipyrim, (7.07) pyrimethanil, (7.08) 3-(5-fluoro-3,3,4,4-tetramethyl-3,4-dihydroisoquinolin-1-yl)quinoline, (7.09) oxytetracycline, (7.10) streptomycin.

8) Inhibitors of ATP production, for example (8.01) fentin acetate, (8.02) fentin chloride, (8.03) fentin hydroxide, (8.04) silthiofam.

9) Inhibitors of cell wall synthesis, for example (9.01) benthiavalicarb, (9.02) dimethomorph, (9.03) flumorph, (9.04) iprovalicarb, (9.05) mandipropamid, (9.06) polyoxins, (9.07) polyoxorim, (9.08) validamycin A, (9.09) valifenalate, (9.10) polyoxin B, (9.11) (2E)-3-(4-tert-butylphenyl)-3-(2-chloropyridin-4-yl)-1-(morpholin-4-yl)prop-2-en-1-one, (9.12) (2Z)-3-(4-tert-butylphenyl)-3-(2-chloropyridin-4-yl)-1-(morpholin-4-yl)prop-2-en-1-one.

10) Inhibitors of lipid and membrane synthesis, for example (10.01) biphenyl, (10.02) chloroneb, (10.03) dicloran, (10.04) edifenphos, (10.05) etridiazole, (10.06) iodocarb, (10.07) iprobenfos, (10.08) isoprothiolane, (10.09) propamocarb, (10.10) propamocarb hydrochloride, (10.11) prothiocarb, (10.12) pyrazophos, (10.13) quintozene, (10.14) tecnazene, (10.15) tolclofos-methyl.

11) Inhibitors of melanin biosynthesis, for example (11.01) carpropamid, (11.02) diclocymet, (11.03) fenoxanil, (11.04) phthalide, (11.05) pyroquilon, (11.06) tricyclazole, (11.07) 2,2,2-trifluoroethyl {3-methyl-1-[(4-methylbenzoyl)amino]butan-2-yl}carbamate.

12) Inhibitors of nucleic acid synthesis, for example (12.01) benalaxyl, (12.02) benalaxyl-M (kiralaxyl), (12.03) bupirimate, (12.04) clozylacon, (12.05) dimethirimol, (12.06) ethirimol, (12.07) furalaxyl, (12.08) hymexazole, (12.09) metalaxyl, (12.10) metalaxyl-M (mefenoxam), (12.11) ofurace, (12.12) oxadixyl, (12.13) oxolinic acid, (12.14) octhilinone.

13) Inhibitors of signal transduction, for example (13.01) chlozolinate, (13.02) fenpiclonil, (13.03) fludioxonil, (13.04) iprodione, (13.05) procymidone, (13.06) quinoxyfen, (13.07) vinclozolin, (13.08) proquinazid.

14) Compounds capable of acting as uncouplers, for example (14.01) binapacryl, (14.02) dinocap, (14.03) ferimzone, (14.04) fluazinam, (14.05) meptyldinocap.

15) Further compounds, for example (15.001) benthiazole, (15.002) bethoxazin, (15.003) capsimycin, (15.004) carvone, (15.005) quinomethionate, (15.006) pyriofenone (chlazafenone), (15.007) cufraneb, (15.008) cyflufenamid, (15.009) cymoxanil, (15.010) cyprosulfamide, (15.011) dazomet, (15.012) debacarb, (15.013) dichlorophen, (15.014) diclomezin, (15.015) difenzoquat, (15.016) difenzoquat metilsulfate, (15.017) diphenylamine, (15.018) Ecomate, (15.019) fenpyrazamine, (15.020) flumetover, (15.021) fluoroimide, (15.022) flusulfamide, (15.023) flutianil, (15.024) fosetyl-aluminium, (15.025) fosetyl-calcium, (15.026) fosetyl-sodium, (15.027) hexachlorobenzene, (15.028) irumamycin, (15.029) methasulfocarb, (15.030) methyl isothiocyanate, (15.031) metrafenone, (15.032) mildiomycin, (15.033) natamycin, (15.034) nickel dimethyldithiocarbamate, (15.035) nitrothal-isopropyl,

(15.036) oxamocarb, (15.037) oxyfenthiin, (15.038) pentachlorophenol and salts, (15.039) phenothrin, (15.040) phosphorous acid and salts thereof, (15.041) propamocarb-fosetylate, (15.042) propanosin-sodium, (15.043) pyrimorph, (15.044) pyrrolnitrin, (15.045) tebufloquin, (15.046) tecloftalam, (15.047) tolnifanid, (15.048) triazoxide, (15.049) trichlamid, (15.050) zarilamid, (15.051) 2-methylpropanoic acid (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl ester, (15.052) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.053) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.054) oxathiapiproline,

(15.055) 1H-imidazole-1-carboxylic acid 1-(4-methoxyphenoxy)-3,3-dimethylbutan-2-yl ester, (15.056) 2,3,5,6-tetrachloro-4-(methylsulfonyl)pyridine, (15.057) 2,3-dibutyl-6-chlorothieno[2,3-d]pyrimidin-4(3H)-one, (15.058) 2,6-dimethyl-1H,5H-[1,4]dithiino[2,3-c:5,6-c′]dipyrrole-1,3,5,7(2H,6H)-tetrone, (15.059) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, (15.060) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, (15.061) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, (15.062) 2-butoxy-6-iodo-3-propyl-4H-chromen-4-one, (15.063) 2-chloro-5-[2-chloro-1-(2,6-difluoro-4-methoxyphenyl)-4-methyl-1H-imidazol-5-yl]pyridine, (15.064) 2-phenylphenol and salts,

(15.065) 3-(4,4,5-trifluoro-3,3-dimethyl-3,4-dihydroisoquinolin-1-yl)quinoline, (15.066) 3,4,5-trichloropyridine-2,6-dicarboxylic acid nitrile, (15.067) 3-chloro-5-(4-chlorophenyl)-4-(2,6-difluorophenyl)-6-methylpyridazine, (15.068) 4-(4-chlorophenyl)-5-(2,6-difluorophenyl)-3,6-dimethylpyridazine, (15.069) 5-amino-1,3,4-thiadiazole-2-thiol, (15.070) 5-chloro-N′-phenyl-N′-(prop-2-in-1-yl)thiophene-2-sulfonohydrazide, (15.071) 5-fluoro-2-[(4-fluorobenzyl)oxy]pyrimidin-4-amine, (15.072) 5-fluoro-2-[(4-methylbenzyl)oxy]pyrimidin-4-amin, (15.073) 5-methyl-6-octyl[1,2,4]triazolo[1,5-a]pyrimidin-7-amin, (15.074) (2Z)-3-amino-2-cyano-3-phenylacrylic acid ethyl ester, (15.075) N′-(4-{[3-(4-chlorobenzyl)-1,2,4-thiadiazol-5-yl]oxy}-2,5-dimethylphenyl)-N-ethyl-N-methylimidoformamide, (15.076) N-(4-chlorobenzyl)-3-[3-methoxy-4-(prop-2-in-1-yloxy)phenyl]propanamide, (15.077) N-[(4-chlorophenyl)(cyano)methyl]-3-[3-methoxy-4-(prop-2-in-1-yloxy)phenyl]propanamide,

(15.078) N-[(5-bromo-3-chloropyridin-2-yl)methyl]-2,4-dichloronicotinamide, (15.079) N-[1-(5-bromo-3-chloropyridin-2-yl)ethyl]-2,4-dichloronicotinamide, (15.080) N-[1-(5-bromo-3-chloropyridin-2-yl)ethyl]-2-fluoro-4-iodonicotinamide, (15.081) N-{(E)-[(cyclopropylmethoxy)imino][6-(difluoromethoxy)-2,3-difluorophenyl]methyl}-2-phenylacetamide, (15.082) N-{(Z)-[(cyclopropylmethoxy)imino][6-(difluoromethoxy)-2,3-difluorophenyl]methyl}-2-phenylacetamide, (15.083) N′-{4-[(3-tert-butyl-4-cyano-1,2-thiazol-5-yl)oxy]-2-chloro-5-methylphenyl}-N-ethyl-N-methylimidoformamide, (15.084) N-methyl-2-(1-{[5-methyl-3-(trifluoromethyl)-1H-pyrazol-1-yl]acetyl}piperidin-4-yl)-N-(1,2,3,4-tetrahydronaphthalin-1-yl)-1,3-thiazole-4-carboxamide, (15.085) N-methyl-2-(1-{[5-methyl-3-(trifluoromethyl)-1H-pyrazol-1-yl]acetyl}piperidin-4-yl)-N-[(1R)-1,2,3,4-tetrahydronaphthalin-1-yl]-1,3-thiazole-4-carboxamide,

(15.086) N-methyl-2-(1-{[5-methyl-3-(trifluoromethyl)-1H-pyrazol-1-yl]acetyl}piperidin-4-yl)-N-[(1S)-1,2,3,4-tetrahydronaphthalin-1-yl]-1,3-thiazole-4-carboxamide, (15.087) {6-[({[(1-methyl-1H-tetrazol-5-yl)(phenyl)methylen]amino}oxy)methyl]pyridin-2-yl}carbamic acid pentyl ester, (15.088) phenazine-1-carboxylic acid, (15.089) quinolin-8-ol, (15.090) quinolin-8-ol sulfate (2:1), (15.091) {6-[({[(1-methyl-1H-tetrazol-5-yl)(phenyl)methylen]amino}oxy)methyl]pyridin-2-yl}carbamic acid tert-butyl ester, (15.092) (5-bromo-2-methoxy-4-methylpyridin-3-yl)(2,3,4-trimethoxy-6-methylphenyl)methanone, (15.093) N-[2-(4-{[3-(4-chlorophenyl)prop-2-yn-1-yl]oxy}-3-methoxyphenyl)ethyl]-N2-(methylsulfonyl)valinamide, (15.094) 4-oxo-4-[(2-phenylethyl)amino]butanoic acid, (15.095) {6-[({[(Z)-(1-methyl-1H-tetrazol-5-yl)(phenyl)methylen]amino}oxy)methyl]pyridin-2-yl}carbamic acid but-3-yn-1-yl ester,

(15.096) 4-amino-5-fluoropyrimidin-2-ol (tautomeric form: 4-amino-5-fluoropyrimidin-2(1H)-one), (15.097) 3,4,5-trihydroxybenzoic acid propyl ester, (15.098) [3-(4-chloro-2-fluorophenyl)-5-(2,4-difluorophenyl)-1,2-oxazol-4-yl](pyridin-3-yl)methanol, (15.099) (S)-[3-(4-chloro-2-fluorophenyl)-5-(2,4-difluorophenyl)-1,2-oxazol-4-yl](pyridin-3-yl)methanol, (15.100) (R)-[3-(4-chloro-2-fluorophenyl)-5-(2,4-difluorophenyl)-1,2-oxazol-4-yl](pyridin-3-yl)methanol, (15.101) 2-fluoro-6-(trifluoromethyl)-N-(1,1,3-trimethyl-2,3-dihydro-1H-inden-4-yl)benzamide, (15.102) 2-(6-benzylpyridin-2-yl)quinazoline, (15.103) 2-[6-(3-fluoro-4-methoxyphenyl)-5-methylpyridin-2-yl]quinazoline, (15.104) 3-(4,4-difluoro-3,3-dimethyl-3,4-dihydroisoquinolin-1-yl)quinoline, (15.105) abscisic acid, (15.106) N′-[5-bromo-6-(2,3-dihydro-1H-inden-2-yloxy)-2-methylpyridin-3-yl]-N-ethyl-N-methylimidoformamide, (15.107) N′-{5-bromo-6-[1-(3,5-difluorophenyl)ethoxy]-2-methylpyridin-3-yl}-N-ethyl-N-methylimidoformamide,

(15.108) N′-{5-bromo-6-[(1R)-1-(3,5-difluorophenyl)ethoxy]-2-methylpyridin-3-yl}-N-ethyl-N-methylimidoformamide, (15.109) N′-{5-bromo-6-[(1S)-1-(3,5-difluorophenyl)ethoxy]-2-methylpyridin-3-yl}-N-ethyl-N-methylimidoformamide, (15.110) N′-{5-bromo-6-[(cis-4-isopropylcyclohexyl)oxy]-2-methylpyridin-3-yl}-N-ethyl-N-methylimidoformamide, (15.111) N′-{5-bromo-6-[(trans-4-isopropylcyclohexyl)oxy]-2-methylpyridin-3-yl}-N-ethyl-N-methylimidoformamide, (15.112) N-cyclopropyl-3-(difluoromethyl)-5-fluoro-N-(2-isopropylbenzyl)-1-methyl-1H-pyrazole-4-carboxamide, (15.113) N-cyclopropyl-N-(2-cyclopropylbenzyl)-3-(difluoromethyl)-5-fluoro-1-methyl-1H-pyrazole-4-carboxamide, (15.114) N-(2-tert-butylbenzyl)-N-cyclopropyl-3-(difluoromethyl)-5-fluoro-1-methyl-1H-pyrazole-4-carboxamide, (15.115) N-(5-chloro-2-ethylbenzyl)-N-cyclopropyl-3-(difluoromethyl)-5-fluoro-1-methyl-1H-pyrazole-4-carboxamide,

(15.116) N-(5-chloro-2-isopropylbenzyl)-N-cyclopropyl-3-(difluoromethyl)-5-fluoro-1-methyl-1H-pyrazole-4-carboxamide, (15.117) N-cyclopropyl-3-(difluoromethyl)-N-(2-ethyl-5-fluorobenzyl)-5-fluoro-1-methyl-1H-pyrazole-4-carboxamide, (15.118) N-cyclopropyl-3-(difluoromethyl)-5-fluoro-N-(5-fluoro-2-isopropylbenzyl)-1-methyl-1H-pyrazole-4-carboxamide, (15.119) N-cyclopropyl-N-(2-cyclopropyl-5-fluorobenzyl)-3-(difluoromethyl)-5-fluoro-1-methyl-1H-pyrazole-4-carboxamide,

(15.120) N-(2-cyclopentyl-5-fluorobenzyl)-N-cyclopropyl-3-(difluoromethyl)-5-fluoro-1-methyl-1H-pyrazole-4-carboxamide, (15.121) N-cyclopropyl-3-(difluoromethyl)-5-fluoro-N-(2-fluoro-6-isopropylbenzyl)-1-methyl-1H-pyrazole-4-carboxamide, (15.122) N-cyclopropyl-3-(difluoromethyl)-N-(2-ethyl-5-methylbenzyl)-5-fluoro-1-methyl-1H-pyrazole-4-carboxamide, (15.123) N-cyclopropyl-3-(difluoromethyl)-5-fluoro-N-(2-isopropyl-5-methylbenzyl)-1-methyl-1H-pyrazole-4-carboxamide,

(15.124) N-cyclopropyl-N-(2-cyclopropyl-5-methylbenzyl)-3-(difluoromethyl)-5-fluoro-1-methyl-1H-pyrazole-4-carboxamide, (15.125) N-(2-tert-butyl-5-methylbenzyl)-N-cyclopropyl-3-(difluoromethyl)-5-fluoro-1-methyl-1H-pyrazole-4-carboxamide, (15.126) N-[5-chloro-2-(trifluoromethyl)benzyl]-N-cyclopropyl-3-(difluoromethyl)-5-fluoro-1-methyl-1H-pyrazole-4-carboxamide, (15.127) N-cyclopropyl-3-(difluoromethyl)-5-fluoro-1-methyl-N-[5-methyl-2-(trifluoromethyl)benzyl]-1H-pyrazole-4-carboxamide, (15.128) N-[2-chloro-6-(trifluoromethyl)benzyl]-N-cyclopropyl-3-(difluoromethyl)-5-fluoro-1-methyl-1H-pyrazole-4-carboxamide, (15.129) N-[3-chloro-2-fluoro-6-(trifluoromethyl)benzyl]-N-cyclopropyl-3-(difluoromethyl)-5-fluoro-1-methyl-1H-pyrazole-4-carboxamide, (15.130) N-cyclopropyl-3-(difluoromethyl)-N-(2-ethyl-4,5-dimethylbenzyl)-5-fluoro-1-methyl-1H-pyrazole-4-carboxamide, (15.131) N-cyclopropyl-3-(difluoromethyl)-5-fluoro-N-(2-isopropylbenzyl)-1-methyl-1H-pyrazole-4-carbothioamide,

(15.132) N′-(2,5-dimethyl-4-phenoxyphenyl)-N-ethyl-N-methylimidoformamide, (15.133) N′-{4-[(4,5-dichloro-1,3-thiazol-2-yl)oxy]-2,5-dimethylphenyl}-N-ethyl-N-methylimidoformamide, (15.134) N-(4-chloro-2,6-difluorophenyl)-4-(2-chloro-4-fluorophenyl)-1,3-dimethyl-1H-pyrazole-5-amine, (15.135) 9-fluoro-2,2-dimethyl-5-(quinolin-3-yl)-2,3-dihydro-1,4-benzoxazepine, (15.136) 2-{2-fluoro-6-[(8-fluoro-2-methylquinolin-3-yl)oxy]phenyl}propan-2-ol, (15.137) 2-{2-[(7,8-difluoro-2-methylquinolin-3-yl)oxy]-6-fluorophenyl}propan-2-ol, (15.138) 4-(2-chloro-4-fluorophenyl)-N-(2-fluorophenyl)-1,3-dimethyl-1H-pyrazole-5-amine, (15.139) 4-(2-chloro-4-fluorophenyl)-N-(2,6-difluorophenyl)-1,3-dimethyl-1H-pyrazole-5-amine, (15.140) 4-(2-chloro-4-fluorophenyl)-N-(2-chloro-6-fluorophenyl)-1,3-dimethyl-1H-pyrazole-5-amine, (15.141) 4-(2-bromo-4-fluorophenyl)-N-(2-chloro-6-fluorophenyl)-1,3-dimethyl-1H-pyrazole-5-amine,

(15.142) N-(2-bromo-6-fluorophenyl)-4-(2-chloro-4-fluorophenyl)-1,3-dimethyl-1H-pyrazole-5-amine, (15.143) 4-(2-bromo-4-fluorophenyl)-N-(2-bromophenyl)-1,3-dimethyl-1H-pyrazole-5-amine, (15.144) 4-(2-bromo-4-fluorophenyl)-N-(2-bromo-6-fluorophenyl)-1,3-dimethyl-1H-pyrazole-5-amine, (15.145) 4-(2-bromo-4-fluorophenyl)-N-(2-chlorophenyl)-1,3-dimethyl-1H-pyrazole-5-amine, (15.146) N-(2-bromphenyl)-4-(2-chloro-4-fluorophenyl)-1,3-dimethyl-1H-pyrazole-5-amine, (15.147) 4-(2-chloro-4-fluorophenyl)-N-(2-chlorophenyl)-1,3-dimethyl-1H-pyrazole-5-amine, (15.148) 4-(2-bromo-4-fluorophenyl)-N-(2,6-difluorophenyl)-1,3-dimethyl-1H-pyrazole-5-amine, (15.149) 4-(2-bromo-4-fluorophenyl)-N-(2-fluorophenyl)-1,3-dimethyl-1H-pyrazole-5-amine, (15.150) N′-(4-{3-[(difluoromethyl)sulfanyl]phenoxy}-2,5-dimethylphenyl)-N-ethyl-N-methylimidoformamide, (15.151) N′-(2,5-dimethyl-4-{3-[(1,1,2,2-tetrafluoroethyl)sulfanyl]phenoxy}phenyl)-N-ethyl-N-methylimidoformamide,

(15.152) N′-(2,5-dimethyl-4-{3-[(2,2,2-trifluoroethyl)sulfanyl]phenoxy}phenyl)-N-ethyl-N-methylimidoformamide, (15.153) N′-(2,5-dimethyl-4-{3-[(2,2,3,3-tetrafluoropropyl)sulfanyl]phenoxy}phenyl)-N-ethyl-N-methylimidoformamide, (15.154) N′-(2,5-dimethyl-4-{3-[(pentafluoroethyl)sulfanyl]phenoxy}phenyl)-N-ethyl-N-methylimidoformamide,

(15.155) N′-(4-{[3-(difluoromethoxy)phenyl]sulfanyl}-2,5-dimethylphenyl)-N-ethyl-N-methylimidoformamide, (15.156) N′-(2,5-dimethyl-4-{[3-(1,1,2,2-tetrafluoroethoxy)phenyl]sulfanyl}phenyl)-N-ethyl-N-methylimidoformamide, (15.157) N′-(2,5-dimethyl-4-{[3-(2,2,2-trifluoroethoxy)phenyl]sulfanyl}phenyl)-N-ethyl-N-methylimidoformamide,

(15.158) N′-(2,5-dimethyl-4-{[3-(2,2,3,3-tetrafluoropropoxy)phenyl]sulfanyl}phenyl)-N-ethyl-N-methylimidoformamide, (15.159) N′-(2,5-dimethyl-4-{[3-(pentafluoroethoxy)phenyl]sulfanyl}phenyl)-N-ethyl-N-methylimidoformamide,

(15.160) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.161) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.162) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.163) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenylmethanesulfonate, (15.164) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethanesulfonate, (15.165) 2-[3,5-bis(difluoromethyl)-1H-pyrazol-1-yl]-1-[4-(4-{(5S)-5-[2-(prop-2-yn-1-yloxy)phenyl]-4,5-dihydro-1,2-oxazol-3-yl}-1,3-thiazol-2-yl)piperidin-1-yl]ethanone,

(15.166) 2-[3,5-bis(difluoromethyl)-1H-pyrazol-1-yl]-1-[4-(4-{(5R)-5-[2-(prop-2-yn-1-yloxy)phenyl]-4,5-dihydro-1,2-oxazol-3-yl}-1,3-thiazol-2-yl)piperidin-1-yl]ethanone, (15.167) 2-[3,5-bis(difluoromethyl)-1H-pyrazol-1-yl]-1-[4-(4-{(5S)-5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.168) 2-[3,5-bis(difluoromethyl)-1H-pyrazol-1-yl]-1-[4-(4-{(5R)-5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.169) 2-[3,5-bis(difluoromethyl)-1H-pyrazol-1-yl]-1-[4-(4-{(5S)-5-[2-chloro-6-(prop-2-yn-1-yloxy)phenyl]-4,5-dihydro-1,2-oxazol-3-yl}-1,3-thiazol-2-yl)piperidin-1-yl]ethanone, (15.170) 2-[3,5-bis(difluoromethyl)-1H-pyrazol-1-yl]-1-[4-(4-{(5R)-5-[2-chloro-6-(prop-2-yn-1-yloxy)phenyl]-4,5-dihydro-1,2-oxazol-3-yl}-1,3-thiazol-2-yl)piperidin-1-yl]ethanone,

(15.171) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenylmethanesulfonate, (15.172) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenylmethanesulfonate, (15.173) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethanesulfonate, (15.174) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethanesulfonate.

Biological Pesticides as Mixing Components

The compounds of the formula (I) can be combined with biological pesticides.

Biological pesticides include especially bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are: Bacillus amyloliquefaciens, strain FZB42 (DSM 231179), or Bacillus cereus, especially B. cereus strain CNCM 1-1562 or Bacillus firmus, strain 1-1582 (Accession number CNCM 1-1582) or Bacillus pumilus, especially strain GB34 (Accession No. ATCC 700814) and strain QST2808 (Accession No. NRRL B-30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421), Bacillus thuringiensis, especially B. thuringiensis subspecies israelensis (serotype H-14), strain AM65-52 (Accession No. ATCC 1276),

or B. thuringiensis subsp. aizawai, in particular strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii, (formerly known as Verticillium lecanii), in particular strain KVO1, Metarhizium anisopliae, in particular strain F52 (DSM3884/ATCC 90448), Metschnikowia fructicola, in particular strain NRRL Y-30752, Paecilomyces fumosoroseus (new: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC1 (Accession Number CBS 122089), Trichoderma harzianum, in particular T. harzianum rifai T39 (Accession Number CNCM 1-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), Pyrethrum/Pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

Safeners as Mixing Components

The compounds of the formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulfonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

Plants and Plant Parts

All plants and plant parts can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya beans, potatoes, sugar beet, sugar cane, tomatoes, peas and other types of vegetable, cotton, tobacco, oilseed rape, and also fruit plants (with the fruits apples, pears, citrus fruits and grapevines). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights.

Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested material and vegetative and generative propagation material, for example cuttings, tubers, rhizomes, slips and seeds.

The treatment according to the invention of the plants and parts of plants with the compounds of the formula (I) is carried out directly or by allowing them to act on the surroundings, habitat or storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and parts thereof in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use.

Plant cultivars are understood to mean plants having new properties (“traits”) and which have been grown by conventional breeding, by mutagenesis or by recombinant DNA techniques. They may be cultivars, varieties, biotypes or genotypes.

Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher yields, higher quality and/or higher nutritional value of the harvested products, better storage life and/or processability of the harvested products.

Further and particularly emphasized examples of such properties are increased resistance of the plants against animal and microbial pests, such as insects, arachnids, nematodes, mites, slugs and snails, owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof), and also increased resistance of the plants against phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain active herbicidal compounds,

for example imidazolinones, sulfonylureas, glyphosates or phosphinothricin (for example the “PAT” gene). The genes which impart the desired properties (“traits”) in question may also be present in combinations with one another in the transgenic plants. Examples of transgenic plants mentioned include the important crop plants, such as cereals (wheat, rice, triticale, barley, rye, oats), maize, soybeans, potatoes, sugar beet, sugar cane, tomatoes, peas and other types of vegetable, cotton, tobacco, oilseed rape and also fruit plants (with the fruits apples, pears, citrus fruits and grapevines), particular emphasis being given to maize, soybeans, wheat, rice, potatoes, cotton, sugar cane, tobacco and oilseed rape. Properties (“traits”) which are particularly emphasized are the increased resistance of the plants to insects, arachnids, nematodes and slugs and snails.

Crop Protection—Types of Treatment

The plants and plant parts are treated with the compounds of the formula (I) directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, additionally by dry seed treatment, liquid seed treatment, slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, i.e. compounds of the formula (I) are applied to the foliage, where treatment frequency and the application rate should be adjusted according to the level of infestation with the pest in question.

In the case of systemically active compounds, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) are introduced in solid form (e.g. in the form of granules) into the locus of the plants. In the case of paddy rice crops, this can also be accomplished by metering the compound of the formula (I) in a solid application form (for example as granules) into a flooded paddy field.

Seed Treatment

The control of animal pests by the treatment of the seed of plants has long been known and is the subject of constant improvements. Nevertheless, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants.

It is additionally desirable to optimize the amount of active ingredient used so as to provide optimum protection for the seed and the germinating plant from attack by animal pests, but without damage to the plant itself by the active ingredient used. In particular, methods for the treatment of seed should also take account of the intrinsic insecticidal or nematicidal properties of pest-resistant or -tolerant transgenic plants in order to achieve optimal protection of the seed and the germinating plant with a minimum expenditure on pesticides.

The present invention therefore in particular also relates to a method for the protection of seed and germinating plants from attack by pests, by treating the seed with one of the compounds of the formula (I). The method according to the invention for protecting seed and germinating plants against attack by pests further comprises a method in which the seed is treated simultaneously in one operation or sequentially with a compound of the formula (I) and a mixing component. It further also comprises a method where the seed is treated at different times with a compound of the formula (I) and a mixing component.

The invention likewise relates to the use of the compounds of the formula (I) for the treatment of seed for protecting the seed and the resulting plant from animal pests.

The invention further relates to seed which has been treated with a compound of the formula (I) for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and a mixing component may optionally be separated by an intermediate layer.

The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages encountered with a systemically acting compound of the formula (I) is the fact that, by treating the seed, not only the seed itself but also the plants resulting therefrom are, after emergence, protected against animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be employed in combination with compositions of signalling technology, leading to better colonization by symbionts such as, for example, rhizobia, mycorrhizae and/or endophytic bacteria or fungi, and/or to optimized nitrogen fixation.

The compounds of the formula (I) are suitable for the protection of seed of any plant variety which is used in agriculture, in greenhouses, in forests or in horticulture. More particularly, this includes seed of cereals (for example wheat, barley, rye, millet and oats), maize, cotton, soya beans, rice, potatoes, sunflowers, coffee, tobacco, canola, oilseed rape, beet (for example sugar beet and fodder beet), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate in this case from microorganisms such as Bacillus, Rhizobium, Pseudomonas, Serratia, Trichoderma, Clavibacter, Glomus or Gliocladium. The present invention is particularly suitable for the treatment of transgenic seed containing at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. Thus, for example, it is possible to use seed which has been harvested, cleaned and dried down to a moisture content which allows storage.

Alternatively, it is also possible to use seed which, after drying, has been treated with, for example, water and then dried again, for example priming. In the case of rice seed, it is also possible to use seed which has been imbibed in water up to a certain stage (pigeon breast stage) for example, which leads to improved germination and more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active compounds which can exhibit phytotoxic effects at certain application rates.

In general, the compounds of the formula (I) are applied to the seed in the form of a suitable formulation. Suitable formulations and processes for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are produced in a known manner, by mixing compounds of the formula (I) with customary additives, for example customary extenders and solvents or diluents, dyes, wetters, dispersants, emulsifiers, antifoams, preservatives, secondary thickeners, stickers, gibberellins and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of active agrochemical ingredients. Alkyl naphthalenesulfonates, such as diisopropyl or diisobutyl naphthalenesulfonates, can be used with preference.

Suitable dispersants and/or emulsifiers which may be present in the seed-dressing formulations usable in accordance with the invention are all nonionic, anionic and cationic dispersants customary for the formulation of active agrochemical ingredients. Nonionic or anionic dispersants or mixtures of nonionic or anionic dispersants can be used with preference. Suitable nonionic dispersants include in particular ethylene oxide/propylene oxide block polymers, alkylphenol polyglycol ethers and tristyrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of active agrochemical ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances which can be used for such purposes in agrochemical compositions.

Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz- and Schadlingsbekaimpfungsmittel”, vol. 2, Springer Verlag, 1970, pp. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute use forms thereof, can also be used to dress seed of transgenic plants.

For the treatment of seed with the seed-dressing formulations usable in accordance with the invention, or use forms prepared therefrom, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed-dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

Animal Health

In the animal health field, i.e. the field of veterinary medicine, the compounds of the formula (I) are active against animal parasites, in particular ectoparasites or endoparasites. The term “endoparasites” includes especially helminths and protozoa, such as coccidia. Ectoparasites are typically and preferably arthropods, especially insects and acarids.

In the field of veterinary medicine, the compounds of the formula (I) having favourable endotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals such as sheep, goats, horses, donkeys, camels, buffalo, rabbits, reindeer, fallow deer, and particularly cattle and pigs; poultry such as turkeys, ducks, geese, and particularly chickens; fish and crustaceans, for example in aquaculture, and also insects such as bees.

Domestic animals include, for example, mammals, such as hamsters, guinea pigs, rats, mice, chinchillas, ferrets, and particularly dogs, cats, caged birds, reptiles, amphibians and aquarium fish.

In a preferred embodiment, the compounds of the formula (I) are administered to mammals.

In another preferred embodiment, the compounds of the formula (I) are administered to birds, namely caged birds and particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal husbandry is enabled and better animal well-being is achievable.

In relation to the field of animal health, the term “control” or “controlling” means that the compounds of the formula (I) are effective in reducing the incidence of the particular parasite in an animal infected with such parasites to an innocuous degree. More specifically, “controlling” in the present context means that the compound of the formula (I) can kill the respective parasite, inhibit its growth, or inhibit its proliferation.

Arthropods include:

from the order Anoplurida, for example Haematopinus spp., Linognathus spp., Pediculus spp., Phtirus spp., Solenopotes spp.; from the order Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; from the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order Siphonapterida, for example Pulex spp., Ctenocephalides spp., Tunga spp., Xenopsylla spp., Ceratophyllus spp.; from the order Heteropterida, for example Cimex spp., Triatoma spp., Rhodnius spp., Panstrongylus spp.; and also nuisance and hygiene pests from the order Blattarida.

Arthropods further include:

from the subclass Acari (Acarina) and the order Metastigmata, for example from the family Argasidae like Argas spp., Ornithodorus spp., Otobius spp., from the family Ixodidae like Ixodes spp., Amblyomma spp., Rhipicephalus (Boophilus) spp., Dermacentor spp., Haemophysalis spp., Hyalomma spp., Rhipicephalus spp. (the original genus of multi-host ticks); from the order Mesostigmata like Dermanyssus spp., Omithonyssus spp., Pneumonyssus spp., Raillietia spp., Pneumonyssus spp., Sternostoma spp., Varroa spp., Acarapis spp.; from the order Actinedida (Prostigmata), for example Acarapis spp., Cheyletiella spp., Omithocheyletia spp., Myobia spp., Psorergates spp., Demodex spp., Trombicula spp., Neotrombiculla spp., Listrophorus spp.; and from the order Acaridida (Astigmata), for example Acarus spp., Tyrophagus spp., Caloglyphus spp.,

Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

Parasitic protozoa include:

Mastigophora (Flagellata), for example Trypanosomatidae, for example Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, for example Trichomonadidae, for example Giardia lamblia, G. canis;

Sarcomastigophora (Rhizopoda) such as Entamoebidae, for example Entamoeba histolytica, Hartmanellidae, for example Acanthamoeba sp., Harmanella sp.; Apicomplexa (Sporozoa), such as Eimeridae, for example Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuemii, Globidium spec., Isospora belli, I. canis, I. felis, I. ohioensis, I. rivolta, I. spec., I. suis, Cystisospora spec.,

Cryptosporidium spec., in particular C. parvum; such as Toxoplasmadidae, for example Toxoplasma gondii, Hammondia heydornii, Neospora caninum, Besnoitia besnoitii; such as Sarcocystidae, for example Sarcocystis bovicanis, S. bovihominis, S. ovicanis, S. ovifelis, S. neurona, S. spec., S. suihominis, such as Leucozoidae, for example Leucozytozoon simondi, such as Plasmodiidae, for example Plasmodium berghei, P. falciparum, P. malariae, P. ovale, P. vivax, P. spec., such as Piroplasmea, for example Babesia argentina, B. bovis, B. canis, B. spec., Theileria parva, Theileria spec., such as Adeleina, for example Hepatozoon canis, H. spec.

Pathogenic endoparasites which are helminths include Platyhelmintha (e.g. Monogenea, cestodes and trematodes), nematodes, Acanthocephala, and Pentastoma. These include:

Monogenea: for example: Gyrodactylus spp., Dactylogyrus spp., Polystoma spp.;

Cestodes: from the order of Pseudophyllidea, for example: Diphyllobothrium spp., Spirometra spp., Schistocephalus spp., Ligula spp., Bothridium spp., Diphlogonoporus spp.;

from the order Cyclophyllida, for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.;

Trematodes: from the class of Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fascioloides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.;

Nematodes: Trichinellida, for example: Trichuris spp., Capillaria spp., Paracapillaria spp., Eucoleus spp., Trichomosoides spp., Trichinella spp.,

from the order Tylenchida, for example: Micronema spp., Strongyloides spp.;

from the order Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.;

from the order Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.;

Acanthocephala: from the order of Oligacanthorhynchida, for example: Macracanthorhynchus spp., Prosthenorchis spp.; from the order Polymorphida for example: Filicollis spp.; from the order Moniliformida for example: Moniliformis spp.;

from the order Echinorhynchida, for example Acanthocephalus spp., Echinorhynchus spp., Leptorhynchoides spp.;

Pentastoma: from the order Porocephalida, for example Linguatula spp.

In the veterinary field and in animal husbandry, the compounds of the formula (I) are administered by methods generally known in the art, such as via the enteral, parenteral, dermal or nasal route in the form of suitable preparations. Administration may be prophylactic or therapeutic.

Thus, one embodiment of the present invention refers to the use of a compound of the formula (I) as a medicament.

A further aspect refers to the use of a compound of the formula (I) as an antiendoparasitic agent, in particular a helminthicidal agent or antiprotozoic agent. Compounds of the formula (I) are suitable for use as an antiendoparasitic agent, especially as a helminthicidal agent or antiprotozoic agent, for example in animal breeding, in animal husbandry, in animal houses and in the hygiene sector.

A further aspect in turn relates to the use of a compound of the formula (I) as an antiectoparasitic agent, in particular an arthropodicide such as an insecticide or an acaricide. A further aspect relates to the use of a compound of the formula (I) as an antiectoparasitic agent, in particular an arthropodicide such as an insecticide or an acaricide, for example in animal husbandry, in animal breeding, in animal houses or in the hygiene sector.

Vector Control

The compounds of the formula (I) can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted either mechanically (for example trachoma by non-stinging flies) to a host or after injection (for example malaria parasites by mosquitoes) into a host.

Examples of vectors and the diseases or pathogens they transmit are:
1) mosquitoes
   Anopheles: malaria, filariasis;
   Culex: Japanese encephalitis, filariasis, other viral diseases, transmission of worms;
   Aedes: yellow fever, dengue fever, filariasis, other viral diseases;
   Simuliidae: transmission of worms, in particular Onchocerca volvulus;
2) Lice: skin infections, epidemic typhus;
3) Fleas: plague, endemic typhus;
4) Flies: sleeping sickness (trypanosomiasis); cholera, other bacterial diseases;
5) Mites: acariosis, epidemic typhus, rickettsialpox, tularaemia, Saint Louis encephalitis, tick-borne encephalitis (TBE), Crimean-Congo haemorrhagic fever, borreliosis;
6) Ticks: borellioses such as Borrelia duttoni, tick-borne encephalitis, Q fever (Coxiella burnetii), babesioses (Babesia canis canis).

Examples of vectors in the context of the present invention are insects, such as aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forests, in gardens and in leisure facilities, and also in the protection of materials and stored products.

Protection of Industrial Materials

The compounds of the formula (I) are suitable for protecting industrial materials against attack or destruction by insects, for example from the orders Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) are present as a ready-to-use pesticide, i.e. it can be applied to the material in question without further modifications. Suitable further insecticides or fungicides are in particular those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active compounds, as antifouling agents.

Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids and mites encountered in enclosed spaces, for example dwellings, factory halls, offices, vehicle cabins. For controlling animal pests, the compounds of the formula (I) are used alone or in combination with other active compounds and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or bait stations.

### DESCRIPTION OF THE PROCESSES AND INTERMEDIATES

The preparation or synthesis and use examples which follow illustrate the invention without limiting it. The products were characterized by ¹H NMR spectroscopy and/or LC-MS (Liquid Chromatography Mass Spectrometry).

The log P values were determined in accordance with OECD Guideline 117 (EC Directive 92/69/EEC) by HPLC (high-performance liquid chromatography) using reversed-phase (RP) columns (C18), by the following methods:

[a] The LC-MS determination in the acidic range is carried out at pH 2.7 with 0.1% aqueous formic acid and acetonitrile (contains 0.1% formic acid) as eluents; linear gradient from 10% acetonitrile to 95% acetonitrile.

[b] LC-MS determination in the neutral range is effected at pH 7.8 with 0.001 molar aqueous ammonium hydrogencarbonate solution and acetonitrile as eluents; linear gradient from 10% acetonitrile to 95% acetonitrile.

Calibration is carried out using unbranched alkan-2-ones (having 3 to 16 carbon atoms) with known log P values (log P values determined on the basis of the retention times by linear interpolation between two successive alkanones).

The NMR spectra were determined using a Bruker Avance 400 fitted with a flow probe head (60 μl volume). In individual cases, the NMR spectra were measured with a Bruker Avance II 600.

The ¹H NMR data of selected examples are noted in the form of 1H-NMR peak lists. For each signal peak, first the □ value in ppm and then the signal intensity in round brackets are listed. The pairs of □ value-signal intensity numbers for different signal peaks are listed with separation from one another by semicolons.

The peak list for one example therefore has the form:

□₁(intensity 1);□₂(intensity 2); . . . ;□ᵢ(intensity i); . . . ;□ₙ(intensity n)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional ¹H NMR interpretation.

Further details of ¹H NMR peak lists can be found in the Research Disclosure Database Number 564025.

### Syntheses of 5-substituted 2-(3-pyridyl)thiazolo[5,4-b]pyridines

### Synthesis of 5-chloro-2-(3-pyridyl)thiazolo[5,4-b]pyridine

### Step 1: N-(2,6-Dichloro-3-pyridyl)pyridine-3-carboxamide

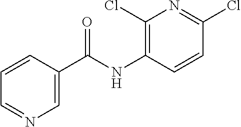 [SMILES: O=C(Nc1ccc(Cl)nc1Cl)c1cccnc1]

At 0° C., a suspension of pyridine-3-carbonyl chloride hydrochloride (16.4 g, 92.0 mmol) in acetonitrile (300 ml) was added to a solution of 3-amino-2,6-dichloropyridine (5.00 g, 30.7 mmol) in acetonitrile (120 ml) and pyridine (20 ml). The reaction mixture was stirred for 16 h at room temperature. Subsequently, water was added and the mixture was made alkaline with aqueous sodium hydroxide solution (1 M) and extracted with ethyl acetate. The combined organic phases were dried with Na₂SO₄ and the solvent was removed under reduced pressure. This gave 7.64 g (100% pure, 93% yield) of N-(2,6-dichloro-3-pyridyl)pyridine-3-carboxamide.

¹H-NMR (400.0 MHz, d₆-DMSO): δ=9.143 (10.4); 9.138 (10.3); 8.814 (6.9); 8.810 (7.7); 8.802 (7.4); 8.798 (7.6); 8.341 (4.0); 8.336 (6.2); 8.331 (4.3); 8.321 (4.4); 8.316 (6.8); 8.311 (4.4); 8.192 (4.4); 8.171 (15.9); 7.683 (16.0); 7.662 (14.9); 7.621 (5.6); 7.609 (5.5); 7.601 (5.4); 7.589 (5.1); 3.335 (158.4); 2.677 (0.6); 2.672 (0.8); 2.668 (0.6); 2.526 (2.0); 2.508 (86.2); 2.503 (113.2); 2.499 (85.0); 2.335 (0.5); 2.330 (0.7); 2.326 (0.5); 1.990 (0.5); 1.259 (0.4); 1.234 (2.9); 0.008 (0.9); −0.000 (25.7); −0.008 (1.1).

### Step 2: 5-Chloro-2-(3-pyridyl)thiazolo[5,4-b]pyridine

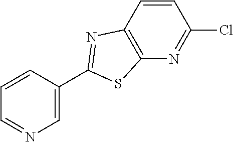 [SMILES: Clc1ccc2nc(-c3cccnc3)sc2n1]

A suspension of N-(2,6-dichloro-3-pyridyl)pyridine-3-carboxamide (881 mg, 3.28 mmol) and 2,4-bis(4-methoxyphenyl)-1,3,2,4-dithiadiphosphetane 2,4-disulfide (1.37 g, 3.28 mmol) in toluene (37 ml) was stirred under reflux for a total of 13 h and at 100° C. for a further 32 h. Subsequently, the solvent was removed under reduced pressure, and CH₂Cl₂ and saturated NaHCO₃ solution were added. The phases were separated, the aqueous phase was extracted with CH₂Cl₂ and the combined organic phases were dried with Na₂SO₄. After the solvent has been removed under reduced pressure, the residue was separated chromatographically by MPLC on silica gel (gradient: ethyl acetate/cyclohexane 0:100→30:70). This gave 325 mg (98% pure, 39% yield) of 5-chloro-2-(3-pyridyl)thiazolo[5,4-b]pyridine.

¹H-NMR (400.0 MHz, d₆-DMSO): δ=9.302 (6.3); 9.300 (7.0); 9.296 (6.9); 9.294 (6.7); 8.814 (6.0); 8.810 (6.6); 8.802 (6.4); 8.798 (6.5); 8.579 (15.0); 8.558 (15.8); 8.506 (3.9); 8.502 (4.6); 8.500 (4.6); 8.496 (3.9); 8.486 (4.1); 8.482 (4.6); 8.480 (5.0); 8.476 (3.9); 8.318 (0.5); 7.761 (16.0); 7.740 (15.1); 7.672 (4.6); 7.670 (4.9); 7.660 (4.5); 7.658 (4.7); 7.652 (4.4); 7.650 (4.6); 7.640 (4.3); 7.638 (4.5); 3.330 (93.2); 2.682 (0.4); 2.677 (0.8); 2.673 (1.1); 2.668 (0.8); 2.664 (0.4); 2.526 (2.7); 2.521 (4.0); 2.513 (58.2); 2.508 (121.6); 2.503 (164.7); 2.499 (121.9); 2.494 (59.5); 2.339 (0.4); 2.335 (0.8); 2.330 (1.1); 2.326 (0.8); 2.321 (0.4); 1.398 (1.6); 1.231 (0.4); 0.000 (1.0).

### Synthesis of 2-(3-pyridyl)-5-(trifluoromethyl)thiazolo[5,4-b]pyridine (Example 10)

### Step 1: N-[2-Chloro-6-(trifluoromethyl)-3-pyridyl]pyridine-3-carboxamide

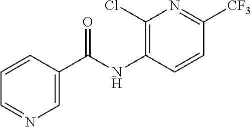 [SMILES: O=C(Nc1ccc(C(F)(F)F)nc1Cl)c1cccnc1]

N-[2-Chloro-6-(trifluoromethyl)-3-pyridyl]pyridine-3-carboxamide was synthesized analogously to the synthesis of N-(2,6-dichloro-3-pyridyl)pyridine-3-carboxamide. To this end, a solution of 2-chloro-6-(trifluoromethyl)pyridine-3-amine (200 mg, 1.02 mmol), pyridine-3-carbonyl chloride hydrochloride (543 mg, 3.05 mmol) and pyridine (0.66 ml, 8.1 mmol) in acetonitrile was stirred at room temperature for 31 h. This gave 218 g (100% pure, 71% yield) of N-[2-chloro-6-(trifluoromethyl)-3-pyridyl]pyridine-3-carboxamide.

¹H-NMR (400.0 MHz, d₆-DMSO): δ=10.696 (12.3); 9.156 (12.2); 9.152 (12.3); 8.833 (8.6); 8.829 (9.1); 8.821 (9.3); 8.817 (8.9); 8.482 (9.6); 8.461 (10.8); 8.359 (5.2); 8.354 (7.1); 8.349 (5.2); 8.339 (5.7); 8.334 (7.4); 8.329 (5.3); 8.318 (0.6); 8.076 (16.0); 8.056 (14.6); 7.640 (6.2); 7.639 (6.2); 7.628 (6.3); 7.627 (6.3); 7.621 (6.3); 7.619 (6.1); 7.609 (5.9); 7.607 (5.7); 4.021 (0.3); 3.333 (126.0); 2.677 (0.8); 2.673 (1.1); 2.668 (0.8); 2.526 (3.0); 2.512 (63.3); 2.508 (121.9); 2.504 (155.9); 2.499 (115.8); 2.495 (59.2); 2.335 (0.8); 2.33 (1.0); 2.326 (0.8); 1.990 (1.4); 1.193 (0.4); 1.176 (0.7); 1.158 (0.4); 0.008 (1.6); −0.000 (37.8); −0.008 (2.0).

### Step 2: 2-(3-Pyridyl)-5-(trifluoromethyl)thiazolo[5,4-b]pyridine (Example 10)

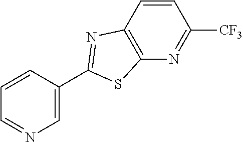 [SMILES: FC(F)(F)c1ccc2nc(-c3cccnc3)sc2n1]

2-(3-Pyridyl)-5-(trifluoromethyl)thiazolo[5,4-b]pyridine was prepared analogously to the synthesis of 5-chloro-2-(3-pyridyl)thiazolo[5,4-b]pyridine. Here, a mixture of N-[2-chloro-6-(trifluoromethyl)-3-pyridyl]pyridine-3-carboxamide (206 mg, 683 mol), 2,4-bis(4-methoxyphenyl)-1,3,2,4-dithiadiphosphetane 2,4-disulfide (283 mg, 700 μmol) and toluene (20 ml) was stirred at 100° C. for 16 h and under reflux for a further 5 h. This gave 116 mg (100% pure, 60% yield) of 2-(3-pyridyl)-5-(trifluoromethyl)thiazolo[5,4-b]pyridine.

¹H-NMR (400.0 MHz, d₆-DMSO): δ=9.354 (8.2); 9.352 (8.9); 9.348 (8.9); 9.346 (8.5); 8.849 (7.2); 8.845 (7.8); 8.837 (7.7); 8.833 (7.7); 8.781 (8.4); 8.780 (8.4); 8.760 (9.1); 8.759 (9.0); 8.561 (4.4); 8.557 (5.5); 8.556 (5.3); 8.551 (4.4); 8.541 (4.8); 8.537 (5.5); 8.535 (5.8); 8.531 (4.5); 8.151 (16.0); 8.130 (15.0); 7.700 (5.4); 7.698 (5.5); 7.688 (5.2); 7.686 (5.3); 7.680 (5.2); 7.678 (5.2); 7.668 (5.1); 7.666 (5.1); 3.338 (38.7); 2.681 (0.3); 2.677 (0.5); 2.672 (0.3); 2.530 (1.2); 2.525 (1.7); 2.517 (26.2); 2.512 (54.4); 2.508 (72.0); 2.503 (51.8); 2.499 (24.7); 2.339 (0.3); 2.334 (0.5); 2.330 (0.3); 1.993 (0.4); 1.225 (1.4); 0.008 (0.9); 0.000 (30.6); −0.009 (1.0).

### Synthesis of methyl 2-(pyridin-3-yl)[1,3]thiazolo[5,4-b]pyridine-5-carboxylate

### Step 1: N-(2,6-Dibromopyridin-3-yl)nicotinamide

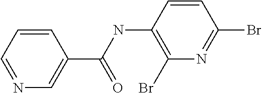 [SMILES: O=C(Nc1ccc(Br)nc1Br)c1cccnc1]

At 0° C., pyridine (18.84 g, 238.17 mmol) and (chloromethylene)dimethylammonium chloride (15.24 g, 119.1 mmol) were added to a solution of pyridine-3-carboxylic acid (9.77 g, 79.4 mmol) in CH₂Cl₂ (500 ml). The mixture was stirred at 0° C. for 30 min, and 2,6-dibromopyridine-3-amine (20.0 g, 79.4 mmol) was then added. The reaction mixture was stirred at room temperature for 16 h, added to a saturated NaHCO₃ solution (500 ml) and extracted with CH₂Cl₂ (2×500 ml). The combined organic phases were washed with water (300 ml) and saturated sodium chloride solution (300 ml), dried with sodium sulfate and filtered, and the solvent was removed under reduced pressure. The residue was purified chromatographically by column chromatography on silica gel (gradient: ethyl acetate/petroleum ether 20:80→33:66). This gave 15.0 g (53% yield) of N-(2,6-dibromopyridin-3-yl)nicotinamide.

### Step 2: 5-Bromo-2-(pyridin-3-yl)[1,3]thiazolo[5,4-b]pyridine

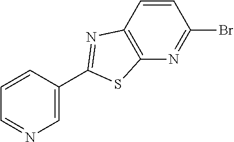 [SMILES: Brc1ccc2nc(-c3cccnc3)sc2n1]

A mixture of N-(2,6-dibromopyridin-3-yl)nicotinamide (20.0 g, 56.0 mmol) and 2,4-bis(4-methoxyphenyl)-1,3,2,4-dithiadiphosphetane 2,4-disulfide (33.99 g, 84.03 mmol) in 1,4-dioxane (300 ml) was stirred at 110° C. for 3 h. The solvent was then removed under reduced pressure and the residue was washed with ethyl acetate (150 ml) and dimethylformamide (30 ml). This gave 13.0 g (79% yield) of 5-bromo-2-(pyridin-3-yl)[1,3]thiazolo[5,4-b]pyridine.

### Step 3: Methyl 2-(pyridin-3-yl)[1,3]thiazolo[5,4-b]pyridine-5-carboxylate

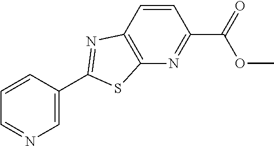 [SMILES: COC(=O)c1ccc2nc(-c3cccnc3)sc2n1]

Triethylamine (13.5 g, 133 mmol) and 1,1′-bis(diphenylphosphino)ferrocene]dichloropalladium(II) (6.51 g, 8.90 mmol) were added to a solution of 5-bromo-2-(pyridin-3-yl)[1,3]thiazolo[5,4-b]pyridine (13.0 g, 44.5 mmol) in THF (300 ml), dimethylformamide (150 ml) and methanol (150 ml). The reaction mixture was stirred under an atmosphere of carbon monoxide (3.1 bar) at 70° C. for 16 h and the solvent was then removed under reduced pressure. The residue was purified chromatographically by column chromatography on silica gel (gradient: ethyl acetate/petroleum ether 50:50→66:33). This gave 4.0 g (33% yield) of methyl 2-(pyridin-3-yl)[1,3]thiazolo[5,4-b]pyridine-5-carboxylate.

MS: m/z 272.0 [M+H⁺]

### General Procedure for the Synthesis of Amides of 2-(pyridin-3-yl)[1,3]thiazolo[5,4-b]pyridine-5-carboxylic Acid

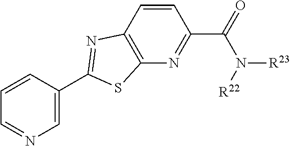

A solution of methyl 2-(pyridin-3-yl)[1,3]thiazolo[5,4-b]pyridine-5-carboxylate (300 mg, 1.11 mmol) and potassium trimethylsilanolate (171 mg, 1.33 mmol) in THF (8 ml) was stirred at 30° C. for 16 h. The solvent was removed under reduced pressure and the residue was taken up in dimethylformamide (5 ml). 1-[Bis(dimethylamine)methylene]-1H-1,2,3-triazole[4,5-b]pyridinium 3-oxide hexafluorophosphate (422 mg, 1.33 mmol) and ethyldiisopropylamine (0.58 ml, 3.33 mmol) were added to this solution and the mixture was stirred at 30° C. for 30 min. The amine in question (1.33 mmol) was then added, the mixture was stirred at 30° C. for a further 16 h and the solvent was removed under reduced pressure. For isolation of the product, the reaction mixture was purified by HPLC.

### Synthesis of 2-(pyridin-3-yl)[1,3]thiazolo[5,4-b]pyridine-5-amine

### Step 1: tert-Butyl [2-(pyridin-3-yl)[1,3]thiazolo[5,4-b]pyridin-5-yl]carbamate

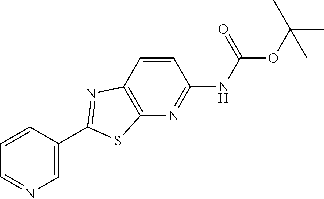 [SMILES: CC(C)(C)OC(=O)Nc1ccc2nc(-c3cccnc3)sc2n1]

Under an atmosphere of nitrogen, a mixture of 5-bromo-2-(pyridin-3-yl)[1,3]thiazolo[5,4-b]pyridine (10.0 g, 34.2 mmol), tert-butyl carbamate (6.63 g, 51.3 mmol), Cs₂CO₃ (33.46 g, 103.7 mmol), 4,5-bis(diphenylphosphino)-9,9-dimethylxanthene (3.96 g, 6.84 mmol) and Pd(OAc)₂ (3.96 g, 6.85 mmol) in 1,4-dioxane (100 ml) was heated at 80° C. for 2 h. The solvent was then removed under reduced pressure. The residue was diluted with water (100 ml) and extracted with ethyl acetate (3×100 ml). The combined organic phases were washed with saturated sodium chloride solution, dried over sodium sulfate and filtered. The solvent was removed under reduced pressure and the residue was separated chromatographically by MPLC on silica gel (gradient: ethyl acetate/cyclohexane 20:80→40:60). This gave 7.00 g (63%) of tert-butyl [2-(pyridin-3-yl)[1,3]thiazolo[5,4-b]pyridin-5-yl]carbamate.

MS: m/z 328.9 [M+H+]

### Step 2: 2-(Pyridin-3-yl)[1,3]thiazolo[5,4-b]pyridin-5-amine

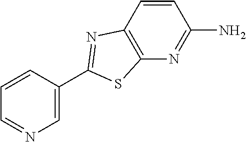 [SMILES: Nc1ccc2nc(-c3cccnc3)sc2n1]

Trifluoroacetic acid (25 ml) was added to a solution of tert-butyl [2-(pyridin-3-yl)[1,3]thiazolo[5,4-b]pyridin-5-yl]carbamate (7.00 g, 21.3 mmol) in CH₂Cl₂ (100 ml). The mixture was stirred at 30° C. for 6 h and the solvent was then removed under reduced pressure. The residue (6.80 g) was used for the next reaction without further purification.

### General Procedure for the Synthesis of Amides of 2-(pyridin-3-yl)[1,3]thiazolo[5,4-b]pyridine-5-amine

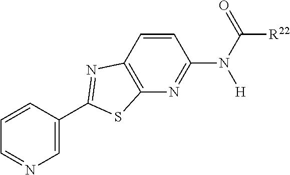

1-Ethyl-3-(3-dimethylaminopropyl)carbodiimide (575 mg, 3.00 mmol) and N,N-dimethylpyridine-4-amine (732 mg, 5.99 mmol) were added to a solution of 2-(pyridin-3-yl)[1,3]thiazolo[5,4-b]pyridine-5-amine (684 mg, 2.00 mmol) and the acid in question (4.00 mmol) in dichloromethane (5 ml). The reaction mixture was stirred at 30° C. for 16 h and the solvent was removed under reduced pressure. Water (10 ml) was added to the residue and the mixture was extracted with ethyl acetate (3×10 ml). The combined organic phases were washed with saturated sodium chloride solution, dried over sodium sulfate and filtered. The solvent was removed under reduced pressure and the residue was separated chromatographically by MPLC on silica gel (gradient: ethyl acetate/cyclohexane 30:70→60:40). This gave the amides of 2-(pyridin-3-yl)[1,3]thiazolo[5,4-b]pyridine-5-amine.

### General Procedure for the Methylation of Secondary Amides of 2-(pyridin-3-yl)[1,3]thiazolo[5,4-b]pyridine-5-amine

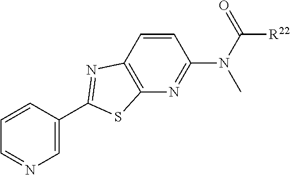

Sodium hydride (1.1 eq.) was added to a solution of the amide in question (200 mg, 1.0 eq.) in dimethylformamide (4 ml), and the mixture was stirred at 30° C. for 30 min. Methyl iodide (1.0 eq.) was then added, and the mixture was stirred at 30° C. for a further 30 min. A saturated aqueous solution of ammonium chloride (1 ml) was added to the reaction mixture and the solvent was removed under reduced pressure. For isolation of the product, the reaction mixture was purified by HPLC.

### General Procedure for the Oxidation of Phenyl Trifluoromethyl Sulfides to Phenyl Trifluoromethyl Sulfoxides

A solution of the trifluoromethyl sulfide (1.0 eq) and meta-chloroperbenzoic acid (1.0 eq) in CH₂Cl₂ (3 ml) was stirred at 30° C. for 16 h. The solvent was removed under reduced pressure and the residue was separated chromatographically by MPLC on silica gel (gradient: methanol/CH₂Cl₂ 0:100→5:95). The product was then purified again by HPLC (mobile phase contains formic acid).

### General Procedure for the Oxidation of Phenyl Trifluoromethyl Sulfides to Phenyl Trifluoromethylsulfones

A solution of the trifluoromethyl sulfide (1.0 eq) and meta-chloroperbenzoic acid (2.0 eq) in CH₂Cl₂ (3 ml) was stirred at 30° C. for 16 h. The solvent was removed under reduced pressure and the residue was separated chromatographically by MPLC on silica gel (gradient: methanol/CH₂Cl₂ 0:100→5:95). The product was then purified again by HPLC (mobile phase contains formic acid).

### Example 1: 5-[2-Fluoro-4-methyl-5-(2,2,2-trifluoroethylsulfanyl)phenyl]-2-(3-pyridyl)thiazol [5,4-b]pyridine

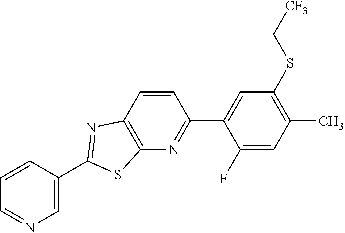 [SMILES: Cc1cc(F)c(-c2ccc3nc(-c4cccnc4)sc3n2)cc1SCC(F)(F)F]

Similar to a reaction procedure from WO 2010/071819, (7 mg, 6 μmol), water (0.65 ml) and 1,2-dimethoxyethane (2.65 ml) were added to a mixture of 5-chloro-2-(3-pyridyl)thiazolo[5,4-b]pyridine (50 mg, 0.20 mmol), [2-fluoro-4-methyl-5-(2,2,2-trifluoroethylsulfanyl)phenyl]boronic acid (97 mg, 0.36 mmol), sodium carbonate (64 mg, 0.61 mmol) and tetrakis(triphenylphosphine)palladium. The reaction mixture was repeatedly flushed with a stream of argon and the vessel was closed. The mixture was heated in a CEM Discover microwave reactor at 140° C. for 40 min and, after cooling to room temperature, filtered through a depth filter which was rinsed with ethyl acetate. The reaction was carried out twice in total and the batches were combined prior to purification. After the solvent has been removed under reduced pressure, the residue was separated chromatographically by MPLC on silica gel (gradient: ethyl acetate/cyclohexane 0:100→30:70).

This gave 96 mg (96% pure, 52% yield) of 5-[2-fluoro-4-methyl-5-(2,2,2-trifluoroethylsulfanyl)phenyl]-2-(3-pyridyl)thiazol[5,4-b]pyridine.

¹H-NMR (400.0 MHz, d₆-DMSO): δ=9.336 (9.6); 9.331 (9.4); 9.330 (9.2); 8.819 (7.7); 8.815 (8.5); 8.807 (8.3); 8.803 (8.5); 8.615 (14.3); 8.593 (16.0); 8.536 (4.4); 8.532 (5.7); 8.530 (5.6); 8.526 (4.5); 8.516 (4.8); 8.512 (5.8); 8.510 (6.2); 8.506 (4.6); 8.318 (0.8); 8.208 (9.7); 8.188 (9.7); 8.040 (6.9); 8.036 (7.9); 8.018 (7.2); 8.014 (6.9); 7.685 (5.6); 7.683 (5.7); 7.673 (5.4); 7.671 (5.5); 7.665 (5.4); 7.663 (5.4); 7.653 (5.3); 7.651 (5.4); 7.423 (8.2); 7.411 (0.5); 7.392 (8.1); 4.008 (4.2); 3.982 (13.4); 3.956 (14.0); 3.930 (4.8); 3.330 (261.5); 2.682 (0.7); 2.677 (1.5); 2.673 (2.1); 2.668 (1.6); 2.664 (0.8); 2.656 (0.3); 2.526 (5.6); 2.521 (8.5); 2.512 (114.8); 2.508 (235.5); 2.504 (315.5); 2.499 (263.2); 2.420 (1.4); 2.368 (0.5); 2.335 (1.8); 2.330 (2.1); 2.326 (1.6); 2.321 (0.9); 1.990 (0.6); 1.398 (4.1); 0.146 (2.5); 0.029 (0.4); 0.008 (19.6); 0.000 (555.1); −0.009 (21.4); −0.033 (0.4); −0.150 (2.6).

### Example 2: 5-[4-Methyl-3-(2,2,2-trifluoroethylsulfanyl)phenyl]-2-(3-pyridyl)thiazolo[5,4-b]pyridine

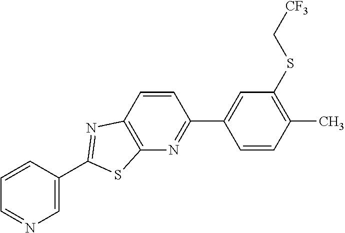 [SMILES: Cc1ccc(-c2ccc3nc(-c4cccnc4)sc3n2)cc1SCC(F)(F)F]

The preparation of 5-[4-methyl-3-(2,2,2-trifluoroethylsulfanyl)phenyl]-2-(3-pyridyl)thiazolo[5,4-b]pyridine was carried out analogously to the synthesis of 5-[2-fluoro-4-methyl-5-(2,2,2-trifluoroethylsulfanyl)phenyl]-2-(3-pyridyl)thiazol[5,4-b]pyridine. Here, 5-chloro-2-(3-pyridyl)thiazolo[5,4-b]pyridine (50 mg, 0.20 mmol), [4-methyl-3-(2,2,2-trifluoroethylsulfanyl)phenyl]boronic acid (91 mg, 0.36 mmol), tetrakis(triphenylphosphine)palladium (7 mg, 6 μmol) and sodium carbonate (64 mg, 0.61 mmol) were used. The reaction was carried out twice and the batches were combined prior to purification. This gave 125 mg of 5-[4-methyl-3-(2,2,2-trifluoroethylsulfanyl)phenyl]-2-(3-pyridyl)thiazolo[5,4-b]pyridine (94% pure, 70% yield).

¹H-NMR (400.0 MHz, d₆-DMSO): δ=9.322 (1.9); 8.808 (1.4); 8.799 (1.4); 8.591 (4.3); 8.581 (0.4); 8.569 (5.1); 8.560 (0.4); 8.516 (1.3); 8.511 (1.9); 8.507 (1.4); 8.496 (1.4); 8.491 (2.0); 8.487 (1.4); 8.317 (0.5); 8.307 (3.9); 8.301 (7.3); 8.279 (4.2); 8.070 (2.2); 8.066 (2.1); 8.050 (2.2); 8.046 (2.2); 7.677 (1.7); 7.666 (1.7); 7.658 (1.6); 7.646 (1.6); 7.456 (3.0); 7.436 (2.8); 4.156 (1.3); 4.130 (4.2); 4.104 (4.4); 4.078 (1.5); 3.331 (95.2); 2.677 (0.4); 2.673 (0.6); 2.668 (0.5); 2.526 (1.4); 2.512 (35.3); 2.508 (70.8); 2.504 (93.0); 2.499 (68.9); 2.495 (34.7); 2.455 (16.0); 2.391 (0.8); 2.340 (0.4); 2.335 (0.5); 2.330 (0.7); 2.326 (0.5); 1.397 (3.2); 0.008 (0.4); 0.000 (11.3); −0.008 (0.5).

### Example 3: 5-[2-Fluoro-4-methyl-5-(2,2,2-trifluoroethylsulfinyl)phenyl]-2-(3-pyridyl)thiazolo[5,4-b]pyridine

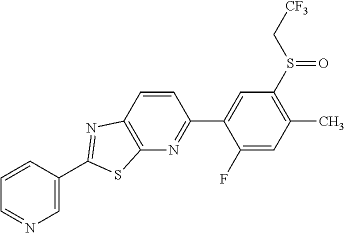 [SMILES: Cc1cc(F)c(-c2ccc3nc(-c4cccnc4)sc3n2)cc1S(=O)CC(F)(F)F]

At 0° C., meta-chloroperbenzoic acid (70% pure, 33 mg, 0.14 mmol) was added to a solution of 6-[2-fluoro-4-methyl-5-(2,2,2-trifluoroethylsulfanyl)phenyl]-2-(3-pyridyl)thiazolo[5,4-b]pyridine (60 mg, 0.13 mmol) in CH₂Cl₂ (2.5 ml). The reaction mixture was stirred at room temperature for 2 h, and saturated sodium carbonate solution was then added. After 15 min, the phases were separated, the aqueous phase was extracted with CH₂Cl₂ and the combined organic phases were dried over sodium sulfate. The solvent was removed under reduced pressure. The crude product was washed with CH₂Cl₂ giving, after drying, 30 mg (97% pure, 48% yield) of 5-[2-fluoro-4-methyl-5-(2,2,2-trifluoroethylsulfinyl)phenyl]-2-(3-pyridyl)thiazolo[5,4-b]pyridine.

¹H-NMR (400.0 MHz, d₆-DMSO): δ=9.341 (3.3); 9.336 (3.3); 8.823 (2.3); 8.820 (2.6); 8.811 (2.5); 8.808 (2.6); 8.654 (3.6); 8.633 (4.0); 8.593 (0.4); 8.581 (3.0); 8.562 (3.0); 8.535 (2.0); 8.530 (1.5); 8.519 (1.5); 8.514 (2.0); 8.510 (1.5); 8.317 (0.4); 8.126 (2.1); 8.122 (2.4); 8.104 (2.1); 8.101 (2.1); 7.687 (1.8); 7.675 (1.9); 7.667 (1.8); 7.655 (1.7); 7.526 (2.5); 7.496 (2.5); 5.757 (2.9); 4.261 (0.4); 4.251 (0.6); 4.232 (1.0); 4.224 (1.7); 4.205 (1.9); 4.197 (1.9); 4.178 (1.7); 4.169 (1.0); 4.150 (0.6); 4.141 (0.4); 3.329 (81.5); 2.672 (1.0); 2.668 (0.8); 2.507 (113.6); 2.503 (144.6); 2.499 (114.9); 2.468 (16.0); 2.429 (0.4); 2.330 (1.0); 0.000 (17.6).

### Example 4: 5-[4-Methyl-3-(2,2,2-trifluoroethylsulfinyl)phenyl]-2-(3-pyridyl)thiazolo[5,4-b]pyridine

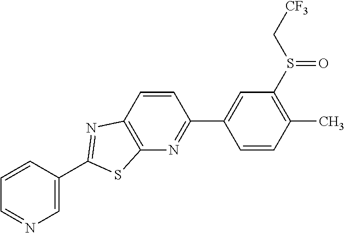 [SMILES: Cc1ccc(-c2ccc3nc(-c4cccnc4)sc3n2)cc1S(=O)CC(F)(F)F]

The preparation of 5-[4-methyl-3-(2,2,2-trifluoroethylsulfinyl)phenyl]-2-(3-pyridyl)thiazolo[5,4-b]pyridine was carried out analogously to the synthesis of 5-[2-fluoro-4-methyl-5-(2,2,2-trifluoroethylsulfinyl)phenyl]-2-(3-pyridyl)thiazolo[5,4-b]pyridine. Here, 5-[4-methyl-3-(2,2,2-trifluoroethylsulfanyl)phenyl]-2-(3-pyridyl)thiazolo[5,4-b]pyridine (88 mg, 0.20 mmol) and meta-chloroperbenzoic acid (70% pure, 50 mg, 0.20 mmol) were used. This gave 27 mg (92% pure, 29% yield) of 5-[4-methyl-3-(2,2,2-trifluoroethylsulfinyl)phenyl]-2-(3-pyridyl)thiazolo[5,4-b]pyridine.

¹H-NMR (400.0 MHz, d₆-DMSO): δ=9.325 (3.3); 9.320 (3.3); 8.813 (2.4); 8.810 (2.6); 8.802 (2.5); 8.798 (2.6); 8.717 (4.0); 8.713 (4.2); 8.629 (3.7); 8.607 (4.3); 8.522 (1.4); 8.517 (2.0); 8.512 (1.5); 8.502 (1.5); 8.497 (2.1); 8.492 (1.4); 8.338 (2.3); 8.331 (5.1); 8.318 (2.6); 8.310 (4.1); 7.680 (1.8); 7.668 (1.8); 7.660 (1.8); 7.648 (1.7); 7.552 (2.9); 7.532 (2.8); 5.758 (0.9); 4.235 (0.7); 4.226 (0.7); 4.208 (2.0); 4.199 (1.9); 4.181 (2.0); 4.172 (2.0); 4.154 (0.7); 4.145 (0.7); 4.136 (0.3); 3.366 (0.3); 3.331 (76.2); 2.672 (0.7); 2.507 (78.8); 2.503 (100.7); 2.499 (78.2); 2.460 (16.0); 2.330 (0.7); 0.000 (2.6).

### Example 6: 2-(3-Pyridyl)-5-pyrrolidin-1-ylthiazolo[5,4-b]pyridine

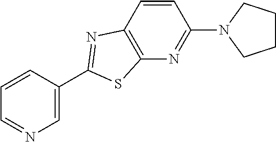 [SMILES: c1cncc(-c2nc3ccc(N4CCCC4)nc3s2)c1]

Pyrrolidine (142 mg, 2 mmol) was added to a solution of 5-chloro-2-(3-pyridyl)thiazolo[5,4-b]pyridine (100 mg, 0.40 mmol) in dimethylformamide (3 ml), and the reaction mixture was stirred at 100° C. for 4 h. After cooling to room temperature, the solvents were removed under reduced pressure and the residue that remained was separated chromatographically by HPLC. This gave 36 mg (95% pure, 32% yield) of 2-(3-pyridyl)-5-pyrimidin-2-ylthiazolo[5,4-b]pyridine.

¹H-NMR (400.0 MHz, d₆-DMSO): δ=9.151 (5.5); 9.147 (5.6); 8.683 (3.9); 8.679 (4.2); 8.671 (4.1); 8.667 (4.1); 8.335 (0.5); 8.327 (2.5); 8.323 (3.4); 8.318 (2.6); 8.314 (1.6); 8.307 (2.7); 8.302 (3.5); 8.298 (2.5); 8.140 (8.2); 8.118 (8.6); 7.580 (3.1); 7.569 (3.1); 7.561 (3.1); 7.549 (2.9); 7.521 (0.4); 6.731 (7.9); 6.708 (7.7); 5.754 (1.8); 3.510 (5.5); 3.494 (14.2); 3.477 (5.8); 3.316 (134.8); 3.141 (0.9); 2.675 (1.7); 2.671 (2.3); 2.666 (1.7); 2.632 (1.8); 2.571 (0.4); 2.567 (0.4); 2.562 (0.4); 2.524 (7.4); 2.510 (135.4); 2.506 (268.7); 2.502 (352.7); 2.497 (264.6); 2.493 (136.1); 2.333 (1.6); 2.328 (2.2); 2.324 (1.7); 2.018 (0.7); 2.003 (6.0); 1.993 (7.0); 1.986 (16.0); 1.980 (7.3); 1.970 (5.9); 1.335 (0.8); 1.259 (0.7); 1.250 (0.6); 1.233 (2.1); 0.854 (0.4); 0.146 (2.1); 0.031 (0.4); 0.022 (0.8); 0.008 (17.8); 0.000 (454.2); −0.008 (23.0); −0.028 (0.7); −0.150 (2.2).

### Example 7: 2-(3-Pyridyl)-5-pyrimidin-2-ylthiazolo[5,4-b]pyridine

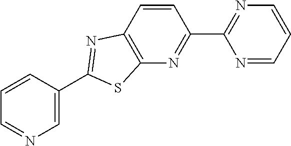 [SMILES: c1cnc(-c2ccc3nc(-c4cccnc4)sc3n2)nc1]

Similar to the reaction procedure from WO 2013/159064, under argon 1,4-dioxane (2 ml) was added to a mixture of 5-chloro-2-(3-pyridyl)thiazolo[5,4-b]pyridine (100 mg, 0.40 mmol), 2-(tributylstannyl)pyrimidine (0.16 ml, 0.50 mmol), copper(I) iodide (23 mg, 0.12 mmol) and tetrakis(triphenylphosphine)palladium (51 mg, 44 μmol). The reaction mixture was stirred at 90° C. for 16 h and, after cooling to room temperature, the solvent was removed under reduced pressure. The residue was purified chromatographically by MPLC on silica gel (gradient: ethyl acetate/cyclohexane 0:100→100:0). The product obtained was dissolved in THF (2 ml), and a solution of potassium fluoride (116 mg, 200 μmol) in water (2 ml) was added. The solution was stirred at room temperature for 15 min and then extracted with ethyl acetate.

The combined organic phases were dried with Na₂SO₄ and the solvent was removed under reduced pressure. The residue was filtered through silica gel, rinsing with ethyl acetate and methanol. This gave 5 mg (100% pure, 4% yield) of 2-(3-pyridyl)-5-pyrimidin-2-ylthiazolo[5,4-b]pyridine.

¹H-NMR (400.0 MHz, d₆-DMSO): δ=9.357 (3.1); 9.352 (3.1); 9.049 (8.9); 9.037 (9.0); 8.829 (2.3); 8.825 (2.5); 8.817 (2.4); 8.813 (2.4); 8.667 (16.0); 8.564 (1.3); 8.560 (1.9); 8.555 (1.4); 8.544 (1.4); 8.539 (1.9); 8.535 (1.3); 8.319 (0.7); 7.690 (1.7); 7.678 (1.7); 7.671 (1.7); 7.659 (1.6); 7.634 (2.5); 7.622 (4.7); 7.610 (2.4); 5.759 (1.0); 3.330 (115.0); 2.676 (1.3); 2.672 (1.7); 2.667 (1.3); 2.525 (4.6); 2.507 (204.2); 2.503 (264.8); 2.498 (196.5); 2.334 (1.3); 2.329 (1.7); 2.325 (1.3); 1.258 (0.5); 1.233 (1.9); 0.852 (0.6); 0.833 (0.3); 0.146 (0.6); 0.008 (4.5); 0.000 (124.1); −0.150 (0.6).

### Example 8: 5-(2-Pyridyl)-2-(3-pyridyl)thiazolo[5,4-b]pyridine

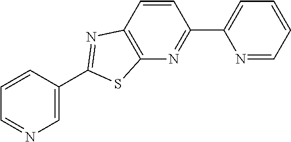 [SMILES: c1ccc(-c2ccc3nc(-c4cccnc4)sc3n2)nc1]

Similar to the reaction procedure from the Journal of Organic Chemistry (2010), 75, 8830-8832, a solution of 2-dicyclohexylphosphino-2′,4′,6′-triisopropylbiphenyl (15 mg, 31 μmol) and tris(dibenzylideneacetone)dipalladium (7.4 mg, 8.1 μmol) in THF (2.3 ml) was stirred under argon at 65° C. for 10 min. 5-Chloro-2-(3-pyridyl)thiazolo[5,4-b]pyridine (100 mg, 0.40 mmol) was then added as a suspension in THF (3 ml). The heating bath was removed and a solution of 2-pyridylzinc bromide in THF (0.5 M, 1.21 ml, 0.6 mmol) was added dropwise. The reaction mixture was stirred at room temperature for 2 h, at 65° C. for 8 h and at room temperature for a further 62 h, and a semisaturated solution of NaHCO₃ was then added. The phases were separated and the aqueous phase was extracted with ethyl acetate. The combined organic phases were washed with water and dried with Na₂SO₄, and the solvent was removed under reduced pressure.

The residue was purified chromatographically by MPLC on silica gel (gradient: ethyl acetate/cyclohexane 0:100→70:30). This gave 19 mg (100% pure, 16% yield) of 5-(2-pyridyl)-2-(3-pyridyl)thiazolo[5,4-b]pyridine.

¹H-NMR (601.6 MHz, CDCl₃): δ=9.345 (6.9); 9.344 (7.1); 9.342 (7.2); 9.341 (6.4); 8.772 (5.8); 8.769 (5.9); 8.764 (6.0); 8.762 (5.7); 8.737 (3.9); 8.736 (4.6); 8.734 (4.6); 8.733 (4.0); 8.729 (4.2); 8.728 (4.6); 8.727 (4.4); 8.725 (3.6); 8.648 (12.4); 8.634 (14.1); 8.511 (4.7); 8.510 (6.8); 8.508 (4.3); 8.498 (4.9); 8.497 (7.1); 8.495 (4.3); 8.426 (15.6); 8.422 (5.2); 8.421 (4.8); 8.418 (3.8); 8.412 (16.0); 8.409 (5.6); 8.408 (5.0); 8.405 (3.7); 7.883 (3.5); 7.880 (3.4); 7.870 (5.6); 7.868 (5.5); 7.857 (3.6); 7.854 (3.4); 7.498 (4.4); 7.496 (4.2); 7.490 (4.5); 7.488 (4.3); 7.484 (4.4); 7.483 (4.1); 7.477 (4.3); 7.475 (4.0); 7.434 (0.3); 7.369 (3.9); 7.368 (3.8); 7.362 (3.9); 7.360 (4.1); 7.357 (4.1); 7.355 (3.7); 7.349 (3.7); 7.347 (3.4); 7.262 (58.9); 7.086 (0.3); 1.594 (59.7); 1.333 (0.7); 1.284 (1.0); 1.254 (1.5); 0.880 (0.3); 0.844 (0.4); 0.839 (0.4); 0.005 (1.5); 0.000 (45.7); −0.006 (1.7).

### Example 9: N-[4-Methyl-3-(2,2,2-trifluoroethylsulfanyl)phenyl]-2-(3-pyridyl)thiazolo[5,4-b]pyridine-5-amine

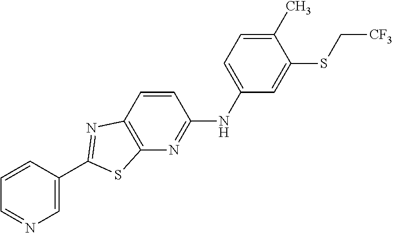 [SMILES: Cc1ccc(Nc2ccc3nc(-c4cccnc4)sc3n2)cc1SCC(F)(F)F]

N-[4-Methyl-3-(2,2,2-trifluoroethylsulfanyl)phenyl]-2-(3-pyridyl)thiazolo[5,4-b]pyridine-5-amine was prepared analogously to the synthesis of 4-[2-(3-pyridyl)thiazolo[5,4-b]pyridin-5-yl]morpholine. Here, 5-chloro-2-(3-pyridyl)thiazolo[5,4-b]pyridine (100 mg, 0.40 mmol), 4-methyl-3-(2,2,2-trifluoroethylsulfanyl)aniline (134 mg, 0.61 mmol), palladium(II) acetate (4.5 mg, 20 μmol), rac-(2,2′-bis(diphenylphosphino)-1,1′-binaphthyl) (5 mg, 8 μmol) and sodium tert-butoxide (58 mg, 0.60 mmol) were employed. This gave 72 mg (94% pure, 38% yield) of N-[4-methyl-3-(2,2,2-trifluoroethylsulfanyl)phenyl]-2-(3-pyridyl)thiazolo[5,4-b]pyridine-5-amine.

¹H-NMR (400.0 MHz, d₆-DMSO): δ=9.638 (3.9); 9.195 (2.9); 9.191 (3.0); 8.717 (2.3); 8.713 (2.4); 8.705 (2.4); 8.701 (2.4); 8.378 (1.2); 8.374 (1.7); 8.368 (1.3); 8.358 (1.4); 8.353 (1.8); 8.348 (1.4); 8.318 (0.4); 8.236 (4.2); 8.214 (4.5); 8.007 (3.2); 8.002 (3.3); 7.607 (1.7); 7.595 (1.6); 7.587 (1.6); 7.573 (2.1); 7.571 (2.0); 7.565 (1.7); 7.550 (2.0); 7.545 (2.0); 7.236 (2.9); 7.215 (2.5); 7.038 (4.0); 7.015 (4.0); 4.056 (0.4); 4.038 (1.1); 4.020 (1.1); 4.002 (0.4); 3.954 (1.2); 3.928 (3.7); 3.902 (3.9); 3.876 (1.3); 3.330 (134.6); 2.676 (0.8); 2.672 (1.1); 2.667 (0.8); 2.525 (3.4); 2.511 (66.4); 2.507 (131.9); 2.503 (172.2); 2.498 (126.1); 2.494 (63.2); 2.338 (16.0); 2.266 (0.5); 1.989 (4.8); 1.397 (0.4); 1.193 (1.3); 1.175 (2.5); 1.157 (1.3); 0.146 (0.9); 0.008 (7.6); 0.000 (197.5); −0.008 (8.7); −0.150 (0.9).

### Example 11: 4-[2-(3-Pyridyl)thiazolo[5,4-b]pyridin-5-yl]morpholine

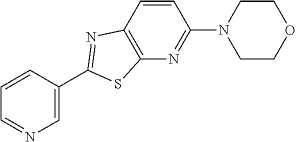 [SMILES: c1cncc(-c2nc3ccc(N4CCOCC4)nc3s2)c1]

Similar to the reaction procedure from WO 2007/148093, toluene (0.75 ml) was added to a mixture of 5-chloro-2-(3-pyridyl)thiazolo[5,4-b]pyridine (100 mg, 0.40 mmol), morpholine (53 μl, 0.61 mmol), palladium(II) acetate (4.5 mg, 20 μmol), rac-(2,2′-bis(diphenylphosphino)-1,1′-binaphthyl) (5 mg, 8 μmol) and sodium tert-butoxide (58 mg, 0.60 mmol). The reaction mixture was freed from dissolved oxygen by passing a stream of argon through the mixture for 20 minutes and then stirred at 110° C. for 16 h. After cooling to room temperature, the mixture was filtered though a depth filter which was subsequently rinsed with ethyl acetate. The solvent was removed under reduced pressure and the residue was separated chromatographically by MPLC on silica gel (gradient: ethyl acetate/cyclohexane 0:100→100:0). This gave 29 mg (100% pure, 24% yield) of 4-[2-(3-pyridyl)thiazolo[5,4-b]pyridin-5-yl]morpholine.

¹H-NMR (400.0 MHz, d₆-DMSO): δ=9.179 (5.8); 9.174 (5.8); 8.709 (4.1); 8.706 (4.4); 8.697 (4.3); 8.694 (4.2); 8.356 (3.6); 8.336 (3.7); 8.317 (0.4); 8.222 (6.4); 8.199 (6.7); 7.598 (3.3); 7.586 (3.4); 7.578 (3.3); 7.566 (3.0); 7.138 (6.3); 7.115 (6.1); 3.745 (9.6); 3.733 (16.0); 3.721 (13.6); 3.609 (13.4); 3.596 (15.7); 3.585 (9.5); 3.332 (188.8); 2.672 (1.2); 2.503 (168.0); 2.330 (1.1); 1.990 (0.5); 0.000 (15.6).

### Example 12: N-methyl-N-[4-methyl-3-(2,2,2-trifluoroethylsulfanyl)phenyl]-2-(3-pyridyl)thiazolo[5,4-b]pyridine-5-amine

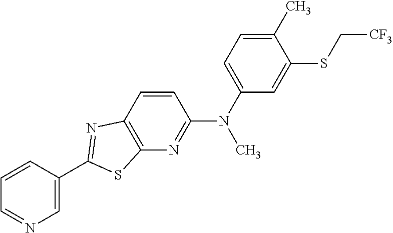 [SMILES: Cc1ccc(N(C)c2ccc3nc(-c4cccnc4)sc3n2)cc1SCC(F)(F)F]

At 0° C., sodium hydride (7.6 mg, 0.19 mmol) was added to a solution of N-[4-methyl-3-(2,2,2-trifluoroethylsulfanyl)phenyl]-2-(3-pyridyl)thiazolo[5,4-b]pyridine-5-amine (59 mg, 0.12 mmol) in tetrahydrofuran (1.2 ml), and the reaction solution was stirred at 0° C. for 15 min. Methyl iodide (16 μl, 0.25 mmol) was added and the reaction mixture was stirred at room temperature overnight. The reaction mixture was then once more cooled to 0° C., sodium hydride (5 mg, 0.2 mmol) and methyl iodide (8 μl, 0.13 mmol) were added and the mixture was stirred at room temperature overnight. For work-up, a saturated aqueous ammonium chloride solution was added to the reaction mixture, the phases were separated and the aqueous phase was extracted repeatedly with ethyl acetate. The combined organic phases were dried with sodium sulfate and the solvents were removed under reduced pressure.

The crude product was separated chromatographically by HPLC (gradient: H₂O/acetonitrile). This gave 4.8 mg (94% pure, 8% yield) of N-methyl-N-[4-methyl-3-(2,2,2-trifluoroethylsulfanyl)phenyl]-2-(3-pyridyl)thiazolo[5,4-b]pyridine-5-amine.

¹H-NMR (601.6 MHz, CDCl₃): δ=9.227 (1.6); 9.224 (1.6); 8.677 (1.1); 8.674 (1.2); 8.669 (1.2); 8.666 (1.1); 8.297 (0.8); 8.293 (1.1); 8.290 (0.8); 8.283 (0.8); 8.280 (1.1); 8.277 (0.8); 7.901 (2.9); 7.885 (2.9); 7.424 (1.0); 7.423 (1.0); 7.413 (2.4); 7.410 (2.9); 7.403 (1.0); 7.402 (0.9); 7.310 (1.5); 7.296 (1.8); 7.264 (3.3); 7.164 (1.4); 7.160 (1.3); 7.151 (1.1); 7.147 (1.1); 6.627 (2.9); 6.611 (2.9); 5.299 (2.2); 3.545 (1.4); 3.538 (16.0); 3.438 (1.1); 3.422 (3.4); 3.406 (3.5); 3.391 (1.2); 2.491 (10.7); 2.442 (1.0); 2.359 (0.7); 1.688 (1.5); 1.254 (1.6); 0.000 (0.9).

### Example 13: 2-(3-Pyridyl)-5-[4-(trifluoromethyl)pyrazol-1-yl]thiazolo[5,4-b]pyridine

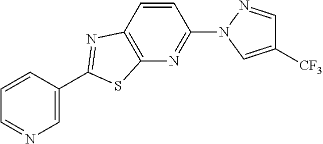 [SMILES: FC(F)(F)c1cnn(-c2ccc3nc(-c4cccnc4)sc3n2)c1]

Similar to the reaction procedure from the Journal of Organic Chemistry (2004), 69, 5578-5587, under argon, 5-chloro-2-(3-pyridyl)thiazolo[5,4-b]pyridine (100 mg, 0.40 mmol), trans-N,N′-dimethylcyclohexane-1,2-diamine (22 μl, 0.14 mmol) and degassed toluene (1 ml) were added to a mixture of copper(I) iodide (13 mg, 68 μmol), 4-(trifluoromethyl)-1H-pyrazole (46 mg, 0.34 mmol) and potassium carbonate (98 mg, 0.71 mmol). The vessel was closed and the reaction mixture was heated in a CEM Discover microwave at 120° C. for 18 h. After cooling to room temperature, ethyl acetate was added and the mixture was filtered through a depth filter, which was subsequently rinsed with ethyl acetate. The solvent was removed under reduced pressure and the residue was separated chromatographically by MPLC on silica gel (gradient: ethyl acetate/cyclohexane 0:100→50:50).

The product was then separated again chromatographically by HPLC (gradient: H₂O/acetonitrile). This gave 10 mg (100% pure, 9% yield) of 2-(3-pyridyl)-5-[4-(trifluoromethyl)pyrazol-1-yl]thiazolo[5,4-b]pyridine. ¹H-NMR (400.0 MHz, d₆-DMSO): δ=9.397 (12.4); 9.334 (8.8); 9.330 (8.9); 8.819 (6.7); 8.816 (7.5); 8.807 (7.2); 8.804 (7.4); 8.747 (14.6); 8.725 (16.0); 8.538 (3.9); 8.533 (5.4); 8.528 (4.1); 8.518 (4.3); 8.512 (5.7); 8.508 (4.3); 8.382 (15.9); 8.317 (2.8); 8.235 (15.5); 8.213 (14.5); 7.992 (0.5); 7.684 (5.1); 7.682 (5.3); 7.672 (4.9); 7.670 (5.1); 7.664 (4.9); 7.662 (5.0); 7.652 (4.7); 7.650 (4.8); 3.329 (899.6); 2.676 (4.4); 2.672 (6.1); 2.667 (4.6); 2.542 (2.4); 2.525 (16.4); 2.520 (24.9); 2.511 (334.7); 2.507 (693.8); 2.503 (925.4); 2.498 (682.8); 2.494 (341.5); 2.334 (4.3); 2.329 (6.0); 2.325 (4.5); 2.075 (0.3); 1.148 (0.6); 0.146 (7.1); 0.032 (1.0); 0.025 (2.1); 0.008 (54.7); 0.000 (1562.0); −0.009 (65.0); −0.029 (1.5); −0.038 (0.8); −0.042 (0.6); −0.050 (0.5); −0.065 (0.4); −0.150 (7.1).

### Example 14: N-Cyclopropyl-2-(pyridin-3-yl)[1,3]thiazolo[5,4-b]pyridine-5-amine

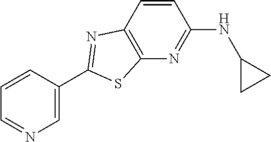 [SMILES: c1cncc(-c2nc3ccc(NC4CC4)nc3s2)c1]

Cyclopropylamine (0.28 ml, 4 mmol) was added to a solution of 5-chloro-2-(3-pyridyl)thiazolo[5,4-b]pyridine (100 mg, 0.40 mmol) in dimethyl sulfoxide (3 ml), and the reaction mixture was heated in a microwave at 150° C. for 1.5 h. After cooling to room temperature, the solvents were removed under reduced pressure and the residue that remained was separated chromatographically by HPLC. This gave 12 mg (99% pure, 11% yield) of N-cyclopropyl-2-(pyridin-3-yl)[1,3]thiazolo[5,4-b]pyridine-5-amine.

¹H-NMR (400 MHz, CDCl₃): δ=9.23 (s, 1H), 8.68-8.67 (m, 1H), 8.29-8.26 (m, 1H), 8.12-8.09 (m, 1H), 7.43-7.40 (m, 1H), 6.96-6.94 (m, 1H), 5.25 (s, 1H), 2.66-2.62 (m, 1H), 0.89-0.86 (m, 2H), 0.65-0.63 (m, 2H).

### Example 16: N-methyl-N-[2- (pyridin-3-yl)[1,3]thiazolo[5,4-b]pyridin-5-yl]cyclopropanecarboxamide

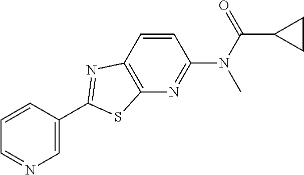 [SMILES: CN(C(=O)C1CC1)c1ccc2nc(-c3cccnc3)sc2n1]

At 0° C., cyclopropanecarbonyl chloride (0.13 g, 1.2 mmol) was added to a solution of N-methyl-2-(pyridin-3-yl)[1,3]thiazolo[5,4-b]pyridine-5-amine (97 mg, 0.40 mmol) and triethylamine (0.12 g, 1.2 mmol) in CH₂Cl₂ (4 ml), and the reaction mixture was stirred at 25° C. for 6 h. The solvents were removed under reduced pressure and the residue that remained was separated chromatographically by HPLC. This gave 64 mg (99% pure, 52% yield) of N-methyl-N-[2-(pyridin-3-yl)[1,3]thiazolo[5,4-b]pyridin-5-yl]cyclopropanecarboxamide.

¹H-NMR (400 MHz, CDCl₃): δ=9.13 (s, 1H), 8.77-8.76 (m, 1H), 8.38-8.31 (m, 2H), 7.59-7.57 (m, 1H), 7.50-7.47 (m, 1H), 3.54 (s, 3H), 1.73-1.70 (m, 1H), 1.18-1.14 (m, 2H), 0.84-0.80 (m, 2H).

### Example 17: N-Ethyl-N-[2-(pyridin-3-yl)[1,3]thiazolo[5,4-b]pyridin-5-yl]methanesulfonamide

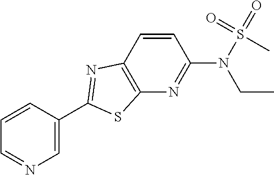 [SMILES: CCN(c1ccc2nc(-c3cccnc3)sc2n1)S(C)(=O)=O]

At −20° C., lithium hexamethyldisilazide (1.2 ml, 1.2 mmol, 1 M in THF) was added to a solution of N-ethyl-2-(pyridin-3-yl)[1,3]thiazolo[5,4-b]pyridine-5-amine (103 mg, 0.40 mmol) in THF (4 ml), and the reaction mixture was stirred for 10 min. Methanesulfonyl chloride (0.14 g, 1.2 mmol) was then added, and the reaction solution was stirred at 25° C. for 6 h. The solvents were removed under reduced pressure and the residue that remained was separated chromatographically by HPLC. This gave 24 mg (99% pure, 18% yield) ofN-ethyl-N-[2-(pyridin-3-yl)[1,3]thiazolo[5,4-b]pyridin-5-yl]methanesulfonamide.

¹H-NMR (400 MHz, CDCl₃): δ=9.29 (s, 1H), 8.77-8.76 (m, 1H), 8.39-8.32 (m, 2H), 7.62-7.59 (m, 1H), 7.50-7.47 (m, 1H), 4.07-4.02 (m, 2H), 3.09 (s, 3H), 1.26-1.22 (m, 3H).

### Example 30: N-Methyl-3-(methylsulfonyl)-N-[2-(pyridin-3-yl)[1,3]thiazolo[5,4-b]pyridin-5-yl]propanamide

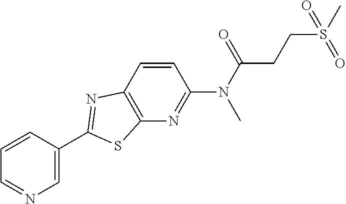 [SMILES: CN(C(=O)CCS(C)(=O)=O)c1ccc2nc(-c3cccnc3)sc2n1]

A solution of 3-(methylsulfanyl)-N-[2-(pyridin-3-yl)[1,3]thiazolo[5,4-b]pyridin-5-yl]propanamide (1.0 eq) and meta-chloroperbenzoic acid (2.0 eq) in CH₂Cl₂ (5 ml) was stirred at 30° C. for 16 h. The solvent was removed under reduced pressure and the residue was purified by HPLC (mobile phase contains formic acid). This gave 3-(methylsulfonyl)-N-[2-(pyridin-3-yl)[1,3]thiazolo[5,4-b]pyridin-5-yl]propanamide.

Sodium hydride (1.1 eq.) was added to a solution of 3-(methylsulfonyl)-N-[2-(pyridin-3-yl)[1,3]thiazolo[5,4-b]pyridin-5-yl]propanamide (1.0 eq.) in dimethylformamide (4 ml), and the mixture was stirred at 0° C. for 30 min. Methyl iodide (1.0 eq.) was then added, and the mixture was stirred at 0° C. for a further 30 min. A saturated aqueous solution of ammonium chloride (1 ml) was added to the reaction mixture and the solvent was removed under reduced pressure. For the isolation of the N-methyl-3-(methylsulfonyl)-N-[2-(pyridin-3-yl)[1,3]thiazolo[5,4-b]pyridin-5-yl]propanamide, the reaction mixture was purified by HPLC (mobile phase contains formic acid).

¹H-NMR (400.0 MHz, DMSO): δ=9.299 (2.1); 9.295 (2.1); 8.802 (1.5); 8.792 (1.5); 8.595 (3.3); 8.573 (3.5); 8.499 (1.1); 8.494 (1.5); 8.489 (1.1); 8.479 (1.2); 8.474 (1.6); 8.469 (1.1); 7.819 (3.2); 7.797 (3.0); 7.669 (1.3); 7.657 (1.3); 7.649 (1.3); 7.637 (1.2); 3.902 (3.7); 3.431 (2.9); 3.423 (14.8); 3.414 (4.1); 3.394 (2.7); 3.330 (150.6); 3.176 (0.7); 3.163 (0.7); 2.991 (16.0); 2.931 (1.6); 2.912 (2.3); 2.893 (1.3); 2.676 (0.6); 2.672 (0.8); 2.667 (0.6); 2.525 (2.2); 2.511 (46.0); 2.507 (94.4); 2.502 (130.3); 2.498 (97.4); 2.494 (47.0); 2.334 (0.5); 2.329 (0.7); 2.325 (0.6); 0.000 (2.9)

Compounds of the formula (I) and also those not embraced by formula (I) are listed in the table below. The compounds not embraced by formula (I) also form part of the subject-matter of the invention.

TABLE 1
Compounds of the formula

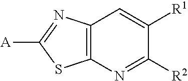

| Com- | | | |
|---|---|---|---|
| pound | | | |
| No. | A | R¹ | R² |
| 1 | 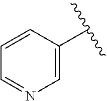 [SMILES: *c1cccnc1] | H | 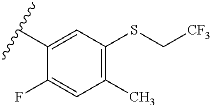 [SMILES: *c1cc(SCC(F)(F)F)c(C)cc1F] |
| 2 | 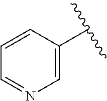 [SMILES: *c1cccnc1] | H | 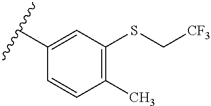 [SMILES: *c1ccc(C)c(SCC(F)(F)F)c1] |
| 3 | 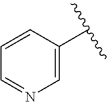 [SMILES: *c1cccnc1] | H | 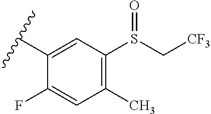 [SMILES: *c1cc(S(=O)CC(F)(F)F)c(C)cc1F] |
| 4 | 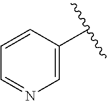 [SMILES: *c1cccnc1] | H | 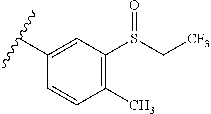 [SMILES: *c1ccc(C)c(S(=O)CC(F)(F)F)c1] |
| 6 | 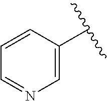 [SMILES: *c1cccnc1] | H | 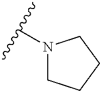 [SMILES: *N1CCCC1] |
| 7 | 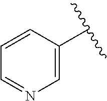 [SMILES: *c1cccnc1] | H | 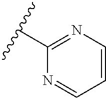 [SMILES: *c1ncccn1] |
| 8 | 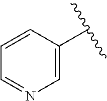 [SMILES: *c1cccnc1] | H | 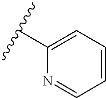 [SMILES: *c1ccccn1] |
| 9 | 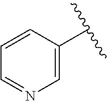 [SMILES: *c1cccnc1] | H | 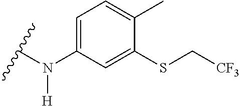 [SMILES: *Nc1ccc(C)c(SCC(F)(F)F)c1] |
| 10 | 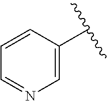 [SMILES: *c1cccnc1] | H | 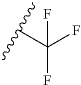 [SMILES: *C(F)(F)F] |
| 11 | 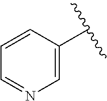 [SMILES: *c1cccnc1] | H | 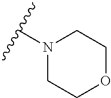 [SMILES: *N1CCOCC1] |
| 12 | 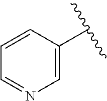 [SMILES: *c1cccnc1] | H | 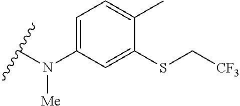 [SMILES: *N(C)c1ccc(C)c(SCC(F)(F)F)c1] |
| 13 | 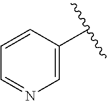 [SMILES: *c1cccnc1] | H | 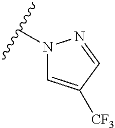 [SMILES: *n1cc(C(F)(F)F)cn1] |
| 14 | 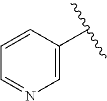 [SMILES: *c1cccnc1] | H | 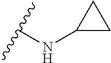 [SMILES: *NC1CC1] |
| 15 | 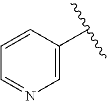 [SMILES: *c1cccnc1] | H | 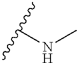 [SMILES: *NC] |
| 16 | 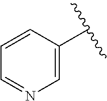 [SMILES: *c1cccnc1] | H | 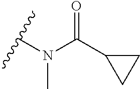 [SMILES: *N(C)C(=O)C1CC1] |
| 17 | 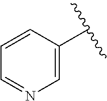 [SMILES: *c1cccnc1] | H | 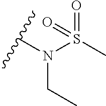 [SMILES: *N(CC)S(C)(=O)=O] |
| 18 | 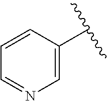 [SMILES: *c1cccnc1] | H | 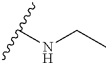 [SMILES: *NCC] |
| 19 | 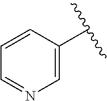 [SMILES: *c1cccnc1] | H | 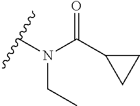 [SMILES: *N(CC)C(=O)C1CC1] |
| 20* | 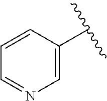 [SMILES: *c1cccnc1] | H | 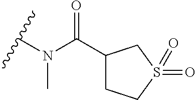 [SMILES: *N(C)C(=O)C1CCS(=O)(=O)C1] |
| 21* | 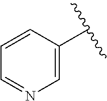 [SMILES: *c1cccnc1] | H | 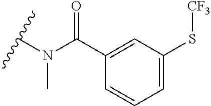 [SMILES: *N(C)C(=O)c1cccc(SC(F)(F)F)c1] |
| 22 | 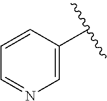 [SMILES: *c1cccnc1] | H | 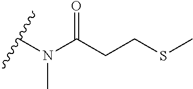 [SMILES: *N(C)C(=O)CCSC] |
| 23* | 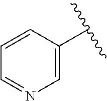 [SMILES: *c1cccnc1] | H | 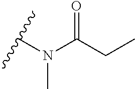 [SMILES: *N(C)C(=O)CC] |
| 24* | 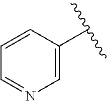 [SMILES: *c1cccnc1] | H | 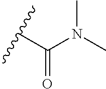 [SMILES: *C(=O)N(C)C] |
| 25* | 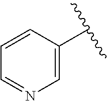 [SMILES: *c1cccnc1] | H | 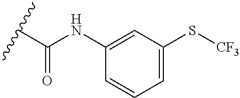 [SMILES: *C(=O)Nc1cccc(SC(F)(F)F)c1] |
| 26* | 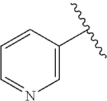 [SMILES: *c1cccnc1] | H | 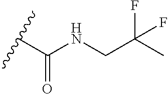 [SMILES: *C(=O)NCC(C)(F)F] |
| 27* | 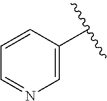 [SMILES: *c1cccnc1] | H | 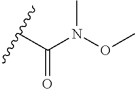 [SMILES: *C(=O)N(C)OC] |
| 28* | 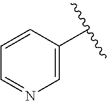 [SMILES: *c1cccnc1] | H | 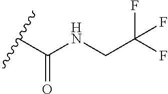 [SMILES: *C(=O)NCC(F)(F)F] |
| 29 | 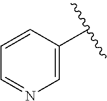 [SMILES: *c1cccnc1] | H | 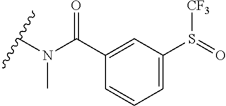 [SMILES: *N(C)C(=O)c1cccc(S(=O)C(F)(F)F)c1] |
| 30 | 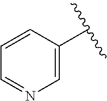 [SMILES: *c1cccnc1] | H | 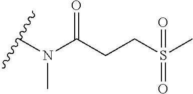 [SMILES: *N(C)C(=O)CCS(C)(=O)=O] |
| 31 | 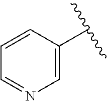 [SMILES: *c1cccnc1] | H | 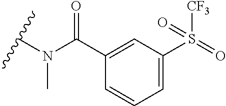 [SMILES: *N(C)C(=O)c1cccc(S(=O)(=O)C(F)(F)F)c1] |
| 32 | 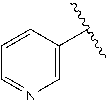 [SMILES: *c1cccnc1] | H | 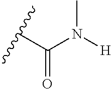 [SMILES: *C(=O)NC] |
| 33 | 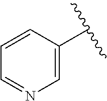 [SMILES: *c1cccnc1] | H | 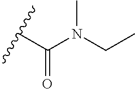 [SMILES: *C(=O)N(C)CC] |
| 34 | 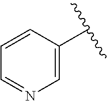 [SMILES: *c1cccnc1] | H | 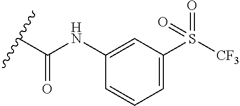 [SMILES: *C(=O)Nc1cccc(S(=O)(=O)C(F)(F)F)c1] |
| 35 | 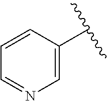 [SMILES: *c1cccnc1] | H | 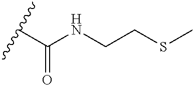 [SMILES: *C(=O)NCCSC] |
| 36 | 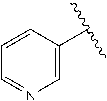 [SMILES: *c1cccnc1] | H | 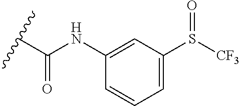 [SMILES: *C(=O)Nc1cccc(S(=O)C(F)(F)F)c1] |
| 37 | 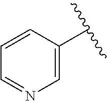 [SMILES: *c1cccnc1] | H | 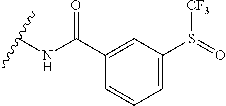 [SMILES: *NC(=O)c1cccc(S(=O)C(F)(F)F)c1] |
| 38 | 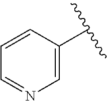 [SMILES: *c1cccnc1] | H | 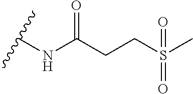 [SMILES: *NC(=O)CCS(C)(=O)=O] |
| 39 | 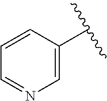 [SMILES: *c1cccnc1] | H | 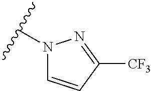 [SMILES: *n1ccc(C(F)(F)F)n1] |
| 40 | 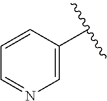 [SMILES: *c1cccnc1] | H | 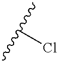 [SMILES: *Cl] |
| 42 | 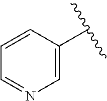 [SMILES: *c1cccnc1] | H | 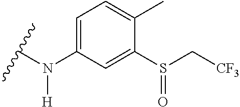 [SMILES: *Nc1ccc(C)c(S(=O)CC(F)(F)F)c1] |

Compounds marked with an * were isolated as salts of formic acid and characterized and biologically tested as such.

TABLE 2
Analytical data for the compounds reported.

| Ex. No. | logP[a] | logP[b] | ¹H NMR [σ (ppm)] or LC-MS [m/z] |
|---|---|---|---|
| 1 | 4.81 | 4.75 | ¹H-NMR(400.0 MHz, d₆-DMSO): δ = 9.336(9.6); 9.331(9.4); |
| | | | 9.330(9.2); 8.819(7.7); 8.815(8.5); 8.807(8.3); 8.803(8.5); 8.615(14.3); |
| | | | 8.593(16.0); 8.536(4.4); 8.532(5.7); 8.530(5.6); 8.526(4.5); 8.516(4.8); |
| | | | 8.512(5.8); 8.510(6.2); 8.506(4.6); 8.318(0.8); 8.208(9.7); 8.188(9.7); |
| | | | 8.040(6.9); 8.036(7.9); 8.018(7.2); 8.014(6.9); 7.685(5.6); 7.683(5.7); |
| | | | 7.673(5.4); 7.671(5.5); 7.665(5.4); 7.663(5.4); 7.653(5.3); 7.651(5.4); |
| | | | 7.423(8.2); 7.411(0.5); 7.392(8.1); 4.008(4.2); 3.982(13.4); 3.956(14.0); |
| | | | 3.930(4.8); 3.330(261.5); 2.682(0.7); 2.677(1.5); 2.673(2.1); 2.668(1.6); |
| | | | 2.664(0.8); 2.656(0.3); 2.526(5.6); 2.521(8.5); 2.512(114.8); |
| | | | 2.508(235.5); 2.504(315.5); 2.499(263.2); 2.420(1.4); 2.368(0.5); |
| | | | 2.335(1.8); 2.330(2.1); 2.326(1.6); 2.321(0.9); 1.990(0.6); 1.398(4.1); |
| | | | 0.146(2.5); 0.029(0.4); 0.008(19.6); 0.000(555.1); −0.009(21.4); |
| | | | −0.033(0.4); −0.150(2.6). |
| 2 | 4.67 | 4.64 | ¹H-NMR(400.0 MHz, d₆-DMSO): δ = 9.322(1.9); 8.808(1.4); |
| | | | 8.799(1.4); 8.591(4.3); 8.581(0.4); 8.569(5.1); 8.560(0.4); 8.516(1.3); |
| | | | 8.511(1.9); 8.507(1.4); 8.496(1.4); 8.491(2.0); 8.487(1.4); 8.317(0.5); |
| | | | 8.307(3.9); 8.301(7.3); 8.279(4.2); 8.070(2.2); 8.066(2.1); 8.050(2.2); |
| | | | 8.046(2.2); 7.677(1.7); 7.666(1.7); 7.658(1.6); 7.646(1.6); 7.456(3.0); |
| | | | 7.436(2.8); 4.156(1.3); 4.130(4.2); 4.104(4.4); 4.078(1.5); 3.331(95.2); |
| | | | 2.677(0.4); 2.673(0.6); 2.668(0.5); 2.526(1.4); 2.512(35.3); 2.508(70.8); |
| | | | 2.504(93.0); 2.499(68.9); 2.495(34.7); 2.455(16.0); 2.391(0.8); |
| | | | 2.340(0.4); 2.335(0.5); 2.330(0.7); 2.326(0.5); 1.397(3.2); 0.008(0.4); |
| | | | 0.000(11.3); −0.008(0.5). |
| 3 | 3.02 | 3.09 | ¹H-NMR(400.0 MHz, d₆-DMSO): δ = 9.341(3.3); 9.336(3.3); |
| | | | 8.823(2.3); 8.820(2.6); 8.811(2.5); 8.808(2.6); 8.654(3.6); 8.633(4.0); |
| | | | 8.593(0.4); 8.581(3.0); 8.562(3.0); 8.535(2.0); 8.530(1.5); 8.519(1.5); |
| | | | 8.514(2.0); 8.510(1.5); 8.317(0.4); 8.126(2.1); 8.122(2.4); 8.104(2.1); |
| | | | 8.101(2.1); 7.687(1.8); 7.675(1.9); 7.667(1.8); 7.655(1.7); 7.526(2.5); |
| | | | 7.496(2.5); 5.757(2.9); 4.261(0.4); 4.251(0.6); 4.232(1.0); 4.224(1.7); |
| | | | 4.205(1.9); 4.197(1.9); 4.178(1.7); 4.169(1.0); 4.150(0.6); 4.141(0.4); |
| | | | 3.329(81.5); 2.672(1.0); 2.668(0.8); 2.507(113.6); 2.503(144.6); |
| | | | 2.499(114.9); 2.468(16.0); 2.429(0.4); 2.330(1.0); 0.000(17.6). |
| 4 | 2.99 | 2.98 | ¹H-NMR(400.0 MHz, d₆-DMSO): δ = 9.325(3.3); 9.320(3.3); |
| | | | 8.813(2.4); 8.810(2.6); 8.802(2.5); 8.798(2.6); 8.717(4.0); 8.713(4.2); |
| | | | 8.629(3.7); 8.607(4.3); 8.522(1.4); 8.517(2.0); 8.512(1.5); 8.502(1.5); |
| | | | 8.497(2.1); 8.492(1.4); 8.338(2.3); 8.331(5.1); 8.318(2.6); 8.310(4.1); |
| | | | 7.680(1.8); 7.668(1.8); 7.660(1.8); 7.648(1.7); 7.552(2.9); 7.532(2.8); |
| | | | 5.758(0.9); 4.235(0.7); 4.226(0.7); 4.208(2.0); 4.199(1.9); 4.181(2.0); |
| | | | 4.172(2.0); 4.154(0.7); 4.145(0.7); 4.136(0.3); 3.366(0.3); 3.331(76.2); |
| | | | 2.672(0.7); 2.507(78.8); 2.503(100.7); 2.499(78.2); 2.460(16.0); |
| | | | 2.330(0.7); 0.000(2.6). |
| 6 | 2.53 | 2.97 | ¹H-NMR(400.0 MHz, d₆-DMSO): δ = 9.151(5.5); 9.147(5.6); |
| | | | 8.683(3.9); 8.679(4.2); 8.671(4.1); 8.667(4.1); 8.335(0.5); 8.327(2.5); |
| | | | 8.323(3.4); 8.318(2.6); 8.314(1.6); 8.307(2.7); 8.302(3.5); 8.298(2.5); |
| | | | 8.140(8.2); 8.118(8.6); 7.580(3.1); 7.569(3.1); 7.561(3.1); 7.549(2.9); |
| | | | 7.521(0.4); 6.731(7.9); 6.708(7.7); 5.754(1.8); 3.510(5.5); 3.494(14.2); |
| | | | 3.477(5.8); 3.316(134.8); 3.141(0.9); 2.675(1.7); 2.671(2.3); 2.666(1.7); |
| | | | 2.632(1.8); 2.571(0.4); 2.567(0.4); 2.562(0.4); 2.524(7.4); 2.510(135.4); |
| | | | 2.506(268.7); 2.502(352.7); 2.497(264.6); 2.493(136.1); 2.333(1.6); |
| | | | 2.328(2.2); 2.324(1.7); 2.018(0.7); 2.003(6.0); 1.993(7.0); 1.986(16.0); |
| | | | 1.980(7.3); 1.970(5.9); 1.335(0.8); 1.259(0.7); 1.250(0.6); 1.233(2.1); |
| | | | 0.854(0.4); 0.146(2.1); 0.031(0.4); 0.022(0.8); 0.008(17.8); |
| | | | 0.000(454.2); −0.008(23.0); −0.028(0.7); −0.150(2.2). |
| 7 | 1.43 | 1.50 | ¹H-NMR(400.0 MHz, d₆-DMSO): δ = 9.357(3.1); 9.352(3.1); |
| | | | 9.049(8.9); 9.037(9.0); 8.829(2.3); 8.825(2.5); 8.817(2.4); 8.813(2.4); |
| | | | 8.667(16.0); 8.564(1.3); 8.560(1.9); 8.555(1.4); 8.544(1.4); 8.539(1.9); |
| | | | 8.535(1.3); 8.319(0.7); 7.690(1.7); 7.678(1.7); 7.671(1.7); 7.659(1.6); |
| | | | 7.634(2.5); 7.622(4.7); 7.610(2.4); 5.759(1.0); 3.330(115.0); 2.676(1.3); |
| | | | 2.672(1.7); 2.667(1.3); 2.525(4.6); 2.507(204.2); 2.503(264.8); |
| | | | 2.498(196.5); 2.334(1.3); 2.329(1.7); 2.325(1.3); 1.258(0.5); 1.233(1.9); |
| | | | 0.852(0.6); 0.833(0.3); 0.146(0.6); 0.008(4.5); |
| | | | 0.000(124.1); −0.150(0.6). |
| 8 | 2.12 | 2.68 | ¹H-NMR(601.6 MHz, CDCl₃): δ = 9.345(6.9); 9.344(7.1); 9.342(7.2); |
| | | | 9.341(6.4); 8.772(5.8); 8.769(5.9); 8.764(6.0); 8.762(5.7); 8.737(3.9); |
| | | | 8.736(4.6); 8.734(4.6); 8.733(4.0); 8.729(4.2); 8.728(4.6); 8.727(4.4); |
| | | | 8.725(3.6); 8.648(12.4); 8.634(14.1); 8.511(4.7); 8.510(6.8); 8.508(4.3); |
| | | | 8.498(4.9); 8.497(7.1); 8.495(4.3); 8.426(15.6); 8.422(5.2); 8.421(4.8); |
| | | | 8.418(3.8); 8.412(16.0); 8.409(5.6); 8.408(5.0); 8.405(3.7); 7.883(3.5); |
| | | | 7.880(3.4); 7.870(5.6); 7.868(5.5); 7.857(3.6); 7.854(3.4); 7.498(4.4); |
| | | | 7.496(4.2); 7.490(4.5); 7.488(4.3); 7.484(4.4); 7.483(4.1); 7.477(4.3); |
| | | | 7.475(4.0); 7.434(0.3); 7.369(3.9); 7.368(3.8); 7.362(3.9); 7.360(4.1); |
| | | | 7.357(4.1); 7.355(3.7); 7.349(3.7); 7.347(3.4); 7.262(58.9); 7.086(0.3); |
| | | | 1.594(59.7); 1.333(0.7); 1.284(1.0); 1.254(1.5); 0.880(0.3); 0.844(0.4); |
| | | | 0.839(0.4); 0.005(1.5); 0.000(45.7); −0.006(1.7). |
| 9 | 3.99 | 4.06 | ¹H-NMR(400.0 MHz, d₆-DMSO): δ = 9.638(3.9); 9.195(2.9); |
| | | | 9.191(3.0); 8.717(2.3); 8.713(2.4); 8.705(2.4); 8.701(2.4); 8.378(1.2); |
| | | | 8.374(1.7); 8.368(1.3); 8.358(1.4); 8.353(1.8); 8.348(1.4); 8.318(0.4); |
| | | | 8.236(4.2); 8.214(4.5); 8.007(3.2); 8.002(3.3); 7.607(1.7); 7.595(1.6); |
| | | | 7.587(1.6); 7.573(2.1); 7.571(2.0); 7.565(1.7); 7.550(2.0); 7.545(2.0); |
| | | | 7.236(2.9); 7.215(2.5); 7.038(4.0); 7.015(4.0); 4.056(0.4); 4.038(1.1); |
| | | | 4.020(1.1); 4.002(0.4); 3.954(1.2); 3.928(3.7); 3.902(3.9); 3.876(1.3); |
| | | | 3.330(134.6); 2.676(0.8); 2.672(1.1); 2.667(0.8); 2.525(3.4); |
| | | | 2.511(66.4); 2.507(131.9); 2.503(172.2); 2.498(126.1); 2.494(63.2); |
| | | | 2.338(16.0); 2.266(0.5); 1.989(4.8); 1.397(0.4); 1.193(1.3); 1.175(2.5); |
| | | | 1.157(1.3); 0.146(0.9); 0.008(7.6); 0.000(197.5); −0.008(8.7); |
| | | | −0.150(0.9). |
| 10 | 2.65 | 2.69 | ¹H-NMR(400.0 MHz, d₆-DMSO): δ = 9.354(8.2); 9.352(8.9); |
| | | | 9.348(8.9); 9.346(8.5); 8.849(7.2); 8.845(7.8); 8.837(7.7); 8.833(7.7); |
| | | | 8.781(8.4); 8.780(8.4); 8.760(9.1); 8.759(9.0); 8.561(4.4); 8.557(5.5); |
| | | | 8.556(5.3); 8.551(4.4); 8.541(4.8); 8.537(5.5); 8.535(5.8); 8.531(4.5); |
| | | | 8.151(16.0); 8.130(15.0); 7.700(5.4); 7.698(5.5); 7.688(5.2); 7.686(5.3); |
| | | | 7.680(5.2); 7.678(5.2); 7.668(5.1); 7.666(5.1); 3.338(38.7); 2.681(0.3); |
| | | | 2.677(0.5); 2.672(0.3); 2.530(1.2); 2.525(1.7); 2.517(26.2); 2.512(54.4); |
| | | | 2.508(72.0); 2.503(51.8); 2.499(24.7); 2.339(0.3); 2.334(0.5); |
| | | | 2.330(0.3); 1.993(0.4); 1.225(1.4); 0.008(0.9); 0.000(30.6); −0.009(1.0). |
| 11 | 2.10 | 1.95 | ¹H-NMR(400.0 MHz, d₆-DMSO): δ = 9.179(5.8); 9.174(5.8); |
| | | | 8.709(4.1); 8.706(4.4); 8.697(4.3); 8.694(4.2); 8.356(3.6); 8.336(3.7); |
| | | | 8.317(0.4); 8.222(6.4); 8.199(6.7); 7.598(3.3); 7.586(3.4); 7.578(3.3); |
| | | | 7.566(3.0); 7.138(6.3); 7.115(6.1); 3.745(9.6); 3.733(16.0); 3.721(13.6); |
| | | | 3.609(13.4); 3.596(15.7); 3.585(9.5); 3.332(188.8); 2.672(1.2); |
| | | | 2.503(168.0); 2.330(1.1); 1.990(0.5); 0.000(15.6). |
| 12 | 4.81 | 4.78 | ¹H-NMR(601.6 MHz, CDCl₃): 8 = 9.227(1.6); 9.224(1.6); 8.677(1.1); |
| | | | 8.674(1.2); 8.669(1.2); 8.666(1.1); 8.297(0.8); 8.293(1.1); 8.290(0.8); |
| | | | 8.283(0.8); 8.280(1.1); 8.277(0.8); 7.901(2.9); 7.885(2.9); 7.424(1.0); |
| | | | 7.423(1.0); 7.413(2.4); 7.410(2.9); 7.403(1.0); 7.402(0.9); 7.310(1.5); |
| | | | 7.296(1.8); 7.264(3.3); 7.164(1.4); 7.160(1.3); 7.151(1.1); 7.147(1.1); |
| | | | 6.627(2.9); 6.611(2.9); 5.299(2.2); 3.545(1.4); 3.538(16.0); 3.438(1.1); |
| | | | 3.422(3.4); 3.406(3.5); 3.391(1.2); 2.491(10.7); 2.442(1.0); 2.359(0.7); |
| | | | 1.688(1.5); 1.254(1.6); 0.000(0.9). |
| 13 | 3.82 | 3.72 | ¹H-NMR(400.0 MHz, d₆-DMSO): δ = 9.397(12.4); 9.334(8.8); |
| | | | 9.330(8.9); 8.819(6.7); 8.816(7.5); 8.807(7.2); 8.804(7.4); 8.747(14.6); |
| | | | 8.725(16.0); 8.538(3.9); 8.533(5.4); 8.528(4.1); 8.518(4.3); 8.512(5.7); |
| | | | 8.508(4.3); 8.382(15.9); 8.317(2.8); 8.235(15.5); 8.213(14.5); |
| | | | 7.992(0.5); 7.684(5.1); 7.682(5.3); 7.672(4.9); 7.670(5.1); 7.664(4.9); |
| | | | 7.662(5.0); 7.652(4.7); 7.650(4.8); 3.329(899.6); 2.676(4.4); 2.672(6.1); |
| | | | 2.667(4.6); 2.542(2.4); 2.525(16.4); 2.520(24.9); 2.511(334.7); |
| | | | 2.507(693.8); 2.503(925.4); 2.498(682.8); 2.494(341.5); 2.334(4.3); |
| | | | 2.329(6.0); 2.325(4.5); 2.075(0.3); 1.148(0.6); 0.146(7.1); 0.032(1.0); |
| | | | 0.025(2.1); 0.008(54.7); 0.000(1562.0); −0.009(65.0); −0.029(1.5); |
| | | | −0.038(0.8); −0.042(0.6); −0.050(0.5); −0.065(0.4); −0.150(7.1). |
| 14 | | | ¹H-NMR(400 MHz, CDCl₃): δ = 9.23 (s, 1 H), 8.68-8.67 (m, 1 H), |
| | | | 8.29-8.26 (m, 1 H), 8.12-8.09 (m, 1 H), 7.43-7.40 (m, 1 H), |
| | | | 6.96-6.94 (m, 1 H), 5.25 (s, 1 H), 2.66-2.62 (m, 1 H), 0.89-0.86 |
| | | | (m, 2 H), 0.65-0.63 (m, 2 H). |
| 15 | 1.34 | | |
| 16 | | | ¹H-NMR(400 MHz, CDCl₃): δ = 9.13 (s, 1 H), 8.77-8.76 (m, 1 H), |
| | | | 8.38-8.31 (m, 2 H), 7.59-7.57 (m, 1 H), 7.50-7.47 (m, 1 H), 3.54 |
| | | | (s, 3 H), 1.73-1.70 (m, 1 H), 1.18-1.14 (m, 2 H), 0.84-0.80 |
| | | | (m, 2 H). |
| 17 | | | ¹H-NMR(400 MHz, CDCl₃): δ = 9.29 (s, 1 H), 8.77-8.76 (m, 1 H), |
| | | | 8.39-8.32 (m, 2 H), 7.62-7.59 (m, 1 H), 7.50-7.47 (m, 1 H), |
| | | | 4.07-4.02 (m, 2 H), 3.09 (s, 3 H), 1.26-1.22 (m, 3 H). |
| 18 | | | ¹H-NMR(400 MHz, CDCl₃): δ = 9.14 (s, 1 H), 8.60-8.58 (m, 1 H), |
| | | | 8.21-8.18 (m, 1 H), 7.94-7.92 (m, 1 H), 7.38-7.32 (m, 1 H), |
| | | | 6.48-6.46 (m, 1 H), 4.64 (s, 1 H), 3.40-3.37 (m, 2 H), 1.25-1.22 (m, 3 H). |
| 19 | | | ¹H-NMR(400 MHz, CDCl₃): δ = 9.24 (s, 1 H), 8.70-8.69 (m, 1 H), |
| | | | 8.32-8.25 (m, 2 H), 7.46-7.39 (m, 2 H), 4.03-3.98 (m, 2 H), |
| | | | 1.50-1.48 (m, 1 H), 1.18-1.15 (m, 3 H), 1.09-1.07 (m, 2 H), 0.71-0.70 |
| | | | (m, 2 H). |
| 20 | 1.37 | | ¹H-NMR(400.0 MHz, d₆-DMSO): δ = 9.314(2.4); 8.816(1.7); |
| | | | 8.807(1.8); 8.628(5.0); 8.606(5.4); 8.510(1.6); 8.505(2.4); 8.501(1.8); |
| | | | 8.491(1.9); 8.485(2.6); 8.481(1.9); 8.310(0.4); 7.779(5.8); 7.758(5.6); |
| | | | 7.678(1.8); 7.666(1.9); 7.658(1.9); 7.646(1.8); 3.902(16.0); 3.585(1.0); |
| | | | 3.565(0.9); 3.508(0.5); 3.392(22.5); 3.352(536.8); 3.291(2.1); |
| | | | 3.278(2.7); 3.257(2.4); 3.240(1.3); 3.225(3.3); 3.208(2.3); 3.202(2.9); |
| | | | 3.191(2.2); 3.176(1.8); 3.169(1.5); 3.058(0.9); 3.034(1.2); 3.025(0.9); |
| | | | 3.014(1.1); 3.001(1.0); 2.982(0.7); 2.872(0.5); 2.860(0.5); 2.678(1.0); |
| | | | 2.673(1.3); 2.669(1.0); 2.527(3.5); 2.513(74.3); 2.509(156.7); |
| | | | 2.504(219.6); 2.500(164.1); 2.495(78.1); 2.336(1.0); 2.331(1.4); |
| | | | 2.326(1.1); 2.309(0.5); 2.293(0.9); 2.277(1.1); 2.264(0.9); 2.246(1.2); |
| | | | 2.222(1.5); 2.213(0.5); 2.203(1.4); 2.189(0.8); 2.179(0.7); 2.170(0.7); |
| | | | 2.143(0.9); 0.000(3.0) |
| 21 | 3.39 | | ¹H-NMR(400.0 MHz, d₆-DMSO): δ = 9.239(2.3); 8.766(1.5); |
| | | | 8.755(1.6); 8.477(2.7); 8.435(12.4); 8.420(1.8); 8.413(1.9); 8.410(2.2); |
| | | | 7.881(1.3); 7.861(1.5); 7.675(1.5); 7.655(3.0); 7.636(1.9); 7.620(1.2); |
| | | | 4.172(16.0); 3.901(7.5); 3.550(0.8); 3.508(0.7); 3.363(521.7); |
| | | | 3.355(877.8); 3.177(0.8); 3.164(0.8); 2.673(1.5); 2.509(170.4); |
| | | | 2.504(235.5); 2.500(187.4); 2.331(1.3); 0.000(2.2) |
| 22 | 1.98 | | ¹H-NMR(400.0 MHz, d₆-DMSO): δ = 9.301(1.3); 8.807(0.9); |
| | | | 8.797(0.9); 8.574(2.8); 8.552(3.0); 8.496(0.9); 8.491(1.5); 8.487(0.9); |
| | | | 8.476(0.9); 8.470(1.4); 7.789(2.5); 7.767(2.4); 7.670(0.9); 7.658(1.0); |
| | | | 7.650(1.0); 7.638(0.9); 3.902(2.8); 3.417(0.4); 3.392(13.0); |
| | | | 3.336(158.8); 3.329(84.5); 2.767(1.4); 2.714(10.8); 2.677(0.5); |
| | | | 2.672(0.6); 2.668(0.4); 2.512(34.0); 2.508(69.0); 2.503(95.5); |
| | | | 2.499(72.2); 2.494(35.4); 2.334(0.4); 2.330(0.5); 2.326(0.4); 2.102(2.6); |
| | | | 2.000(16.0); 0.000(1.7) |
| 23 | 1.71 | | ¹H-NMR(400.0 MHz, d₆-DMSO): δ = 9.295(2.6); 8.804(1.8); |
| | | | 8.795(1.8); 8.548(6.3); 8.526(6.8); 8.491(1.7); 8.486(2.5); 8.481(1.8); |
| | | | 8.471(1.8); 8.466(2.6); 8.461(1.7); 7.794(5.6); 7.772(5.4); 7.669(1.9); |
| | | | 7.656(1.9); 7.649(1.9); 7.637(1.8); 3.902(4.8); 3.509(0.4); 3.381(33.0); |
| | | | 3.344(337.1); 2.677(0.7); 2.673(1.0); 2.669(0.7); 2.526(2.7); |
| | | | 2.512(56.7); 2.508(118.3); 2.504(166.3); 2.499(127.2); 2.495(63.3); |
| | | | 2.459(2.0); 2.441(6.0); 2.422(6.1); 2.404(2.0); 2.335(0.7); 2.331(0.9); |
| | | | 2.326(0.7); 1.042(7.8); 1.024(16.0); 1.005(7.4); 0.000(1.1) |
| 24 | 1.22 | | ¹H-NMR(400.0 MHz, d₆-DMSO): δ = 9.332(1.3); 8.827(0.9); |
| | | | 8.817(0.9); 8.612(3.9); 8.591(4.1); 8.537(0.9); 8.532(1.1); 8.527(0.9); |
| | | | 8.517(0.9); 8.512(1.2); 8.507(0.8); 7.801(4.0); 7.780(3.8); 7.690(0.9); |
| | | | 7.678(0.9); 7.670(0.9); 7.658(0.8); 3.902(2.5); 3.359(205.8); 3.170(0.6); |
| | | | 3.063(14.1); 3.001(16.0); 2.868(1.7); 2.678(0.3); 2.673(0.5); 2.669(0.4); |
| | | | 2.527(1.3); 2.513(28.2); 2.509(59.8); 2.504(84.0); 2.500(62.4); |
| | | | 2.495(29.6); 2.335(0.3); 2.331(0.5); 2.326(0.4); 1.275(0.6); 1.260(1.1); |
| | | | 1.245(0.9); 0.000(1.3) |
| 25 | 4.33 | | ¹H-NMR(400.0 MHz, d₆-DMSO): δ = 11.171(13.2); 9.386(7.1); |
| | | | 8.851(4.9); 8.840(5.0); 8.732(13.9); 8.711(16.0); 8.587(3.8); 8.582(5.8); |
| | | | 8.577(4.4); 8.567(4.2); 8.562(6.0); 8.558(4.4); 8.459(11.7); 8.395(15.4); |
| | | | 8.374(14.2); 8.312(0.5); 8.186(5.6); 8.166(6.1); 7.708(4.6); 7.696(4.7); |
| | | | 7.688(4.7); 7.676(4.2); 7.586(5.2); 7.566(11.4); 7.546(7.8); 7.496(8.3); |
| | | | 7.477(5.1); 4.100(0.3); 3.902(14.2); 3.762(0.3); 3.722(0.4); 3.711(0.4); |
| | | | 3.698(0.4); 3.688(0.4); 3.657(0.5); 3.638(0.5); 3.600(0.6); 3.569(0.7); |
| | | | 3.508(1.4); 3.344(1201.2); 3.216(0.3); 3.176(1.0); 3.164(0.9); |
| | | | 2.678(2.3); 2.673(3.3); 2.669(2.5); 2.623(0.4); 2.612(0.5); 2.509(393.5); |
| | | | 2.504(550.6); 2.500(436.2); 2.335(2.1); 2.331(3.0); 2.326(2.3); |
| | | | 1.249(0.4); 1.232(0.8); 0.000(8.0) |
| 26 | 2.09 | | ¹H-NMR(400.0 MHz, d₆-DMSO): δ = 9.358(4.8); 9.308(1.8); |
| | | | 9.292(3.6); 9.276(1.8); 8.840(3.2); 8.829(3.3); 8.677(7.2); 8.655(8.0); |
| | | | 8.558(3.4); 8.537(3.6); 8.293(7.7); 8.271(7.1); 7.696(2.8); 7.684(2.9); |
| | | | 7.676(2.9); 7.665(2.6); 3.902(6.4); 3.835(2.2); 3.818(2.3); 3.800(4.5); |
| | | | 3.784(4.5); 3.765(2.4); 3.749(2.3); 3.608(0.4); 3.540(0.5); 3.508(0.7); |
| | | | 3.347(684.3); 3.171(0.7); 2.674(1.5); 2.580(0.3); 2.509(182.9); |
| | | | 2.504(257.9); 2.500(208.8); 2.335(1.0); 2.331(1.4); 2.327(1.2); |
| | | | 2.282(0.6); 1.700(7.4); 1.653(16.0); 1.605(8.0); 1.208(0.6); 1.191(0.7); |
| | | | 0.000(3.0) |
| 27 | 1.39 | | ¹H-NMR(400.0 MHz, d₆-DMSO): δ = 9.334(4.4); 8.830(3.1); |
| | | | 8.820(3.2); 8.621(9.5); 8.600(10.3); 8.536(2.5); 8.531(4.1); 8.527(3.1); |
| | | | 8.516(2.9); 8.511(4.4); 8.507(3.2); 8.259(0.4); 7.856(2.8); 7.835(2.7); |
| | | | 7.688(3.1); 7.676(3.3); 7.668(3.4); 7.656(3.2); 4.563(0.5); 3.902(6.2); |
| | | | 3.705(16.0); 3.510(0.5); 3.343(495.0); 3.170(2.0); 2.991(0.4); |
| | | | 2.869(0.8); 2.857(0.8); 2.678(1.2); 2.673(1.5); 2.669(1.2); 2.526(4.1); |
| | | | 2.513(87.0); 2.508(182.5); 2.504(257.3); 2.499(197.4); 2.335(1.0); |
| | | | 2.331(1.4); 2.326(1.1); 0.000(2.0) |
| 28 | 2.17 | | ¹H-NMR(400.0 MHz, d₆-DMSO): δ = 9.640(2.6); 9.624(5.1); |
| | | | 9.607(2.5); 9.363(6.8); 8.843(4.7); 8.833(4.8); 8.689(9.4); 8.668(10.5); |
| | | | 8.563(4.8); 8.543(5.0); 8.303(10.4); 8.282(9.5); 7.698(3.8); 7.686(4.0); |
| | | | 7.679(4.0); 7.666(3.6); 4.167(1.6); 4.144(5.0); 4.127(5.3); 4.120(5.6); |
| | | | 4.103(5.0); 4.079(1.7); 3.902(16.0); 3.511(0.5); 3.337(468.1); |
| | | | 3.332(548.5); 3.170(0.9); 2.672(2.1); 2.503(356.0); 2.330(2.0); |
| | | | 1.234(0.4); 0.000(5.0) |
| 29 | 2.23 | | ¹H-NMR(400.0 MHz, d₆-DMSO): δ = 8.801(2.9); 8.436(2.8); |
| | | | 8.413(3.8); 8.392(1.7); 7.965(1.6); 7.945(1.8); 7.902(0.5); 7.889(0.3); |
| | | | 7.711(1.7); 7.692(2.0); 7.634(1.8); 7.623(1.7); 7.614(2.6); 7.588(1.1); |
| | | | 7.560(3.3); 7.527(3.1); 7.518(2.0); 7.505(3.1); 7.499(3.0); 7.480(1.2); |
| | | | 3.902(5.4); 3.550(16.0); 3.330(260.8); 3.269(0.4); 3.176(0.5); |
| | | | 3.163(0.5); 2.672(0.9); 2.503(158.8); 2.330(0.9); 0.000(1.6) |
| 30 | 1.25 | | ¹H-NMR(400.0 MHz, d₆-DMSO): δ = 9.299(2.1); 9.295(2.1); |
| | | | 8.802(1.5); 8.792(1.5); 8.595(3.3); 8.573(3.5); 8.499(1.1); 8.494(1.5); |
| | | | 8.489(1.1); 8.479(1.2); 8.474(1.6); 8.469(1.1); 7.819(3.2); 7.797(3.0); |
| | | | 7.669(1.3); 7.657(1.3); 7.649(1.3); 7.637(1.2); 3.902(3.7); 3.431(2.9); |
| | | | 3.423(14.8); 3.414(4.1); 3.394(2.7); 3.330(150.6); 3.176(0.7); |
| | | | 3.163(0.7); 2.991(16.0); 2.931(1.6); 2.912(2.3); 2.893(1.3); 2.676(0.6); |
| | | | 2.672(0.8); 2.667(0.6); 2.525(2.2); 2.511(46.0); 2.507(94.4); |
| | | | 2.502(130.3); 2.498(97.4); 2.494(47.0); 2.334(0.5); 2.329(0.7); |
| | | | 2.325(0.6); 0.000(2.9) |
| 31 | 1.54 | | ¹H-NMR(400.0 MHz, d₆-DMSO): δ = 8.800(2.2); 8.444(2.7); |
| | | | 8.422(2.9); 8.406(1.3); 8.391(1.3); 7.965(1.2); 7.945(1.3); 7.864(1.2); |
| | | | 7.844(1.5); 7.778(1.3); 7.759(1.9); 7.722(2.5); 7.687(1.7); 7.667(2.4); |
| | | | 7.648(1.0); 7.624(1.1); 7.607(1.4); 7.588(1.0); 7.550(2.8); 7.528(2.8); |
| | | | 4.110(0.5); 4.098(0.4); 3.902(6.4); 3.565(16.0); 3.348(372.1); |
| | | | 3.268(0.7); 3.176(3.3); 3.164(3.3); 2.673(0.9); 2.669(0.7); 2.512(54.0); |
| | | | 2.508(110.0); 2.504(151.7); 2.499(117.6); 2.495(60.1); 2.335(0.6); |
| | | | 2.331(0.8); 2.326(0.6); 0.000(1.0) |
| 32 | 1.35 | | ¹H-NMR(400.0 MHz, d₆-DMSO): δ = 9.353(3.1); 9.023(2.0); |
| | | | 9.011(2.0); 8.837(2.2); 8.829(2.3); 8.647(6.7); 8.625(7.6); 8.547(2.9); |
| | | | 8.527(3.1); 8.258(7.0); 8.236(6.4); 7.691(2.1); 7.680(2.4); 7.672(2.4); |
| | | | 7.660(2.2); 3.903(4.9); 3.339(296.0); 3.171(1.3); 2.992(0.6); |
| | | | 2.869(16.0); 2.857(16.0); 2.673(1.1); 2.543(1.0); 2.508(130.2); |
| | | | 2.504(180.0); 2.500(141.4); 2.331(1.0); 1.232(0.4); 1.214(0.4); |
| | | | 0.000(2.8) |
| 33 | 1.53 | | ¹H-NMR(400.0 MHz, d₆-DMSO): δ = 9.332(2.7); 8.829(1.9); |
| | | | 8.820(2.0); 8.608(4.4); 8.588(4.6); 8.586(4.6); 8.529(1.8); 8.524(2.7); |
| | | | 8.520(1.9); 8.509(1.9); 8.504(2.7); 8.500(1.9); 7.792(2.8); 7.782(3.6); |
| | | | 7.771(2.7); 7.761(3.4); 7.685(2.0); 7.673(2.1); 7.666(2.0); 7.654(1.8); |
| | | | 3.902(4.0); 3.558(1.0); 3.540(2.8); 3.522(2.9); 3.504(1.1); 3.340(362.1); |
| | | | 3.310(5.2); 3.292(3.8); 3.274(1.4); 3.029(16.0); 2.970(13.5); 2.869(0.3); |
| | | | 2.857(0.3); 2.678(0.7); 2.673(1.0); 2.669(0.7); 2.508(116.8); |
| | | | 2.504(164.6); 2.499(128.9); 2.335(0.6); 2.331(0.9); 2.326(0.7); |
| | | | 1.202(3.0); 1.184(6.4); 1.167(3.1); 1.154(3.6); 1.137(7.6); 1.119(3.3); |
| | | | 0.000(2.6) |
| 34 | 2.10 | | ¹H-NMR(400.0 MHz, d₆-DMSO): δ = 11.355(11.6); 9.384(0.4); |
| | | | 8.957(10.8); 8.936(0.4); 8.848(0.4); 8.768(8.5); 8.746(9.7); 8.719(0.5); |
| | | | 8.644(11.3); 8.564(0.3); 8.473(6.3); 8.457(6.5); 8.419(9.9); 8.397(9.1); |
| | | | 8.312(0.7); 8.293(5.6); 8.272(6.0); 8.111(5.9); 8.091(6.4); 7.900(1.4); |
| | | | 7.755(4.0); 7.735(8.8); 7.715(6.0); 7.689(4.9); 7.671(6.6); 7.650(10.0); |
| | | | 7.629(5.4); 7.567(0.6); 7.546(0.9); 7.527(0.4); 4.105(0.8); 4.095(0.9); |
| | | | 3.902(16.0); 3.716(0.4); 3.704(0.4); 3.676(0.4); 3.662(0.4); 3.623(0.4); |
| | | | 3.554(0.6); 3.542(0.7); 3.509(1.3); 3.340(958.2); 3.213(0.5); 3.177(4.7); |
| | | | 3.163(4.6); 2.673(3.4); 2.633(0.3); 2.503(584.4); 2.331(3.2); 1.233(1.2); |
| | | | 0.852(0.4); 0.831(0.4); 0.000(6.9) |
| 35 | 2.03 | | ¹H-NMR(400.0 MHz, d₆-DMSO): δ = 9.352(1.1); 9.166(0.4); |
| | | | 9.152(0.9); 9.137(0.4); 8.837(0.8); 8.827(0.8); 8.659(2.4); 8.638(2.7); |
| | | | 8.555(0.6); 8.550(0.9); 8.545(0.7); 8.535(0.7); 8.530(1.0); 8.525(0.6); |
| | | | 8.272(2.6); 8.251(2.4); 7.693(0.7); 7.681(0.8); 7.673(0.7); 7.661(0.7); |
| | | | 3.902(1.7); 3.565(0.8); 3.549(1.6); 3.530(1.6); 3.514(0.8); 3.346(153.5); |
| | | | 2.724(1.6); 2.705(2.3); 2.688(1.5); 2.678(0.4); 2.673(0.5); 2.669(0.3); |
| | | | 2.543(0.3); 2.526(1.2); 2.513(25.2); 2.508(52.8); 2.504(74.2); |
| | | | 2.499(56.0); 2.495(27.1); 2.331(0.4); 2.116(16.0); 0.000(1.2) |
| 36 | 3.09 | | ¹H-NMR(400.0 MHz, d₆-DMSO): δ = 11.332(8.4); 11.193(0.3); |
| | | | 9.382(5.3); 8.849(3.7); 8.839(3.7); 8.760(0.4); 8.739(8.4); 8.718(9.3); |
| | | | 8.646(7.3); 8.583(3.7); 8.563(3.9); 8.454(0.5); 8.404(9.3); 8.382(8.5); |
| | | | 8.310(0.5); 8.293(3.7); 8.272(3.9); 7.757(3.3); 7.737(7.3); 7.717(4.7); |
| | | | 7.708(3.2); 7.696(3.2); 7.688(3.3); 7.676(3.1); 7.649(5.1); 7.630(3.4); |
| | | | 3.902(16.0); 3.706(0.4); 3.509(1.6); 3.357(1127.4); 3.177(1.2); |
| | | | 3.164(1.1); 2.678(1.6); 2.674(2.2); 2.509(269.0); 2.505(368.5); |
| | | | 2.501(284.8); 2.336(1.5); 2.332(2.0); 1.233(1.1); 0.000(3.9) |
| 37 | 2.61 | | ¹H-NMR(601.6 MHz, d₆-DMSO): δ = 11.548(2.0); 8.828(6.7); |
| | | | 8.826(10.9); 8.824(6.7); 8.585(8.5); 8.570(11.0); 8.467(13.4); |
| | | | 8.452(11.2); 8.409(6.0); 8.408(6.0); 8.407(5.6); 8.400(16.0); |
| | | | 8.397(12.7); 8.291(5.9); 8.280(4.9); 8.278(6.0); 8.275(4.5); 7.996(5.9); |
| | | | 7.982(8.6); 7.981(9.0); 7.966(5.5); 7.735(5.8); 7.722(10.7); 7.709(5.1); |
| | | | 7.632(5.5); 7.621(6.3); 7.619(6.2); 7.608(5.1); 3.321(548.2); 2.617(2.2); |
| | | | 2.614(2.9); 2.611(2.1); 2.523(5.5); 2.520(6.8); 2.517(6.9); 2.508(164.2); |
| | | | 2.505(336.0); 2.502(450.5); 2.499(336.3); 2.496(163.1); 2.389(2.1); |
| | | | 2.386(2.8); 2.383(2.1); 1.233(0.4); 0.000(5.1) |
| 38 | 1.18 | | ¹H-NMR(601.6 MHz, d₆-DMSO): δ = 11.109(2.1); 9.259(2.2); |
| | | | 9.258(2.3); 9.255(2.3); 9.254(2.2); 8.771(1.9); 8.768(2.0); 8.763(2.0); |
| | | | 8.760(2.0); 8.502(3.5); 8.487(4.3); 8.457(1.1); 8.454(1.4); 8.453(1.3); |
| | | | 8.450(1.1); 8.444(1.2); 8.441(1.4); 8.440(1.5); 8.437(1.1); 8.339(1.1); |
| | | | 8.325(0.9); 7.638(1.3); 7.637(1.3); 7.630(1.3); 7.629(1.3); 7.625(1.4); |
| | | | 7.624(1.3); 7.617(1.3); 7.616(1.3); 3.481(1.8); 3.468(3.6); 3.456(2.1); |
| | | | 3.323(117.5); 3.040(16.0); 2.967(1.8); 2.954(3.0); 2.942(1.7); |
| | | | 2.617(0.4); 2.614(0.5); 2.611(0.4); 2.584(0.6); 2.542(0.9); 2.523(0.9); |
| | | | 2.520(1.1); 2.517(1.1); 2.508(28.3); 2.505(59.2); 2.502(80.0); |
| | | | 2.499(58.9); 2.496(27.9); 2.389(0.4); 2.386(0.5); 2.383(0.4); 0.000(1.1) |
| 39 | 3.79 | 3.83 | ¹H-NMR(601.6 MHz, CDCl3): δ = 9.317(0.8); 9.314(0.8); 8.782(0.6); |
| | | | 8.779(0.6); 8.774(0.6); 8.771(0.6); 8.695(0.7); 8.692(0.8); 8.460(1.3); |
| | | | 8.445(1.5); 8.404(0.4); 8.401(0.5); 8.398(0.4); 8.391(0.4); 8.388(0.5); |
| | | | 8.385(0.4); 8.273(1.5); 8.258(1.3); 7.506(0.5); 7.499(0.5); 7.498(0.5); |
| | | | 7.4934(0.5); 7.4925(0.5); 7.4854(0.4); 7.4845(0.4); 7.262(15.5); |
| | | | 6.776(1.0); 6.772(1.0); 5.301(0.5); 1.574(20.4); 1.423(0.5); 1.336(0.5); |
| | | | 1.333(0.5); 1.285(1.2); 1.254(16.0); 1.173(0.4); 1.160(0.3); 1.155(0.3); |
| | | | 1.106(0.5); 0.900(0.4); 0.892(1.1); 0.880(1.6); 0.868(1.0); 0.856(0.6); |
| | | | 0.840(1.1); 0.829(0.9); 0.714(0.4); 0.000(3.6) |
| 40 | 2.26 | 2.33 | ¹H-NMR(400.0 MHz, d₆-DMSO): δ = 9.302(6.3); 9.300(7.0); |
| | | | 9.296(6.9); 9.294(6.7); 8.814(6.0); 8.810(6.6); 8.802(6.4); 8.798(6.5); |
| | | | 8.579(15.0); 8.558(15.8); 8.506(3.9); 8.502(4.6); 8.500(4.6); 8.496(3.9); |
| | | | 8.486(4.1); 8.482(4.6); 8.480(5.0); 8.476(3.9); 8.318(0.5); 7.761(16.0); |
| | | | 7.740(15.1); 7.672(4.6); 7.670(4.9); 7.660(4.5); 7.658(4.7); 7.652(4.4); |
| | | | 7.650(4.6); 7.640(4.3); 7.638(4.5); 3.330(93.2); 2.682(0.4); 2.677(0.8); |
| | | | 2.673(1.1); 2.668(0.8); 2.664(0.4); 2.526(2.7); 2.521(4.0); 2.513(58.2); |
| | | | 2.508(121.6); 2.503(164.7); 2.499(121.9); 2.494(59.5); 2.339(0.4); |
| | | | 2.335(0.8); 2.330(1.1); 2.326(0.8); 2.321(0.4); 1.398(1.6); 1.231(0.4); |
| | | | 0.000(1.0) |
| 42 | 2.75 | 2.77 | ¹H-NMR(601.6 MHz, CDCl3): δ = 9.251(2.6); 9.248(2.6); 8.710(1.9); |
| | | | 8.707(2.0); 8.702(2.0); 8.699(1.9); 8.320(1.3); 8.3174(1.7); 8.3167(1.6); |
| | | | 8.314(1.3); 8.307(1.3); 8.304(1.7); 8.303(1.7); 8.301(1.3); 8.151(4.3); |
| | | | 8.136(4.6); 7.969(3.3); 7.965(3.5); 7.886(1.7); 7.882(1.6); 7.872(1.8); |
| | | | 7.868(1.7); 7.450(1.4); 7.449(1.4); 7.442(1.4); 7.441(1.4); 7.437(1.5); |
| | | | 7.436(1.5); 7.432(1.3); 7.429(1.4); 7.428(1.4); 7.280(2.5); 7.261(188.5); |
| | | | 7.085(1.1); 6.923(4.6); 6.913(2.7); 6.908(4.9); 3.488(1.4); 3.471(4.3); |
| | | | 3.455(4.6); 3.438(1.6); 2.379(16.0); 1.584(0.4); 1.558(335.1); |
| | | | 1.530(0.4); 1.284(0.4); 1.254(3.2); 0.880(0.5); 0.005(0.8); 0.000(25.5); |
| | | | −0.006(0.9) |

### BIOLOGICAL EXAMPLES

Myzus persicae—Spray Test
Solvent: 78 parts by weight of acetone
   1.5 parts by weight of dimethylformamide
Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) infested by all stages of the green peach aphid (Myzus persicae) are sprayed with an active compound formulation of the desired concentration.

After 6 days, the efficacy in % is determined. 100% means that all the aphids have been killed; 0% means that no aphids have been killed.

In this test, for example, the following compounds from the preparation examples showed an efficacy of 100% at an application rate of 500 g/ha: 1, 5, 24, 25, 26, 27, 29

In this test, for example, the following compounds from the preparation examples showed an efficacy of 90% at an application rate of 500 g/ha: 2, 4, 11, 12, 20, 22, 28, 30, 31, 35, 41

Tetranychus urticae—Spray Test, OP-Resistant
Solvent: 78.0 parts by weight of acetone
   1.5 parts by weight of dimethylformamide
Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water.

Discs of bean leaves (Phaseolus vulgaris) infested with all stages of the greenhouse red spider mite (Tetranychus urticae) are sprayed with an active compound formulation of the desired concentration.

After 6 days, the efficacy in % is determined. 100% means that all the spider mites have been killed; 0% means that no spider mites have been killed.

In this test, for example, the following compounds from the preparation examples showed an efficacy of 90% at an application rate of 500 g/ha: 9, 10

Myzus persicae—Spray Test
Solvent: 7 parts by weight of dimethylformamide
Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water. If the addition of ammonium salts or/and penetrants is required, these are each added in a concentration of 1000 ppm to the formulation solution.

Bell pepper plants (Capsicum annuum) severely infested with the green peach aphid (Myzus persicae) are treated by spraying with the active compound formulation in the desired concentration.

After 6 days, the kill in % is determined. 100% means that all of the aphids have been killed; 0% means that none of the aphids have been killed.

In this test, for example, the following compounds from the preparation examples showed an efficacy of 95% at an application rate of 100 ppm: 6

In this test, for example, the following compounds from the preparation examples showed an efficacy of 100% at an application rate of 4 ppm: 1,2

In this test, for example, the following compounds from the preparation examples showed an efficacy of 97% at an application rate of 4 ppm: 3

In this test, for example, the following compounds from the preparation examples showed an efficacy of 95% at an application rate of 4 ppm: 4

In this test, for example, the following compounds from the preparation examples showed an efficacy of 80% at an application rate of 4 ppm: 8

Aphis gossypii—Spray Test
Solvent: 7 parts by weight of dimethylformamide
Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the formulation is diluted with emulsifier-containing water. If the addition of ammonium salts or/and penetrants is required, these are each added in a concentration of 1000 ppm to the formulation solution.

Cotton plants (Gossypium hirsutum) which are heavily infested by the cotton aphid (Aphis gossypii) are sprayed with an active compound formulation of the desired concentration.

After 6 days, the kill in % is determined. 100% means that all the aphids have been killed; 0% means that no aphids have been killed.

In this test, for example, the following compounds from the preparation examples showed an efficacy of 80% at an application rate of 4 ppm: 7, 24

1. Compound of formula (I) 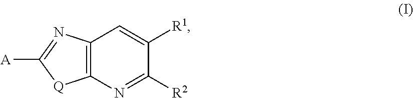
in which
A represents an A radical from the group consisting of (A-b) to (A-f) 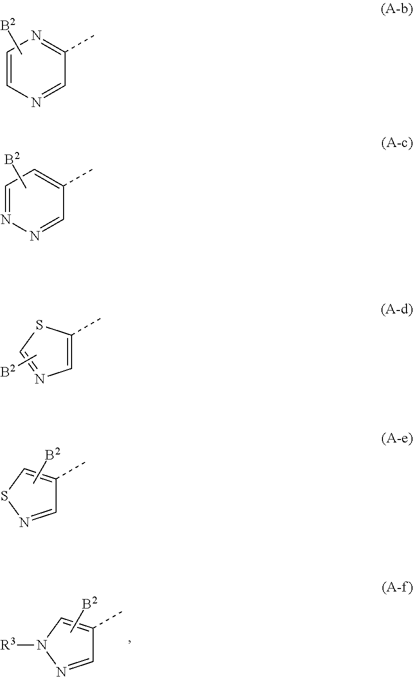
where the broken line denotes the bond to the carbon atom of the bicyclic system of the formula (I) and
B² represents a radical from the group consisting of hydrogen, halogen, cyano, nitro, alkyl, haloalkyl, alkoxy, haloalkoxy and in each case optionally substituted cycloalkyl and cycloalkenyl,
Q represents oxygen or sulfur,
R¹ represents a radical from the group consisting of hydrogen, alkyl, alkoxy and cyano,
R² a) represents a B radical from the group consisting of 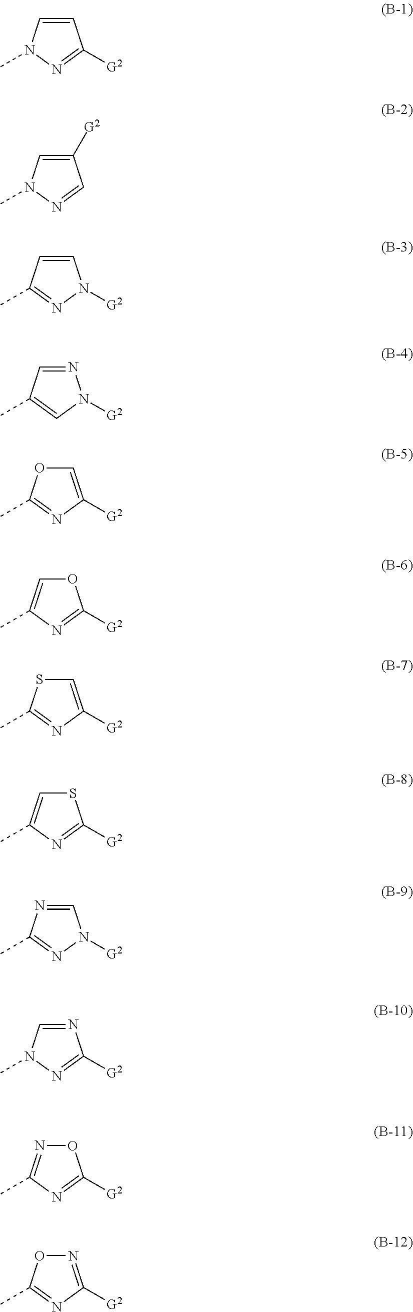 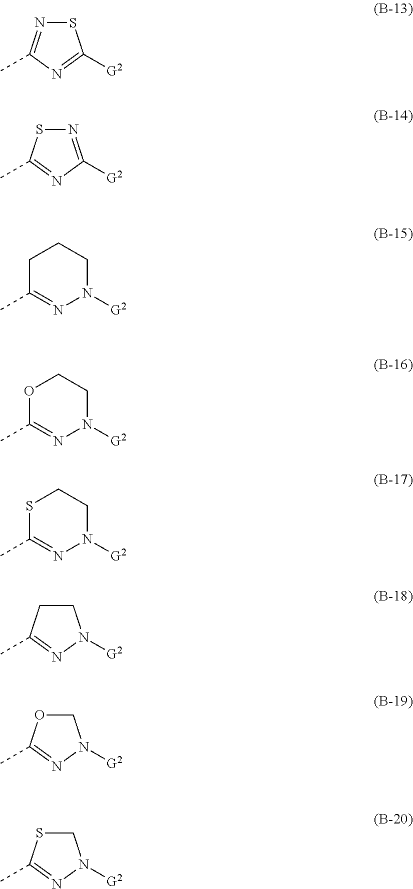 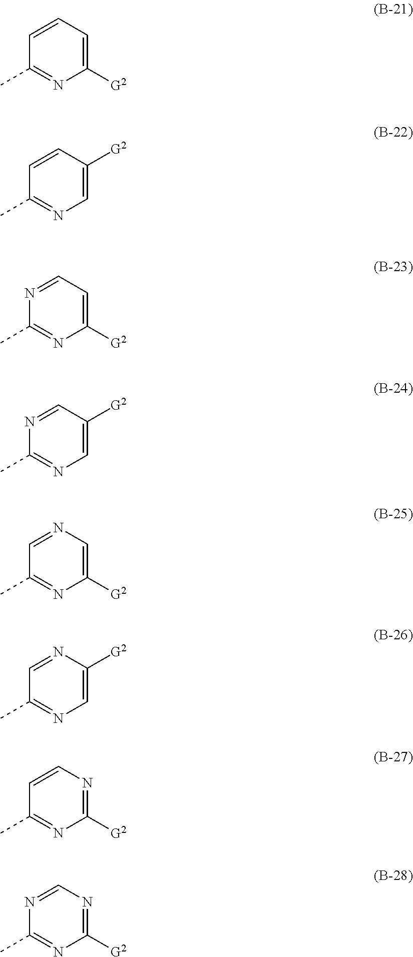 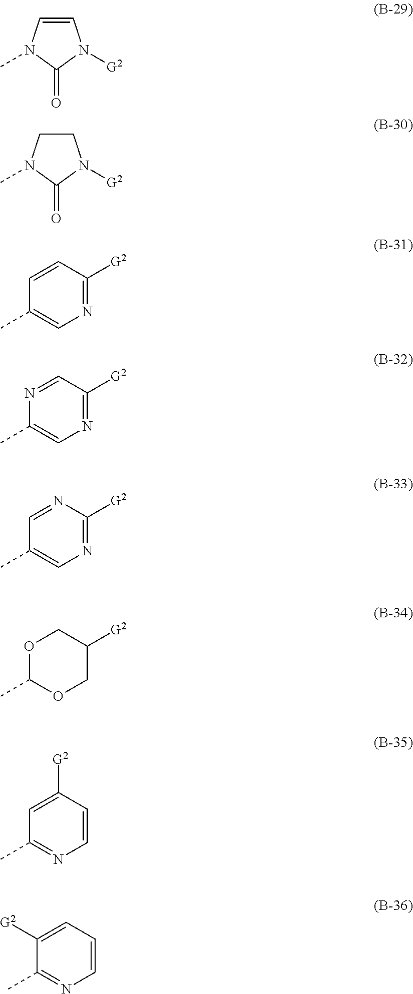
where the broken line denotes the bond to the carbon atom of the bicyclic system of the formula (I), or
R² b) represents a D radical from the group consisting of (D-1) to (D-3) 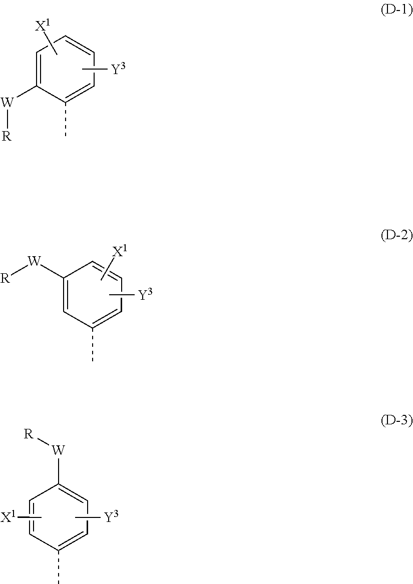
where the broken line denotes the bond to the carbon atom of the bicyclic system of the formula (I), or
R² c) represents a radical of the formula 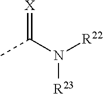
where the broken line denotes the bond to the carbon atom of the bicyclic system of the formula (I), or
R² d) represents a radical of the formula 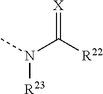
where the broken line denotes the bond to the carbon atom of the bicyclic system of the formula (I), or
R² e) represents an F radical from the group consisting of (F-1) to (F-11) 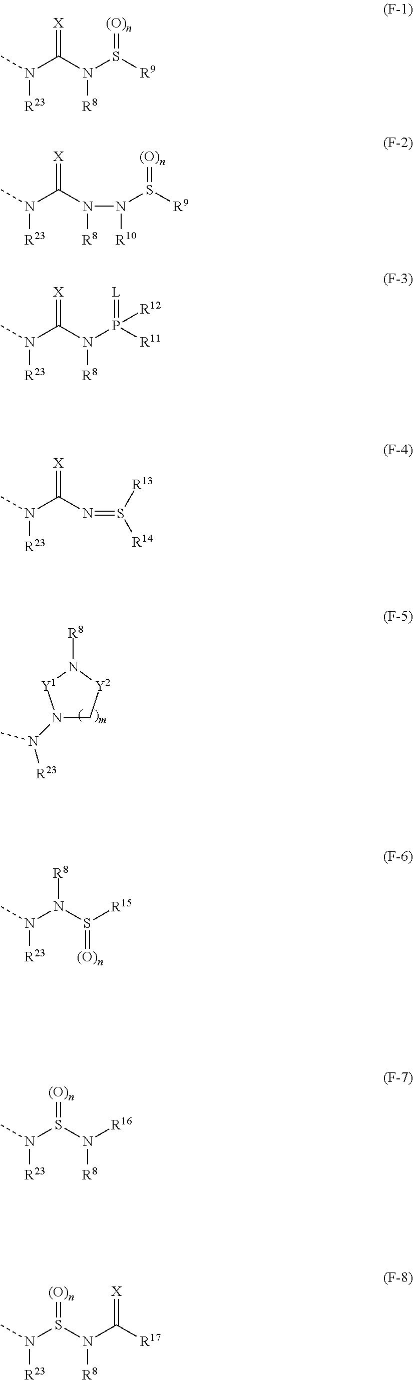 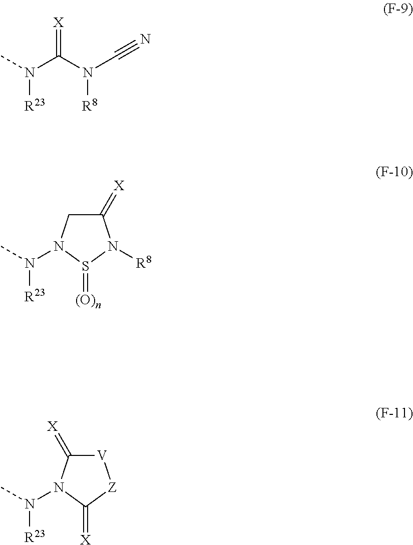
where the broken line denotes the bond to the carbon atom of the bicyclic system of the formula (I), or
R² f) represents a radical from the group consisting of haloalkyl and carboxyl,
R² g) represents a radical of the formula 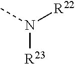
where the broken line denotes the bond to the carbon atom of the bicyclic system of the formula (I),
in which
G² represents hydrogen or a radical from the group consisting of halogen, nitro, amino, cyano, alkylamino, haloalkylamino, dialkylamino, alkyl, haloalkyl, saturated or unsaturated cycloalkyl which is optionally substituted and optionally interrupted by one or more heteroatoms, cycloalkylalkyl, alkoxy, haloalkoxy, alkoxyalkyl, halogenated alkoxyalkyl, alkylthioalkyl, alkylsulfinylalkyl, alkylsulfonylalkyl, bis(alkoxy)alkyl, bis(haloalkoxy)alkyl, alkoxy(alkylsulfanyl)alkyl, alkoxy(alkylsulfinyl)alkyl, alkoxy(alkylsulfonyl)alkyl, bis(alkylsulfanyl)alkyl, bis(haloalkylsulfanyl)alkyl, bis(hydroxyalkylsulfanyl)alkyl, alkoxycarbonyl, alkoxycarbonylalkyl, alpha-hydroxyiminoalkoxycarbonylalkyl, alpha-alkoxyiminoalkoxycarbonylalkyl, C(X²)NR³R⁴, NR⁶R⁷, alkylthio, alkylsulfinyl, alkylsulfonyl, haloalkylthio, haloalkylsulfinyl, haloalkylsulfonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by alkyl, haloalkyl, alkoxy and alkoxyalkyl), phenyl (which for its part may be substituted by halogen, cyano, nitro, alkyl and haloalkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, alkyl, haloalkyl, alkoxy, haloalkoxy, alkoxyalkyl, alkylthio, alkylthioalkyl and cycloalkyl) and the heteroarylalkyl radicals triazolylalkyl, pyridylalkyl, pyrimidylalkyl and oxadiazolylalkyl (which for their part may be substituted by halogen and alkyl),
or
G² represents a C radical from the group consisting of (C-1) to (C-9) 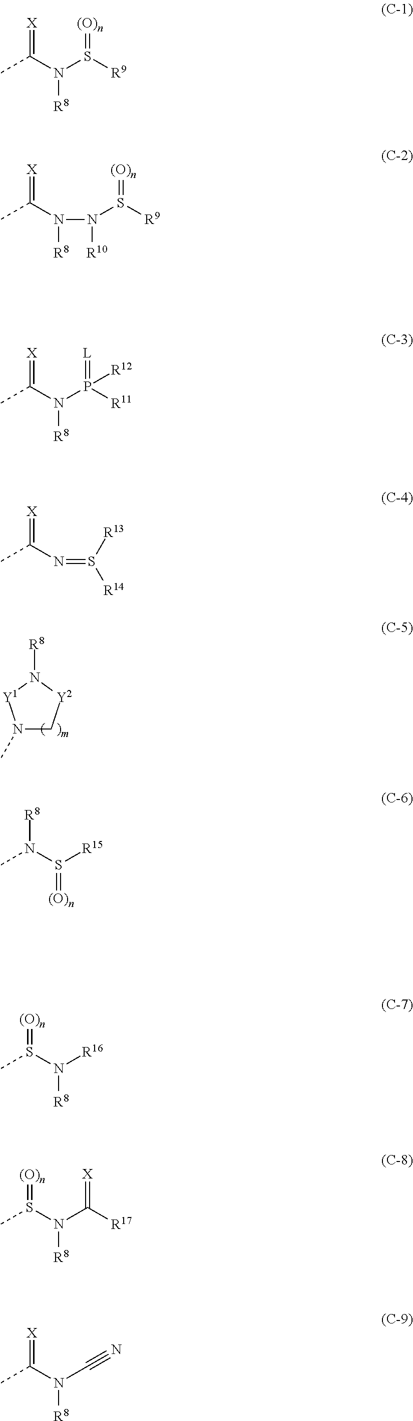
where the broken line denotes the bond to the B radicals,
X represents oxygen or sulfur,
X represents oxygen or sulfur,
X¹ represents a radical from the group consisting of hydrogen, halogen, cyano, nitro, alkyl, haloalkyl, cycloalkyl, alkoxy and haloalkoxy,
X² represents oxygen, sulfur, NR⁵ or NOH,
L represents oxygen or sulfur,
V—Z represents R²⁴CH—CHR²⁵ or R²⁴C═CR²⁵,
n represents 1 or 2,
m represents 1, 2, 3 or 4,
R represents NR¹⁸R¹⁹, or represents an in each case optionally substituted radical from the group consisting of alkyl, alkenyl, alkynyl, alkoxyalkyl, alkyl-S-alkyl, alkyl-S(O)-alkyl, alkyl-S(O)₂-alkyl, R¹⁸—CO-alkyl, NR¹⁸R¹⁹—CO-alkyl, cycloalkyl, cycloalkenyl, cycloalkylalkyl, cycloalkenylalkyl, heterocyclyl, heterocyclylalkyl, phenyl, phenylalkyl, hetaryl and hetarylalkyl,
R³ represents hydrogen or alkyl,
R⁴ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, alkylsulfinylalkyl, alkylsulfonylalkyl, aryl, arylalkyl and hetarylalkyl,
R⁵ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, aryl, arylalkyl and hetarylalkyl, or
R³ and R⁴ together with the nitrogen atom to which they are attached form a ring which may contain one or more further heteroatoms from the group consisting of nitrogen, oxygen and sulfur, or R³ and R⁵ together with the nitrogen atoms to which they are attached form a ring,
R⁶ represents hydrogen or alkyl,
R⁷ represents a radical from the group consisting of hydrogen, alkyl, haloalkyl, cyanoalkyl, alkynyl, cycloalkyl, cycloalkylalkyl, alkoxy, haloalkoxy, alkoxyalkyl, alkylthioalkyl, alkylsulfinylalkyl, alkylsulfonylalkyl, alkoxycarbonyl, alkoxycarbonylalkyl, alkylthioalkyl, aryl, arylalkyl and hetarylalkyl, or
R⁶ and R⁷ together with the nitrogen atom to which they are attached form a ring which may contain one or more further heteroatoms from the group consisting of nitrogen, oxygen and sulfur,
R⁸ represents a radical from the group consisting of hydrogen, alkyl, haloalkyl, cyanoalkyl, alkoxy, haloalkoxy, alkenyl, alkoxyalkyl, in each case optionally halogen-substituted alkylcarbonyl and alkylsulfonyl, optionally halogen-substituted alkoxycarbonyl, optionally halogen-, alkyl-, alkoxy-, haloalkyl- and cyano-substituted cycloalkylcarbonyl, or a cation, or an optionally alkyl- or arylalkyl-substituted ammonium ion,
R⁹ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R⁹ in the radicals (C-1) and (F-1), together with the N—S(O)n group to which they are attached, may also form a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁰ represents hydrogen or alkyl,
R⁸ and R¹⁰ in the radicals (C-2) and (F-2), together with the nitrogen atoms to which they are attached, may also be a saturated or unsaturated and optionally substituted 4- to 8-membered ring which may contain at least one further heteroatom from the group consisting of sulfur, oxygen (where oxygen atoms must not be directly adjacent to one another) and nitrogen and/or at least one carbonyl group,
R⁹ and R¹⁰ in the radicals (C-2) and (F-2) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹¹ represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹² represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹¹ and R¹² in the radicals (C-3) and (F-3) may also form, together with the phosphorus atom to which they are bonded, a saturated or unsaturated and optionally substituted 5- to 7-membered ring which may contain one or two heteroatoms from the group consisting of oxygen (where oxygen atoms must not be immediately adjacent to one another) and sulfur,
R¹³ represents an in each case optionally substituted radical from the group consisting of alkyl, alkenyl, alkynyl, phenyl and phenylalkyl,
R¹⁴ represents an in each case optionally substituted radical from the group consisting of alkyl, alkenyl, alkynyl, phenyl and phenylalkyl,
R¹⁵ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁵ in the radicals (C-6) and (F-6) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁶ represents a radical from the group consisting of hydrogen, in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁶ in the radicals (C-7) and (F-7) may also form, together with the nitrogen atom to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁷ represents a radical from the group consisting of in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁷ in the radicals (C-8) and (F-8) may also form, together with the N—C(X) group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁸ represents a radical from the group consisting of hydrogen, hydroxy, in each case optionally substituted alkyl, alkoxy, alkoxyalkyl, alkylthioalkyl, alkylsulfinylalkyl, alkylsulfonylalkyl, alkylcarbonyl, alkoxycarbonyl, alkenyl and alkynyl, in each case optionally substituted cycloalkyl, cycloalkylalkyl, cycloalkenyl and cycloalkenylalkyl, in which the rings may contain at least one heteroatom from the group consisting of sulfur, oxygen (where oxygen atoms must not be directly adjacent to one another) and nitrogen, in each case optionally substituted aryl, arylalkyl, heteroaryl and heteroarylalkyl and an optionally substituted amino group,
R¹⁹ represents a radical from the group consisting of hydrogen, represents an alkali metal or alkaline earth metal ion or represents an ammonium ion which is optionally mono- to tetrasubstituted by C₁-C₄-alkyl or represents an in each case optionally halogen- or cyano-substituted radical from the group consisting of alkyl, alkoxy, alkoxyalkyl, alkylthioalkyl, alkylsulfinylalkyl and alkylsulfonylalkyl,
Y¹ and Y² independently of one another represent C═O or S(O)₂,
Y³ represents a radical from the group consisting of hydrogen, halogen, cyano, alkyl, cycloalkyl, haloalkyl, alkoxy, haloalkoxy and NR²⁰R²¹,
W represents a radical from the group consisting of O, S, SO and SO₂,
R²² represents a radical from the group consisting of hydrogen, alkyl, haloalkyl, cyanoalkyl, alkenyl, haloalkenyl, alkynyl, haloalkynyl, alkoxy, haloalkoxy, optionally halogen-substituted alkoxyalkyl, optionally halogen-substituted bis(alkoxy)alkyl, optionally halogen-substituted alkylsulfanylalkyl, optionally halogen-substituted alkylcarbonylalkyl, optionally halogen-substituted alkylsulfinylalkyl, optionally halogen-substituted alkylsulfonylalkyl, dialkylaminosulfanylalkyl, dialkylaminosulfinylalkyl, dialkylaminosulfonylalkyl, optionally halogen-substituted alkoxycarbonyl, optionally halogen-substituted alkoxycarbonylalkyl, optionally halogen-substituted alkynyloxy, optionally halogen-substituted alkynyloxycarbonyl, dialkylaminocarbonyl, N-alkyl-N-cycloalkylaminocarbonyl, dialkylaminocarbonylalkyl, N-alkyl-N-cycloalkylaminocarbonylalkyl, heterocyclylcarbonylalkyl, alkylsulfanyl, haloalkylsulfanyl, alkylsulfinyl, haloalkylsulfinyl, alkylsulfonyl, haloalkylsulfonyl, cycloalkyl which is optionally substituted by halogen, cyano, nitro, alkyl, cycloalkyl, haloalkyl, alkoxy, haloalkoxy, alkoxycarbonyl, haloalkoxycarbonyl or hetaryl (which for its part is optionally substituted by alkyl or halogen), cycloalkylcarbonyl which is optionally substituted by halogen, cyano, nitro, alkyl, haloalkyl, cycloalkyl, alkoxy, haloalkoxy, alkoxycarbonyl, haloalkoxycarbonyl or hetaryl (which for its part is optionally substituted by alkyl or halogen), cycloalkylalkyl which is optionally substituted by halogen, cyano, nitro, alkyl, haloalkyl, cycloalkyl, alkoxy, haloalkoxy, alkoxycarbonyl, haloalkoxycarbonyl or hetaryl (which for its part is optionally substituted by alkyl or halogen), optionally substituted heterocyclyl, heterocyclylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulfinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxyalkyl, alkenyl, alkynyl, cycloalkylalkyl, alkylcarbonyl, alkoxycarbonyl or aminocarbonyl, aryl which is optionally substituted by halogen, cyano, nitro, hydroxy, amino, alkyl, haloalkyl, cycloalkyl (which is optionally substituted), alkoxy or haloalkoxy, arylalkyl which is optionally substituted by halogen, cyano (also in the alkyl moiety), nitro, hydroxy, amino, alkyl, cycloalkyl (which is optionally substituted), haloalkyl, alkoxy or haloalkoxy, hetarylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulfinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxyalkyl, alkenyl, alkynyl, cycloalkylalkyl, alkylcarbonyl, alkoxycarbonyl or aminocarbonyl, or
R²² represents a D radical from the group consisting of (D-1) to (D-3) 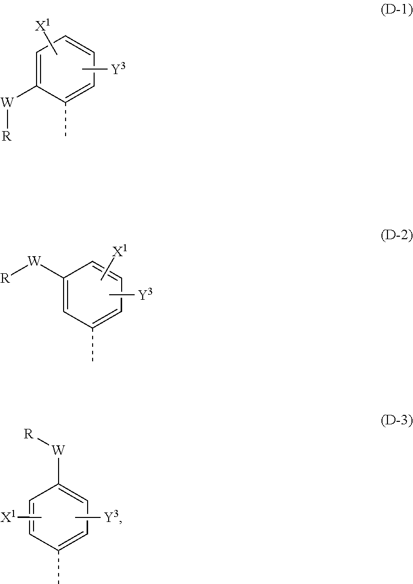
an E radical from the group consisting of (E-1) to (E-11) 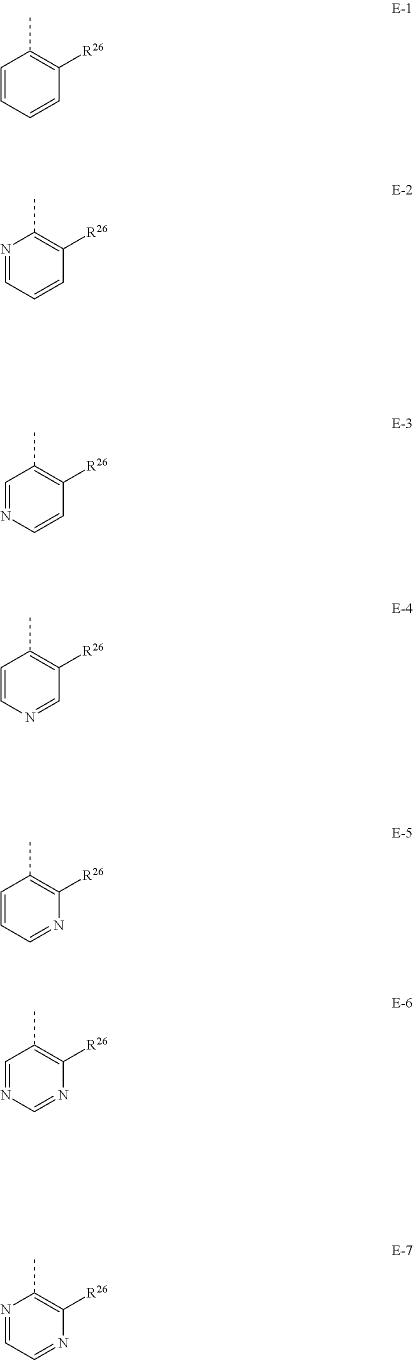 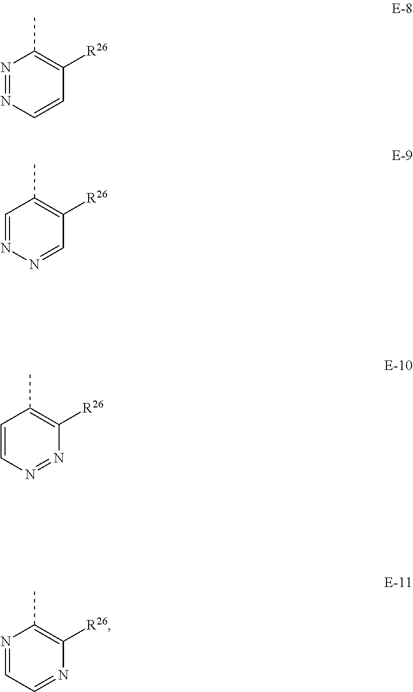
of the group consisting of (E-18) to (E-51) 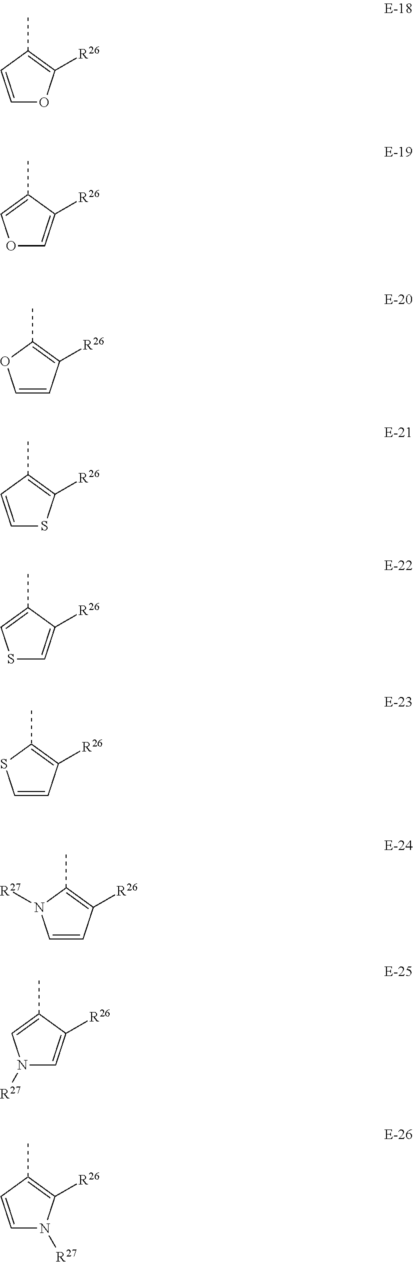 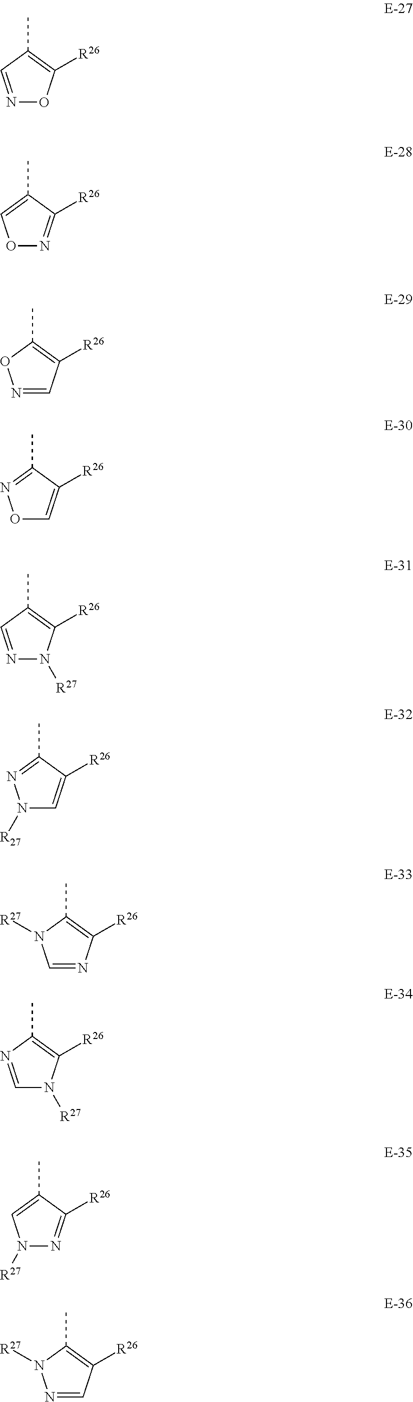 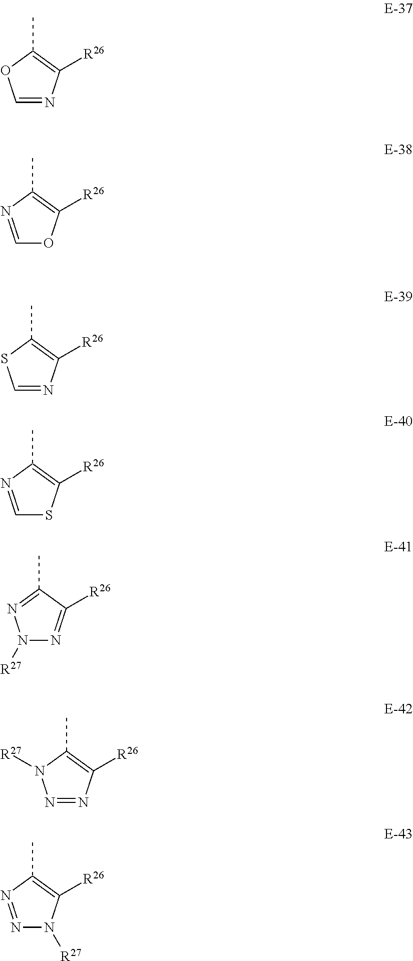 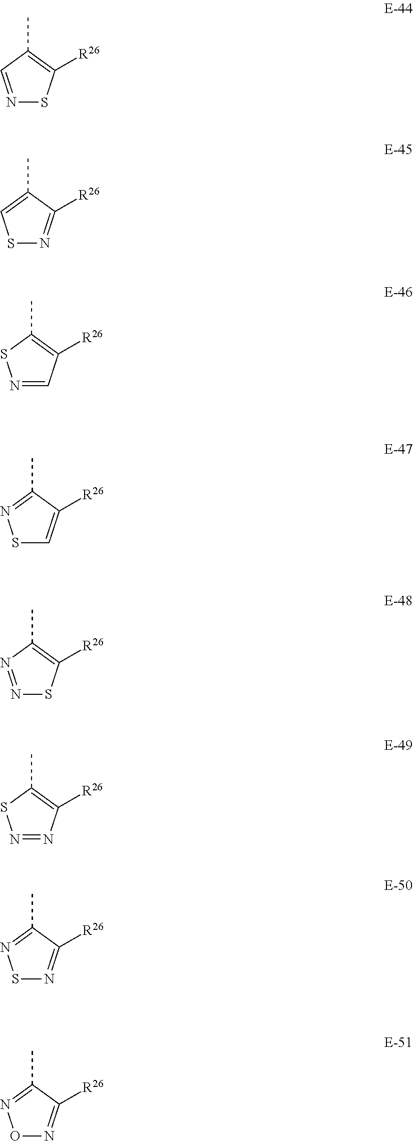
or
in the case R²=d),
R²² also represents an E radical from the group consisting of E-12 to E-17 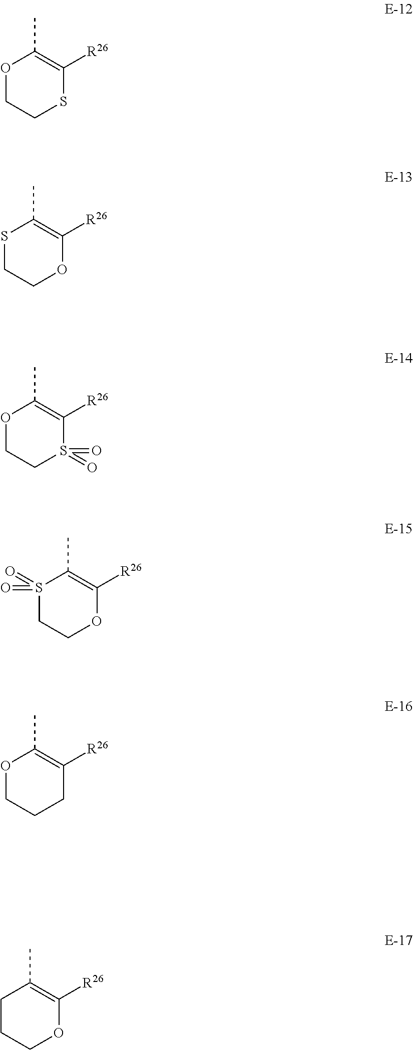
where here and below, the broken line denotes the bond to the appropriate atom in the radicals c), d) and g),
R²⁰ represents a radical from the group consisting of hydrogen, halogen, cyano, nitro, amino, hydroxy and in each case optionally substituted alkyl, alkenyl, alkynyl, cycloalkyl, cycloalkylalkyl, alkoxy, alkenyloxy, alkynyloxy, cycloalkyloxy, alkylcarbonyloxy, alkenylcarbonyloxy, alkynylcarbonyloxy, cycloalkylcarbonyloxy, alkoxycarbonyloxy, alkylsulfonyloxy, alkylamino, alkenylamino, alkynylamino, cycloalkylamino, alkylthio, haloalkylthio, alkenylthio, alkynylthio, cycloalkylthio, alkylsulfinyl, alkylsulfonyl, alkylcarbonyl, alkoxyiminoalkyl, alkoxycarbonyl, aminocarbonyl, alkylaminocarbonyl, dialkylaminocarbonyl, aminothiocarbonyl, alkylaminosulfonyl, alkylsulfonylamino, alkylcarbonylamino, alkenylcarbonylamino, alkynylcarbonylamino, cycloalkylcarbonylamino, alkoxycarbonylamino, alkylthiocarbonylamino, bicycloalkyl, aryl, aryloxy, heteroaryl and heteroaryloxy, where the substituents are independently of one another selected from halogen, cyano, nitro, hydroxy, amino, alkyl and haloalkyl,
R²¹ represents a radical from the group consisting of hydrogen, alkyl, cycloalkyl, haloalkyl, alkenyl, alkynyl, cycloalkylalkyl, cyanoalkyl, alkylcarbonyl, alkenylcarbonyl, haloalkylcarbonyl, haloalkenylcarbonyl, alkoxyalkyl, alkoxycarbonyl, alkylsulfonyl and haloalkylsulfonyl,
R²³ represents a radical from the group consisting of hydrogen, alkyl, alkenyl, alkynyl, cycloalkyl, cycloalkenyl, alkoxy, alkenyloxy, alkynyloxy, cycloalkyloxy, alkylthioalkyl, alkenylthioalkyl, cyanoalkyl, alkoxyalkyl,
or, if R²=c) or g),
R²² and R²³ together with the nitrogen atom to which they are attached form a ring which may contain one or more further heteroatoms from the group consisting of nitrogen, oxygen and sulfur, and
R²⁴ represents hydrogen or an in each case optionally substituted radical from the group consisting of alkyl, alkenyl, alkynyl, phenyl and phenylalkyl and
R²⁵ represents hydrogen or an in each case optionally substituted radical from the group consisting of alkyl, alkenyl, alkynyl, phenyl and phenylalkyl,
R²⁶ represents a radical from the group consisting of hydrogen, alkyl, haloalkyl, alkenyl, alkynyl, cycloalkyl, cycloalkylalkyl, alkoxyalkyl, alkylsulfanyl, haloalkylsulfanyl, alkylsulfinyl, haloalkylsulfinyl, alkylsulfonyl, alkylthioalkyl, alkylsulfinylalkyl, alkylsulfonylalkyl and cyanoalkyl.
R²⁷ represents hydrogen, alkyl, haloalkyl, alkenyl, alkynyl, cycloalkyl, cycloalkylalkyl, alkoxyalkyl, alkylthioalkyl, alkylsulfinylalkyl, alkylsulfonylalkyl or cyanoalkyl and
compounds of the formula (I) in which
A represents the A radical 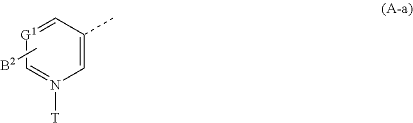
where the broken line denotes the bond to the carbon atom of the bicyclic system of the formula (I) and
G¹ represents N or C—B¹,
B¹ represents a radical from the group consisting of hydrogen, halogen, cyano, nitro, alkyl, haloalkyl, alkoxy, haloalkoxy and in each case optionally substituted cycloalkyl and cycloalkenyl,
B² represents a radical from the group consisting of hydrogen, halogen, cyano, nitro, alkyl, haloalkyl, alkoxy, haloalkoxy and in each case optionally substituted cycloalkyl and cycloalkenyl,
T represents oxygen or an electron pair,
Q represents oxygen or sulfur,
R¹ represents a radical from the group consisting of hydrogen, alkyl, alkoxy and cyano,
R² a) represents a B radical from the group consisting of 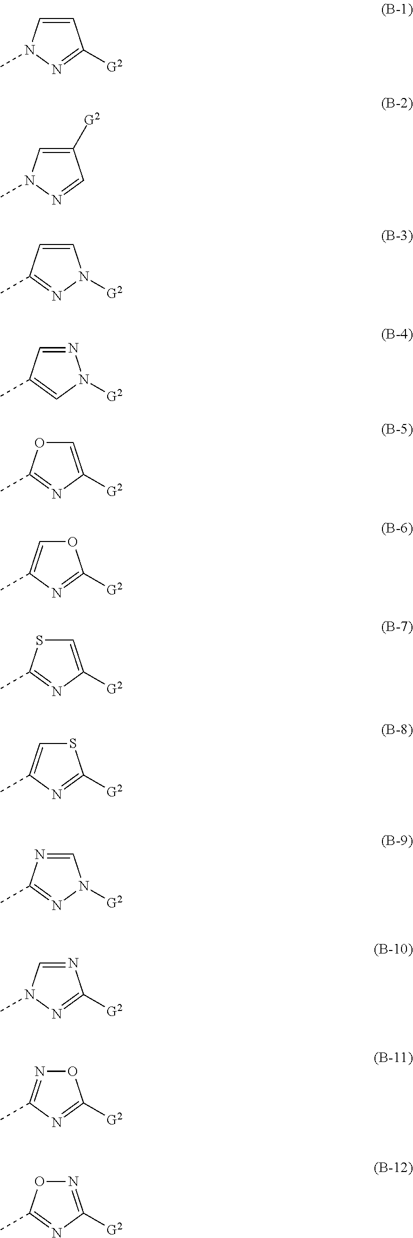 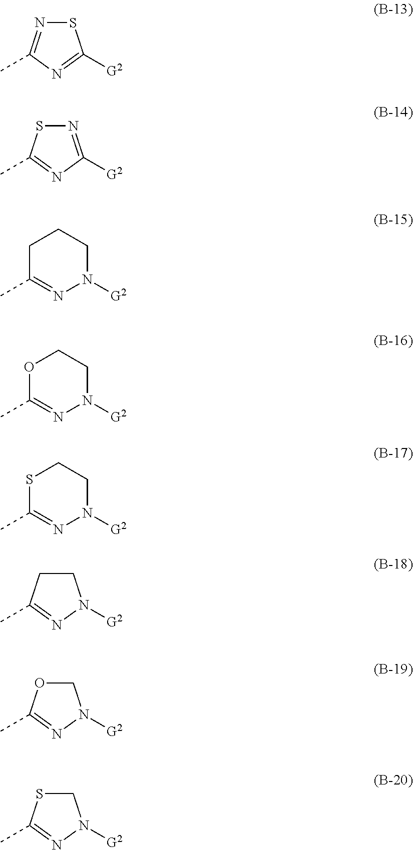 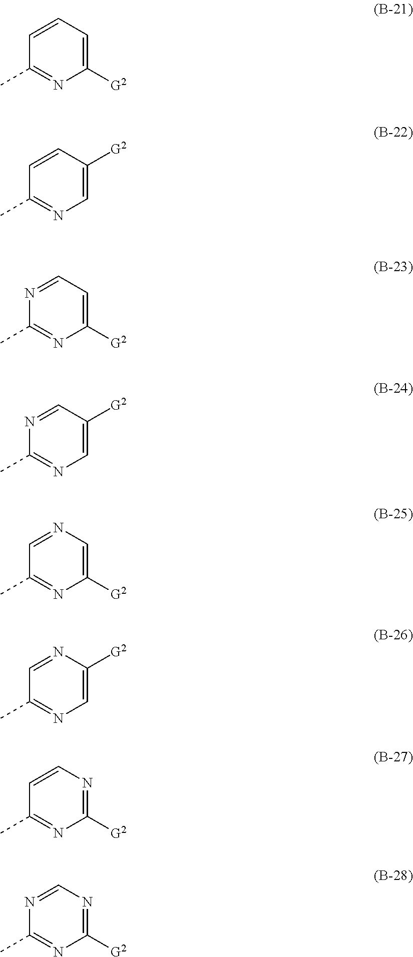 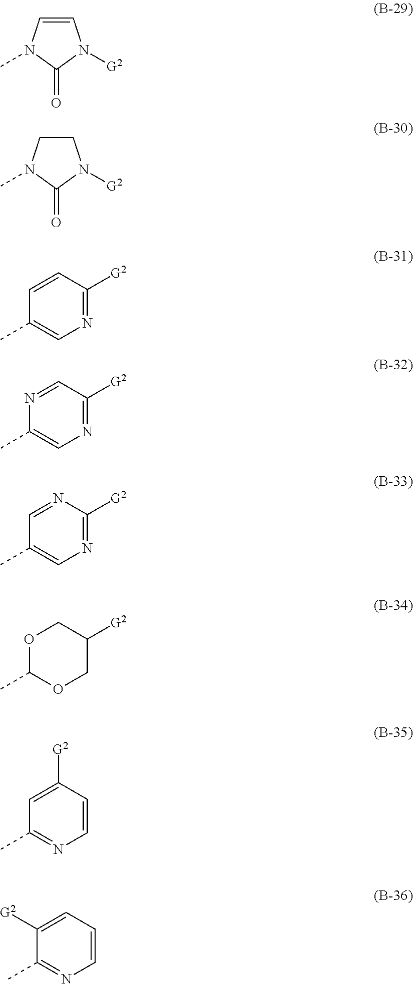
where the broken line denotes the bond to the carbon atom of the bicyclic system of the formula (I), or
R² c) represents a radical of the formula 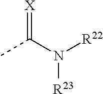
where the broken line denotes the bond to the carbon atom of the bicyclic system of the formula (I), or
R² d) represents a radical of the formula 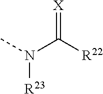
where the broken line denotes the bond to the carbon atom of the bicyclic system of the formula (I), or
R² e) represents an F radical from the group consisting of 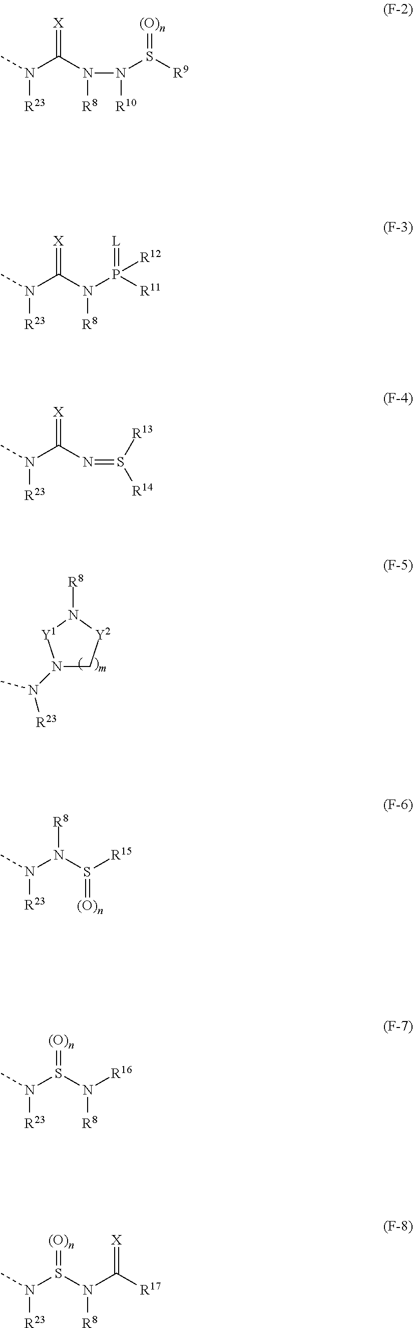 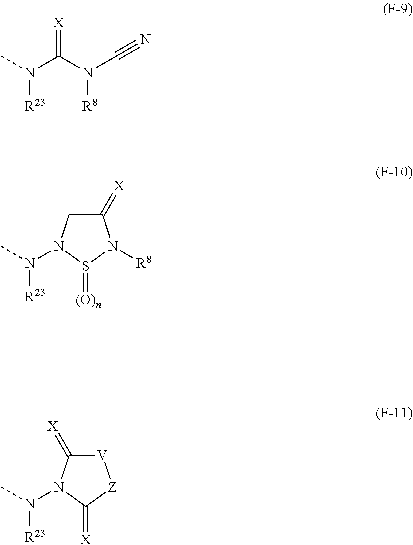
where the broken line denotes the bond to the carbon atom of the bicyclic system of the formula (I), or
R² f) represents a radical from the group consisting of haloalkyl and carboxyl,
R² g) represents a radical of the formula 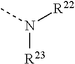
where the broken line denotes the bond to the carbon atom of the bicyclic system of the formula (I), where
G² represents hydrogen or a radical from the group consisting of halogen, nitro, amino, cyano, alkylamino, haloalkylamino, dialkylamino, alkyl, haloalkyl, saturated or unsaturated cycloalkyl which is optionally substituted and optionally interrupted by one or more heteroatoms, cycloalkylalkyl, alkoxy, haloalkoxy, alkoxyalkyl, halogenated alkoxyalkyl, alkylthioalkyl, alkylsulfinylalkyl, alkylsulfonylalkyl, bis(alkoxy)alkyl, bis(haloalkoxy)alkyl, alkoxy(alkylsulfanyl)alkyl, alkoxy(alkylsulfinyl)alkyl, alkoxy(alkylsulfonyl)alkyl, bis(alkylsulfanyl)alkyl, bis(haloalkylsulfanyl)alkyl, bis(hydroxyalkylsulfanyl)alkyl, alkoxycarbonyl, alkoxycarbonylalkyl, alpha-hydroxyiminoalkoxycarbonylalkyl, alpha-alkoxyiminoalkoxycarbonylalkyl, C(X²)NR³R⁴, NR⁶R⁷, alkylthio, alkylsulfinyl, alkylsulfonyl, haloalkylthio, haloalkylsulfinyl, haloalkylsulfonyl, the heterocyclyl radicals dioxanyl, dioxolanyl, dioxepanyl, dioxocanyl, oxathianyl, oxathiolanyl, oxathiepanyl, oxathiocanyl, dithianyl, dithiolanyl, dithiepanyl, dithiocanyl, oxathianyl oxide, oxathiolanyl oxide, oxathiepanyl oxide, oxathiocanyl oxide, oxathianyl dioxide, oxathiolanyl dioxide, oxathiepanyl dioxide, oxathiocanyl dioxide, morpholinyl, triazolinonyl, oxazolinyl, dihydrooxadiazinyl, dihydrodioxazinyl, dihydrooxazolyl, dihydrooxazinyl and pyrazolinonyl (which for their part may be substituted by alkyl, haloalkyl, alkoxy and alkoxyalkyl), phenyl (which for its part may be substituted by halogen, cyano, nitro, alkyl and haloalkyl), the heteroaryl radicals pyridyl, pyridyl N-oxide, pyrimidyl, imidazolyl, pyrazolyl, oxazolyl, thiazolyl, furanyl, thienyl, triazolyl, tetrazolyl, oxadiazolyl, thiadiazolyl, pyrazinyl, triazinyl, tetrazinyl and isoquinolinyl (which for their part may be substituted by halogen, nitro, alkyl, haloalkyl, alkoxy, haloalkoxy, alkoxyalkyl, alkylthio, alkylthioalkyl and cycloalkyl) and the heteroarylalkyl radicals triazolylalkyl, pyridylalkyl, pyrimidylalkyl and oxadiazolylalkyl (which for their part may be substituted by halogen and alkyl), or
G² represents a C radical from the group consisting of (C-1) to (C-9) 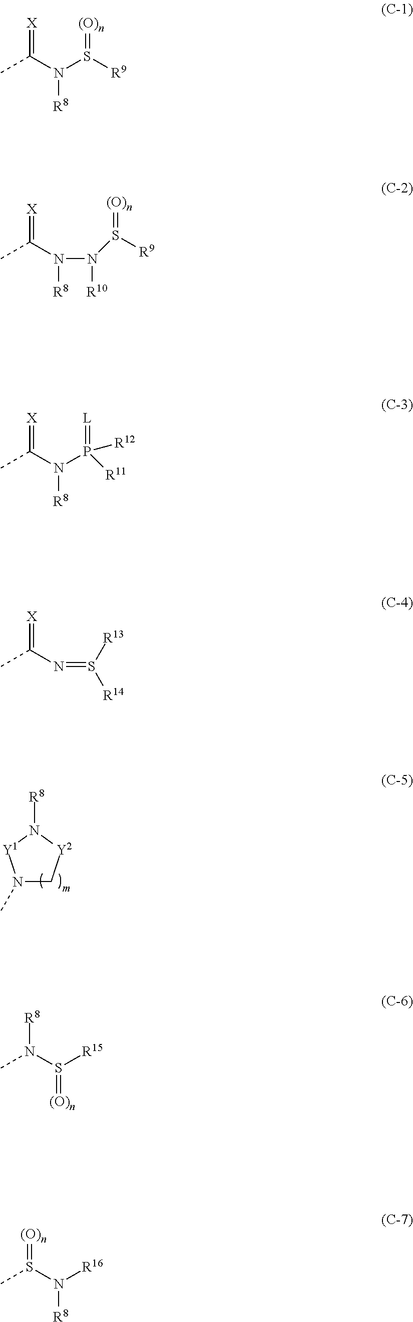 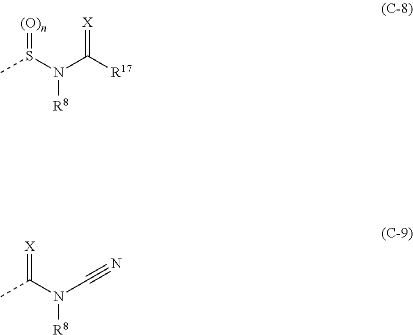
where the broken line represents the bond to the B radicals,
X represents oxygen or sulfur,
X² represents oxygen, sulfur, NR⁵ or NOH,
L represents oxygen or sulfur,
V—Z represents R²⁴CH—CHR²⁵ or R²⁴C═CR²⁵,
n represents 1 or 2,
m represents 1, 2, 3 or 4,
R³ represents hydrogen or alkyl,
R⁴ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, alkylsulfinylalkyl, alkylsulfonylalkyl, aryl, arylalkyl and hetarylalkyl,
R⁵ represents a radical from the group consisting of hydrogen, alkyl, haloalkyl, cyanoalkyl, alkynyl, cycloalkyl, cycloalkylalkyl, alkoxyalkyl, alkoxycarbonyl, alkoxycarbonylalkyl, alkylthioalkyl, aryl, arylalkyl and hetarylalkyl, or
R³ and R⁴ together with the nitrogen atom to which they are attached form a ring which may contain one or more further heteroatoms from the group consisting of nitrogen, oxygen and sulfur, or
R³ and R⁵ together with the nitrogen atoms to which they are attached form a ring,
R⁶ represents hydrogen or alkyl,
R⁷ represents a radical from the group consisting of hydrogen, alkyl, haloalkyl, cyanoalkyl, alkynyl, cycloalkyl, cycloalkylalkyl, alkoxy, haloalkoxy, alkoxyalkyl, alkylthioalkyl, alkylsulfinylalkyl, alkylsulfonylalkyl, alkoxycarbonyl, alkoxycarbonylalkyl, alkylthioalkyl, aryl, arylalkyl and hetarylalkyl, or
R⁶ and R⁷ together with the nitrogen atom to which they are attached form a ring which may contain one or more further heteroatoms from the group consisting of nitrogen, oxygen and sulfur,
R⁸ represents a radical from the group consisting of hydrogen, alkyl, haloalkyl, cyanoalkyl, alkoxy, haloalkoxy, alkenyl, alkoxyalkyl, in each case optionally halogen-substituted alkylcarbonyl and alkylsulfonyl, optionally halogen-substituted alkoxycarbonyl, optionally halogen-, alkyl-, alkoxy-, haloalkyl- and cyano-substituted cycloalkylcarbonyl, or a cation, or an optionally alkyl- or arylalkyl-substituted ammonium ion,
R⁹ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R⁹ in the radical (C-1), together with the N—S(O)n group to which they are attached, may also form a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁰ represents hydrogen or alkyl,
R⁸ and R¹⁰ in the radicals (C-2) and (F-2), together with the nitrogen atoms to which they are attached, may also be a saturated or unsaturated and optionally substituted 4- to 8-membered ring which may contain at least one further heteroatom from the group consisting of sulfur, oxygen (where oxygen atoms must not be directly adjacent to one another) and nitrogen and/or at least one carbonyl group,
R⁹ and R¹⁰ in the radicals (C-2) and (F-2) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹¹ represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹² represents an in each case optionally substituted radical from the group consisting of alkyl, alkenyl, alkynyl, alkoxy, alkenyloxy, alkynyloxy, cycloalkyl, cycloalkyloxy, cycloalkenyloxy, cycloalkylalkoxy, alkylthio, alkenylthio, phenoxy, phenylthio, benzyloxy, benzylthio, heteroaryloxy, heteroarylthio, heteroarylalkoxy and heteroarylalkylthio,
R¹¹ and R¹² in the radicals (C-3) and (F-3) may also form, together with the phosphorus atom to which they are bonded, a saturated or unsaturated and optionally substituted 5- to 7-membered ring which may contain one or two heteroatoms from the group consisting of oxygen (where oxygen atoms must not be directly adjacent to one another) and sulfur,
R¹³ represents an in each case optionally substituted radical from the group consisting of alkyl, alkenyl, alkynyl, phenyl and phenylalkyl,
R¹⁴ represents an in each case optionally substituted radical from the group consisting of alkyl, alkenyl, alkynyl, phenyl and phenylalkyl,
R¹⁵ represents a radical from the group consisting of in each case optionally substituted alkyl, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁵ in the radicals (C-6) and (F-6) may also form, together with the N—S(O)n group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁶ represents a radical from the group consisting of hydrogen, in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁶ in the radicals (C-7) and (F-7) may also form, together with the nitrogen atom to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
R¹⁷ represents a radical from the group consisting of in each case optionally substituted alkyl, alkoxy, alkenyl and alkynyl, in each case optionally substituted cycloalkyl, cycloalkylalkyl and cycloalkenyl, in which the rings may contain at least one heteroatom from the group consisting of sulfur, oxygen (where oxygen atoms must not be directly adjacent to one another) and nitrogen, in each case optionally substituted aryl, heteroaryl, arylalkyl and heteroarylalkyl and an optionally substituted amino group,
R⁸ and R¹⁷ in the radicals (C-8) and (F-8) may also form, together with the N—C(X) group to which they are attached, a saturated or unsaturated and optionally substituted 4- to 8-membered ring which may contain one or more further heteroatoms from the group consisting of sulfur, oxygen (where oxygen atoms must not be directly adjacent to one another) and nitrogen and/or at least one carbonyl group,
Y¹ and Y² independently of one another represent C═O or S(O)₂,
R²² in the case that R² represents g), represents a radical from the group consisting of hydrogen, alkyl, haloalkyl, cyanoalkyl, alkenyl, haloalkenyl, alkynyl, haloalkynyl, alkoxy, haloalkoxy, optionally halogen-substituted alkoxyalkyl, optionally halogen-substituted bis(alkoxy)alkyl, optionally halogen-substituted alkylsulfanylalkyl, optionally halogen-substituted alkylcarbonylalkyl, optionally halogen-substituted alkylsulfinylalkyl, optionally halogen-substituted alkylsulfonylalkyl, dialkylaminosulfanylalkyl, dialkylaminosulfinylalkyl, dialkylaminosulfonylalkyl, optionally halogen-substituted alkoxycarbonylalkyl, optionally halogen-substituted alkynyloxy, dialkylaminocarbonylalkyl, N-alkyl-N-cycloalkylaminocarbonylalkyl, heterocyclylcarbonylalkyl, alkylsulfanyl, haloalkylsulfanyl, alkylsulfinyl, haloalkylsulfinyl, alkylsulfonyl, haloalkylsulfonyl, cycloalkyl which is optionally substituted by halogen, cyano, nitro, alkyl, cycloalkyl, haloalkyl, alkoxy, haloalkoxy, alkoxycarbonyl, haloalkoxycarbonyl or hetaryl (which for its part is optionally substituted by alkyl or halogen), cycloalkylalkyl which is optionally substituted by halogen, cyano, nitro, alkyl, haloalkyl, cycloalkyl, alkoxy, haloalkoxy, alkoxycarbonyl, haloalkoxycarbonyl or hetaryl (which for its part is optionally substituted by alkyl or halogen), optionally substituted heterocyclyl, heterocyclylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulpinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxyalkyl, alkenyl, alkynyl, cycloalkylalkyl, alkylcarbonyl, alkoxycarbonyl or aminocarbonyl, aryl which is optionally substituted by halogen, cyano, nitro, hydroxy, amino, alkyl, haloalkyl, cycloalkyl (which is optionally substituted), alkoxy or haloalkoxy, arylalkyl which is optionally substituted by halogen, cyano (also in the alkyl moiety), nitro, hydroxy, amino, alkyl, cycloalkyl (which is optionally substituted), haloalkyl, alkoxy or haloalkoxy, hetarylalkyl which is optionally substituted by halogen, cyano (also in the alkyl moiety), nitro, hydroxy, alkyl, haloalkyl, cycloalkyl (which is optionally substituted), alkoxy, haloalkoxy, alkylthio, haloalkylthio, alkylsulfinyl, alkylsulfonyl, haloalkylsulfinyl, haloalkylsulfonyl, amino, alkylamino, dialkylamino, alkylcarbonylamino, alkoxycarbonylamino, alkoxyalkyl, haloalkoxyalkyl, alkenyl, alkynyl, cycloalkylalkyl, alkylcarbonyl, alkoxycarbonyl or aminocarbonyl, or, in the case that R² represents c), d) or g),
R²² represents a D radical from the group consisting of (D-1) to (D-3) 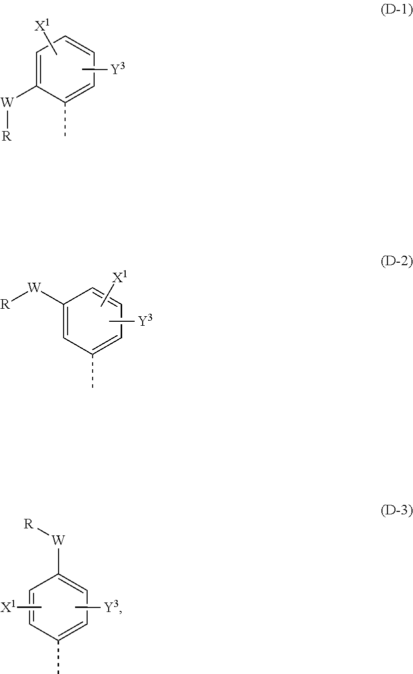
in which
X¹ represents a radical from the group consisting of hydrogen, halogen, cyano, nitro, alkyl, haloalkyl, cycloalkyl, alkoxy and haloalkoxy,
R represents NR¹⁸R¹⁹, or represents an in each case optionally substituted radical from the group consisting of alkyl, alkenyl, alkynyl, alkoxyalkyl, alkyl-S-alkyl, alkyl-S(O)-alkyl, alkyl-S(O)₂-alkyl,
R¹⁸—CO-alkyl, NR¹⁸R¹⁹—CO-alkyl, cycloalkyl, cycloalkenyl, cycloalkylalkyl, cycloalkenylalkyl, heterocyclyl, heterocyclylalkyl, phenyl, phenylalkyl, hetaryl and hetarylalkyl,
Y³ represents a radical from the group consisting of hydrogen, halogen, cyano, alkyl, cycloalkyl, haloalkyl, alkoxy, haloalkoxy and NR²⁰R²¹,
W represents a radical from the group consisting of O, S, SO and SO₂,
R²² furthermore represents an E radical from the group consisting of (E-1) to (E-11) 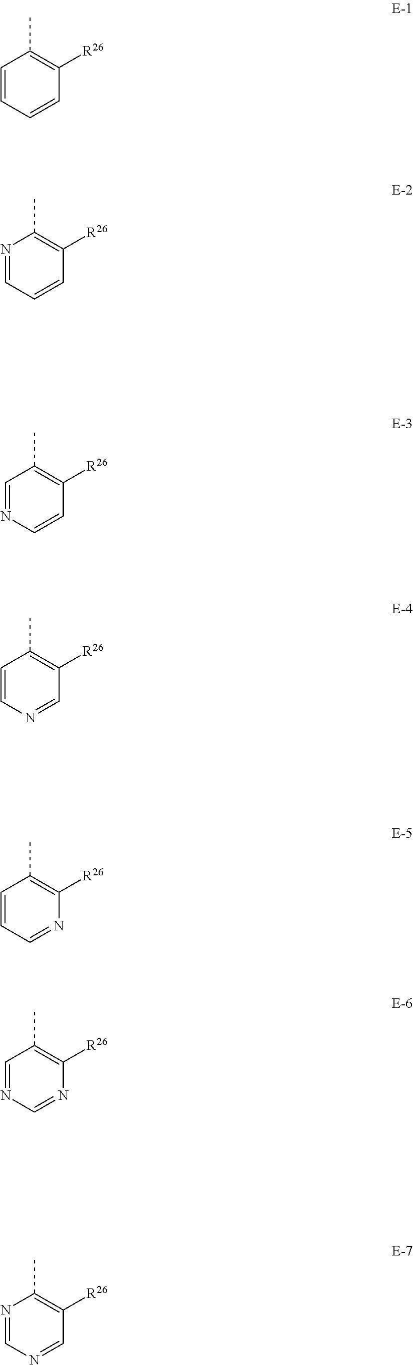 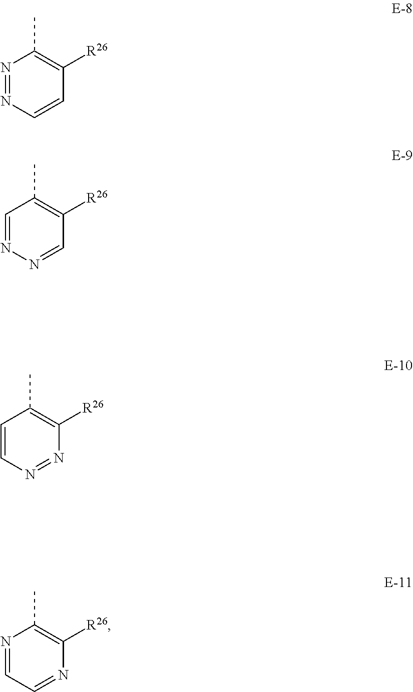
of the group consisting of (E-18) to (E-51) 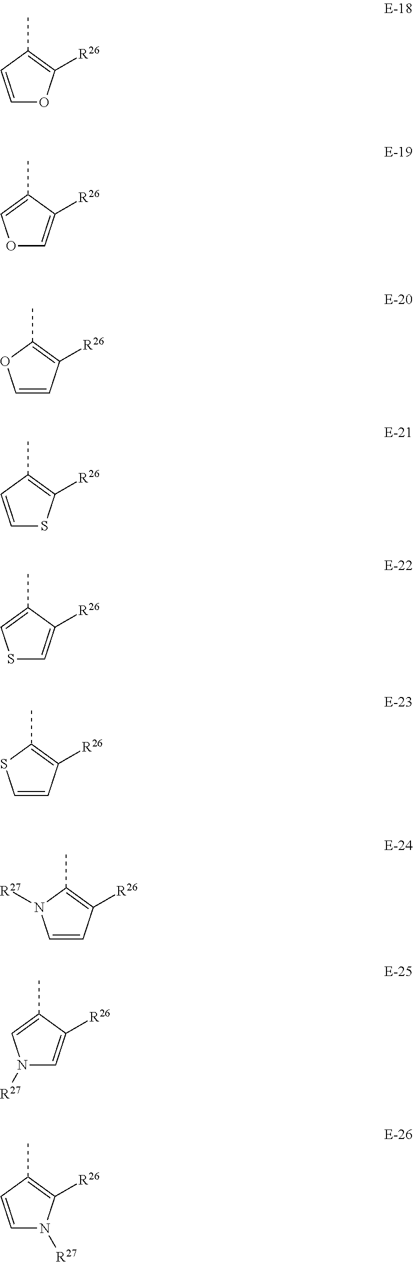 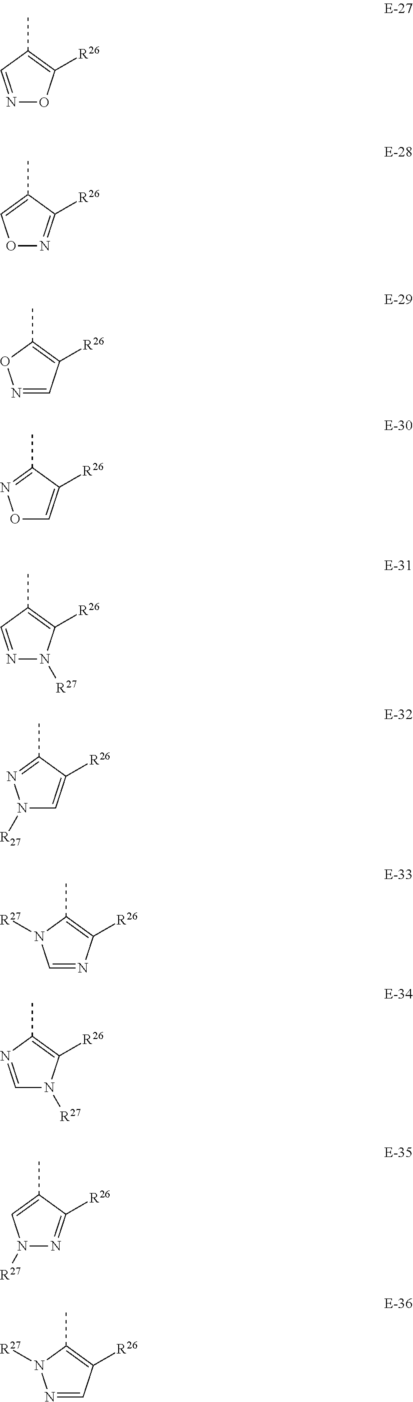 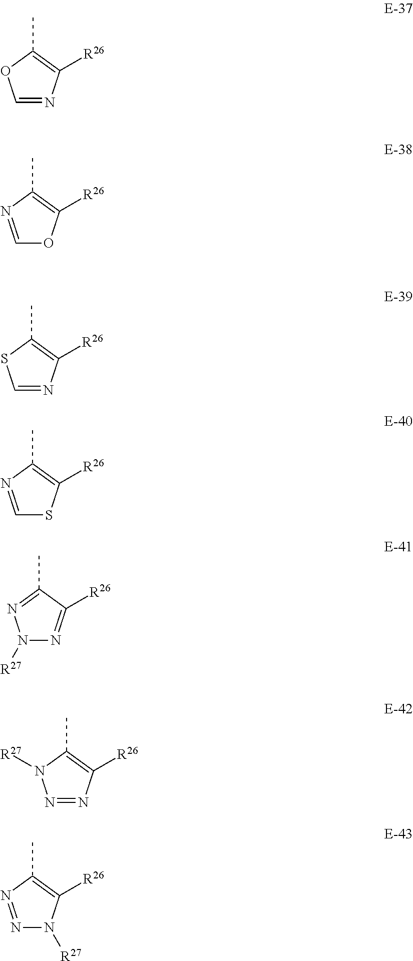 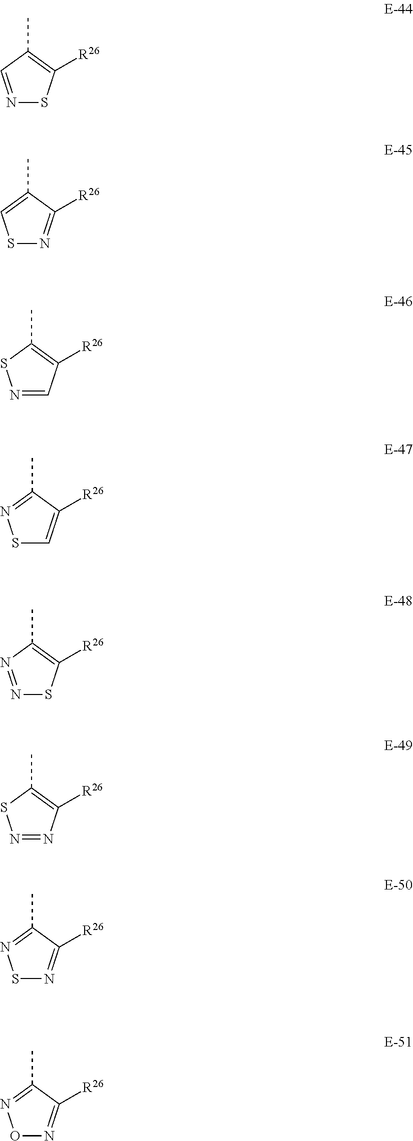
or, in the case that R²=d),
R²² also represents an E radical from the group consisting of E-12 to E-17 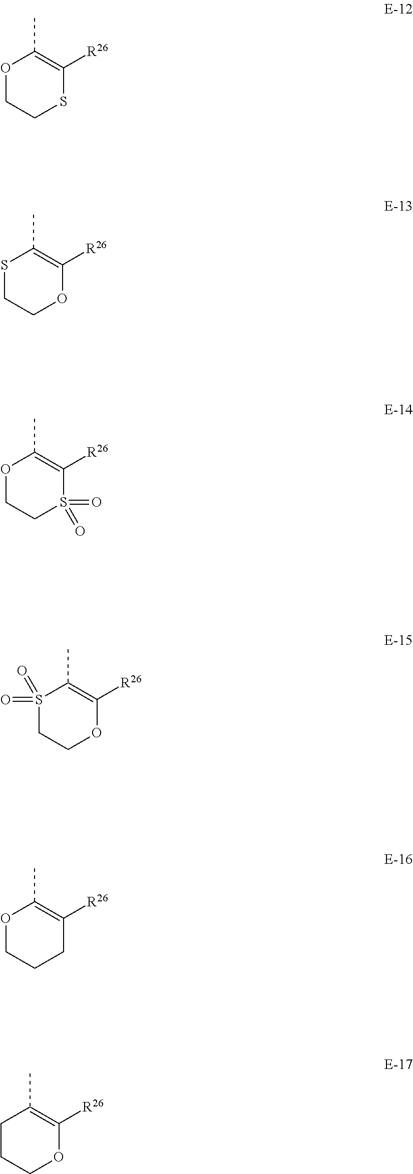
R¹⁸ represents a radical from the group consisting of hydrogen, hydroxy, in each case optionally substituted alkyl, alkoxy, alkoxyalkyl, alkylthioalkyl, alkylsulfinylalkyl, alkylsulfonylalkyl, alkylcarbonyl, alkoxycarbonyl, alkenyl and alkynyl, in each case optionally substituted cycloalkyl, cycloalkylalkyl, cycloalkenyl and cycloalkenylalkyl, in which the rings may contain at least one heteroatom from the group consisting of sulfur, oxygen (where oxygen atoms must not be directly adjacent to one another) and nitrogen, in each case optionally substituted aryl, arylalkyl, heteroaryl and heteroarylalkyl and an optionally substituted amino group,
R¹⁹ represents a radical from the group consisting of hydrogen, represents an alkali metal or alkaline earth metal ion or represents an ammonium ion which is optionally mono- to tetrasubstituted by C₁-C₄-alkyl or represents an in each case optionally halogen- or cyano-substituted radical from the group consisting of alkyl, alkoxy, alkoxyalkyl, alkylthioalkyl, alkylsulfinylalkyl and alkylsulfonylalkyl,
R²⁰ represents a radical from the group consisting of hydrogen, halogen, cyano, nitro, amino, hydroxy and in each case optionally substituted alkyl, alkenyl, alkynyl, cycloalkyl, cycloalkylalkyl, alkoxy, alkenyloxy, alkynyloxy, cycloalkyloxy, alkylcarbonyloxy, alkenylcarbonyloxy, alkynylcarbonyloxy, cycloalkylcarbonyloxy, alkoxycarbonyloxy, alkylsulfonyloxy, alkylamino, alkenylamino, alkynylamino, cycloalkylamino, alkylthio, haloalkylthio, alkenylthio, alkynylthio, cycloalkylthio, alkylsulfinyl, alkylsulfonyl, alkylcarbonyl, alkoxyiminoalkyl, alkoxycarbonyl, aminocarbonyl, alkylaminocarbonyl, dialkylaminocarbonyl, aminothiocarbonyl, alkylaminosulfonyl, alkylsulfonylamino, alkylcarbonylamino, alkenylcarbonylamino, alkynylcarbonylamino, cycloalkylcarbonylamino, alkoxycarbonylamino, alkylthiocarbonylamino, bicycloalkyl, aryl, aryloxy, heteroaryl and heteroaryloxy, where the substituents are independently of one another selected from halogen, cyano, nitro, hydroxy, amino, alkyl and haloalkyl,
R²¹ represents a radical from the group consisting of hydrogen, alkyl, cycloalkyl, haloalkyl, alkenyl, alkynyl, cycloalkylalkyl, cyanoalkyl, alkylcarbonyl, alkenylcarbonyl, haloalkylcarbonyl, haloalkenylcarbonyl, alkoxyalkyl, alkoxycarbonyl, alkylsulfonyl and haloalkylsulfonyl,
R²³ represents a radical from the group consisting of hydrogen, alkyl, alkenyl, alkynyl, cycloalkyl, cycloalkenyl, alkoxy, alkenyloxy, alkynyloxy, cycloalkyloxy, alkylthioalkyl, alkenylthioalkyl, cyanoalkyl, alkoxyalkyl,
or, if R²=g),
R²² and R²³ together with the nitrogen atom to which they are attached form a ring which may contain one or more further heteroatoms from the group consisting of nitrogen, oxygen and sulfur, and
R²⁴ represents hydrogen or an in each case optionally substituted radical from the group consisting of alkyl, alkenyl, alkynyl, phenyl and phenylalkyl and
R²⁵ represents hydrogen or an in each case optionally substituted radical from the group consisting of alkyl, alkenyl, alkynyl, phenyl and phenylalkyl,
R²⁶ represents a radical from the group consisting of hydrogen, alkyl, haloalkyl, alkenyl, alkynyl, cycloalkyl, cycloalkylalkyl, alkoxyalkyl, alkylsulfanyl, haloalkylsulfanyl, alkylsulfinyl, haloalkylsulfinyl, alkylsulfonyl, alkylthioalkyl, alkylsulfinylalkyl, alkylsulfonylalkyl and cyanoalkyl.
R²⁷ represents hydrogen, alkyl, haloalkyl, alkenyl, alkynyl, cycloalkyl, cycloalkylalkyl, alkoxyalkyl, alkylthioalkyl, alkylsulfinylalkyl, alkylsulfonylalkyl or cyanoalkyl.

2. Compound of formula (I) as claimed in claim 1, in which
A represents the A radical (A-a) 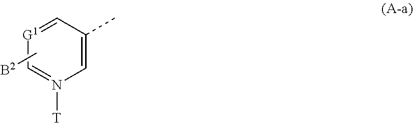
where the broken line denotes the bond to the carbon atom of the bicyclic system of the formula (I),
G¹ represents C—B¹,
B¹ represents hydrogen,
B² represents hydrogen,
T represents an electron pair,
R¹ represents hydrogen,
R² a) represents one of the radicals below 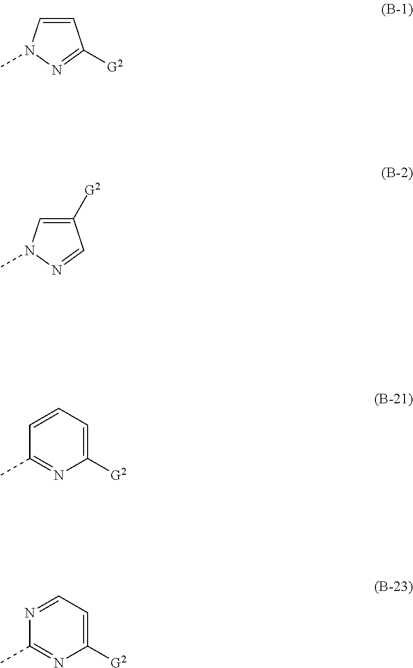
where the broken line denotes the bond to the carbon atom of the bicyclic system of the formula (I), or
R² c) represents a radical of the formula 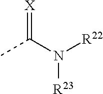
where the broken line denotes the bond to the carbon atom of the bicyclic system of the formula (I), or
R² d) represents a radical of the formula 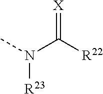
where the broken line denotes the bond to the carbon atom of the bicyclic system of the formula (I), or
R² f) represents C₁-C₆-haloalkyl or
R² g) represents a radical of the formula 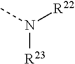
where the broken line in each case denotes the bond to the carbon atom of the bicyclic system of the formula (I), in which
G² represents a radical from the group consisting of hydrogen, C₁-C₄-alkyl and halo-C₁-C₄-alkyl,
R²² in the case that R² represents g), represents a radical from the group consisting of methyl, ethyl, isopropyl, n-propyl, n-butyl, isobutyl, sec-butyl, tert-butyl, 2,2,2-trifluoroethyl, 2,2-difluoroethyl, 2,2-difluoro-n-propyl, methyl sulfanylmethyl, methylsulfanylethyl, methylsulfanyl-n-propyl, methyl sulfonyl, ethylsulfonyl, methyl sulfonylmethyl, ethyl sulfonylmethyl, isopropylsulfonylmethyl, cyclopropyl and, in the case that R² represents c), d) or g),
R²² represents (D-2) 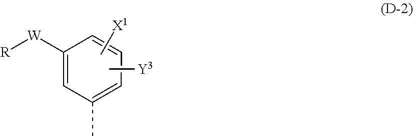
in which
X¹ represents a radical from the group consisting of hydrogen, fluorine, chlorine and bromine,
R represents C₁-C₄-alkyl, optionally mono-, di-, tri-, tetra- or pentasubstituted by fluorine, chlorine,
W represents a radical from the group consisting of S, SO and SO₂,
Y³ represents hydrogen or methyl and
R²³ represents hydrogen or C₁-C₆-alkyl or, in the case that R² represents g),
R²² and R²³ together with the nitrogen atom to which they are attached represent pyrrolidinyl or morpholinyl.

3. A composition, comprising a content of at least one compound of the formula (I) as claimed claim 1 and one or more customary extenders and/or surfactants.

4. A compound of the formula (I) as claimed in claim 1 or a composition thereof for controlling pests.

5. A compound of the formula 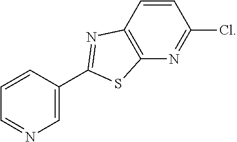 [SMILES: Clc1ccc2nc(-c3cccnc3)sc2n1]
